(19) United States
(12) Patent Application Publication
(10) Pub. No.: US 2016/0296499 A1
(43) Pub. Date: Oct. 13, 2016

(54) Spiroindoline Antiparasitic Derivatives

(71) Applicant: ZOETIS SERVICES LLC, Florham Park, NJ (US)

(72) Inventors: John Adam Wendt, Kalamazoo, MI (US); Mark Cox, Kalamazoo, MI (US); Susan M.K. Sheehan, Galesburg, MI (US); Michael P. Curtis, Portage, MI (US); Tomasz Respondek, Kalamazoo, MI (US); Richard Andrew Ewin, Kalamazoo, MI (US); Graham M. Kyne, Kalamazoo, MI (US); Paul D. Johnson, Kalamazoo, MI (US)

(21) Appl. No.: 15/102,384

(22) PCT Filed: Dec. 22, 2014
(86) PCT No.: PCT/US2014/071874

§ 371 Date: Jun. 7, 2016

Related U.S. Application Data
(60) Provisional application No. 61/920,551, filed on Dec. 24, 2013.

(51) Int. Cl. A61K 31/435; C07D 491/20; A61K 31/444; C07D 513/20; A01N 43/90; A61K 31/501; C07D 471/10; C07D 471/20; A61K 31/506; A61K 31/4709; A61K 45/06; A61K 31/497

(52) U.S. Cl. CPC A61K 31/435; A61K 45/06; C07D 491/20; A61K 31/444; C07D 513/20; A61K 31/497; A61K 31/501; C07D 471/10; C07D 471/20; A61K 31/506; A61K 31/4709; A01N 43/90

(57) ABSTRACT

The invention describes novel spiropiperidines of Formula (1A), (1B), and (1C) stereoisomers thereof, veterinarily acceptable salts thereof, compositions thereof, processes for making, and their use in animals as an antiparasitic. The variables A, R¹, R², R³, R⁴, v, m, 5, and n are as described herein.

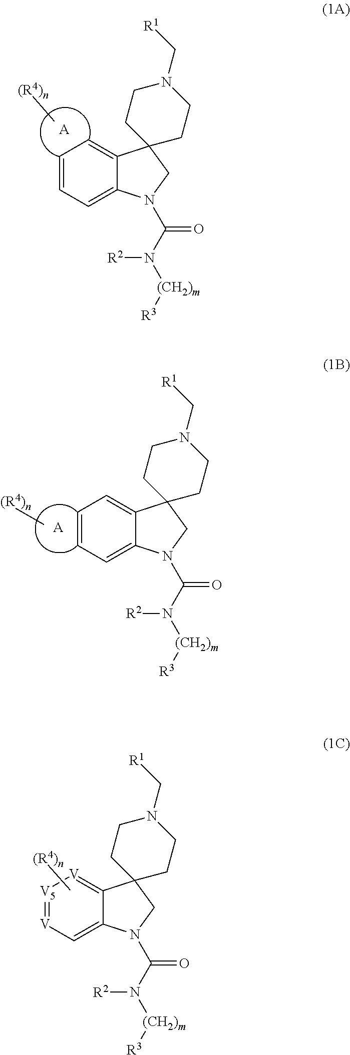

### FIELD OF THE INVENTION

This invention describes novel bicyclic and cyclic spiroindoline piperadine derivatives, stereoisomers thereof, and veterinary acceptable salts thereof, having parasiticidal activity. The invention also relates to processes of making the spiroindoline piperidine derivatives, compositions and methods of use thereof.

### BACKGROUND

There is a need for improved antiparasitic agents for use with animals, and in particular there is a need for improved endoparasiticides and ectoparasiticides. Furthermore there is a need for improved topical and oral products with convenient administration and which contain one or more of such antiparasitic derivatives which can be used to effectively treat ectoparasites, such as insects (e.g., fleas, lice, and flies) and acarids (e.g., mites and ticks); and endoparasites, such as helminths (nematodes, cestodes, and trematodes). The novel derivatives of the instant invention are particularly useful for the treatment of parasitic infections in animals.

There are many known drugs (or “anthelmintic agents”) available to treat various helminth parasite infections, see, e. g., McKellar, Q. A., et al., “Veterinary anthelmintics: old and new,” Review: Trends in Parasitology, 20(10), 456-61 (October 2004). While many parasitic infections can be treated with known drugs, evolutionary development of resistance by the parasites can render such drugs obsolete over time, see, e.g., Jabbar, A., et al., “Anthelmintic resistance: the state of play revisited,” Life Sciences, 79, 2413-31 (2006). In addition, known drugs may have other deficiencies, such as limited spectrum of activity and the need for repeated treatments.

Spiroindoline-piperidine derivatives have been described in WO2003/106457. However, no bicyclic-spiropiperidines were exemplified. Further, WO2005/058897 describes spiroindoline-piperidines, for example, thousands of prophetically described methyl, hydroxyl, or fluoro substituted piperidines. Five compounds were actually prepared and tested, none of which carry the urea moiety. Further, there is no indication in the application how to prepare the bicyclic compounds or biological data to support said bicyclics. Additionally, WO2011/095581 describes non-urea monocyclic spiroindoline-piperidines and WO2013/017678 claims bicyclic compounds, however, these bicyclics are linked by a sulphur atom. These application publications do not exemplify the compounds of the instant invention. Further, and unexpectedly, Applicants discovered that the urea-methylene moiety and the non-substituted piperidine ring, excluding the N-linked substitution, have reduced binding affinity for the bovine VAChT receptor while maintaining nematocidal activity, thereby reducing and/or preventing mammalian toxicity and morbidity.

Despite the availability of effective, broad spectrum antiparasitic agents, there remains a need for a safer, convenient, efficacious, selective, and environmentally friendly product that will overcome the ever-present threat of resistance development. The invention overcomes one or more of the various disadvantages of, or improves upon, the properties of existing compounds.

### SUMMARY

The invention provides Formula (1A), (1B), and (1C) compounds, stereoisomers (including all enantiomers and diastereomers, thereof), and veterinary acceptable salts thereof, that act as ectoparasiticides and endoparasiticides; therefore may be used to prevent, treat, repel, and control acarids and insect infection and infestation in animals. Compounds of Formula (1A) and Formula (1B) are described herein as bicyclic whereas compound of Formula (1C) are described herein as mono-cyclics. In addition, the invention contemplates the control and prevention of tick borne diseases, for example, Lyme disease, canine and bovine anaplasmosis, canine ehrlichiosis, canine rickettsiosis, canine and bovine babesiosis, epizootic bovine abortion, theileriosis, and other parasitic borne diseases, e.g., leishmaniasis and demodicosis.

In one aspect of the invention, there is provided a compound of Formula (1A), Formula (1B), and Formula (1C)

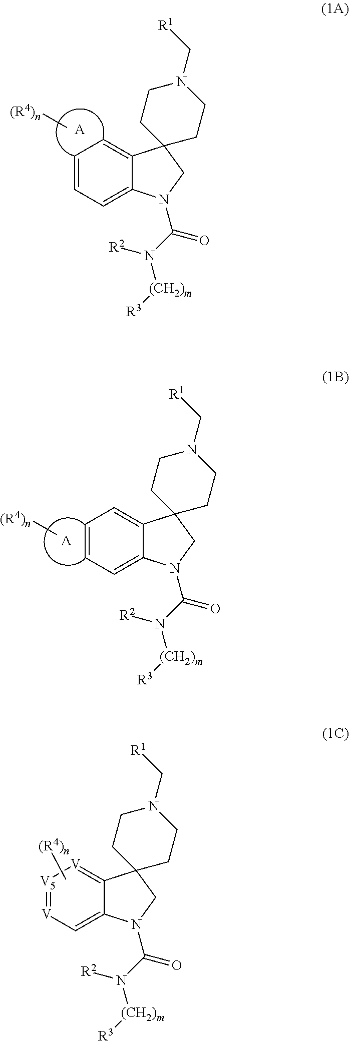

wherein

A is a 5- or 6-membered partially saturated or saturated heterocyclic ring, or a 5- to 6-membered heteroaryl ring, or a 5- to 6-membered partially saturated or saturated carbocyclic ring, wherein the heterocyclic and heteroaryl ring each contain at least 1 to 3 heteroatoms selected from N, O, or S;

v is CH or N, wherein only one of v can be N;

R¹ is selected from the group consisting of C₀-C₃alkylaryl, C₀-C₃alkylheteroaryl, C₀-C₃alkylcycloalkyl, C₀-C₃alkylheterocycle, C₂-C₄alkenylaryl, C₂-C₄alkenylheteroaryl, C₂-C₄alkenylcycloalkyl, and C₂-C₄alkenylheterocycle; wherein each cycloalkyl, aryl, heteroaryl, or heterocycle R¹ moiety is individually and optionally substituted with at least one substituent selected from the group consisting of cyano, halo, C₁-C₆haloalkyl, C₁-C₆alkyl, C₁-C₆alkoxy, and C₁-C₆haloalkoxy;

R² is selected from the group consisting of hydrogen, C₁-C₆alkyl, C₁-C₆haloalkyl, and C₁-C₆haloalkoxy;

R³ is selected from the group consisting of C₁-C₆alkyl, C₃-C₆cycloalkyl, aryl, heteroaryl, and heterocycle, wherein said R³ cycloalkyl, aryl, heteroaryl, and heterocycle moieties are each individually and optionally substituted with at least one substituent selected from the group consisting of halo, hydroxyl, —NR⁵R⁶, nitro, cyano, C₁-C₆haloalkyl, C₁-C₆alkyl, C₁-C₆alkoxy, C₁-C₆haloalkoxy, and isoxazole, wherein the isoxazole can be further substituted with at least one methyl;

R⁴ is halo, C₁-C₆alkyl, C₁-C₆haloalkyl, C₁-C₆alkoxy, C₁-C₆haloalkoxy, cyano, C₃-C₆cycloalkyl, NR⁵R⁶, S(O)₂CF₃, S(O)₂CH₃, SCF₃, SF₅, nitro, phenyl, pyridin-2(1H)-one, heterocycle, and heteroaryl, and wherein the phenyl and heteroaryl moieties can be further optionally substituted with at least one substituent selected from the group consisting of halo, cyano, C₁-C₆haloalkyl, C₁-C₆alkyl, and C₁-C₆alkoxy;

R⁵ and R⁶ are each independently selected from selected from H and C₁-C₆alkyl;

m is the integer 1, 2, 3, or 4;

n is the integer 0, 1, 2, 3, or 4 and when n is 2, 3, or 4, each R⁴ may be identical or different from each other;

stereoisomers thereof, and veterinary acceptable salts thereof, with the proviso that when n is the integer 1, then R⁴ is not fluoro or chloro at ring position 5 of Formula (1C).

In another aspect of the invention is a Formula (1A) compound

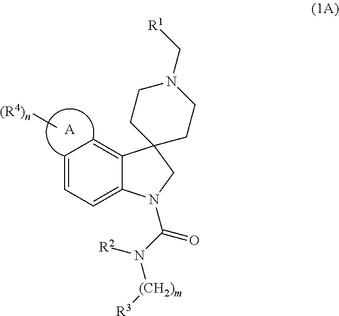

wherein each of A, R¹, R², R³, R⁴, m, and n are as defined herein, stereoisomers thereof, and veterinary acceptable salts thereof.

In another aspect of the invention is a Formula (1B) compound

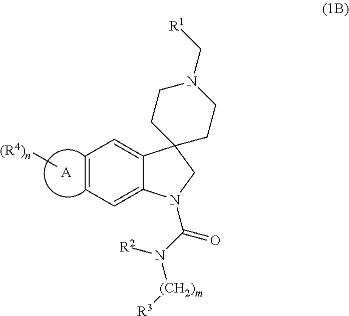

wherein each of A, R¹, R², R³, R⁴, m, and n are as defined herein, stereoisomers thereof, and veterinary acceptable salts thereof.

In one aspect of the invention, ring A, of Formula (1A) or Formula (1B) is selected from the group consisting of

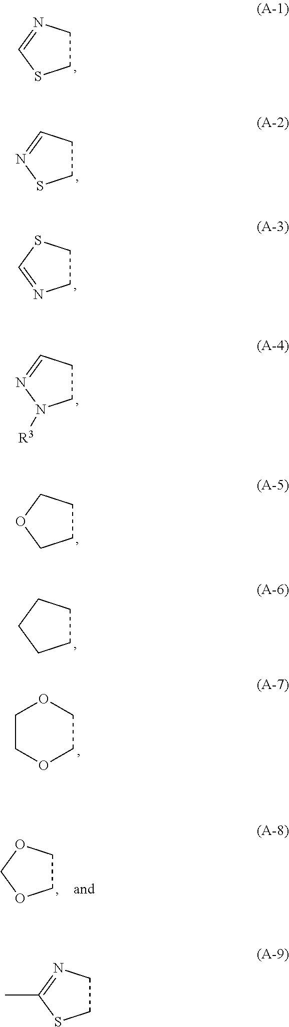

wherein R³ is as defined herein, and the broken line (----) represents the point of attachment to the phenyl ring of the indoline moiety. In yet another aspect of the invention, ring A, of Formula (1A) or Formula (1B) is (A-1). In yet another aspect, ring A of Formula (1A) or Formula (1B) is (A-2). In yet another aspect, ring A of Formula (1A) or Formula (1B) is (A-3). In yet another aspect, ring A of Formula (1A) or Formula (1B) is (A-4). In yet another aspect, ring A of Formula (1A) or Formula (1B) is (A-5). In yet another aspect, ring A of Formula (1A) or Formula (1B) is (A-6). In yet another aspect, ring A of Formula (1A) or Formula (1B) is (A-7). In yet another aspect, ring A of Formula (1A) or Formula (1B) is (A-8). In yet another aspect, ring A of Formula (1A) or Formula (1B) is (A-9).

In another aspect of the invention, particularly for compounds of Formula (1A) and Formula (1B), R¹ is selected from the group consisting of C₀-C₃alkylaryl, C₀-C₃alkylheteroaryl, C₀-C₃alkylheterocycle, C₀-C₃alkylcycloalkyl, C₂-C₄alkenylaryl, C₂-C₄alkenylheteroaryl, C₂-C₄alkenylcycloalkyl, and C₂-C₄alkenylheterocycle; wherein each C₀-C₃alkyl- or C₂-C₄alkenyl-aryl, -heteroaryl, -cycloalkyl, or -heterocycle R¹ moiety is individually and optionally substituted with at least one substituent as described herein. In another aspect, R¹ is C₀-C₃alkylaryl, wherein the aryl moiety is optionally substituted with at least one substituent as described herein. In another aspect, the R¹ aryl moiety of C₀-C₃alkylaryl is C₀-C₃alkylphenyl or C₀-C₃alkylnaphthyl, wherein said phenyl or naphthyl moiety is optionally substituted with at least one substituent as described herein. In another aspect, the R¹ aryl moiety of C₀-C₃alkylaryl is C₀-C₃alkylphenyl, wherein said phenyl moiety is optionally substituted with at least one substituent as described herein. In another aspect, the R¹ aryl moiety of C₀-C₃alkylaryl is C₀-C₃alkylnaphthyl, wherein said naphthyl moiety is optionally substituted with at least one substituent as described herein. In another aspect, the R¹ heteroaryl moiety of C₀-C₃alkylheteroaryl is C₀-C₃alkylpyridinyl, C₀-C₃alkylquinolinyl, or C₀-C₃alkylisoquinolinyl; wherein said pyridinyl, quinolinyl, or isoquinolinyl moiety is optionally substituted with at least one substituent as described herein. In another aspect, the R¹ heteroaryl moiety of C₀-C₃alkylheteroaryl is C₀-C₃alkylpyridinyl which is optionally substituted with at least one substituent as described herein. In another aspect, the R¹ heteroaryl moiety of C₀-C₃alkylheteroaryl is C₀alkylpyridinyl which is optionally substituted with at least one substituent as described herein. In another aspect, the R¹ heteroaryl moiety of C₀-C₃alkylheteroaryl is C₀-C₃alkylquinolinyl which is optionally substituted with at least one substituent as described herein. In another aspect, the R¹ heteroaryl moiety of C₀-C₃alkylheteroaryl is C₀alkylquinolinyl which is optionally substituted with at least one substituent as described herein. In another aspect, R¹ is selected from the group consisting of C₂-C₄alkenylaryl, C₂-C₄alkenylheteroaryl, C₂-C₄alkenylcycloalkyl, and C₂-C₄alkenylheterocycle; wherein each C₂-C₄alkenyl-aryl, -heteroaryl, -cycloalkyl, or -heterocycle R¹ moiety is individually and optionally substituted with at least one substituent as described herein. In another aspect, R¹ is selected from the group consisting of C₂-C₄alkenylaryl, C₂-C₄alkenylheteroaryl, and C₂-C₄alkenylheterocycle; wherein each C₂-C₄alkenyl-aryl, -heteroaryl, or -heterocycle R¹ moiety is individually and optionally substituted with at least one substituent as described herein. In another aspect, R¹ is selected from the group consisting of C₂-C₄alkenylaryl and C₂-C₄alkenylheteroaryl, wherein each C₂-C₄alkenyl-aryl and -heteroaryl R¹ moiety are individually and optionally substituted with at least one substituent as described herein. In yet another aspect, R¹ is selected from the group consisting of C₂-C₄alkenylaryl, wherein the aryl moiety is phenyl or naphthyl, either of which is optionally substituted with at least one substituent as described herein. In yet another aspect, R¹ is a C₂alkenylaryl, wherein the aryl moiety is optionally substituted with at least one substituent as described herein. In yet another aspect, R¹ is a C₂alkenylaryl, wherein the aryl moiety is phenyl or naphthyl, either of which is optionally substituted with at least one substituent as described herein. In yet another aspect, R¹ is a C²alkenylaryl, wherein the aryl moiety is phenyl which is optionally substituted with at least one substituent as described herein. In yet another aspect, R¹ is a C²alkenylaryl, wherein the aryl moiety is naphthyl which is optionally substituted with at least one substituent as described herein. In another aspect, R¹ is selected from the group consisting of C₂-C₄alkenylheteroaryl, wherein the heteroaryl moiety is optionally substituted by at least one substituent as described herein. In another aspect, R¹ is selected from the group consisting of C₂-C₄alkenylheteroaryl, wherein the heteroaryl moiety is pyridinyl, quinolinyl, or isoquinolinyl, wherein the pyridinyl, quinolinyl, or isoquinolinyl moiety is optionally substituted by at least one substituent as described herein. In another aspect, R¹ is a C₂alkenylheteroaryl, wherein the heteroaryl moiety is pyridinyl, quinolinyl, or isoquinolinyl, wherein the pyridinyl, quinolinyl, or isoquinolinyl moiety is optionally substituted by at least one substituent as described herein. In another aspect, R¹ is a C₂alkenylheteroaryl, wherein the heteroaryl moiety is pyridinyl or quinolinyl, wherein the pyridinyl and quinolinyl moiety is optionally substituted by at least one substituent as described herein. In another aspect, R¹ is a C₂alkenylheteroaryl, wherein the heteroaryl moiety is pyridinyl which is optionally substituted by at least one substituent as described herein. In another aspect, R¹ is a C₂alkenylheteroaryl, wherein the heteroaryl moiety is quinolinyl which is optionally substituted by at least one substituent as described herein. In yet another aspect, R¹ is selected from the group consisting of C₂₋C₄alkenylheterocycle, wherein the heterocycle moiety is optionally substituted with at least one substituent as described herein. In yet another aspect, R¹ is selected from the group consisting of C₂alkenylheterocycle, wherein the heterocycle moiety is optionally substituted with at least one substituent as described herein. The at least one optional substituent for the R¹ alkylaryl, alkylheteroaryl, alkylcycloalkyl, alkyl heterocycle, alkenylaryl, alkenylheteroaryl, alkenylcycloalkyl, and alkenylheterocycle moieties are selected from the group consisting of halo, C₁-C₄alkyl, C₁-C₄alkoxy, cyano, and C₁-C₄haloalkyl. Preferred optional substituents are selected from the group consisting of chloro, fluoro, bromo, iodo, methyl, ethyl, propyl, isopropyl, methoxy, ethoxy, cyano, and —CF₃. More preferred optional substituents are selected from the group consisting of chloro, fluoro, bromo, methyl, methoxy, cyano, and —CF₃. Even more preferred optional substituents are selected from the group consisting of chloro, fluoro, methoxy, cyano, and —CF₃. If any of the R¹ aryl, heteroaryl, cycloalkyl, or heterocycle moieties are substituted with more than one substituent, as described herein, then the substituents can be the same or different.

In another aspect of the invention, particularly for compounds of Formula (1A) and Formula (1B), R² is selected from the group consisting of hydrogen, C₁-C₆alkyl, C₁-C₆haloalkyl, and —OC₁-C₆haloalkyl. In another aspect of the invention, R² is selected from the group consisting of hydrogen, C₁-C₆alkyl, and C₁-C₆haloalkyl. In another aspect, R² is selected from the group consisting of hydrogen and C₁-C₆alkyl. In another aspect, R² is hydrogen or methyl. In another aspect of the invention, R² is hydrogen.

In another aspect of the invention, particularly for compounds of Formula (1A) and Formula (1B), R³ is selected from the group consisting of C₁-C₆alkyl, C₃-C₆cycloalkyl, aryl, heteroaryl, heterocycle, and pyridine-2(1H)-one, wherein the aryl, heteroaryl, and heterocycle moieties are each individually and optionally substituted by at least one substituent as described herein. In another aspect, R³ is selected from the group consisting of C₁-C₆alkyl, cyclopropyl, cyclobutyl, cyclopentyl, cyclohexyl, tetrahydrofuran, phenyl, pyrazolyl, oxazolyl, thiazolyl, pyridinyl, pyridazinyl, pyrimidinyl, pyrazinyl, and pyridine-2(1H)-one, all of which are optionally substituted with at least one substituent as described herein. In another aspect, R³ is selected from the group consisting of isopropyl, isobutyl, cyclopropyl, cyclobutyl, cyclopentyl, cyclohexyl, tetrahydrofuran, phenyl, pyrazolyl, oxazolyl, thiazolyl, pyridinyl, pyridazinyl, pyrimidinyl, pyrazinyl, and pyridin-2(1H)-one, all of which are optionally substituted with at least one substituent as described herein. In another aspect, R³ is selected from the group consisting of isopropyl, cyclopropyl, cyclopentyl, cyclohexyl, tetrahydrofuran, pyridine-2(1H)-one, phenyl, pyrazolyl, oxazolyl, thiazolyl, pyridinyl, pyridazinyl, pyrimidinyl, and pyrazinyl, all of which are optionally substituted with at least one substituent as described herein. In another aspect, R³ is selected from the group consisting of isopropyl, cyclopropyl, cyclopentyl, cyclohexyl, tetrahydrofuran, and pyridine-2(1H)-one. In another aspect, R³ is selected from the group consisting phenyl, pyrazolyl, oxazolyl, isoxazolyl, thiazolyl, pyridinyl, pyridazinyl, pyrimidinyl, and pyrazinyl, all of which are optionally substituted with at least one substituent as described herein. The optional substitutions for the R³ cycloalkyl, aryl, heteroaryl, and heterocycle moieties are individually selected from the group consisting of halo, hydroxyl, —NR⁵R⁶, nitro, and C₁-C₆alkyl. The preferred optional R³ cycloalkyl, aryl, heteroaryl, and heterocycle moiety substituents include chloro, fluoro, bromo, iodo, nitro, —N(CH₃)₂, hydroxyl, —NHC(O)CH₃, and methyl. If any of the R³ aryl, heteroaryl, cycloalkyl, or heterocycle moieties are substituted with more than one substituent, as described herein, then the substituents can be the same or different.

In another aspect of the invention, particularly for compounds of Formula (1A) and Formula (1B), R⁴ is selected from halo, C₁-C₆alkyl, C₁-C₆haloalkyl, and C₁-C₆alkoxy. In yet another aspect, R⁴ is selected from chloro, fluoro, methyl, ethyl, —CF₃, methoxy, and ethoxy. In yet another aspect, R⁴ is selected from chloro, fluoro, and methyl. In yet another aspect, R⁴ is methyl. In another aspect of the invention, n is the integer 0. In another aspect, n is the integer 1. In another aspect, n is the integer 2. In another aspect, n is the integer 3. In another aspect, n is the integer 4. If n is the integer 2, 3, or 4, then each R⁴ substituent may be identical to, or different from each other.

In another aspect of the invention, particularly for compounds of Formula (1A) and Formula (1B), m is the integer 1. In yet another aspect, m is the integer 2. In yet another aspect, m is the integer 3. In yet another aspect, m is the integer 4. The preferred integer of m is 1.

In another aspect of the invention, are Formula (1A) compounds selected from:
(E)-N-((2-chloropyridin-4-yl)methyl)-1′-(3-(3,4-dichlorophenyl)allyl)-6,8-dihydrospiro[furo[3,4-g]indole-3,4′-piperidine]-1(2H)-carboxamide;
(E)-N-((2-chloropyridin-4-yl)methyl)-1-(3-(3,4-dichlorophenyl)allyl)-2′,3′-dihydrospiro[piperidine-4,9′-[1,4]dioxino[2,3-e]indole]-7′(8′H)-carboxamide;
(E)-1-(3-(3,4-dichlorophenyl)allyl)-N-(pyridin-4-ylmethyl)spiro[piperidine-4,8′-thiazolo[4,5-e]indole]-6′(7′H)-carboxamide;
(E)-1-(3-(3,4-dichlorophenyl)allyl)-N-(pyrimidin-2-ylmethyl)spiro[piperidine-4,8′-thiazolo[4,5-e]indole]-6′(7′H)-carboxamide;
(E)-N-benzyl-1-(3-(3,4-dichlorophenyl)allyl)spiro[piperidine-4,8′-thiazolo[4,5-e]indole]-6′(7′H)-carboxamide;
(E)-1-(3-(3,4-dichlorophenyl)allyl)-N-(4-fluorobenzyl)spiro[piperidine-4,8′-thiazolo[4,5-e]indole]-6′(7′H)-carboxamide;
(E)-1-(3-(3,4-dichlorophenyl)allyl)-N-(4-nitrobenzyl)spiro[piperidine-4,8′-thiazolo[4,5-e]indole]-6′(7′H)-carboxamide;
(E)-1-(3-(3,4-dichlorophenyl)allyl)-N-(4-hydroxybenzyl)spiro[piperidine-4,8′-thiazolo[4,5-e]indole]-6′(7′H)-carboxamide;
(E)-1-(3-(3,4-dichlorophenyl)allyl)-N-(pyrazin-2-ylmethyl)spiro[piperidine-4,8′-thiazolo[4,5-e]indole]-6′(7′H)-carboxamide;
(E)-N-(4-chlorobenzyl)-1-(3-(3,4-dichlorophenyl)allyl)spiro[piperidine-4,8′-thiazolo[4,5-e]indole]-6′(7′H)-carboxamide;
(E)-N-((2-chlorothiazol-5-yl)methyl)-1-(3-(3,4-dichlorophenyl)allyl)-spiro[piperidine-4,8′-thiazolo[4,5-e]indole]-6′(7′H)-carboxamide;
(E)-1-(3-(3,4-dichlorophenyl)allyl)-N-(thiazol-5-ylmethyl)spiro[piperidine-4,8′-thiazolo[4,5-e]indole]-6′(7′H)-carboxamide;
(E)-1-(3-(3,4-dichlorophenyl)allyl)-N-(thiazol-2-ylmethyl)spiro[piperidine-4,8′-thiazolo[4,5-e]indole]-6′(7′H)-carboxamide;
(E)-1-(3-(3,4-dichlorophenyl)allyl)-N-(thiazol-4-ylmethyl)spiro[piperidine-4,8′-thiazolo[4,5-e]indole]-6′(7′H)-carboxamide;
(E)-N-(cyclohexylmethyl)-1-(3-(3,4-dichlorophenyl)allyl)spiro[piperidine-4,8′-thiazolo[4,5-e]indole]-6′(7′H)-carboxamide;
(E)-1-(3-(3,4-dichlorophenyl)allyl)-N-((1-methyl-1H-pyrazol-4-yl)methyl)spiro[piperidine-4,8′-thiazolo[4,5-e]indole]-6′(7′H)-carboxamide;
(E)-1-(3-(3,4-dichlorophenyl)allyl)-N-((1-methyl-1H-pyrazol-5-yl)methyl)spiro[piperidine-4,8′-thiazolo[4,5-e]indole]-6′(7′H)-carboxamide;
(E)-1-(3-(3,4-dichlorophenyl)allyl)-N-((1-methyl-1H-pyrazol-3-yl)methyl)spiro[piperidine-4,8′-thiazolo[4,5-e]indole]-6′(7′H)-carboxamide;
(E)-1-(3-(3,4-dichlorophenyl)allyl)-N-(oxazol-4-ylmethyl)spiro[piperidine-4,8′-thiazolo[4,5-e]indole]-6′(7′H)-carboxamide;
(E)-1-(3-(3,4-dichlorophenyl)allyl)-N-(oxazol-5-ylmethyl)spiro[piperidine-4,8′-thiazolo[4,5-e]indole]-6′(7′H)-carboxamide;
(E)-1-(3-(3,4-dichlorophenyl)allyl)-N-isobutylspiro[pipendine-4,8′-thiazolo[4,5-e]indole]-6′(7′H)-carboxamide;
(E)-N-(cyclopropylmethyl)-1-(3-(3,4-dichlorophenyl)allyl)spiro[pipendine-4,8′-thiazolo[4,5-e]indole]-6′(7′H)-carboxamide;
(E)-N-(cyclopentylmethyl)-1-(3-(3,4-dichlorophenyl)allyl)spiro[pipendine-4,8′-thiazolo[4,5-e]indole]-6′(7′H)-carboxamide;
(+/−)(E)-1-(3-(3,4-dichlorophenyl)allyl)-N-((tetrahydrofuran-2-yl)methyl)spiro[pipendine-4,8′-thiazolo[4,5-e]indole]-6′(7′H)-carboxamide;
(+/−)(E)-1-(3-(3,4-dichlorophenyl)allyl)-N-((tetrahydrofuran-3-yl)methyl)spiro[pipendine-4,8′-thiazolo[4,5-e]indole]-6′(7′H)-carboxamide;
(E)-1-(3-(3,4-dichlorophenyl)allyl)-N-(pyridin-3-ylmethyl)spiro[piperidine-4,8′-thiazolo[4,5-e]indole]-6′(7′H)-carboxamide;
(E)-1-(3-(3,4-dichlorophenyl)allyl)-N-(pyridazin-4-ylmethyl)spiro[piperidine-4,8′-thiazolo[4,5-e]indole]-6′(7′H)-carboxamide;
(E)-N-((2-chloropyridin-4-yl)methyl)-1-(3-(4-fluorophenyl)allyl)spiro[piperidine-4,8′-thiazolo[4,5-e]indole]-6′(7′H)-carboxamide;
(E)-1-(3-(4-chlorophenyl)allyl)-N-((2-chloropyridin-4-yl)methyl)spiro[piperidine-4,8′-thiazolo[4,5-e]indole]-6′(7′H)-carboxamide;
(E)-N-((2-chloropyridin-4-yl)methyl)-1-(3-(3,4-dichlorophenyl)allyl)-spiro[piperidine-4,8′-thiazolo[4,5-e]indole]-6′(7′H)-carboxamide;
(E)-N-((2-chloropyridin-4-yl)methyl)-1-(3-(5-(trifluoromethyl)pyridin-2-yl)allyl)spiro[pipendine-4,8′-thiazolo[4,5-e]indole]-6′(7′H)-carboxamide;
(E)-N-((2-chloropyridin-4-yl)methyl)-1-(3-(4-methoxyphenyl)allyl)spiro[piperidine-4,8′-thiazolo[4,5-e]indole]-6′(7′H)-carboxamide;
(E)-1-(3-(4-chlorophenyl)allyl)-N-((2-chloropyridin-4-yl)methyl)-2′-methylspiro[piperidine-4,8′-thiazolo[4,5-e]indole]-6′(7′H)-carboxamide;
(E)-N-((2-chloropyridin-4-yl)methyl)-1-(3-(4-fluorophenyl)allyl)-2′-methylspiro[piperidine-4,8′-thiazolo[4,5-e]indole]-6′(7′H)-carboxamide;
(E)-N-((2-chloropyridin-4-yl)methyl)-2′-methyl-1-(3-(5-(trifluoromethyl)pyridin-2-yl)allyl)spiro[piperidine-4,8′-thiazolo[4,5-e]indole]-6′(7′H)-carboxamide;
(E)-N-((2-chloropyridin-4-yl)methyl)-1-(3-(4-methoxyphenyl)allyl)-2′-methylspiro[piperidine-4,8′-thiazolo[4,5-e]indole]-6′(7′H)-carboxamide;
(E)-N-((2-bromopyridin-4-yl)methyl)-1-(3-(3,4-dichlorophenyl)allyl)-spiro[piperidine-4,8′-thiazolo[4,5-e]indole]-6′(7′H)-carboxamide;
(E)-N-((2-chloropyridin-4-yl)methyl)-1′-(3-(4-fluorophenyl)allyl)spiro[isothiazolo-[4,5-e]indole-8,4′-piperidine]-6(7H)-carboxamide;
(E)-N-((2-chloropyridin-4-yl)methyl)-1′-(3-(5-(trifluoromethyl)pyridin-2-yl)allyl)spiro[isothiazolo[4,5-e]indole-8,4′-piperidine]-6(7H)-carboxamide;
N-((2-chloropyridin-4-yl)methyl)-1′-((6-fluoroquinolin-2-yl)methyl)spiro[isothiazolo[4,5-e]indole-8,4′-piperidine]-6(7H)-carboxamide;
(E)-1-(3-(4-chlorophenyl)allyl)-N-((2-chloropyridin-4-yl)methyl)-3′-methyl-3′H-spiro[piperidine-4,8′-pyrrolo[3,2-e]indazole]-6′(7′H)-carboxamide;
(E)-N-((2-chloropyridin-4-yl)methyl)-1-(3-(4-fluorophenyl)allyl)-3′-methyl-3′H-spiro[piperidine-4,8′-pyrrolo[3,2-e]indazole]-6′(7′H)-carboxamide;
(E)-N-((2-chloropyridin-4-yl)methyl)-3′-methyl-1-(3-(5-(trifluoromethyl)pyridin-2-yl)allyl)-3′H-spiro[piperidine-4,8′-pyrrolo[3,2-e]indazole]-6′(7′H)-carboxamide; and
(E)-N-((2-chloropyridin-4-yl)methyl)-1-(3-(4-methoxyphenyl)allyl)-3′-methyl-3′H-spiro[piperidine-4,8′-pyrrolo[3,2-e]indazole]-6′(7′H)-carboxamide, stereoisomers thereof, and veterinary acceptable salts thereof. In another aspect of the invention are the (Z) isomers of the compounds of Formula (1A).

In another aspect of the invention, are Formula (1B) compounds selected from:
(E)-N-((2-chloropyridin-4-yl)methyl)-1′-(3-(3,4-dichlorophenyl) allyl)-5,7-dihydrospiro[furo[3,4-f]indole-3,4′-piperidine]-1 (2H)-carboxamide;
(E)-1′-(3-(4-chlorophenyl)allyl)-N-((2-fluoropyridin-4-yl)methyl)-5,7-dihydrospiro[furo[3,4-f]indole-3,4′-piperidine]-1 (2H)-carboxamide;
(E)-1′-(3-(4-chlorophenyl)allyl)-N-((2-chlorothiazol-5-yl)methyl)-5,7-dihydrospiro[furo[3,4-f]indole-3,4′-piperidine]-1 (2H)-carboxamide;
(E)-N-((2-chloropyridin-4-yl)methyl)-1′-(3-(4-fluorophenyl)allyl)-5,7-dihydrospiro[furo[3,4-f]indole-3,4′-piperidine]-1 (2H)-carboxamide;
(E)-N-((2-chloropyridin-4-yl)methyl)-1′-(3-(4-cyanophenyl)allyl)-5,7-dihydrospiro[furo[3,4-f]indole-3,4′-piperidine]-1 (2H)-carboxamide;
(E)-1-(3-(4-chlorophenyl)allyl)-N-((2-chloropyridin-4-yl)methyl)spiro[piperidine-4,7′-[1,3]dioxolo[4,5-f]indole]-5′(6′H)-carboxamide;
(E)-N-((2-chloropyridin-4-yl)methyl)-1-(3-(3,4-dichlorophenyl)allyl)-2′,2′-dimethylspiro[piperidine-4,7′-[1,3]dioxolo[4,5-f]indole]-5′(6′H)-carboxamide;
(E)-1-(3-(4-chlorophenyl)allyl)-N-((2-chloropyridin-4-yl)methyl)-2′,2′-dimethylspiro[piperidine-4,7′-[1,3]dioxolo[4,5-f]indole]-5′(6′H)-carboxamide;
(E)-N-((2-chloropyridin-4-yl)methyl)-1-(3-(3,4-dichlorophenyl)allyl)-2′,3′-dihydrospiro[piperidine-4,8′-[1,4]dioxino[2,3-f]indole]-6′(7′H)-carboxamide; and
(E)-N-((2-chloropyridin-4-yl)methyl)-1′-(3-(3,4-dichloro phenyl)allyl)-2,5,6,7-tetrahydro-1H-spiro[cyclopenta[f]indole-3,4′-piperidine]-1-carboxamide, stereoisomers thereof, and veterinary acceptable salts thereof. In another aspect of the invention are the (Z) isomers of the compounds of Formula (1B).

In another aspect of the invention is a compound of Formula (1C),

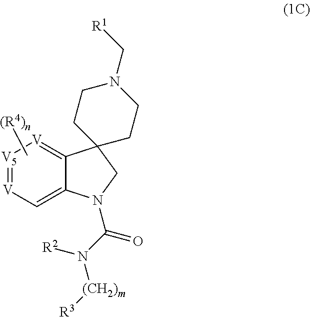

and wherein R¹, R², R³, R⁴, v, m, and n are as defined herein, stereoisomers thereof, and veterinary acceptable salts thereof, with the proviso that when n is the integer 1, then R⁴ is not fluoro or chloro at ring position 5.

In another aspect of the invention, is a compound of Formula (1C) that is a compound of Formula (1C.a), (1C.b), (1C.c) and (1C.d), stereoisomers thereof, and veterinary acceptable salts thereof,

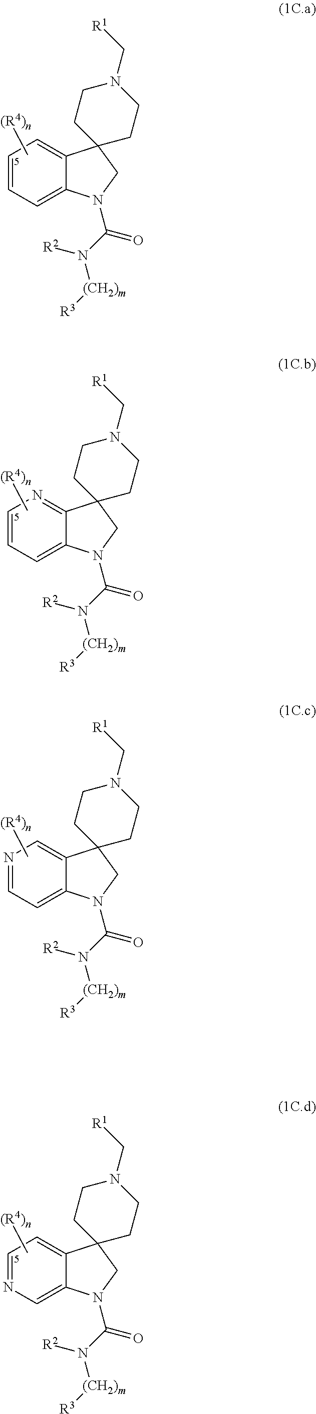

and wherein R¹, R², R³, R⁴, m, and n are as defined herein, stereoisomers thereof, and veterinary acceptable salts thereof, with the proviso that when n is the integer 1, then R⁴ is not fluoro or chloro at ring position 5.

In another aspect, is a compound of Formula (1C.a), stereoisomers thereof, and veterinary acceptable salts thereof. In another aspect, is a compound of Formula (1C.b), stereoisomers thereof, and veterinary acceptable salts thereof. In another aspect, is a compound of Formula (1C.c), stereoisomers thereof, and veterinary acceptable salts thereof. In another aspect, is a compound of Formula (1C.d), stereoisomers thereof, and veterinary acceptable salts thereof.

In another aspect of the invention, particularly for compounds of Formula (1C), R¹ is selected from the group consisting of C₀-C₃alkylaryl, C₀-C₃alkylheteroaryl, C₀-C₃alkylheterocycle, C₀-C₃alkylcycloalkyl, C₂-C₄alkenylaryl, C₂-C₄alkenylheteroaryl, C₂-C₄alkenylcycloalkyl, and C₂-C₄alkenylheterocycle; wherein each C₀-C₃alkyl- or C₂-C₄alkenyl-aryl, -heteroaryl, -cycloalkyl, or -heterocycle R¹ moiety is individually and optionally substituted with at least one substituent as described herein. In another aspect of the invention, R¹ is selected from the group consisting of C₀-C₃alkylaryl, C₀-C₃alkylheteroaryl, C₂-C₄alkenylaryl, and C₂-C₄alkenylheteroaryl, wherein each C₀-C₃alkyl- or C₂-C₄alkenyl-aryl, or -heteroaryl R¹ moiety is individually and optionally substituted with at least one substituent as described herein. In another aspect, R¹ is C₀-C₃alkylaryl, wherein the aryl moiety is optionally substituted with at least one substituent as described herein. In another aspect, the R¹ aryl moiety of C₀-C₃alkylaryl is C₀-C₃alkylphenyl or C₀-C₃alkylnaphthyl, wherein said phenyl or naphthyl moiety is optionally substituted with at least one substituent as described herein. In another aspect, the R¹ aryl moiety of C₀-C₃alkylaryl is C₀-C₃alkylphenyl, wherein said phenyl moiety is optionally substituted with at least one substituent as described herein. In another aspect, the R¹ aryl moiety of C₀-C₃alkylaryl is phenyl, wherein said phenyl is optionally substituted with at least one substituent as described herein. In another aspect, the R¹ aryl moiety of C₀-C₃alkylaryl is C₀-C₃alkylnaphthyl, wherein said naphthyl moiety is optionally substituted with at least one substituent as described herein. In another aspect, the R¹ aryl moiety of C₀-C₃alkylaryl is naphthyl, wherein said naphthyl moiety is optionally substituted with at least one substituent as described herein. In another aspect, the R¹ heteroaryl moiety of C₀-C₃alkylheteroaryl is C₀-C₃alkylpyridinyl or C₀-C₃alkylquinolinyl, wherein said pyridinyl or quinolinyl moiety is optionally substituted with at least one substituent as described herein. In another aspect, the R¹ heteroaryl moiety of C₀-C₃alkylheteroaryl is C₀-C₃alkylpyridinyl, wherein said pyridinyl moiety is optionally substituted with at least one substituent as described herein. In another aspect, the R¹ heteroaryl moiety of C₀-C₃alkylheteroaryl is C₀-C₃alkylquinolinyl, wherein said quinolinyl moiety is optionally substituted with at least one substituent as described herein. In another aspect, the R¹ heteroaryl moiety of C₀-C₃alkylheteroaryl is quinolinyl, wherein said quinoline is optionally substituted with at least one substituent as described herein. In another aspect, R¹ is selected from the group consisting of C₂-C₄alkenylaryl and C₂-C₄alkenylheteroaryl, wherein each C₂-C₄alkenyl-aryl and -heteroaryl R¹ moiety is individually and optionally substituted with at least one substituent as described herein. In yet another aspect, R¹ is selected from the group consisting of C₂-C₄alkenylaryl, wherein the aryl moiety is phenyl or naphthyl, which are optionally substituted with at least one substituent as described herein. In yet another aspect, R¹ is selected from the group consisting of C₂-C₄alkenylaryl, wherein the aryl moiety is phenyl which is optionally substituted with at least one substituent as described herein. In yet another aspect, R¹ is selected from the group consisting of C₂-C₄alkenylaryl, wherein the aryl moiety is naphthyl which is optionally substituted with at least one substituent as described herein. In yet another aspect, R¹ is selected from the group consisting of C₂alkenylaryl, wherein the aryl moiety is optionally substituted with at least one substituent as described herein. In yet another aspect, R¹ is selected from the group consisting of C₂alkenylaryl, wherein the aryl moiety is phenyl or naphthyl which are optionally substituted with at least one substituent as described herein. In yet another aspect, R¹ is selected from the group consisting of C₂alkenylaryl, wherein the aryl moiety is phenyl which is optionally substituted with at least one substituent as described herein. In yet another aspect, R¹ is selected from the group consisting of C₂alkenylaryl, wherein the aryl moiety is naphthyl which is optionally substituted with at least one substituent as described herein. In another aspect, R¹ is selected from the group consisting of C₂-C₄alkenylheteroaryl, wherein the heteroaryl moiety is optionally substituted by at least one substituent as described herein. In another aspect, R¹ is selected from the group consisting of C₂-C₄alkenylheteroaryl, wherein the heteroaryl moiety is pyridinyl or quinolinyl which are optionally substituted by at least one substituent as described herein. In another aspect, R¹ is selected from the group consisting of C₂alkenylheteroaryl, wherein the heteroaryl moiety is pyridinyl which is optionally substituted by at least one substituent as described herein. In another aspect, R¹ is selected from the group consisting of C₂alkenylheteroaryl, wherein the heteroaryl moiety is quinolinyl which is optionally substituted by at least one substituent as described herein. At least one optional substituent for the R¹ alkylaryl, alkylheteroaryl, alkylcycloalkyl, alkyl heterocycle, alkenylaryl, alkenylheteroaryl, alkenylcycloalkyl, and alkenylheterocycle moieties are selected from the group consisting of halo, C₁-C₆alkyl, C₁-C₆alkoxy, cyano, C₁-C₆haloalkoxy, and C₁-C₆haloalkyl. Preferred optional substituents for the R¹ aryl and heteroaryl moieties are selected from the group consisting of chloro, fluoro, bromo, methyl, ethyl, propyl, isopropyl, methoxy, ethoxy, cyano, —CF₃, and —OCF₃. More preferred optional substituents for the R¹ aryl and heteroaryl moieties are selected from the group consisting of chloro, fluoro, bromo, methyl, methoxy, cyano, —CF₃, and —OCF₃. If any of the R¹ aryl or heteroaryl moieties are substituted with more than one substituent, as described herein, the substituents can be the same or different.

In another aspect of the invention, particularly for compounds of Formula (1C), R² is selected from the group consisting of hydrogen, C₁-C₆alkyl, C₁-C₆haloalkyl, and C₁-C₆haloalkoxy. In another aspect of the invention, R² is selected from the group consisting of hydrogen and C₁-C₆alkyl. In another aspect, R² is selected from the group consisting of hydrogen and methyl. In another aspect, R² is hydrogen. In another aspect, R² is methyl.

In another aspect of the invention, particularly for compounds of Formula (1C), R³ is selected from the group consisting of C₃-C₆cycloalkyl, aryl, heteroaryl, and heterocycle. In another aspect, R³ is selected from the group consisting of cyclohexyl, phenyl, pyrazolyl, oxazolyl, thiazolyl, pyridinyl, pyridazinyl, pyrazinyl, and pyrimidinyl, all of which are optionally substituted with at least one substituent as described herein. In another aspect, R³ is selected from the group consisting of cyclohexyl, phenyl, pyrazolyl, oxazolyl, thiazolyl, pyridinyl, pyridazinyl, and pyrimidinyl, all of which are optionally substituted with at least one substituent as described herein. In another aspect, R³ is selected from the group consisting of thiazolyl, pyridinyl, pyridazinyl, and pyrimidinyl, all of which are optionally substituted with at least one substituent as described herein. In another aspect, R³ is selected from the group consisting of thiazolyl, pyridinyl, and pyridazinyl, all of which are optionally substituted with at least one substituent as described herein. In another aspect, R³ is thiazolyl, which is optionally substituted with at least one substituent as described herein. In another aspect, R³ is pyridinyl, which is optionally substituted with at least one substituent as described herein. In another aspect, R³ is pyridazinyl, which is optionally substituted with at least one substituent as described herein. The optional substitutions for the R³ cycloalkyl, aryl, heteroaryl, and heterocycle moieties are individually selected from the group consisting of halo, C₁-C₆haloalkyl, C₁-C₆alkoxy, and isoxazole, wherein said isoxazole can be further substituted with at least one methyl. The preferred R³ cycloalkyl, aryl, heteroaryl, and heterocycle moiety substituents include chloro, fluoro, methoxy, —CF₃, and isoxazole optionally substituted with at least one methyl.

In another aspect of the invention, particularly for compounds of Formula (1C), R⁴ is individually and optionally selected from halo, C₁-C₆alkyl, C₁-C₆haloalkyl, C₁-C₆alkoxy, C₁-C₆haloalkoxy, cyano, nitro, —SO₂CF₃, —SO₂CH₃, —SCF₃, cyclopropyl, SF₅, NR⁵R⁶, phenyl, pyrimidine, pyridine, thiazole, pyrazole, pyrrolidine, pyridine-2(1H)-one, isoxazole, and wherein the phenyl, pyrimidine, pyridine, thiazole, pyrrolidine, and isoxazole substituents can be further optionally substituted with at least one substitute selected from the group consisting of methyl, methoxy, cyano, and —CF₃. In another aspect, n is the integer 0. In another aspect, n is the integer 1. In another aspect of the invention, n is the integer 2. In another aspect of the invention, n is the integer 3. In another aspect, n is the integer 4. When n is the integer 22, 3, or 4, each R⁴ may be identical or different from each other. R⁵ and R⁶ are as described herein.

In one aspect of the invention, particularly for compounds of Formula (1C), m is the integer 1. In yet another aspect, m is the integer 2. In yet another aspect, m is the integer 3. In yet another aspect, m is the integer 4. The preferred integer of m is 1.

In yet another aspect of the invention are compounds of Formula (1C) selected from the group consisting of:
(E)-N-((2-chloropyridin-4-yl)methyl)-1′-(3-(3,4-dichlorophenyl)allyl)-5-(trifluoromethoxy)spiro[indoline-3,4′-piperidine]-1-carboxamide;
(E)-N-((2-chloropyridin-4-yl)methyl)-1′-(3-(3,4-dichlorophenyl)allyl)-4-(trifluoromethyl)spiro[indoline-3,4′-piperidine]-1-carboxamide;
(E)-7-chloro-1′-(3-(4-chlorophenyl)allyl)-N-((2-chlorothiazol-5-yl)methyl)-4-(trifluoromethoxy)spiro[indoline-3,4′-piperidine]-1-carboxamide;
(E)-7-chloro-N-((2-chloropyridin-4-yl)methyl)-1′-(3-(4-fluorophenyl)allyl)-4-(trifluoromethoxy)spiro[indoline-3,4′-piperidine]-1-carboxamide;
(E)-4,6-dichloro-1′-(3-(4-chlorophenyl)allyl)-N-((2-chloropyridin-4-yl)methyl)spiro[indoline-3,4′-piperidine]-1-carboxamide;
(E)-4,6-dichloro-1′-(3-(4-chlorophenyl)allyl)-N-((2-fluoropyridin-4-yl)methyl)spiro[indoline-3,4′-piperidine]-1-carboxamide;
(E)-4,6-dichloro-1′-(3-(4-chlorophenyl)allyl)-5-fluoro-N-((2-fluoropyridin-4-yl)methyl)spiro[indoline-3,4′-piperidine]-1-carboxamide;
(E)-4,5-dichloro-1′-(3-(4-chlorophenyl)allyl)-N-((2-fluoropyridin-4-yl)methyl)spiro[indoline-3,4′-piperidine]-1-carboxamide;
(E)-4-chloro-1′-(3-(4-chlorophenyl)allyl)-5-fluoro-N-((2-fluoropyridin-4-yl)methyl)spiro[indoline-3,4′-piperidine]-1-carboxamide;
(E)-4,5-dichloro-1′-(3-(4-chlorophenyl)allyl)-N-((2-chlorothiazol-5-yl)methyl)spiro[indoline-3,4′-piperidine]-1-carboxamide;
(E)-N-((2-chloropyridin-4-yl)methyl)-1-(3-(3,4-dichlorophenyl)allyl)-6′-methoxyspiro[piperidine-4,3′-pyrrolo[3,2-c]pyridine]-1′(2′H)-carboxamide;
(E)-N-((2-chloropyridin-4-yl)methyl)-5′-cyano-1-(3-(3,4-dichlorophenyl)allyl)-spiro[piperidine-4,3′-pyrrolo[2,3-c]pyridine]-1′(2′H)-carboxamide;
(E)-N-((2-chloropyridin-4-yl)methyl)-5′-cyano-1-(3-(3,4-dichlorophenyl)allyl)spiro[piperidine-4,3′-pyrrolo[3,2-b]pyridine]-1′(2′H)-carboxamide;
(E)-N-((2-chloropyridin-4-yl)methyl)-1-(3-(3,4-dichlorophenyl)allyl)-4′-methoxyspiro[piperidine-4,3′-pyrrolo[3,2-c]pyridine]-1′(2′H)-carboxamide;
(E)-6-chloro-1′-(3-(4-chlorophenyl)allyl)-5-fluoro-N-((2-fluoropyridin-4-yl)methyl)spiro[indoline-3,4′-piperidine]-1-carboxamide;
(E)-5,6-dichloro-1′-(3-(4-chlorophenyl)allyl)-N-((2-fluoropyridin-4-yl)methyl)spiro[indoline-3,4′-piperidine]-1-carboxamide;
(E)-5-chloro-1′-(3-(4-chlorophenyl)allyl)-6-fluoro-N-((2-fluoropyridin-4-yl)methyl)spiro[indoline-3,4′-piperidine]-1-carboxamide;
(E)-5-chloro-N-((2-chloropyridin-4-yl)methyl)-1′-(3-(4-cyanophenyl)allyl)-6-(trifluoromethyl)spiro[indoline-3,4′-piperidine]-1-carboxamide;
(E)-1′-(3-(4-chlorophenyl)allyl)-N-((2-fluoropyridin-4-yl)methyl)-6-(2-(trifluoromethoxy)phenyl)spiro[indoline-3,4′-piperidine]-1-carboxamide;
(E)-1′-(3-(4-chlorophenyl)allyl)-N-((2-chloropyridin-4-yl)methyl)-5-(2-oxopyridin-1(2H)-yl)spiro[indoline-3,4′-piperidine]-1-carboxamide;
(E)-1′-(3-(4-chlorophenyl)allyl)-N-((2-fluoropyridin-4-yl)methyl)-5-(2-oxopyridin-1(2H)-yl)spiro[indoline-3,4′-piperidine]-1-carboxamide;
(E)-1′-(3-(4-chlorophenyl)allyl)-N-((2-chloropyridin-4-yl)methyl)-5-(pyrrolidin-1-yl)spiro[indoline-3,4′-piperidine]-1-carboxamide;
(E)-1′-(3-(4-chlorophenyl)allyl)-N-((2-fluoropyridin-4-yl)methyl)-5-(trifluoromethoxy)spiro[indoline-3,4′-piperidine]-1-carboxamide;
(E)-1′-(3-(4-chlorophenyl)allyl)-N-((2-chloropyridin-4-yl)methyl)-5-(trifluoromethylsulfonyl)spiro[indoline-3,4′-piperidine]-1-carboxamide;
(E)-1′-(3-(4-chlorophenyl)allyl)-N-((2-chloropyridin-4-yl)methyl)-5-(methylsulfonyl)spiro[indoline-3,4′-piperidine]-1-carboxamide;
(E)-1′-(3-(4-chlorophenyl)allyl)-N-((2-chloropyridin-4-yl)methyl)-5-(trifluoromethylthio)spiro[indoline-3,4′-piperidine]-1-carboxamide;
(E)-1′-(3-(4-chlorophenyl)allyl)-N-((2-chloropyridin-4-yl)methyl)-5-(thiazol-2-yl)spiro[indoline-3,4′-piperidine]-1-carboxamide;
(E)-1′-(3-(4-chlorophenyl)allyl)-N-((2-fluoropyridin-4-yl)methyl)-5-(trifluoromethyl)spiro[indoline-3,4′-piperidine]-1-carboxamide;
(E)-1′-(3-(4-chlorophenyl)allyl)-N-((2-fluoropyridin-4-yl)methyl)-5-(2-oxo-1,2-dihydropyridin-4-yl)spiro[indoline-3,4′-piperidine]-1-carboxamide;
(E)-1′-(3-(4-chlorophenyl)allyl)-5-cyclopropyl-N-((2-fluoropyridin-4-yl)methyl)spiro[indoline-3,4′-piperidine]-1-carboxamide;
(E)-1′-(3-(4-chlorophenyl)allyl)-N-((2-chlorothiazol-5-yl)methyl)-5-(trifluoromethyl)spiro[indoline-3,4′-piperidine]-1-carboxamide;
(E)-1-(3-(4-chlorophenyl)allyl)-N-((2-chloropyridin-4-yl)methyl)-5′-cyanospiro[piperidine-4,3′-pyrrolo[2,3-c]pyridine]-1′(2′H)-carboxamide;
(E)-1-(3-(4-chlorophenyl)allyl)-N-((2-chloropyridin-4-yl)methyl)-5′-cyanospiro[piperidine-4,3′-pyrrolo[3,2-b]pyridine]-1′(2′H)-carboxamide;
(E)-1′-(3-(4-chlorophenyl)allyl)-N-((2-chloropyridin-4-yl)methyl)-4-(trifluoromethoxy)spiro[indoline-3,4′-piperidine]-1-carboxamide;
(E)-1′-(3-(4-chlorophenyl)allyl)-N-((2-chloropyridin-4-yl)methyl)-4-(trifluoromethylthio)spiro[indoline-3,4′-piperidine]-1-carboxamide;
(E)-1′-(3-(4-chlorophenyl)allyl)-4-fluoro-N-((2-fluoropyridin-4-yl)methyl)-6-(trifluoromethyl)spiro[indoline-3,4′-piperidine]-1-carboxamide;
(E)-1′-(3-(4-chlorophenyl)allyl)-N-((2-fluoropyridin-4-yl)methyl)-4-phenylspiro[indoline-3,4′-piperidine]-1-carboxamide;
(E)-1′-(3-(4-chlorophenyl)allyl)-N-((2-fluoropyridin-4-yl)methyl)-4-(trifluoromethyl)spiro[indoline-3,4′-piperidine]-1-carboxamide;
(E)-1′-(3-(4-chlorophenyl)allyl)-4-cyclopropyl-N-((2-fluoropyridin-4-yl)methyl)spiro[indoline-3,4′-piperidine]-1-carboxamide;
(E)-1′-(3-(4-chlorophenyl)allyl)-N-((2-fluoropyridin-4-yl)methyl)-4-(trifluoromethoxy)spiro[indoline-3,4′-piperidine]-1-carboxamide;
(E)-1′-(3-(4-chlorophenyl)allyl)-N-((2-chlorothiazol-5-yl)methyl)-4-(trifluoromethoxy)spiro[indoline-3,4′-piperidine]-1-carboxamide;
(E)-N-((2-chloropyridin-4-yl)methyl)-1′-(3-(4-cyanophenyl)allyl)-6-(2-(trifluoromethyl)phenyl)spiro[indoline-3,4′-piperidine]-1-carboxamide;
(E)-N-((2-chloropyridin-4-yl)methyl)-1′-(3-(4-cyanophenyl)allyl)-5-nitrospiro[indoline-3,4′-piperidine]-1-carboxamide;
(E)-N-((2-chloropyridin-4-yl)methyl)-1′-(3-(4-cyanophenyl)allyl)-7-fluoro-4-(trifluoromethyl)spiro[indoline-3,4′-piperidine]-1-carboxamide;
(E)-N-((2-chloropyridin-4-yl)methyl)-1′-(3-(4-cyanophenyl)allyl)-4-(trifluoromethyl)spiro-[indoline-3,4′-piperidine]-1-carboxamide;
(E)-1′-(3-(4-fluorophenyl)allyl)-N-((2-fluoropyridin-4-yl)methyl)-6-(trifluoromethoxy)-spiro[indoline-3,4′-pipendine]-1-carboxamide;
(E)-1′-(3-(4-fluorophenyl)allyl)-N-((2-fluoropyridin-4-yl)methyl)-6-(trifluoromethyl)spiro-[indoline-3,4′-piperidine]-1-carboxamide;
(E)-4-bromo-N-((2-chloropyridin-4-yl)methyl)-1′-(3-(3,4-dichlorophenyl)allyl)spiro-[indoline-3,4′-piperidine]-1-carboxamide;
4-bromo-1′-[(E)-3-(3,4-dichlorophenyl)allyl]-N-[(2-fluoro-4-pyridyl)methyl]spiro[indoline-3,4′-piperidine]-1-carboxamide;
5-bromo-1′-[(E)-3-(3,4-dichlorophenyl)allyl]-N-[(2-fluoro-4-pyridyl)methyl]spiro[indoline-3,4′-piperidine]-1-carboxamide;
5-bromo-1′-[(E)-3-(4-chlorophenyl)allyl]-N-[(2-fluoro-4-pyridyl)methyl]spiro[indoline-3,4′-piperidine]-1-carboxamide;
5-bromo-1′-[(E)-3-(4-fluorophenyl)allyl]-N-[(2-fluoro-4-pyridyl)methyl]spiro[indoline-3,4′-piperidine]-1-carboxamide;
5-bromo-N-[(2-fluoro-4-pyridyl)methyl]-1′-[(E)-3-[4-(trifluoromethyl)phenyl]allyl]-spiro[indoline-3,4′-piperidine]-1-carboxamide;
5-(3-cyanophenyl)-1′-[(E)-3-(3,4-dichlorophenyl)allyl]-N-[(2-fluoro-4-pyridyl)methyl]spiro-[indoline-3,4′-piperidine]-1-carboxamide
1′-[(E)-3-(4-chlorophenyl)allyl]-5-(6-cyano-3-pyridyl)-N-[(2-fluoro-4-pyridyl)methyl]spiro-[indoline-3,4′-piperidine]-1-carboxamide;
1′-[(E)-3-(3,4-dichlorophenyl)allyl]-N-[(2-fluoro-4-pyridyl)methyl]-5-(3-pyridyl)spiro-[indoline-3,4′-piperidine]-1-carboxamide;
1′-[(E)-3-(3,4-dichlorophenyl)allyl]-N-[(2-fluoro-4-pyridyl)methyl]-5-(1H-pyrazol-4-yl)spiro[indoline-3,4′-piperidine]-1-carboxamide;
1′-[(E)-3-(3,4-dichlorophenyl)allyl]-N-[(2-fluoro-4-pyridyl)methyl]-5-pyrimidin-5-yl-spiro[indoline-3,4′-piperidine]-1-carboxamide;
5-cyano-1′-[(E)-3-(3, 4-dichlorophenyl)allyl]-N-[(2-fluoro-4-pyridyl)methyl]spiro[indoline-3,4′-piperidine]-1-carboxamide;
1′-[(E)-3-(3, 4-dichlorophenyl)allyl]-N-[(2-fluoro-4-pyridyl)methyl]-4-methyl-spiro[indoline-3,4′-pipendine]-1-carboxamide;
1′-[(E)-3-(4-chlorophenyl)allyl]-5-(5-cyano-3-pyridyl)-N-[(2-fluoro-4-pyridyl)methyl]spiro[indoline-3,4′-piperidine]-1-carboxamide;
5-(6-cyano-3-pyridyl)-1′-[(E)-3-(3,4-dichlorophenyl)allyl]-N-[(2-fluoro-4-pyridyl)methyl]spiro[indoline-3,4′-piperidine]-1-carboxamide;
5-cyano-N-[(2-fluoro-4-pyridyl)methyl]-1′-[(2-methoxy-8-methyl-7-quinolyl)methyl]spiro[indoline-3,4′-piperidine]-1-carboxamide;
N-[(2-chloro-4-pyridyl)methyl]-5-cyano-1′-[(2-methoxy-8-methyl-7-quinolyl)methyl]spiro[indoline-3,4′-piperidine]-1-carboxamide;
1′-[(E)-3-(4-chlorophenyl)allyl]-N-[(2-chloro-4-pyridyl)methyl]-5-(6-methoxy-3-pyridyl)spiro[indoline-3,4′-piperidine]-1-carboxamide;
N-[(2-chloro-4-pyridyl)methyl]-5-(6-cyano-3-pyridyl)-1′-[(E)-3-[4-(trifluoromethyl)phenyl]allyl]spiro[indoline-3,4′-piperidine]-1-carboxamide;
(E)-7-chloro-1′-(3-(4-chlorophenyl)allyl)-N-((2-chloropyridin-4-yl)methyl)-4-(trifluoromethoxy)spiro[indoline-3,4′-piperidine]-1-carboxamide;
(E)-1′-(3-(4-chlorophenyl)allyl)-N-((2-chloropyridin-4-yl)methyl)-4-fluoro-6-(trifluoromethyl)spiro[indoline-3,4′-piperidine]-1-carboxamide;
(E)-N-((2-chloropyridin-4-yl)methyl)-1′-(3-(3,4-dichlorophenyl)allyl)-5-methylspiro[indoline-3,4′-piperidine]-1-carboxamide;
(E)-N-((2-chloropyridin-4-yl)methyl)-6-(trifluoromethyl)-1′-(3-(5-(trifluoromethyl)pyridin-2-yl)allyl)spiro[indoline-3,4′-piperidine]-1-carboxamide;
(E)-N-((2-chloropyridin-4-yl)methyl)-1′-(3-(4-methoxyphenyl)allyl)-6-(trifluoromethyl)spiro[indoline-3,4′-piperidine]-1-carboxamide;
(E)-1′-(3-(4-chlorophenyl)allyl)-N-((2-chloropyridin-4-yl)methyl)-5-fluoro-6-(trifluoromethyl)spiro[indoline-3,4′-piperidine]-1-carboxamide;
(E)-1′-(3-(4-chlorophenyl)allyl)-N-((2-fluoropyridin-4-yl)methyl)-6-(trifluoromethoxy)spiro[indoline-3,4′-piperidine]-1-carboxamide;
(E)-5-chloro-N-((2-chloropyridin-4-yl)methyl)-6-fluoro-1′-(3-(4-(trifluoromethyl)phenyl)allyl)spiro[indoline-3,4′-piperidine]-1-carboxamide;
(E)-N-((2-chloropyridin-4-yl)methyl)-1′-(3-(3,4-dichlorophenyl)allyl)-5-fluoro-6-(trifluoromethyl)spiro[indoline-3,4′-piperidine]-1-carboxamide;
(E)-N-((2-chloropyridin-4-yl)methyl)-1′-(3-(4-(trifluoromethoxy)phenyl)allyl)-6-(trifluoromethyl)spiro[indoline-3,4′-piperidine]-1-carboxamide;
(E)-4-chloro-N-((2-chloropyridin-4-yl)methyl)-1′-(3-(3,4-dichlorophenyl)allyl) spiro[indoline-3,4′-piperidine]-1-carboxamide;
(E)-N-((2-chloropyridin-4-yl)methyl)-5-cyano-1′-(3-(3,4-dichlorophenyl)allyl)-6-(trifluoromethyl)spiro[indoline-3,4′-piperidine]-1-carboxamide;
(E)-N-((2-chloropyridin-4-yl)methyl)-1′-(3-(3,4-dichlorophenyl)allyl)-5-(trifluoromethyl)spiro[indoline-3,4′-pipendine]-1-carboxamide;
(E)-5-chloro-N-((2-chloropyridin-4-yl)methyl)-1′-(3-(3,4-dichlorophenyl)allyl)-6-(trifluoromethyl)spiro[indoline-3,4′-pipendine]-1-carboxamide;
(E)-4,5-dichloro-N-((2-chloropyridin-4-yl)methyl)-1′-(3-(3,4-dichlorophenyl)allyl)spiro[indoline-3,4′-pipendine]-1-carboxamide;
(E)-N-((2-chloropyridin-4-yl)methyl)-1′-(3-(3,4-dichlorophenyl)allyl)-5-(dimethylamino)spiro[indoline-3,4′-pipendine]-1-carboxamide;
(E)-N-((2-chloropyridin-4-yl)methyl)-6-(trifluoromethyl)-1′-(3-(4-(trifluoromethyl)phenyl)allyl)spiro[indoline-3,4′-pipendine]-1-carboxamide;
(E)-4-chloro-1′-(3-(4-chlorophenyl)allyl)-N-((2-chloropyridin-4-yl)methyl)-5-fluorospiro[indoline-3,4′-pipendine]-1-carboxamide;
(E)-5,6-dichloro-N-((2-chloropyridin-4-yl)methyl)-1′-(3-(4-fluorophenyl)allyl)spiro[indoline-3,4′-pipendine]-1-carboxamide;
(E)-5,6-dichloro-1′-(3-(4-chlorophenyl)allyl)-N-((2-chloropyridin-4-yl)methyl)spiro[indoline-3,4′-pipendine]-1-carboxamide;
(E)-4,5-dichloro-N-((2-chloropyridin-4-yl)methyl)-1′-(3-(4-fluorophenyl)allyl)spiro[indoline-3,4′-pipendine]-1-carboxamide;
(E)-4,5-dichloro-1′-(3-(4-chlorophenyl)allyl)-N-((2-chloropyridin-4-yl)methyl)spiro[indoline-3,4′-piperidine]-1-carboxamide;
(E)-6-chloro-N-((2-chloropyridin-4-yl)methyl)-5-fluoro-1′-(3-(4-(trifluoromethyl)phenyl)allyl)spiro[indoline-3,4′-pipendine]-1-carboxamide;
(E)-4-chloro-N-((2-chloropyridin-4-yl)methyl)-1′-(3-(3,4-dichlorophenyl)allyl)-5-fluorospiro[indoline-3,4′-pipendine]-1-carboxamide;
(E)-1′-(3-(4-chlorophenyl)allyl)-N-((2-chloropyridin-4-yl)methyl)-5-(trifluoromethoxy)spiro[indoline-3,4′-pipendine]-1-carboxamide;
(E)-7-chloro-1′-(3-(4-chlorophenyl)allyl)-N-((2-fluoropyridin-4-yl)methyl)-4-(trifluoromethoxy)spiro[indoline-3,4′-pipendine]-1-carboxamide;
(E)-1′-(3-(4-chlorophenyl)allyl)-N-((2-chloropyridin-4-yl)methyl)-4-(trifluoromethyl)spiro[indoline-3,4′-piperidine]-1-carboxamide;
(E)-6-chloro-1′-(3-(4-chlorophenyl)allyl)-N-((2-chloropyridin-4-yl)methyl)-5-(trifluoromethoxy)spiro[indoline-3,4′-pipendine]-1-carboxamide;
(E)-N-((2-chloropyridin-4-yl)methyl)-5-(trifluoromethyl)-1′-(3-(5-(trifluoromethyl)pyridin-2-yl)allyl)spiro[indoline-3,4′-pipendine]-1-carboxamide;
(E)-N-((2-chloropyridin-4-yl)methyl)-4-(trifluoromethyl)-1′-(3-(4-(trifluoromethyl)phenyl)allyl)spiro[indoline-3,4′-piperidine]-1-carboxamide;
(E)-N-((2-chloropyridin-4-yl)methyl)-1′-(3-(4-fluorophenyl)allyl)-4-(trifluoromethyl)spiro[indoline-3,4′-piperidine]-1-carboxamide;
(E)-N-((2-chloropyridin-4-yl)methyl)-1′-(3-(4-fluorophenyl)allyl)-5-(trifluoromethyl)spiro[indoline-3,4′-piperidine]-1-carboxamide;
(E)-1′-(3-(4-chlorophenyl)allyl)-N-((2-chloropyridin-4-yl)methyl)-5-fluoro-4-methylspiro[indoline-3,4′-piperidine]-1-carboxamide;
(E)-4-bromo-1′-(3-(4-chlorophenyl)allyl)-N-((2-chloropyridin-4-yl)methyl)spiro[indoline-3,4′-piperidine]-1-carboxamide;
(E)-4-chloro-N-((2-chloropyridin-4-yl)methyl)-1′-(3-(4-cyanophenyl)allyl)-5-fluorospiro[indoline-3,4′-piperidine]-1-carboxamide;
(E)-4-chloro-N-((2-chloropyridin-4-yl)methyl)-5-fluoro-1′-(3-(4-fluorophenyl)allyl)spiro[indoline-3,4′-piperidine]-1-carboxamide;
(E)-4-chloro-1′-(3-(4-chlorophenyl)allyl)-N-((2-chloropyridin-4-yl)methyl)-5-(trifluoromethoxy)spiro[indoline-3,4′-piperidine]-1-carboxamide;
(E)-1′-(3-(4-chlorophenyl)allyl)-N-((2-chloropyridin-4-yl)methyl)-5-(trifluoromethyl)spiro[indoline-3,4′-piperidine]-1-carboxamide;
(E)-4,6-dichloro-N-((2-chloropyridin-4-yl)methyl)-1′-(3-(4-fluorophenyl)allyl)spiro[indoline-3,4′-piperidine]-1-carboxamide;
(E)-1′-(3-(4-chlorophenyl)allyl)-N-((2-chloropyridin-4-yl)methyl)-6-(methylsulfonyl)spiro[indoline-3,4′-piperidine]-1-carboxamide;
(E)-N-((2-chloropyridin-4-yl)methyl)-1′-(3-(4-fluorophenyl)allyl)-5-(trifluoromethoxy)spiro[indoline-3,4′-piperidine]-1-carboxamide;
(E)-4-chloro-N-((2-chloropyridin-4-yl)methyl)-5-fluoro-1′-(3-(4-(trifluoromethyl)phenyl)allyl)spiro[indoline-3,4′-piperidine]-1-carboxamide;
(E)-1′-(3-(4-chlorophenyl)allyl)-N-((2-chloropyridin-4-yl)methyl)-4-cyanospiro[indoline-3,4′-piperidine]-1-carboxamide;
(E)-1′-(3-(4-chlorophenyl)allyl)-N-((2-chloropyridin-4-yl)methyl)-4-cyano-5-fluorospiro[indoline-3,4′-piperidine]-1-carboxamide;
(E)-N-((2-chloropyridin-4-yl)methyl)-1′-(3-(3,4-dichlorophenyl)allyl)-5-methoxyspiro[indoline-3,4′-piperidine]-1-carboxamide;
(E)-N-((2-chloropyridin-4-yl)methyl)-1′-(3-(4-methoxyphenyl)allyl)-5-(trifluoromethyl)spiro[indoline-3,4′-piperidine]-1-carboxamide;
(E)-1′-(3-(4-chlorophenyl)allyl)-N-((2-chloropyridin-4-yl)methyl)-5-(pentafluorosulfide)spiro[indoline-3,4′-piperidine]-1-carboxamide;
(E)-N-((2-chloropyridin-4-yl)methyl)-5-(trifluoromethyl)-1′-(3-(4-(trifluoromethyl)phenyl)allyl)spiro[indoline-3,4′-piperidine]-1-carboxamide;
(E)-5,7-dichloro-N-((2-chloropyridin-4-yl)methyl)-1′-(3-(3,4-dichlorophenyl)allyl)spiro[indoline-3,4′-piperidine]-1-carboxamide;
(E)-N-((2-chloropyridin-4-yl)methyl)-1′-(3-(4-(trifluoromethoxy)phenyl)allyl)-5-(trifluoromethyl)spiro[indoline-3,4′-piperidine]-1-carboxamide;
(E)-1′-(3-(4-chlorophenyl)allyl)-N-((2-chloropyridin-4-yl)methyl)-5-fluoro-4-(trifluoromethyl)spiro[indoline-3,4′-piperidine]-1-carboxamide;
(E)-N-((2-chloropyridin-4-yl)methyl)-1′-(3-(4-fluorophenyl)allyl)-4-(trifluoromethoxy)spiro[indoline-3,4′-piperidine]-1-carboxamide;
(E)-1′-(3-(4-fluorophenyl)allyl)-5-(trifluoromethoxy)-N-((2-(trifluoromethyl)pyridin-4-yl)methyl)spiro[indoline-3,4′-piperidine]-1-carboxamide;
(E)-1′-(3-(4-cyanophenyl)allyl)-5-(trifluoromethoxy)-N-((2-(trifluoromethyl)pyridin-4-yl)methyl)spiro[indoline-3,4′-piperidine]-1-carboxamide;
(E)-5-(trifluoromethoxy)-1′-(3-(4-(trifluoromethyl)phenyl)allyl)-N-((2-(trifluoromethyl)pyridin-4-yl)methyl)spiro[indoline-3,4′-piperidine]-1-carboxamide;
(E)-5-(trifluoromethoxy)-1′-(3-(4-(trifluoromethoxy)phenyl)allyl)-N-((2-(trifluoromethyl)pyridin-4-yl)methyl)spiro[indoline-3,4′-piperidine]-1-carboxamide;
(E)-1′-(3-(4-chlorophenyl)allyl)-5-(trifluoromethoxy)-N-((2-(trifluoromethyl)pyridin-4-yl)methyl)spiro[indoline-3,4′-piperidine]-1-carboxamide;
(E)-N-((2-chloropyridin-4-yl)methyl)-5-(trifluoromethoxy)-1′-(3-(4-(trifluoromethoxy)phenyl)allyl)spiro[indoline-3,4′-piperidine]-1-carboxamide;
(E)-N-((2-chloropyridin-4-yl)methyl)-5-(trifluoromethoxy)-1′-(3-(4-(trifluoromethyl)phenyl)allyl)spiro[indoline-3,4′-piperidine]-1-carboxamide;
(E)-N-((2-chloropyridin-4-yl)methyl)-5-(trifluoromethoxy)-1′-(3-(5-(trifluoromethyl)pyridin-2-yl)allyl)spiro[indoline-3,4′-piperidine]-1-carboxamide;
(E)-1′-(3-(4-chlorophenyl)allyl)-N-((2-chloropyridin-4-yl)methyl)-5-(trifluoromethoxy)spiro[indoline-3,4′-piperidine]-1-carboxamide;
(E)-1′-(3-(4-chlorophenyl)allyl)-5-(3,5-dimethylisoxazol-4-yl)-N-((2-(3,5-dimethylisoxazol-4-yl)pyridin-4-yl)methyl)spiro[indoline-3,4′-piperidine]-1-carboxamide;
(E)-1′-(3-(4-chlorophenyl)allyl)-N-((2-chloropyridin-4-yl)methyl)-5-(3,5-dimethylisoxazol-4-yl)spiro[indoline-3,4′-piperidine]-1-carboxamide; and
(E)-1′-(3-(4-chlorophenyl)allyl)-N-((2-chlorothiazol-5-yl)methyl)-5-(trifluoromethoxy)spiro[indoline-3,4′-piperidine]-1-carboxamide stereoisomers thereof, and veterinary acceptable salts thereof. The (Z) isomers of these Formula (1C) compounds are also contemplated.

In another aspect of the invention are preferred compounds of Formula (1C) selected from the group consisting of:
(E)-N-((2-chloropyridin-4-yl)methyl)-1′-(3-(3,4-dichlorophenyl)allyl)-5-(trifluoromethoxy)spiro[indoline-3,4′-piperidine]-1-carboxamide;
(E)-5,6-dichloro-1′-(3-(4-chlorophenyl)allyl)-N-((2-fluoropyridin-4-yl)methyl)spiro[indoline-3,4′-piperidine]-1-carboxamide;
(E)-1′-(3-(4-chlorophenyl)allyl)-N-((2-fluoropyridin-4-yl)methyl)-6-(trifluoromethoxy)spiro[indoline-3,4′-piperidine]-1-carboxamide;
(E)-5-(trifluoromethoxy)-1′-(3-(4-(trifluoromethoxy)phenyl)allyl)-N-((2-(trifluoromethyl)pyridin-4-yl)methyl)spiro[indoline-3,4′-piperidine]-1-carboxamide; and
(E)-1′-(3-(4-chlorophenyl)allyl)-N-((2-chlorothiazol-5-yl)methyl)-5-(trifluoromethoxy)spiro[indoline-3,4′-piperidine]-1-carboxamide; stereoisomers thereof, and veterinary acceptable salts thereof. The (Z) isomers of these compounds are also contemplated.

In another aspect, Applicant includes the following 5′ chloro compounds that were shown to have nematodal activity and VAChT specificity. These additional compounds include:
5-chloro-N-((2-chloropyridin-4-yl)methyl)-1′-((2-methoxyquinolin-3-yl)methyl)spiro[indoline-3,4′-piperidine]-1-carboxamide;
5-chloro-N-((2-chloropyridin-4-yl)methyl)-1′-((6-fluoronaphthalen-2-yl)methyl)spiro[indoline-3,4′-piperidine]-1-carboxamide;
(E)-5-chloro-N-((2-methoxypyridin-4-yl)methyl)-1′-(3-(4-(trifluoromethyl)phenyl)-allyl)spiro[indoline-3,4′-piperidine]-1-carboxamide;
(E)-5-chloro-N-((2-fluoropyridin-4-yl)methyl)-1′-(3-(4-(trifluoromethyl)phenyl)-allyl)spiro[indoline-3,4′-piperidine]-1-carboxamide;
(E)-5-chloro-1′-(3-(4-chlorophenyl)allyl)-N-((2-chloropyridin-4-yl)methyl)-N-methylspiro[indoline-3,4′-piperidine]-1-carboxamide; and (E)-5-chloro-1′-(3-(4-chlorophenyl)allyl)-N-(1-(pyridin-4-yl)ethyl)spiro[indoline-3,4′-piperidine]-1-carboxamide, stereoisomers thereof, and veterinary acceptable salts thereof. The (Z) isomers of these 5′ chloro compounds are also contemplated.

In another aspect of the invention, is a veterinary composition that comprises a Formula (1A), (1B), or (1C) compound, stereoisomers thereof, and veterinarily acceptable salt thereof. In another aspect of the invention, is a veterinary composition that comprises a Formula (1A) compound, stereoisomers thereof, and veterinarily acceptable salt thereof. In another aspect of the invention, is a veterinary composition that comprises a Formula (1B) compound, stereoisomers thereof, and veterinarily acceptable salt thereof. In another aspect of the invention, is a veterinary composition that comprises a Formula (1C) compound, stereoisomers thereof, and veterinarily acceptable salt thereof. In yet another aspect of the invention, is a veterinary composition that comprises a therapeutic amount of a) a Formula (1A), Formula (1B), or Formula (1C) compound, stereoisomers thereof, and veterinarily acceptable salt thereof, and (b) a veterinarily acceptable excipient. In yet another aspect of the invention, is a veterinary composition that comprises a therapeutic amount of a) a Formula (1A) compound, stereoisomers thereof, and veterinarily acceptable salt thereof, and (b) a veterinarily acceptable excipient. In yet another aspect of the invention, is a veterinary composition that comprises a therapeutic amount of a) a Formula (1B) compound, stereoisomers thereof, and veterinarily acceptable salt thereof, and (b) a veterinarily acceptable excipient. In yet another aspect of the invention, is a veterinary composition that comprises a therapeutic amount of a) Formula (1C) compound, stereoisomers thereof, and veterinarily acceptable salt thereof, and (b) a veterinarily acceptable excipient.

The composition comprising a Formula (1A), (1B), or (1C) compound, stereoisomers thereof, and veterinary acceptable salts thereof, or a composition comprising a therapeutic amount of a Formula (1A), (1B), or (1C) compound, stereoisomers thereof, and veterinary acceptable slats thereof, and further comprising a veterinary acceptable excipient, may comprise at least one additional veterinary agent. Preferred additional veterinary agents include other known parasiticides. Examples of additional veterinary agents include, but are not limited to: amitraz, aminoacetonitriles, anthelmintics (e.g., albendazole, cambendazole, fenbendazole, flubendazole, mebendazole, cyclic octadepsipeptides, oxfendazole, oxibendazole, paraherquamide, parbendazole, piperazines, praziquantel, thiabendazole, tetramisole, triclabendazole, levamisole, pyrantel (including the salt forms (e.g., pamoate, citrate, and tartrate)), oxantel, morantel, and the like), macrocyclic lactones and derivatives thereof (e.g., abamectin, doramectin, emamectin, eprinomectin, ivermectin, moxidectin, selamectin, dimadectin, latidectin, lepimectin, milbemycin, milbemycin oxime, and the like), demiditraz, cyclic octadepsipeptides (e.g., emodepside), diethylcarbamazine, fipronil, hydroprene, kinoprene, methoprene, lufenuron, metaflumizone, niclosamide, permethrin, pyrethrins, pyriproxyfen, closantel, clorsulon, praziquantel, novaluron, fluazuron, spinosad, isoxazolines, for example, sarolaner ((S)-1-(5′-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)-2-(methylsulfonyl)ethan-1-one), fluralaner (4-(5-(3,5-dichlorophenyl)-5-(trifluoromethyl)-4,5-dihydroisoxazol-3-yl)-2-methyl-N-(2-oxo-2-((2,2,2-trifluoroethyl)amino)ethyl)benzamide), afoxolaner (4-(5-(3-chloro-5-(trifluoromethyl)phenyl)-5-(trifluoromethyl)-4,5-dihydroisoxazol-3-yl)-N-(2-oxo-2-((2,2,2-trifluoroethyl)amino)ethyl)-1-naphthamide), lotilaner (3-methyl-N-{2-oxo-2-[(2,2,2-trifluoroethyl)amino]ethyl}-5-[(5S)-5-(3,4,5-trichlorophenyl)-5-(trifluoromethyl)-4,5-dihydro-1,2-oxazol-3-yl]thiophene-2-carboxamide); and mixtures thereof.

In yet another aspect of the invention is the use of a Formula (1A), Formula (1B), or Formula (1C) compound, stereoisomers, and veterinarily acceptable salt thereof, for the manufacture of a medicament. In yet another aspect of the invention is the use of a Formula (1A), Formula (1B), or Formula (1C) compound, stereoisomers, and veterinarily acceptable salt thereof, and a veterinary acceptable excipient, for the manufacture of a medicament.

In yet another aspect of the invention is a method for treating a parasitic infection in an animal that includes the step of administering to said animal, in need of such treatment, a therapeutically effective amount of a Formula (1A), Formula (1B), or Formula (1C) compound of the invention, stereoisomers thereof, and/or or veterinarily acceptable salt thereof. In yet another aspect of the invention is a method for treating a parasitic infection in an animal that includes the step of administering to said animal, in need of such treatment, a therapeutically effective amount of a Formula (1A), Formula (1B), or Formula (1C) compound of the invention, stereoisomers thereof, and veterinarily acceptable salt thereof, and a veterinary acceptable excipient. In one aspect, the animal is a mammal, specifically a companion animal (for example, dog, cat, or horse) or livestock (for example, sheep, goat, cattle, and pig). In another aspect, the animal is a bird, specifically, fowl (for example, chicken, turkey, duck, and geese). In another aspect, the animal is a fish. The compounds of the invention, and compositions thereof, can be administered to the animal orally, topically, or by injection, for example intramuscular, intravenous, and subcutaneous injection.

In yet another aspect of the invention is a method for treating a parasitic infection in an animal that includes the step of administering to said animal, in need of such treatment, a therapeutically effective amount of a Formula (1A), Formula (1B), or Formula (1C) compound of the invention, stereoisomers thereof, and/or veterinarily acceptable salt thereof, in combination with at least one additional veterinary agent. The Formula (1A), Formula (1B), or Formula (1C) compound can be administered with at least one additional veterinary agent by administering them a) together in a single veterinary composition; or (b) two or more separate veterinary compositions comprising (i) a first composition comprising a Formula (1A), Formula (1B), or Formula (1C) compound of the invention, stereoisomers thereof, veterinarily acceptable salt thereof, and a veterinarily acceptable excipient, and (ii) a second composition comprising at least one additional veterinary agent, as described herein and a veterinarily acceptable excipient, and optionally, (iii) a third (or more) composition comprising at least one additional veterinary agent, as described herein and a veterinarily acceptable excipient. The veterinary compositions may be administered simultaneously or sequentially and in any order.

In yet another aspect of the invention, is a method of administering a composition comprising a compound of Formula (1A), (1B), or (1C), stereoisomers thereof, and veterinary acceptable salts thereof, to an inimal in need thereof, wherein the composition is administered topically, orally, or parenterally, for example, intravenously, intramuscularly, or subcutaneously.

### DEFINITIONS

For purposes of the invention, as described and claimed herein, the following terms and phrases are defined as follows:

“Additional veterinary agent(s)” as used herein, unless otherwise indicated, refers to other veterinary or pharmaceutical compounds or products that provide a therapeutically effective amount of said agents that are useful for the treatment of a parasitic infection in an animal, as described herein.

“Alkoxy”, as used herein, unless otherwise indicated, refers to an oxygen moiety having a further alkyl substituent. The alkyl portion (i.e., alkyl moiety) of an alkoxy group has the same definition as below. Non-limiting examples include: methoxy, ethoxy, and the like.

“Alkyl”, as used herein, unless otherwise indicated, refers to saturated monovalent hydrocarbon alkane radicals of the general formula CₙH₂ₙ₊₁. The alkane radical may be straight or branched and may be unsubstituted or substituted. For example, the term “(C₁-C₆)alkyl” refers to a monovalent, straight or branched aliphatic group containing 1 to 6 carbon atoms. Non-exclusive examples of (C₁-C₆) alkyl groups include, but are not limited to methyl, ethyl, propyl, isopropyl, sec-butyl, t-butyl, n-propyl, n-butyl, i-butyl, s-butyl, n-pentyl, 1-methylbutyl, 2-methylbutyl, 3-methylbutyl, neopentyl, 3,3-dimethylpropyl, 2-methylpentyl, hexyl, and the like. The alkyl moiety may be attached to the chemical moiety by any one of the carbon atoms of the aliphatic chain. Alkyl groups are optionally substituted as described herein. Further when used in compound words such as alkylaryl, said alkyl moiety has the same meaning as herein defined and may be attached to the chemical moiety by any one of the carbon atoms of the aliphatic chain. Non-limiting examples of the compound word, alkylaryl include: C₁alkylaryl is —C-aryl, C₂alkylaryl is —C—C-aryl, C₀aryl is aryl (i.e., wherein aryl is as defined herein, for example, phenyl, naphthyl, and the like). Non-limiting examples of the compound word, alkylheteroaryl include: C₁alkylheteroaryl is —C-heteroaryl, C₂alkylheteroaryl is —C—C-heteroarylaryl, C₀heteroaryl is heteroaryl (i.e., wherein heteroaryl is as defined herein, for example, pyridinyl, thiazolyl, quinolinyl, and the like). The aryl and heteroaryl moieties are optionally substituted as described herein.

“Alkenyl” as used herein, unless otherwise indicated, refers to a straight or branched aliphatic hydrocarbon chain having 2- to 6-carbon atoms and containing at least one carbon-carbon double bond (for example —C═C—, or —C—C═C—, and the like). Non-exclusive examples of alkenyl include: ethenyl, 1-propenyl, 2-propenyl, isopropenyl, 1-butenyl, 2-butenyl, 3-butenyl, 2-pentenyl, and the like. Further when used in compound words such as alkenylaryl, said alkenyl moiety has the same meaning as herein defined and may be attached to the chemical moiety by any one of the carbon atoms of the aliphatic chain. Non-limiting examples of the compound word, alkenylaryl include: C₂alkenylaryl is —C═C-aryl, C₃alkenylaryl is —C—C═C-aryl (i.e., wherein aryl is as defined herein, for example phenyl, naphthyl, and the like). Non-limiting examples of the compound word, alkenylheteroaryl include: C₂alkenylheteroaryl is —C═C-heteroaryl, C₃alkenylheteroaryl is —C—C═C-heteroaryl (i.e., wherein heteroaryl is as defined herein, for example pyridinyl, thiazolyl, quinolinyl, and the like). The aryl and heteroaryl moieties are optionally substituted as described herein.

“Alkynyl” as used herein, unless otherwise indicated, refers to straight or branched aliphatic hydrocarbon chain having 2- to 6-carbon atoms and containing at least one carbon-carbon triple bond (for example, —C≡C— or —C—C≡C—, and the like). Non-exclusive examples of alkynyl include: ethynyl, 2-propynyl, 1-methyl-2-propynyl, 2-butynyl, 3-butynyl, 2-methyl-3-butynyl, and the like.

“Animal(s)”, as used herein, unless otherwise indicated, refers to an individual animal that is a mammal, bird, or fish. Specifically, mammal refers to a vertebrate animal that is human and non-human, which are members of the taxonomic class Mammalia. Non-exclusive examples of non-human mammals include companion animals and livestock. Non-exclusive examples of a companion animal include: dog, cat, and horse. A preferred companion animal is a dog. Another preferred companion animal is a cat. Another preferred companion animal is a horse. Non-exclusive examples of livestock include: swine, camel, rabbits, goat, sheep, deer, elk, cattle, and bison. Preferred livestock is cattle and swine. Specifically, bird refers to a vertebrate animal of the taxonomic class Ayes. Birds are feathered, winged, bipedal, endothermic, and egg-laying. Non-exclusive examples of bird include, poultry (e.g., chicken, turkey, duck, and geese), all of which are also referred to herein as fowl. Specifically, fish refers to the taxonomic class Chondrichthyes (cartilaginous fishes, e.g., sharks and rays) and Osteichthyes (bony fishes) which live in water, have gills or mucus-covered skin for respiration, fins, and may have scales. Non-exclusive examples of fish include shark, salmon, trout, whitefish, catfish, tilapia, sea bass, tuna, halibut, turbot, flounder, sole, striped bass, eel, yellowtail, grouper, and the like.

“Aryl”, as used herein, unless otherwise indicated, refers to a 5- to 7-membered aromatic monocyclic or 8-10 membered fused aromatic bicyclic ring structure. Examples of aryl include phenyl and naphthyl. The aryl group may be attached to the chemical moiety by any one of the carbon atoms within the monocyclic or fused ring. Further when used in compound words such as alkylaryl, said alkyl and aryl moiety have the same meaning as herein defined and may be attached to the chemical moiety by any one of the carbon atoms of the aliphatic chain. For example, C₀alkylaryl is aryl, C₁alkylaryl is —C-aryl, C₂alkylaryl is —C—C-aryl, C₂alkenylaryl is —C═C-aryl, and the like. The aryl moieties are optionally substituted as described herein.

“Carbocyclic” (“carbocycle”), as used herein, unless otherwise indicated, refers to a partially saturated or saturated 5- to 6-membered ring containing only carbon atoms and can be monocyclic or part of a fused ring or spiro ring moiety. Examples of carbocyclic rings include cyclopentane, cyclopentene, cyclohexane, and cyclohexene. The carbocyclic moieties are optionally substituted as described herein.

“Chiral”, as used herein, unless otherwise indicated, refers to the structural characteristic of a molecule that makes it impossible to superimpose it on its mirror image, (e.g., “R” and “S” enantiomers). The term is sometimes depicted as an asterisk (i.e.,*) in the Examples and preparations which refers to the chiral center which includes both the S and R enantiomers.

“Compounds of the invention”, as used herein, unless otherwise indicated, refers to Formula (1A), Formula (1B), or Formula (1C) compounds, stereoisomers thereof, and veterinarily acceptable salts thereof.

“Cycloalkyl”, as used herein, unless otherwise indicated, includes fully saturated or partially saturated 3- to 6-membered monocyclic rings containing carbon atoms. Non-limiting examples of partially saturated cycloalkyls include: cyclopropene, cyclobutene, cycloheptene, cyclohexene, cyclohepta-1,3-diene, cyclohexa-1,3-diene, and the like. Saturated cycloalkyls include cyclopropyl, cyclobutyl, cyclopentyl, and cyclohexyl. The cycloalkyl group may be attached to the chemical moiety by any one of the carbon atoms within the carbocyclic ring. Cycloalkyl groups are optionally substituted with at least one substituent. Further when used in compound words such as alkylcycloalkyl, said alkyl and cycloalkyl moiety has the same meaning as herein defined and may be attached to the chemical moiety by any one of the carbon atoms of the aliphatic chain. For example, C₀alkylcycloalkyl is cycloalkyl (for example, cyclopropyl, cyclobutyl, cyclopentyl, cyclohexyl), C₁alkylcycloalkyl is CH₂cycloalkyl (for example (—C-cyclopropyl, —C-cyclpentyl, and the like), C₂alkylcycloalkyl is —C—C-cyclopropyl, —C—C-cyclobutyl, and the like). The cycloalkyl moieties are optionally substituted as described herein.

“E/Z Notation” or “E and Z geometric (diastereomer) isomer(s)”, as used herein, unless otherwise indicated, refers to the International Union of Pure and Applied Chemistry (IUPAC) preferred method of describing the stereochemistry of double bonds in organic chemistry. It is an extension of cis/trans notation that can be used to describe double bonds having three or four substituents. Following a set of defined rules (Cahn-Ingold-Prelog priority rules), each substituent on a double-bond is assigned a priority. If the two groups of higher priority are on opposite sides of the double bond, the bond is assigned the configuration E (from entgegen, the German word for “opposite”). If the two groups of higher priority are on the same side of the double bond, the bond is assigned the configuration Z (from zusammen, the German word for “together”). Therefore, each of the respective “Z” isomers of the “E” isomers described herein, are contemplated in this invention.

“Halogen” or “halo”, as used herein, unless otherwise indicated, refers to fluorine, chlorine, bromine and iodine. Further, when used in compound words such as “haloalkyl”, “haloalkoxy”, “haloalkenyl”, or “haloalkynyl”, said alkyl, alkoxy, alkenyl, and alkynyl may be partially or fully substituted with halogen atoms which may be the same or different and said alkyl, alkoxy, alkenyl, and alkynyl moiety has the same meaning as above and may be attached to the chemical moiety by any one of the carbon atoms of the aliphatic chain. Examples of “haloalkyl” include F₃C—, ClCH₂—, CF₃CH₂— and CF₃CCl₂—, and the like. The term “haloalkoxy” is defined analogously to the term “haloalkyl”. Examples of “haloalkoxy” include CF₃O—, CCl₃CH₂O—, HCF₂CH₂CH₂O— and CF₃CH₂O—, and the like. The term “haloalkenyl is also defined analogously to the term “haloalkyl” except that the aliphatic chain contains at least one carbon-carbon double bond. Examples of “haloalkenyl” include CF₃—C═C—, Cl₃C—C═C—, HF₂C—C═C— and F₃C—C═C—, and the like. The term “haloalkynyl” is also defined analogously to the term “haloalkyl” except that the aliphatic chain contains at least one carbon-carbon triple bond. Examples of “haloalkynyl” include F₃C—C≡C—, Cl₃C—C≡C—, HF₂C—C≡C—, and the like.

“Heteroaryl”, as used herein, unless otherwise indicated, refers to a 5- to 7-membered monocyclic aromatic ring or an 8- to 10-membered fused bicyclic aromatic ring where said monocyclic- and fused bicyclic-ring moiety contains one or more heteroatoms each independently selected from N, O, or S, preferably from one to four heteroatoms. Non-exclusive examples of monocyclic heteroaryls include pyrrolyl, furanyl, thiophenyl, pyrazolyl, imidazolyl, triazolyl, tetrazolyl, thiazolyl, isoxazolyl, oxazolyl, oxadiazolyl, thiadiazolyl, pyridinyl, pyridazinyl, pyrimidinyl, pyrazinyl, and the like. Non-exclusive examples of fused bicyclic heteroaryls include: benzofuranyl, benzothiophenyl, indolyl, benzimidazolyl, indazolyl, benzotriazolyl, thieno[2,3-c]pyridine, thieno[3,2-b]pyridine, benzo[1,2,5]thiadiazole, quinolinyl, isoquinolinyl, and the like. The heteroaryl group may be attached to the chemical moiety by any one of the carbon atoms or nitrogen heteroatoms within the monocyclic or fused ring. Further when used in compound words such as alkylheteroaryl, said alkyl and heteroaryl moiety have the same meaning as herein defined and may be attached to the chemical moiety by any one of the carbon atoms of the aliphatic chain. For example, C₀alkylheteroaryl is heteroaryl, C₁alkylheteroaryl is —C-heteroaryl, C₂alkylheteroaryl is —C—C-heteroaryl, C₂alkenylheteroaryl is —C═C— heteroaryl and the like. The heteroaryl moieties are optionally substituted as described herein.

“Heterocycle”, as used herein, unless otherwise indicated, refers to a partially saturated or saturated 3- to 7-membered monocyclic ring containing one or more heteroatoms each independently selected from N, O, or S, preferably from one to four heteroatoms. Non-exclusive examples of heterocycle include oxirane, thiarane, aziridine, oxetane, azetidine, thiatane, tetrahydrofuran, tetrahydrothiophene, pyrrolidine, tetrahydropyrane, piperidine, piperazine, tetrahydropyridine, 2H-azirine, 2,3-dihydro-azete, 3,4-dihydro-2H-pyrrole, and the like. The heterocycle group may be attached to the chemical moiety by any one of the carbon atoms or nitrogen heteroatoms within the ring. Further when used in compound words such as alkylheterocycle, said alkyl and heterocycle moiety have the same meaning as herein defined and may be attached to the chemical moiety by any one of the carbon atoms of the aliphatic chain. For example, C₀alkylheterocycle is heterocycle, C₁alkylheterocycle is —C-heterocycle, C₂alkylheterocycle is —C—C-heterocycle, and the like. The heterocycle moieties are optionally substituted as described herein.

“Optionally substituted”, is used herein interchangeably with the phrase substituted or unsubstituted. Unless otherwise indicated, an optionally substituted group may have a substituent at each substitutable position of the group, and each substitution is independent of the other. An optionally substituted group also may have no substituents. Therefore, the phrase “optionally substituted with at least one substituent” means that the number of substituents may vary from zero up to a number of available positions for substitution.

“Parasite(s)”, as used herein, unless otherwise indicated, refers to endoparasites and ectoparasites. Endoparasites are parasites that live within the body of its host and include helminths (e.g., trematodes, cestodes, and nematodes) and protozoa. Ectoparasites are organisms of the Arthropoda phylum (e.g., arachnids (e.g., ticks, and mites), insects (e.g., mosquitoes, fleas, lice, midges, and biting flies), and crustaceans (e.g., copepods-sea lice) which feed through or upon the skin of its host.

“Therapeutically effective amount”, as used herein, unless otherwise indicated, refers to an amount of the compounds of the invention that (i) treat the particular parasitic infection or infestation, (ii) attenuates, ameliorates, or eliminates one or more symptoms of the particular parasitic infection or infestation, or (iii) prevents or delays the onset of one or more symptoms of the particular parasitic infection or infestation described herein.

“Treatment”, “treating”, and the like, as used herein, unless otherwise indicated, refers to reversing, alleviating, or inhibiting the parasitic infection, infestation, or condition. As used herein, these terms also encompass, depending on the condition of the animal, preventing the onset of a disorder or condition, or of symptoms associated with a disorder or condition, including reducing the severity of a disorder or condition or symptoms associated therewith prior to affliction with said infection or infestation. Thus, treatment can refer to administration of the compounds of the invention to an animal that is not at the time of administration afflicted with the infection or infestation. Treating also encompasses preventing the recurrence of an infection or infestation or of symptoms associated therewith as well as references to “control” (e.g., kill, repel, expel, incapacitate, deter, eliminate, alleviate, minimize, and eradicate).

“Veterinary acceptable” as used herein, unless otherwise indicated, indicates that the substance or composition must be compatible chemically and/or toxicologically, with the other ingredients comprising a formulation, composition, and/or the animal being treated therewith. The term also contemplates “pharmaceutical or pharmaceutically” acceptable.

### DETAILED DESCRIPTION

The invention provides Formula (1A), Formula (1B), or Formula (1C) compounds, stereoisomers thereof, including enantiomers, (E) and (Z) geometric isomers, and diastereomers, veterinarily acceptable salts thereof, as well as veterinary compositions that are useful as antiparasitic agents for animals, in particular, compounds that act as ectoparasiticides and endoparasiticides.

Compounds of the invention may be synthesized by synthetic routes that include processes analogous to those well known in the chemical arts, particularly in light of the description contained herein. The starting materials are generally available from commercial sources such as Aldrich Chemicals (Milwaukee, Wis.) or are readily prepared using methods well known to those skilled in the art (e.g., prepared by methods generally described in Louis F. Fieser and Mary Fieser, “Reagents for Organic Synthesis”, 1; 19, Wiley, New York (1967, 1999 ed.), or Beilsteins Handbuch der organischen Chemie, 4, Aufl. ed. Springer-Verlag, Berlin, including supplements (also available via the Beilstein online database)). For illustrative purposes, the reaction schemes depicted below demonstrate potential routes for synthesizing compounds of the invention, and key intermediates. For a more detailed description of the individual reaction steps, see the Examples section below. A skilled artisan will appreciate that other suitable starting materials, reagents, and synthetic routes may be used to synthesize the compounds of the invention and a variety of derivatives thereof. Further, many of the compounds prepared by the methods described below can be further modified in light of this disclosure using conventional chemistry well known to the skilled artisan.

Compounds of the invention described herein exist as stereoisomers. The E and Z configurations are based upon knowledge of known geometric chemistry. Unless specified otherwise, it is intended that all isomeric forms of the compounds of the invention as well as mixtures thereof, including racemic mixtures, form part of the invention.

Isomeric mixtures can be separated into their individual enantiomers on the basis of their physical chemical differences by methods well known to those skilled in the art, such as chromatography and/or fractional crystallization. A more detailed description of techniques that can be used to resolve stereoisomers of compounds from their racemic mixture can be found in Jean Jacques Andre Collet, Samuel H. Wilen, Enantiomers, Racemates and Resolutions, John Wiley and Sons, Inc. (1981).

Compounds of this invention can exist as one or more stereoisomers. The various stereoisomers include enantiomers, diastereomers and atropisomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. The compounds of the invention may be present as a mixture of stereoisomers, individual stereo isomers or as an optically active form.

For illustrative purposes, the reaction schemes depicted below demonstrate potential routes for synthesizing key intermediates and compounds of the invention. For a more detailed description of the individual reaction steps, see the Examples section below. Those skilled in the art will appreciate that other suitable starting materials, reagents, and synthetic routes may be used to synthesize the intermediates and compounds of the invention and a variety of derivatives thereof. Further, many of the compounds prepared by the methods described below can be further modified in light of this disclosure using conventional chemistry. Schemes 1-3 outline the general procedures useful for the preparation and isolation of compounds of the invention. It is to be understood, however, that the invention, as fully described herein and as recited in the claims, is not intended to be limited by the details of the following schemes or modes of preparation.

In the preparation of compounds of the invention, protection of remote functionality of intermediates from undesired reactions can be accomplished with a protecting group. The term “protecting group” or “Pg” refers to a substituent that is commonly employed to block or protect a particular functionality while reacting other functional groups on the compound. For example, an amine-protecting group is a substituent attached to an amine that blocks or protects the amine-functionality of the compound or intermediate. Suitable amine protecting groups include: 1-tert-butyloxycarbonyl (Boc), acyl groups including: formyl, acetyl, chloroacetyl, trichloro-acetyl, o-nitrophenylacetyl, o-nitrophenoxyacetyl, trifluoroacetyl, acetoacetyl, 4-chlorobutyryl, isobutyryl, o-nitrocinnamoyl, picolinoyl, acylisothiocyanate, aminocaproyl, benzoyl, and the like; and acyloxy groups including: methoxycarbonyl, 9-fluorenyl-methoxycarbonyl, 2,2,2-trifluoroethoxycarbonyl, 2-trimethylsilylethxoycarbonyl, vinyloxycarbonyl, allyloxycarbonyl, 1,1-dimethyl-propynyloxycarbonyl, benzyloxy-carbonyl, p-nitrobenzyloxycarbony, 2,4-dichlorobenzyloxycarbonyl, and the like. Similarly, diphenylmethane and benzylcarbamates can be used as amine protecting groups. Suitable protecting groups and their respective uses are readily determined by one skilled in the art. For a general description of protecting groups and their use, see T. W. Greene, Protective Groups in Organic Synthesis, John Wiley & Sons, New York, 1991.

In the Schemes (1-3) and Examples (1-189) below, the following catalysts/reactants and miscellaneous abbreviations include: mobile phase (MP); retention time (r.t.); room temperature (RT); equivalent (eq); approximately (˜); round bottom flask (RBF); N,N-dimethyl formamide (DMF); acetonitrile (ACN or Acn); triethylamine (TEA or Et₃N); trifluoroacetic acid (TFA); ethanol (EtOH), methanol (MeOH), isopropyl magnesium chloride (iPrMgCl); dichloromethane (DCM); palladium (Pd); trichloromethane or chloroform (CHCl₃); ammonium hydroxide (NH₄OH); diethyl ether (Et₂O); methyl tertiary butyl ether (MTBE); sodium hydroxide (NaOH); hydrochloric acid (HCl); sodium hydride (NaH); Trideuterio(trideuteriomethylsulfinyl)methane or ((methyl-d3)sulfinyl)methane-d3 (DMSO-d6); magnesium sulfate (MgSO₄); dimethylsulfoxide (DMSO); tetrahydrofuran (THF); sodium bicarbonate (NaHCO₃); potassium acetate (KOAc); ethyl acetate (EtOAc); triphenylphosphorane (Ph₃P); sodium sulfate (Na₂SO₄); nitrogen gas (N₂); 1,2-dichloro ethane (DCE); acetic acid (AcOH); silica cyanoborohydride (Si—CBH); azobisisobutyronitrile (AIBN); potassium fluoride (KF); [1,1′-Bis(diphenyl-phosphino)ferrocene]dichloropalladium(II) (PdCl₂(dppf)₂); and tert-butyloxycarbonyl (boc protecting group).

### Schemes

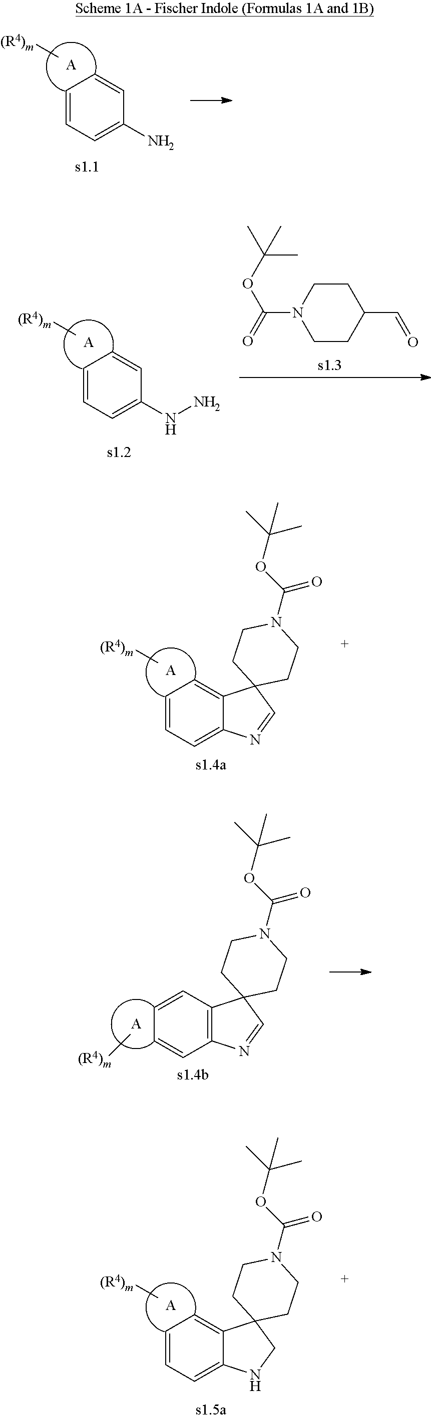

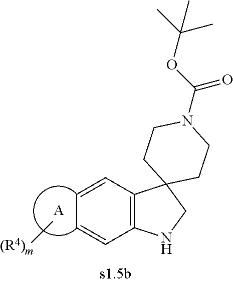

Scheme 1A describes the Fischer indole synthesis scheme. A and R⁴, and n are as defined herein. The ratio of isomeric products (analogs) varies depending on the bicyclic starting material.

The commercially available aniline (s1.1) is converted to the corresponding hydrazine (s1.2) by diazotization at 0° C. using nitrous acid (prepared in situ from sodium nitrite and a mineral acid, typically hydrochloric acid), the intermediate diazonium salt is then reduced without isolation using stannous chloride dihydrate in concentrated hydrochloric acid at 0° C. Cyclization to the 3H-indole (s1.4a and s1.4b) is accomplished by the Fischer indole synthesis of the hydrazine (s1.2) and the commercially available boc-protected 4-formylpiperidine (s1.3) in chloroform with catalytic ethanol and trifluoroacetic acid at 0° C. Reduction to the indoline (s1.4a and s1.4b) is done using sodium borohydride in ethanol at room temperature to afford s1.5a and s1.5b.

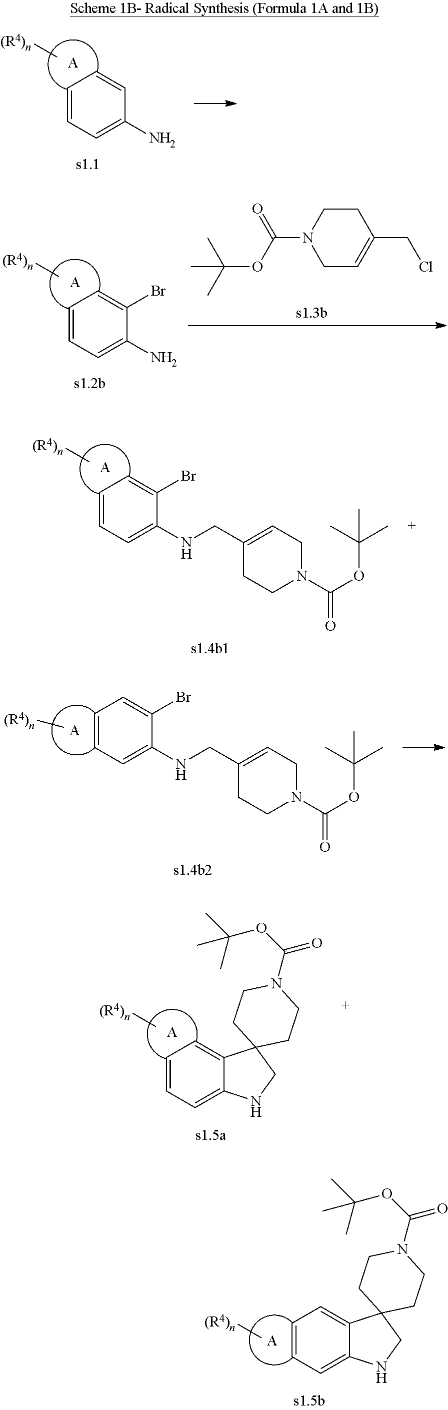

Scheme 1B describes the radical synthesis scheme. A and R4, and n are as defined herein. The ratio of isomeric products (analogs) varies depending on the bicyclic starting material.

The commercially available aniline (s1.1) is converted to the corresponding brominated aniline (s1.2b) by bromination at 0° C. using N-bromosuccinamide in a polar solvent such as acetonitrile. Cyclization to the indoline (s1.5a and s1.5b) is accomplished by firstly alkyation of arylbromide (s1.2b) with commercially available tert-butyl 4-(chloromethyl)-5,6-dihydropyridine-1(2H)-carboxylate (s1.3b) using typically sodium hydride and a polar aprotic solvent such as DMF, followed secondly by radical cyclization of alkene (s.1.4b1 and s.1.4b2) using azobisisobutyronitrile and tributyltin hydride in toluene under elevated temperatures such as 90° C. to afford indolines s1.5a and s1.5b.

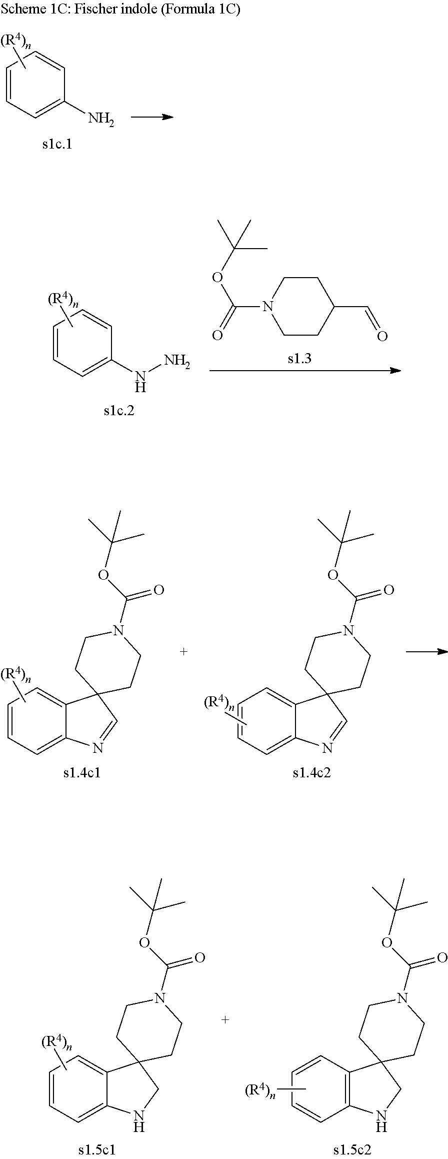

Scheme 10 describes the Fischer indole synthesis scheme. R⁴ and n are as defined herein. The ratio of isomeric products (analogs) varies depending on the bicyclic starting material.

The commercially available aniline (s1c.1) is converted to the corresponding hydrazine (s1c.2) by diazotization at 0° C. using nitrous acid (prepared in situ from sodium nitrite and a mineral acid, typically hydrochloric acid), the intermediate diazonium salt is then reduced without isolation using stannous chloride dihydrate in concentrated hydrochloric acid at 0° C. Cyclization to the 3H-indole (s1.4c1 and s1.4c2) is accomplished by the Fischer indole synthesis of the hydrazine (s1c.2) and the commercially available boc-protected 4-formylpiperidine (s1.3) in chloroform with catalytic ethanol and trifluoroacetic acid at 0° C. Reduction to the indoline is done using sodium borohydride in ethanol at room temperature to afford s1.5c1 and s1.5c2.

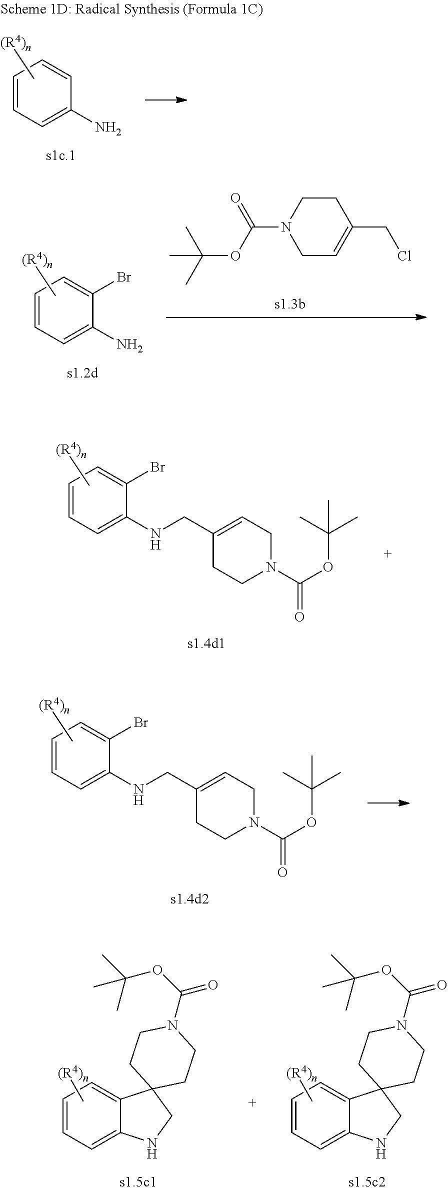

Scheme 1D describes the radical synthesis scheme. R4 and n are as defined herein. The ratio of isomeric products (analogs) varies depending on the bicyclic starting material.

The commercially available aniline (s1c.1) is converted to the corresponding brominated aniline (s1.2d) by bromination at 0° C. using N-bromosuccinamide in a polar solvent such as acetonitrile. Cyclization to the indoline (s1.4d1 and s1.4d2) is accomplished by firstly alkyation of arylbromide (s1.2d) with commercially available tert-butyl 4-(chloromethyl)-5,6-dihydropyridine-1(2H)-carboxylate (s1.3b) using typically sodium hydride and a polar aprotic solvent such as DMF, followed secondly by radical cyclization of alkene (s1.4d1 and s1.4d2) using azobisisobutyronitrile and tributyltin hydride in toluene under elevated temperatures such as 90° C. to afford indolines s1.5c1 and s1.5c2.

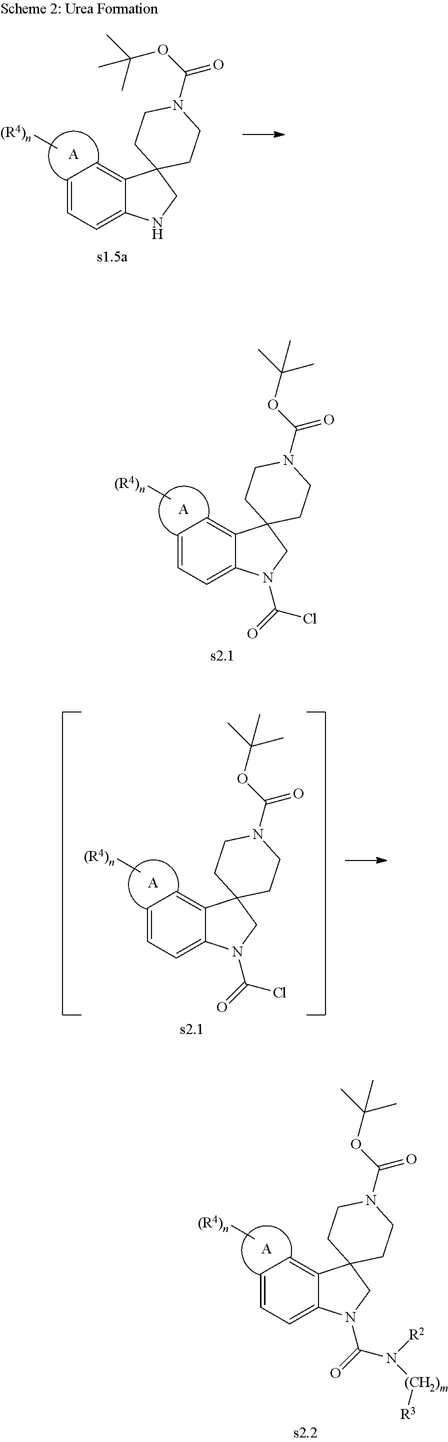

A, R2, R3, R4, n and m are as defined herein. The ureas (s2.2) are synthesized by formation of an intermediate carbamoyl chloride (s2.1) using phosgene, and pyridine or potassium carbonate or triethylamine as base in dichloromethane or DCE as solvent at 0° C. followed by addition of a commercially available amine at 0° C. and then warming to room temperature. Similarly, the s1.4b isomer can be used to prepare the urea isomer.

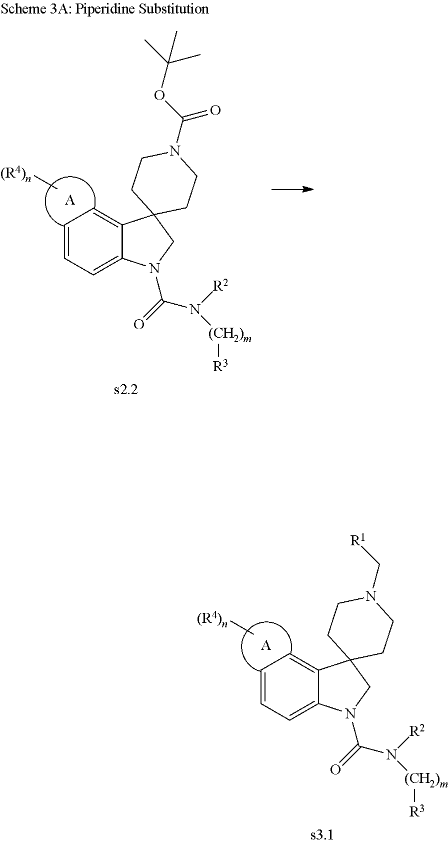

A, R¹, R², R³, (R⁴), n, and m are as defined herein. Deprotection of the BOC group on s2.2 is accomplished by treatment with trifluoroacetic acid in dichloromethane at room temperature. This amine was then alkylated using a reductive amination procedure with either a readily prepared intermediate (Intermediates 1.17 or 1.18) or a commercially available aldehyde. Mixing of the amine salt and the aldehyde with sodium triacetoxyborohydride and triethylamine in an organic solvent (typically dimethylformamide or dichloromethane) at room temperature affords the piperidine substitute s3.1. Similarly, the s1.5b isomer can be used to prepare the other respective isomer. These products can also be prepared by direct alkylation of the amine using a standard alkylating agent (e.g., alkyl halide, triflate, sulphate, tosylate, or any other known leaving group) in the presence of a base in an organic solvent.

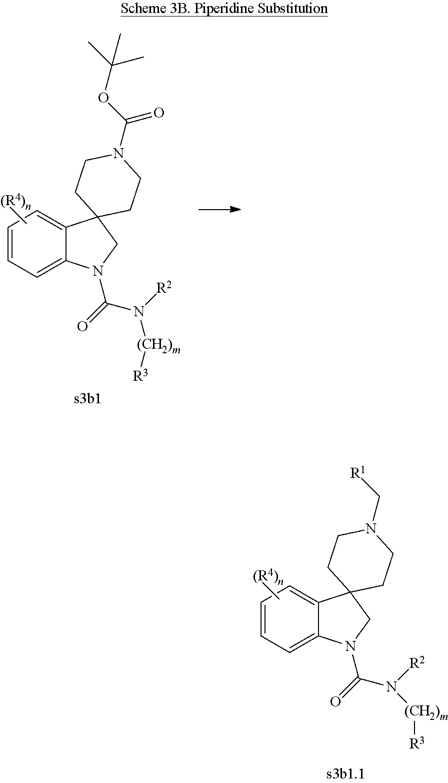

R¹, R², R³, (R⁴), n, and m are as defined herein. Deprotection of the BOC group on s3b1 is accomplished by treatment with trifluoroacetic acid in dichloromethane at room temperature. This amine was then alkylated using a reductive amination procedure with either a readily prepared intermediate (Intermediates 1.17 or 1.18) or a commercially available aldehyde. Mixing of the amine salt and the aldehyde with sodium triacetoxyborohydride and triethylamine in an organic solvent (typically dimethylformamide or dichloromethane) at room temperature affords the piperidine substituted s3b1.1. These products can also be prepared by direct alkylation of the amine using a standard alkylating agent (e.g., alkyl halide, triflate, sulphate, tosylate, or any other known leaving group) in the presence of a base in an organic solvent.

One skilled in the art will recognize that, in some cases, after the introduction of a given reagent as it is depicted in the schemes, it may be necessary to perform additional routine synthetic steps not described in detail to complete the synthesis of Formula (1A), Formula (1B), or Formula (1C) compounds.

The invention includes all veterinarily acceptable isotopically-labelled Formula (1A), Formula (1B), or Formula (1C) compounds wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, and sulphur, such as ³⁵S.

The skilled person will appreciate that the compounds of the invention could be made by methods other than those herein described as incorporated herein by reference, by adaptation of the methods herein described and/or adaptation of methods known in the art, for example the art described herein, or using standard textbooks such as “Comprehensive Organic Transformations—A Guide to Functional Group Transformations”, RC Larock, Wiley-VCH (1999 or later editions).

The Formula (1A), Formula (1B), and Formula (1C) compounds are useful as antiparasitic agents, therefore, another aspect of the invention is a veterinary composition comprising a therapeutically effective amount of a Formula (1A) or (1B) compound, stereoisomers thereof, and a veterinarily acceptable excipient. The compounds (Formula (1A), Formula (1B), and Formula (1C)) of the invention (including the compositions and processes used therein) may also be used in the manufacture of a medicament for the therapeutic applications described herein.

The compound of the invention can be administered alone or in a formulation appropriate to the specific use envisaged, the particular species of host animal being treated and the parasite involved. Generally, it will be administered as a formulation in association with one or more veterinarily acceptable excipients. The term “excipient”, is used herein to describe any ingredient other than the compound of the invention or any additional veterinary (e.g., antiparasitic) agent. The choice of excipient(s) will to a large extent depend on factors such as the particular mode of administration, the effect of the excipient(s) on solubility and stability, and the nature of the dosage form. In addition to the excipient(s), the amount of the compound of the invention that is administered and the dosage regimen for treating a condition or disorder with the compound depends on a variety of factors, including the age, weight, sex and medical condition of the animal, the severity of the disease, the route and frequency of administration, and thus may vary widely.

In one aspect, the veterinary composition comprises a Formula (1A), Formula (1B), or Formula (1C) compound with a veterinary acceptable excipient. The concentration range will vary depending on the composition (e.g., oral, topical, or injectable). For an oral dose, the range of active (i.e., compound of the invention) is about 0.1 to 50 mg/kg, preferably from about 0.5 to 25 mg/kg, and even more preferably from about 0.5 to 10 mg/kg, and most preferably from about 1 to 5 mg/kg. For a topical solution, the range of active is about 0.1 to 1000 mg/mL, and preferably from about 0.5 to 500 mg/mL, and more preferably from about 1 to 250 mg/mL, and even more preferably from about 2 to 200 mg/mL. Depending upon the final volumes of the topical solution(s), the concentration of the active can change from that described above. Generally, injectable doses tend to be, but not always, lower in concentration.

The formulations can be prepared using conventional dissolution and mixing procedures. Such compositions and methods for their preparation may be found, for example, in ‘Remington's Veterinary Sciences’, 19th Edition (Mack Publishing Company, 1995; and “Veterinary Dosage Forms: Tablets, Vol. 1”, by H. Lieberman and L. Lachman, Marcel Dekker, N.Y., 1980 (ISBN 0-8247-6918-X).

A typical formulation is prepared by mixing a Formula (1A), Formula (1B), or Formula (1C) compound with at least one veterinary acceptable excipient. Suitable excipients are well known to those skilled in the art and include materials such as carbohydrates, waxes, water soluble and/or swellable polymers, hydrophilic or hydrophobic materials, gelatin, oils, solvents, water, and the like. The particular excipient(s) will depend upon the means and purpose for which the compound of the invention is being applied. Solvents are generally selected based on solvents recognized by persons skilled in the art as safe to be administered to an animal. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the invention or veterinary composition thereof) or aid in the manufacturing of the veterinary product (i.e., medicament). The compound of the invention will typically be formulated into veterinary dosage forms to provide an easily controllable dosage form for administration.

The Formula (1A), Formula (1B), or Formula (1C) compounds can be administered orally by capsule, bolus, tablet, powders, lozenges, chews, multi and nanoparticulates, gels, solid solution, films, sprays, liquid form, or admixed with food. Oral administration is the preferred method of administration and as such it is desirable to develop active Formula (1A), Formula (1B), and Formula (1C) compounds that are particularly suited to such formulations. Such formulations may be employed as fillers in soft or hard capsules, tablets, or soft or hard chews, and typically comprise a carrier, for example, pregelatinized starch, partially gelatinized starch, methylcellulose, carboxymethylcellulose, hydroxyethylcellulose, sugars (e.g., lactose (hydrous and anhydrous), glucose, sucrose, mannose, and the like); solvents, for example, water, ethanol, polyethylene glycol, N-methylpyrrolidone, propylene glycol, or a suitable oil (e.g., castor, peanut, almond, cotton, and the like), and the like; and one or more emulsifying agents and/or suspending agents. Tablets and chews may also be formulated with a flavor enhancer to increase palatability, for example, meat flavors (chicken, liver, beef, pork) that may be naturally based animal products or artificially derived meat flavorants. The flavors may also include vegetable, peanut butter, fruit, and other flavorants. Liquid forms include suspensions, solutions, syrups, drenches and elixirs. Liquid formulations may also be prepared by the reconstitution of a solid, for example, from a sachet. Oral drenches are commonly prepared by dissolving or suspending the active ingredient in a suitable medium. Feed admixtures can be prepared for livestock. Oral formulations can comprise from about 0.1 mg/kg to 50 mg/kg of a Formula (1A), Formula (1B), or Formula (1C) compound, and preferably about 0.5 mg/kg to 30 mg/kg. Depending upon the host specie treated and the parasite being treated, dose adjustments can be made.

The compounds may be administered topically to the skin or mucosa, that is dermally or transdermally. Typical formulations for this purpose include pour-on, spot-on, multi-spot-on, stripe-on, comb-on, roll-on, dip, spray, mousse, shampoo, powder formulation, gels, hydrogels, lotions, solutions, creams, ointments, dusting powders, dressings, foams, films, skin patches, wafers, implants, sponges, fibers, bandages and micro emulsions. Liposomes may also be used. Typical carriers include alcohol, water, mineral oil, liquid petrolatum, white petrolatum, glycerin, N-methyl formamide, glycol ethers, for example, ethylene glycol monomethyl ether, ethylene glycol monoethyl ether, diethylene glycol monomethyl ether, diethylene glycol monomethyl ether, and the like, dialkylethers, for example, ethylene glycol dimethyl ether, diethylene glycol dimethyl ether, and the like, polyethylene glycol, propylene glycol, and the like. Penetration enhancers may be incorporated—see, for example, J Pharm Sci, 88 (10), 955-958 by Finnin and Morgan (October 1999). Pour-on or spot-on formulations may be prepared by dissolving the active ingredients in an acceptable liquid carrier vehicle such as butyl digol, liquid paraffin or a non-volatile ester, optionally with the addition of a volatile component such as propan-2-ol or a glycol ether. Alternatively, pour-on, spot-on or spray formulations can be prepared by encapsulation, to leave a residue of active agent on the surface of the animal, this effect may ensure that the Formula (1A), Formula (1B), or Formula (1C) compound has increased persistence of action and are more durable, for example they may be more water fast. Topical formulations of the combination contemplated herein can comprise from about 0.5 mg/kg to 50 mg/kg of a Formula (1A), Formula (1B), or Formula (1C) compound, and preferably about 1 mg/kg to 10 mg/kg. The compositions suitable for spot-on application according to the invention can be prepared by conventional mixing means. The volume of the applied composition can be from about 0.1 mL/kg to 5 mL/kg and preferably from about 0.5 mL/kg to 3 mL/kg. Similarly, dose can be adjusted.

The compounds of the present invention can also be administered topically via a support matrix for example, a synthetic or natural resin, plastic, cloth, leather, or other such polymeric system in the shape of a collar or ear tag. Said collar or ear tag may be coated, impregnated, layered, by any means so as to provide a veterinarily or pharmaceutically acceptable amount of a compound of the present invention alone, or with a veterinarily or pharmaceutically acceptable excipient, diluent, or carrier, and optionally an additional veterinary agent, or veterinarily or pharmaceutically acceptable salt thereof.

Agents may be added to the formulations of the present invention to improve the persistence of such formulations on the surface of the animal to which they are applied, for example to improve their persistence on the coat of the animal. It is particularly preferred to include such agents in a formulation which is to be applied as a pour-on or spot-on formulation. Examples of such agents include acrylic copolymers and in particular fluorinated acrylic copolymers. A particular suitable reagent is the trademark reagent “Foraperle” (Redline Products Inc, Texas, USA). Certain topical formulations may include unpalatable additives to minimize oral exposure.

Injectable formulations may be prepared in the form of a sterile solution, which may contain other substances, for example enough salts or glucose to make the solution isotonic with blood. Acceptable liquid carriers include vegetable oils such as sesame oil, glycerides such as triacetin, esters such as benzyl benzoate, isopropyl myristate and fatty acid derivatives of propylene glycol, as well as organic solvents such as pyrrolidin-2-one and glycerol formal. The formulations are prepared by dissolving or suspending compounds of the present invention alone or with an additional veterinary agent in the liquid carrier such that the final formulation contains from about 0.01 to 50% by weight of the active ingredients, preferably from about 0.01% to about 10% by weight of the active ingredients.

Suitable devices for injection include needle (including micro needle) injectors, needle-free injectors and infusion techniques. Subcutaneous formulations are typically aqueous solutions which may contain excipients such as salts, carbohydrates and buffering agents (preferably to a pH of from 3 to 9), but, for some applications, they may be more suitably formulated as a sterile non-aqueous solution or as a dry powder form to be used in conjunction with a suitable vehicle such as sterile, pyrogen-free water. An injectable composition comprises a composition that is formulated for intravenous injection, intramuscular injection, or subcutaneous injection. The preparation of subcutaneous formulations under sterile conditions, for example, by lyophilisation, may readily be accomplished using standard veterinary techniques well known to those skilled in the art. The solubility of compounds of Formula (1A), Formula (1B), or Formula (1C) used in the preparation of subcutaneous solutions may be increased by the use of appropriate formulation techniques, such as the incorporation of solubility-enhancing agents.

Such formulations are prepared in a conventional manner in accordance with standard medicinal or veterinary practice. Further, these formulations will vary with regard to the weight of active compound contained therein, depending on the species of host animal to be treated, the severity and type of infection or infestation, and the body weight of the animal.

The Formula (1A), Formula (1B), and Formula (1C) compounds of the invention can be administered orally by capsule, bolus, tablet, powders, lozenges, chews, multi and nanoparticulates, gels, solid solution, films, sprays, or liquid form. This is a preferred method of administration and as such it is desirable to develop the compound for oral administration. Such formulations may be employed as fillers in soft or hard capsules, soft or hard palatable chews, which typically comprise an excipient, for example, water, ethanol, polyethylene glycol, N-methylpyrrolidone, propylene glycol, methylcellulose, or a suitable oil, and one or more emulsifying agents, flavorants, and/or suspending agents. Liquid forms include suspensions, solutions, syrups, drenches and elixirs. Liquid formulations may also be prepared by the reconstitution of a solid, for example, from a sachet. Oral drenches are commonly prepared by dissolving or suspending the compound of the invention in a suitable medium (e.g. triethylene glycol, benzyl alcohol, and the like). The compound of the invention can also be formulated with a food substance, e.g., a dietary admixture (food pellets or powder for birds).

The compound of the invention can be administered topically to the skin or mucosa, that is dermally or transdermally. This is another preferred method of administration and as such it is desirable to develop the compound of the invention to be suited to such formulations, for example liquid forms. Typical formulations for this purpose include pour-on, spot-on, multi-spot-on, stripe-on, comb-on, roll-on, dip, spray, mousse, shampoo, powder formulation, gels, hydrogels, lotions, solutions, creams, ointments, dusting powders, dressings, foams, films, skin patches, wafers, implants, sponges, fibers, bandages and micro emulsions. Liposomes may also be used. Typical excipients include alcohol, water, mineral oil, liquid petrolatum, white petrolatum, glycerin, N-methyl formamide, glycol monomethyl ethers, polyethylene glycol, propylene glycol, and the like. Penetration enhancers may be incorporated—see, for example, J Pharm Sci, 88 (10), 955-958 by Finnin and Morgan (October 1999). Pour-on or spot-on formulations may be prepared by dissolving the active ingredients in an acceptable liquid excipient such as butyl digol, liquid paraffin or a non-volatile ester, optionally with the addition of a volatile component such as propan-2-ol or a glycol ether. Alternatively, pour-on, spot-on or spray formulations can be prepared by encapsulation, to leave a residue of active agent on the surface of the animal, this effect may ensure that the compound of the invention has increased persistence of action and is more durable, for example it may be more water-fast. Topical formulations contemplated herein can comprise from about 0.1 mg/kg to 50 mg/kg of a compound of the invention, and more preferably from about 1 mg/kg to 10 mg/kg of a compound of the invention, and even more preferably, from 1 mg/kg to 5 mg/kg.

The Formula (1A), Formula (1B), and Formula (1C) compounds of the invention can also be administered topically via a support matrix for example, a synthetic or natural resin, plastic, cloth, leather, or other such polymeric system in the shape of a collar or ear tag. Said collar or ear tag may be coated, impregnated, layered, by any means so as to provide a veterinarily acceptable amount of a compound of the invention alone, or with a veterinarily acceptable excipient(s), and optionally an additional veterinary agent, or veterinarily acceptable salt thereof. Such formulations are prepared in a conventional manner in accordance with standard medicinal or veterinary practice. Further, these formulations will vary with regard to the weight of active compound contained therein, depending on the species of host animal to be treated, the severity and type of infection or infestation, and the body weight of the animal. The volume of the applied composition can be from about 0.2 mL/kg to 5 mL/kg and preferably from about 1 mL/kg to 3 mL/kg.

Agents may be added to the formulations of the invention to improve the persistence of such formulations on the surface of the animal to which they are applied, for example to improve their persistence on the coat of the animal. It is particularly preferred to include such agents in a formulation which is to be applied as a pour-on or spot-on formulation. Examples of such agents include acrylic copolymers and in particular fluorinated acrylic copolymers. A particular suitable reagent is the trademark reagent “Foraperle” (Redline Products Inc, Texas, USA). Certain topical formulations may include unpalatable additives to minimize oral exposure.

Injectable (e.g., subcutaneous and parenteral) formulations may be prepared in the form of a sterile solution, which may contain other substances, for example enough salts or glucose to make the solution isotonic with blood. Acceptable liquid excipients include vegetable oils such as sesame oil, glycerides such as triacetin, esters such as benzyl benzoate, isopropyl myristate and fatty acid derivatives of propylene glycol, as well as organic solvents such as pyrrolidin-2-one and glycerol formal. The formulations are prepared by dissolving or suspending compounds of the invention alone or with an additional veterinary agent in the liquid excipient such that the final formulation contains from about 0.01 to 30% by weight of the active ingredients.

Suitable devices for injectable administration include needle (including micro needle) injectors, needle-free injectors and infusion techniques. Injectable formulations are typically aqueous solutions which may contain excipients such as salts, carbohydrates and buffering agents (preferably to a pH of from 3 to 9), but, for some applications, they may be more suitably formulated as a sterile non-aqueous solution or as a dry powder form to be used in conjunction with a suitable vehicle such as sterile, pyrogen-free water. The preparation of injectable formulations under sterile conditions, for example, by lyophilisation, may readily be accomplished using standard veterinary techniques well known to those skilled in the art. The solubility of a compound of the invention used in the preparation of an injectable solution may be increased by the use of appropriate formulation techniques, such as the incorporation of solubility-enhancing agents.

Administration of the compound of the instant invention is contemplated to be once a month. However, an extended duration formulation may allow for dosing once every 2, 3, 4, 5, 6, or 12 months. Dosing of the compounds of the instant invention can also be daily, weekly, or at least once every two weeks.

Such formulations are prepared in a conventional manner in accordance with standard medicinal or veterinary practice. Further, these formulations will vary with regard to the weight of active compound contained therein, depending on the species of host animal to be treated, the severity and type of infection or infestation, and the body weight of the animal.

The composition of the invention may be administered alone, as described above, or in combination with at least one other additional antiparasitic agent to form a multi-component parasiticide giving an even broader spectrum of pharmaceutical and/or veterinary utility. Thus, the invention also envisions a combination veterinary composition comprising an effective amount of the compound of the invention in combination with at least one other additional antiparasitic agent and can further comprise at least one veterinarily acceptable excipient(s).

The following list of additional veterinary agents together with which the compound of the invention can be used is intended to illustrate the possible combinations, but not to impose any limitation. Non-limiting examples of additional veterinary agents include: amitraz, arylpyrazoles, amino acetonitriles, anthelmintics (e.g., albendazole, cambendazole, dichlorvos, fenbendazole, flubendazole, levamisole, mebendazole, monepantel, morantel, cyclic octadepsipeptides, oxantel, oxfendazole, oxibendazole, paraherquamide, parbendazole, piperazines, praziquantel, pyrantel, thiabendazole, tetramisole, triclabendazole, and the like), avermectins and derivatives thereof (e.g., abamectin, doramectin, emamectin, eprinomectin, ivermectin, moxidectin, selamectin, milbemycin, milbemycin oxime, and the like), DEET, demiditraz, diethylcarbamazine, fipronil, insect growth regulators (e.g., lufenuron, novaluron, hydroprene, kinoprene, methoprene, and the like), metaflumizone, niclosamide, nitenpyram, permethrin, pyrethrins, pyriproxyfen, spinosad, and the like. In certain instances, combinations of a compound of the invention with at least one additional veterinary agent can result in a greater-than-additive effect. Non-limiting examples of combinations include, but are not limited to: compound of the invention with pyrantel, compound of the invention with macrocyclic lactone, combination of the invention with macrocyclic lactone and levamisole, compound of the invention with macrocyclic lactone and pyrantel.

The veterinary composition for application to an animal may be packaged in a variety of ways depending upon the method used for administering the compound of the invention or combination, thereof. Generally, an article for distribution includes a container having deposited therein the veterinary composition in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The compounds of the invention (including the compositions and processes used therein) may also be used in the manufacture of a medicament for the therapeutic applications described herein.

The compounds of the invention, stereoisomers thereof, and compositions comprising a therapeutically effective amount of a Formula (1A) or Formula (1B) compound and a veterinarily acceptable excipient(s) are useful as ectoparasiticides for the control and treatment of infections or infestations manifested by said ectoparasite in an animal. The compounds of the invention have utility as an ectoparasiticide, in particular, as an acaricide and insecticide. They may, in particular, be used in the fields of veterinary medicine, livestock husbandry and the maintenance of public health: against acarids, insects, and copepods which are parasitic upon vertebrates, particularly warm-blooded vertebrates, including companion animals, livestock, and fowl and cold-blooded vertebrates like fish. Some non-limiting examples of ectoparasites include: ticks (e.g., Ixodes spp., (e.g., I. scapularis, I. ricinus, I. hexagonus), Rhipicephalus spp. (e.g., R. sanguineus), Boophilus spp., Amblyomma spp. (e.g., A. maculatum, A. triste, A. parvum, A. ovale, A. oblongoguttatum, A. aureolatum, A. cajennense, A. americanum), Hyalomma spp., Haemaphysalis spp., Dermacentor spp. (e.g., D. variabilis, D. andersoni, D. marginatus), Ornithodorus spp., and the like); mites (e.g., Dermanyssus spp., Cheyletiella spp., Sarcoptes spp. (e.g., S. scabiei), Psoroptes spp. (e.g., P. bovis), Otodectes spp., Chorioptes spp., Demodex spp., (e.g., D. folliculorum, D. canis, and D. brevis) and the like); chewing and sucking lice (e.g., Damalinia spp., Linognathus spp., Haematopinus spp., Solenoptes spp., Trichodectes spp., Felicola spp., and the like); fleas (e.g., Ctenocephalides spp., and the like); biting flies, midges, and mosquitoes (e.g., Tabanus spp., Haematobia spp., Musca spp., Stomoxys spp., Cochliomyia spp., Simuliidae spp., Ceratopogonidae spp., Psychodidae spp., Aedes spp., Culex spp., Anopheles spp., and the like); bed bugs (e.g., insects within the genus Cimex and family Cimicidae); grubs (e.g., Dermatobia spp., Hypoderma bovis, H. lineatum); and copepods (e.g., sea lice within the Order Siphonostomatoida, including genera Lepeophtheirus and Caligus).

Compounds of the invention can also be used for the treatment of endoparasites, for example, cestodes (tapeworms), nematodes (round worms), and trematodes (flukes). Non-exlusive examples of the nematodes include roundworms, hookworms, whipworms, and heart worms. Non-exclusive examples of the gastrointestinal roundworms include: Ostertagia ostertagi (including inhibited larvae), O. lyrata, Haemonchus placei, H. similis, H. contortus, Toxascaris leonine, Toxocara canis, T. cati, Trichostrongylus axei, T. colubriformis, T. longispicularis, Cooperia oncophora, C. pectinata, C. punctata, C. surnabada (syn. mcmasteri), C. spatula, Ascaris suum, Hyostrongylus rubidus, Bunostomum phlebotomum, Capillaria bovis, B. trigonocephalum, Strongyloides papillosus, S. ransomi, Oesophagostomum radiatum, O. dentatus, O. columbianum, O. quadrispinulatum, Trichuris spp., and the like. Non-exclusive examples of hookworm (e.g., Ancylostoma caninum, A. tubaeforme, A. braziliense, Uncinaria stenocephala, and the like); lungworm (e.g., Dictyocaulus viviparus and Metastrongylus spp); eyeworm (e.g., Thelazia spp.); parasitic stage grubs (e.g., Hypoderma bovis, H. lineatum, Dermatobia hominis); kidneyworms (e.g., Stephanurus dentatus); screw worm (e.g., Cochliomyia hominivorax (larvae); filarial nematodes of the super-family Filarioidea and the Onchocercidae Family. Non-limiting examples of filarial nematodes within the Onchocercidae Family include the genus Brugia spp. (i.e., B. malayi, B. pahangi, B. timori, and the like), Wuchereria spp. (i.e., W. bancrofti, and the like), Dirofilaria spp. (D. immitis, D. ursi, D. tenuis, D. spectans, D. lutrae, and the like), Dipetalonema spp. (i.e., D reconditum, D. repens, and the like), Onchocerca spp. (i.e., O. gibsoni, O. gutturosa, O. volvulus, and the like), Elaeophora spp. (E.bohmi, E. elaphi, E. poeli, E. sagitta, E. schneideri, and the like), Mansonella spp. (i.e., M. ozzardi, M. perstans, and the like), and Loa spp. (i.e., L. loa). Non-exclusive examples of cestodes include: Taenia saginata, T. solium, T. taeniaformis, Hymenolepsis nana, H. diminuta, Dipylidium caninum; Diphyllobothrium latum; Echinococcus spp., Mesocestoides spp., and Spirometra spp. Non-exclusive examples of trematodes include: Paragonimus kellicotti, Alaria spp., Nanophyetus salmincola, Heterobiharzia Americana, Platynosomum fastosum, Schistosoma spp., and Fasciola spp.

The compounds of the invention and compositions comprising compounds of the invention in conjunction with at least one other veterinary agent are of particular value in the control of ectoparasites, endoparasites, and insects which are injurious to, or spread or act as vectors of diseases in companion animals, livestock, birds, and fish. The ectoparasites, insects, and endoparasites which can be treated with a combination of a Formula (1A), Formula (1B), or Formula (1C) compound and an additional veterinary agent include those as herein before described and including helminthes of the phylum platyhelminthes (e.g., trematodes, eucestoda, and cestoda), and nemathelminthes (e.g., nematodes).

Any of the compounds of the invention, or a suitable combination of a compound of the invention and optionally, with at least one additional veterinary agent may be administered directly to the animal and/or indirectly by applying it to the local environment in which the animal dwells (such as bedding, enclosures, and the like). Direct administration includes contacting the skin, fur, or feathers of a subject animal with the compound(s), or by feeding or injecting the compounds into the animal.

The Formula (1A), Formula (1B), and Formula (1C) compounds, stereoisomers thereof, and combinations with at least one additional veterinary agent, as described herein, are of value for the treatment and control of the various lifecycle stages of insects and parasites including egg, nymph, larvae, juvenile and adult stages.

The invention also relates to a method of administering a compound of the invention alone or in combination with at least one additional veterinary agent, and optionally a veterinarily acceptable excipient(s) to animals in good health comprising the application to said animal to reduce or eliminate the potential for human parasitic infection or infestation from parasites carried by the animal and to improve the environment in which the animals inhabit.

The reactions set forth below were done generally under a positive pressure of argon or nitrogen or with a drying tube, at ambient temperature (unless otherwise stated), in anhydrous solvents, and the reaction flasks were fitted with rubber septa for the introduction of substrates and reagents via syringe. Glassware was oven dried and/or heat dried. Analytical thin layer chromatography (TLC) was performed using glass-backed silica gel 60 F 254 precoated plates and eluted with appropriate solvent ratios (v/v). Reactions were assayed by TLC or LCMS and terminated as judged by the consumption of starting material. Visualization of the TLC plates was done with UV light (254 nM wavelength) or with an appropriate TLC visualizing solvent and activated with heat. Flash column chromatography (Still et al., J. Org. Chem. 43, 2923, (1978)) was performed using silica gel (RediSep Rf) or various MPLC systems, such as Biotage or ISCO purification system.

Conventional methods and/or techniques of separation and purification known to one of ordinary skill in the art can be used to isolate the compounds of the invention, as well as the various intermediates related thereto. Such techniques will be well-known to one of ordinary skill in the art and may include, for example, all types of chromatography (high pressure liquid chromatography (HPLC), column chromatography using common adsorbents such as silica gel, and thin-layer chromatography (TLC), recrystallization, and differential (i.e., liquid-liquid) extraction techniques.

The compound structures in the examples below were confirmed by one or more of the following methods: proton magnetic resonance spectroscopy, and mass spectroscopy. Proton magnetic resonance (¹H NMR) spectra were determined using a Bruker spectrometer operating at a field strength of 400 megahertz (MHz). Chemical shifts are reported in parts per million (PPM, δ) downfield from an internal tetramethylsilane standard. Mass spectra (MS) data were obtained using Agilent mass spectrometer with atmospheric pressure chemical ionization. Method: Acquity UPLC with chromatography performed on a Waters BEH C18 column (2.1×50 mm, 1.7 μm) at 50° C. The mobile phase was a binary gradient of acetonitrile (containing 0.1% trifluoroacetic acid) and water (5-100%).

Embodiments of the invention are illustrated by the following Examples. It is to be understood, however, that the embodiments of the invention are not limited to the specific details of these Examples, as other variations thereof will be known, or apparent in light of the instant disclosure, to one of ordinary skill in the art.

### EXAMPLES

The following examples provide a more detailed description of the process conditions for preparing compounds of the invention. It is to be understood, however, that the invention, as fully described herein and as recited in the claims, is not intended to be limited by the details of the following schemes or modes of preparation. Analytical data for the respective examples is in Table 1.

### Preparation of Intermediates

### Intermediate 1.1: 6-hydrazino-1,3-benzothiazole

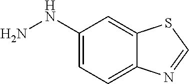 [SMILES: NNc1ccc2ncsc2c1]

To a stirred solution of 1,3-benzothiazole-6-amine (1.0 g, 6.6 mmol) in concentrated hydrochloric acid (2.0 mL) at 0° C. was added a solution of sodium nitrite (500 mg, 7.3 mmol) in water (1.0 mL) dropwise. After stirring at 0° C. for 45 minutes, a solution of stannous chloride dihydrate (3.5 g, 15 mmol) in concentrated hydrochloric acid (3.5 mL) was added dropwise. After complete addition, the solution was allowed to warm to room temperature and stirred for 2 hours. The mixture was cooled to 0° C. and basified to pH˜13 with concentrated aqueous sodium hydroxide solution. The aqueous layer was extracted with chloroform (2×50 mL). The organic layer was washed with brine, dried over anhydrous sodium sulfate and concentrated to a red solid. The crude product was purified by flash chromatography on silica gel. Elution with CHCl₃:MeOH:NH₄OH (97:2.6:0.4) afforded 750 mg (68%) of the title compound as a pink solid: ¹H NMR (400 MHz, CDCl₃) δ 8.75 (s, 1H), 7.95 (d, 1H, J=8.8 Hz), 7.43 (d, 1H, J=2.0 Hz), 6.96 (dd, 1H, J=8.8, 2.0 Hz), 5.50-5.30 (br s, 1H), 3.80-3.65 (br s, 2H); MS (ESI+) for C₇H₇N₃S m/z 166.1 (M+H)⁺.

### Intermediate 1.2: Preparation of tert-butyl 6′,7′-dihydro-1H-spiro[piperidine-4,8′-[1,3]thiazolo[5,4-e]indole-1-carboxylate

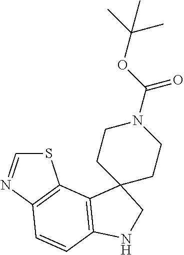 [SMILES: CC(C)(C)OC(=O)N1CCC2(CC1)CNc1ccc3ncsc3c12]

To a stirred solution of 6-hydrazino-1,3-benzothiazole (300 mg, 1.82 mmol) in chloroform (40 mL) at 0° C. were added a solution of tert-butyl 4-formylpiperidine-1-carboxylate (388 mg, 1.82 mmol) in chloroform (1.0 mL). Ethanol (40 μL, 0.7 mmol) was added, followed by the drop-wise addition of trifluoroacetic acid (430 μL, 5.5 mmol). After complete addition, the mixture was stirred at 0° C. for 30 minutes, followed by warming to 50° C. and stirring overnight. Next day, additional ethanol (40 μL, 0.70 mmol) and trifluoroacetic acid (130 μL, 1.8 mmol) were added and stirring was continued for an additional 24 hours. The reaction was quenched by cooling to room temperature and adding ammonium hydroxide (3.0 mL of a 10% aqueous solution) and two chips of ice. The mixture was stirred for 10 minutes, followed by extracting the mixture with ethyl acetate (25 mL). The organic layer was washed with brine, dried over anhydrous sodium sulfate and concentrated in vacuo to afford crude tert-butyl 1H-spiro[piperidine-4,8′-[1,3]thiazolo[5,4-e]indole]-1-carboxylate (75% pure based on integrations of the ¹H NMR spectrum).

To a stirred solution of the crude product mixture from above (600 mg, 1.3 mmol) in ethanol (20 mL) was added sodium borohydride (300 mg, 7.9 mmol).

The mixture was stirred at room temperature for 3 hours. Water (10 mL) was added, and the mixture was stirred for 15 minutes. The solvent was removed in vacuo, and the remaining residue was partitioned between water (20 mL) and chloroform (50 mL). The aqueous layer was extracted with chloroform (30 mL), and the combined organics were washed with brine, dried over anhydrous magnesium sulfate and concentrated in vacuo to a red solid. The solid wash triturated in ether to afford a precipitate. The precipitate was filtered and washed with ether and dried in vacuo to afford 280 mg (62%) of the title compound. Alternatively, the crude product could be purified by flash chromatography on silica gel. Off-white solid: ¹H NMR (400 MHz, DMSO-d₆) δ 8.94 (s, 1H), 7.70 (d, 1H, J=8.4 Hz), 6.77 (d, 1H, J=8.4 Hz), 5.98 (s, 1H), 4.05-3.95 (br m, 2H), 3.55 (s, 2H), 3.0-2.80 (br m, 2H), 1.95-1.85 (m, 2H), 1.60 (br d, 2H, J=13.2 Hz), 1.46 (s, 9H); MS (ESI+) for C₁₈H₂₃N₃O₂S m/z 346.1 [M+H]⁺.

### Intermediate 1.3: 5-Hydrazino-1,3-benzothiazole

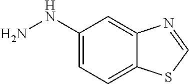 [SMILES: NNc1ccc2scnc2c1]

The compound (870 mg, 78%) was prepared from 1,3-benzothiazole-5-amine hydrochloride (1.25 g, 6.7 mmol), sodium nitrite (0.47 g, 6.9 mmol) and concentrated hydrochloric acid (11 mL), followed by subsequent treatment with stannous chloride dihydrate (4.96 g, 21.8 mmol) in a manner similar to that described for Intermediate 1.1. Yellow solid, ¹H NMR (400 MHz, CDCl₃) δ 8.99 (s, 1H), 7.76 (d, 1H, J=8.8 Hz), 7.60 (d, 1H, J=2.3 Hz), 6.99 (dd, 1H, J=8.8, 2.3 Hz), 5.50-5.30 (br s, 1H), 3.80-3.65 (br s, 2H); MS (ESI+) for C₇H₇N₃S m/z 166.1 (M+H)⁺.

### Intermediate 1.4: tert-butyl 6′,7′-dihydro-1H-spiro[piperidine-4,8′-[1,3]thiazolo[4,5-e]indole]-1-carboxylate

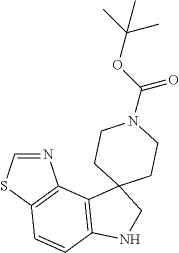 [SMILES: CC(C)(C)OC(=O)N1CCC2(CC1)CNc1ccc3scnc3c12]

The compound (380 mg, 47%) was prepared from 5-hydrazino-1,3-benzothiazole (400 mg, 2.4 mmol), tert-butyl 4-formylpiperidine-1-carboxylate (520 mg, 2.4 mmol), TFA (746 uL, 9.7 mmol) and EtOH (53 μL, 0.91 mmol), followed by workup and subsequent treatment with sodium borohydride (400 mg, 10.6 mmol) in a manner similar to that described for Intermediate 1.2. Yellow solid, ¹H NMR (400 MHz, CDCl₃) δ 8.97 (s, 1H), 7.65 (d, 1H, J=8.4 Hz), 6.87 (d, 1H, J=8.4 Hz), 4.32-4.10 (br s, 2H), 3.95-3.90 (br s, 1H), 3.66 (s, 2H), 3.0-2.80 (br s, 2H), 2.72-2.62 (br m, 2H), 1.77 (br d, 2H, J=13.2 Hz), 1.51 (s, 9H); MS (ESI+) for C₁₈H₂₃N₃O₂S m/z 246.1 [M-Boc+2H]⁺, 290.1 [M-^{t}Bu+2H]⁺.

### Intermediate 1.5: 5-hydrazino-2-methyl-1,3-benzothiazole

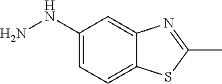

The compound (3.8 g, 70%) was prepared from 2-methyl-1,3-benzothiazole-5-amine (5.0 g, 30.4 mmol), sodium nitrite (2.2 g, 32.0 mmol) and concentrated hydrochloric acid (50 mL), followed by subsequent treatment with stannous chloride dihydrate (22.7 g, 99.7 mmol) in a manner similar to that described for Intermediate 1.1. Yellow solid, ¹H NMR (400 MHz, CDCl₃) δ 7.62 (d, 1H, J=8.4 Hz), 7.42 (d, 1H, J=2.4 Hz), 6.88 (dd, 1H, J=8.4, 2.4 Hz), 5.40-5.30 (br s, 1H), 3.72-3.58 (br s, 2H); 2.82 (s, 3H); MS (ESI+) for C₈H₉N₃S m/z 180.1 [M+H]⁺.

### Intermediate 1.6: tert-butyl 2′-methyl-6′,7′-dihydro-1H-spiro[piperidine-4,8′-[1,3]thiazolo[4,5-e]indole]-1-carboxylate

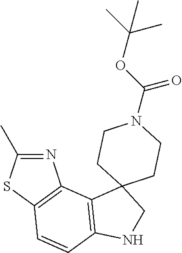 [SMILES: Cc1nc2c3c(ccc2s1)NCC31CCN(C(=O)OC(C)(C)C)CC1]

The compound (1.50 g, 20%) was prepared from 5-hydrazino-2-methyl-1,3-benzothiazole (3.8 g, 21 mmol), tert-butyl 4-formylpiperidine-1-carboxylate (4.5 g, 21 mmol), TFA (5.71 mL, 74.2 mmol) and EtOH (460 μL, 7.9 mmol), followed by workup and subsequent treatment with sodium borohydride (3.0 g, 79 mmol) in a manner similar to that described for Intermediate 1.2. White solid, ¹H NMR (400 MHz, CDCl₃) δ 7.49 (d, 1H, J=8.0 Hz), 6.75 (d, 1H, J=8.0 Hz), 4.30-4.10 (br s, 2H), 3.87-3.83 (br s, 1H), 3.62 (s, 2H), 2.96-2.82 (br m, 2H), 2.80 (s, 3H), 2.72-2.62 (br m, 2H), 1.73 (br d, 2H, J=12.4 Hz), 1.53 (s, 9H); MS (ESI+) for C₁₉H₂₅N₃O₂S m/z 260.1 (M-Boc+2H)+, 304.1 (M-^{t}Bu+2H)+, 360.1 [M+H]⁺.

### Intermediate 1.7: 5-hydrazino-1,2-benzisothiazole

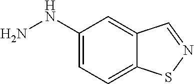 [SMILES: NNc1ccc2sncc2c1]

The compound (870 mg, 78%) was prepared from 1,2-benzisothiazole-5-amine (3.0 g, 20 mmol), sodium nitrite (1.45 g, 21 mmol) and concentrated hydrochloric acid (33 mL), followed by subsequent treatment with stannous chloride dihydrate (15 g, 66 mmol) in a manner similar to that described for Intermediate 1.1. Yellow solid, ¹H NMR (400 MHz, CDCl₃) δ 8.79 (s, 1H), 7.77 (d, 1H, J=8.8 Hz), 7.49 (d, 1H, J=1.6 Hz), 7.06 (dd, 1H, J=8.8, 1.6 Hz), 5.50-5.36 (br s, 1H), 3.80-3.60 (br s, 2H); MS (ESI+) for C₇H₇N₃S m/z 166.1 [M+H]⁺.

### Intermediate 1.8: tert-butyl 6,7-dihydro-1′H-spiro[isothiazolo[4,5-e]indole-8,4′-piperidine]-1′-carboxylate

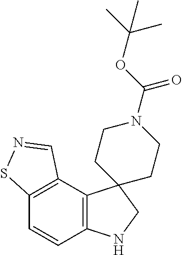 [SMILES: CC(C)(C)OC(=O)N1CCC2(CC1)CNc1ccc3sncc3c12]

The compound (2.0 g, 41%) was prepared from 5-hydrazino-1,2-benzisothiazole (2.35 g, 14.2 mmol), tert-butyl 4-formylpiperidine-1-carboxylate (3.03 g, 14.2 mmol), TFA (4.38 mL, 56.9 mmol) and EtOH (300 μL, 5.0 mmol), followed by workup and subsequent treatment with sodium borohydride (1.50 g, 40.0 mmol) in a manner similar to that described for intermediate 1.2. Yellow solid, ¹H NMR (400 MHz, CDCl₃) δ 9.00 (d, 1H, J=0.8 Hz), 7.68 (dd, 1H, J=8.4, 0.8 Hz), 6.99 (d, 1H, J=8.4 Hz), 4.35-4.13 (br s, 2H), 3.97-3.93 (br s, 1H), 3.68 (s, 2H), 3.0-2.80 (br s, 2H), 2.35-2.20 (br m, 2H), 1.87 (br d, 2H, J=12.8 Hz), 1.54 (s, 9H); MS (ESI+) for C₁₈H₂₃N₃O₂S m/z 246.2 [M-Boc+2H]⁺, 290.1 [M-^{t}Bu+2H]⁺, 346.2 [M+H]⁺.

### Intermediate 1.9: 5-hydrazino-1-methyl-1H-indazole

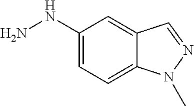

The compound (2.35 g, 69%) was prepared from 1-methyl-1H-indazol-5-amine (3.0 g, 20 mmol), sodium nitrite (1.52 g, 22 mmol) and concentrated hydrochloric acid (33 mL), followed by subsequent treatment with stannous chloride dihydrate (15.3 g, 67.3 mmol) in a manner similar to that described for intermediate 1.1. Yellow solid, ¹H NMR (400 MHz, CDCl₃) δ 7.86 (d, 1H, J=0.8 Hz), 7.28 (d, 1H, J=8.8 Hz), 7.11 (dd, 1H, J=2.4, 0.8 Hz), 6.96 (dd, 1H, J=8.8, 2.4 Hz), 5.40-4.80 (br s, 1H), 4.06 (s, 3H), 3.80-3.50 (br s, 2H); MS (ESI+) for C₈H₁₀N₄ m/z 163.1 [M+H]⁺.

### Intermediate 1.10: tert-butyl 3′-methyl-6′,7′-dihydro-1H,3′H-spiro[piperidine-4,8′-pyrrolo[3,2-e]indazole]-1-carboxylate

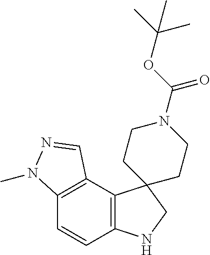 [SMILES: Cn1ncc2c3c(ccc21)NCC31CCN(C(=O)OC(C)(C)C)CC1]

The compound (1.7 g, 36%) was prepared from 5-hydrazino-1-methyl-1H-indazole (2.35 g, 14.0 mmol), tert-butyl 4-formylpiperidine-1-carboxylate (3.09 g, 14.5 mmol), TFA (4.33 mL, 56.2 mmol) and EtOH (300 μL, 5.0 mmol), followed by workup and subsequent treatment with sodium borohydride (1.50 g, 40.0 mmol) in a manner similar to that described for Intermediate 1.2. White solid, ¹H NMR (400 MHz, CDCl₃) δ 7.89 (d, 1H, J=0.8 Hz), 7.16 (dd, 1H, J=8.4, 0.8 Hz), 6.92 (d, 1H, J=8.4 Hz), 4.30-4.10 (br s, 2H), 4.05 (s, 3H), 3.80-3.67 (br s, 1H), 3.61 (s, 2H), 2.95-2.80 (br s, 2H), 2.30-2.15 (br m, 2H), 1.81 (br d, 2H, J=13.2 Hz), 1.53 (s, 9H); MS (ESI+) for C₁₉H₂₆N₄O₂ m/z 246.2 287.1 [M-^{t}Bu+2H]⁺, 343.2 [M+H]⁺.

### Intermediate 1.11: 1,3-dihydro-2-benzofuran-5-ylhydrazine

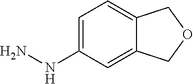 [SMILES: NNc1ccc2c(c1)COC2]

The compound (2.20 g, 40%) was prepared from 1,3-dihydro-2-benzofuran-5-amine (5.0 g, 37 mmol), sodium nitrite (3.10 g, 44 mmol), concentrated hydrochloric acid (13 mL) and water (26 mL), followed by subsequent treatment with stannous chloride dihydrate (25 g, 110 mmol) in a manner similar to that described for Intermediate 1.1. Yellow solid, ¹H NMR (400 MHz, CDCl₃) δ 7.10 (d, 1H, J=8.0 Hz), 6.76 (d, 1H, J=2.3 Hz), 6.74 (dd, 1H, J=8.0, 2.4 Hz), 5.40-5.10 (br s, 1H), 5.08 (s, 2H), 5.07 (s, 2H), 3.80-3.40 (br s, 2H); MS (ESI+) for C₈H₁₀N₂O m/z 151.1 [M+H]⁺.

### Intermediates 1.12a and 1.12b: tert-butyl 1,3,6,7-tetrahydro-1′H-spiro[furo[3,4-e]indole-8,4′-piperidine]-1′-carboxylate (1.12a) and tert-butyl 1,2,5,7-tetrahydro-1′H-spiro[furo[3,4-f]indole-3,4′-piperidine]-1′-carboxylate (1.12b)

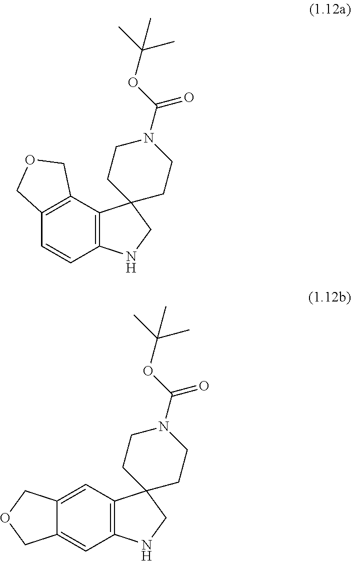 [SMILES: CC(C)(C)OC(=O)N1CCC2(CC1)CNc1ccc3c(c12)COC3 ; CC(C)(C)OC(=O)N1CCC2(CC1)CNc1cc3c(cc12)COC3]

The intermediates 1.12a (333 mg, 7%) and 1.12b (1.3 g, 26%) were prepared from 1,3-dihydro-2-benzofuran-5-ylhydrazine (2.20 g, 15 mmol), tert-butyl 4-formylpiperidine-1-carboxylate (3.1 g, 15 mmol), TFA (4.5 mL, 58 mmol) and EtOH (300 μL, 5.0 mmol), followed by workup and subsequent treatment with sodium borohydride (2.0 g, 53.0 mmol) in a manner similar to that described for Intermediate 1.2.

Minor isomer 1.12a: White solid, ¹H NMR (400 MHz, CDCl₃) δ 6.93 (d, 1H, J=10.4 Hz), 6.59 (d, 1H, J=10.4 Hz), 5.12 (s, 2H), 5.00 (s, 2H), 4.25-4.05 (br s, 2H), 3.85-3.75 (br s, 1H), 3.54 (s, 2H), 2.90-2.70 (br m, 2H), 1.85-1.65 (br m, 4H), 1.50 (s, 9H); MS (ESI+) for C₁₉H₂₆N₂O₃ m/z 275.1 [M-^{t}Bu+2H]⁺, 331.2 [M+H]⁺.

Major isomer 1.12b: White solid, ¹H NMR (400 MHz, CDCl₃) δ 6.89 (s, 1H), 6.51 (s, 1H), 5.02 (s, 4H), 4.15-4.0 (br s, 2H), 3.80-3.70 (br s, 1H), 3.53 (s, 2H), 3.00-2.85 (br m, 2H), 1.85-1.65 (br m, 4H), 1.50 (s, 9H); MS (ESI+) for C₁₉H₂₆N₂O₃ m/z 275.1 [M-^{t}Bu+2H]⁺, 331.2 [M+H]⁺.

### Intermediate 1.13: 2,3-dihydro-1H-inden-5-ylhydrazine

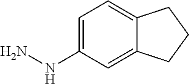 [SMILES: NNc1ccc2c(c1)CCC2]

The compound (3.5 g, 74%) was prepared from indan-5-amine (3.6 g, 27 mmol), sodium nitrite (1.96 g, 28.4 mmol) and concentrated hydrochloric acid (20 mL), followed by subsequent treatment with stannous chloride dihydrate (20.3 g, 89.2 mmol) in a manner similar to that described for intermediate 1.1. Brown solid, ¹H NMR (400 MHz, CDCl₃) δ 7.11 (d, 1H, J=8.4 Hz), 6.78 (s, 1H), 6.63 (d, 1H, J=8.4 Hz), 4.50-3.50 (br s, 3H), 3.00-2.70 (m, 4H), 2.20-2.00 (s, 2H); MS (ESI+) for C₉H₁₂N₂ m/z 149.1 [M+H]⁺.

### Intermediates 1.14a and 1.14b: tert-butyl 3,6,7,8-tetrahydro-1′H,2H-spiro[cyclopenta[e]indole-1,4′-piperidine]-1′-carboxylate (1.14a) and tert-butyl 2,5,6,7-tetrahydro-1′H,2H-spiro[cyclopenta[f]indole-3,4′-piperidine]-1′-carboxylate (1.14b)

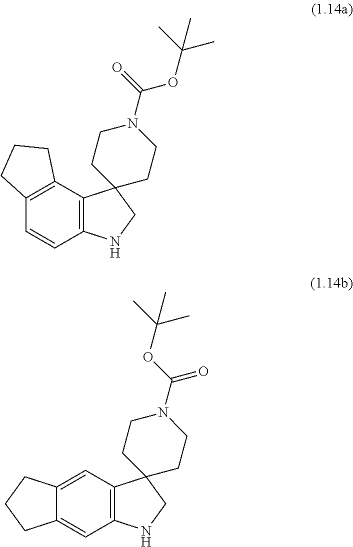 [SMILES: CC(C)(C)OC(=O)N1CCC2(CC1)CNc1ccc3c(c12)CCC3 ; CC(C)(C)OC(=O)N1CCC2(CC1)CNc1cc3c(cc12)CCC3]

The intermediates 1.14a and 1.14b (2.0 g, 30%, inseparable 1:2 mixture of isomers) were prepared from 2,3-dihydro-1H-inden-5-ylhydrazine (3.5 g, 20 mmol), tert-butyl 4-formylpiperidine-1-carboxylate (4.3 g, 20 mmol), TFA (4.6 mL, 60 mmol) and EtOH (400 μL, 7.0 mmol), followed by workup and subsequent treatment with sodium borohydride (1.5 g, 40.0 mmol) in a manner similar to that described in Intermediate 1.2. Yellow powder, ¹H NMR (400 MHz, CDCl₃) δ 6.96 (d, 0.25H, J=7.6 Hz, minor isomer), 6.93 (s, 0.75H, major isomer), 6.59 (s, 0.75H, major isomer), 6.51 (d, 0.25H, J=7.6 Hz), 4.20-4.00 (br s, 2H), 3.65-3.55 (br s, 1H), 3.51 (s, 0.25 H, minor isomer), 3.50 (0.75H, major isomer), 3.00-2.85 (br m, 2H), 2.85-2.75 (m, 4H), 2.12-2.02 (m, 2H), 1.85-1.64 (m, 4H), 1.50 (s, 9H); MS (ESI+) for C₂₀H₂₈N₂O₂ m/z 273.2 [M-^{t}Bu+2H]⁺, 329.3 [M+H]⁺.

### Intermediate 1.15: 2,3-dihydro-1,4-benzodioxin-6-ylhydrazine

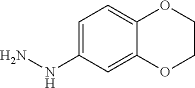 [SMILES: NNc1ccc2c(c1)OCCO2]

The compound (5.6 g, 81%) was prepared from 2,3-dihydro-1,4-benzodioxin-6-amine (6.0 g, 40 mmol), sodium nitrite (3.3 g, 45 mmol), concentrated hydrochloric acid (12 mL), and water (30 mL), followed by subsequent treatment with stannous chloride dihydrate (27 g, 120 mmol) in a manner similar to that described for Intermediate 1.1. Red oil, ¹H NMR (400 MHz, CDCl₃) δ 6.77 (d, 1H, J=8.8 Hz), 6.42 (d, 1H, J=2.8), 6.34 (dd, 1H, J=8.8, 2.8 Hz), 5.00-4.50 (br s, 1H), 4.28-4.19 (m, 4H), 4.20-3.20 (br s, 2H); MS (ESI+) for C₈H₁₀N₂O₂ m/z 167.1 [M+H]⁺.

### Intermediates 1.16a and 1.16b: tert-butyl 2,3,7,8-tetrahydro-1′H-spiro[1,4-dioxino[2,3-e]indole-9,4′-piperidine]-1′-carboxylate (1.16a) and tert-butyl 2,3,6,7-tetrahydro-1′H-spiro[1,4-dioxino[2,3-f]indole-8,4′-piperidine]-1′-carboxylate (1.16b)

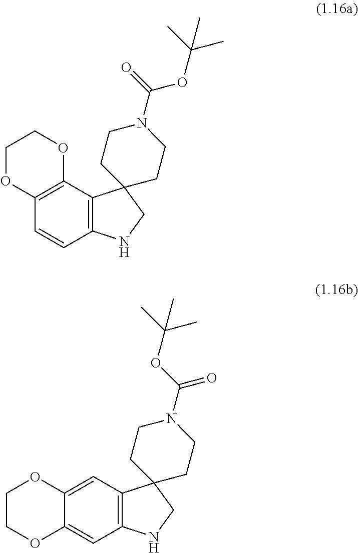 [SMILES: CC(C)(C)OC(=O)N1CCC2(CC1)CNc1ccc3c(c12)OCCO3 ; CC(C)(C)OC(=O)N1CCC2(CC1)CNc1cc3c(cc12)OCCO3]

The compounds 1.16a and 1.16b (2.0 g, 26%, inseparable 1:2 mixture of isomers) were prepared from 2,3-dihydro-1,4-benzodioxin-6-ylhydrazine (2.3 g, 14 mmol), tert-butyl 4-formylpiperidine-1-carboxylate (3.0 g, 14 mmol), TFA (3.2 mL, 42 mmol) and ethanol (300 μL, 5.0 mmol), followed by workup and subsequent treatment with sodium borohydride (2.5 g, 66.0 mmol) in a manner similar to that described for Intermediate 1.2. Orange foam, ¹H NMR (400 MHz, CDCl₃) δ 6.61 (d, 0.25H, J=8.4 Hz, minor isomer), 6.60 (s, 0.75H, major isomer), 6.24 (s, 0.75H, major isomer), 6.19 (d, 0.25H, J=8.4 Hz, minor isomer), 4.28-4.20 (m, 4H), 4.15-3.95 (br s, 2H), 3.50 (s, 0.25 H, minor isomer), 3.44 (0.75H, major isomer), 3.00-2.70 (br m, 2H), 2.40-2.20 (br s, 1H), 1.80-1.60 (m, 4H), 1.51 (s, 9H); MS (ESI+) for C₁₉H₂₆N₂O₄ m/z 291.2 [M-^{t}Bu+2H]⁺, 346.2 [M+H]⁺.

### Intermediate 1.17: (E)-3-(5-(trifluoromethyl)pyridin-2-yl)acrylaldehyde

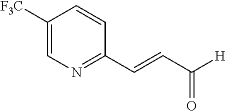 [SMILES: O=C/C=C/c1ccc(C(F)(F)F)cn1]

To 2-bromo-5-trifluoromethylpyridine (295 g, 1.3 mol) in DCM (4 L) at −2° C. was added iPrMgCl (2M in Et₂O, 750 ml, 1.5 mol) over 3 minutes then stirred at 0-6° C. for 40 minutes [jacket at 0° C., mild and gradual exotherm took pot temp to 6° C. maximum after ˜15 minutes]. The mixture was cooled to −20° C. then DMF (200 ml, 2.6 mol) was added in one portion [exotherm to 6° C.]. The mixture was slowly re-cooled to 0° C. over 20 minutes then quenched by addition of 1.5 L saturated NaHCO3 in one portion [temperature to 12° C.]. The mixture was stirred at 12° C. for 15 minutes then filtered through a celite pad. The layers were separated. The filtered solids were washed with 1 L DCM and this was then used to re-extract the aqueous layer. The combined organics were dried over MgSO4, filtered through a pad of 1 kg silica, washed with 5 L DCM and evaporated (bath temp 35° C.). The brown oil was dissolved in 2 L hexane and washed with 2×1 L 12% brine to remove DMF. The organics were filtered through a pad of MgSO4 and concentrated to low volume. This oil was distilled at 35° C. and 20 inches Hg to remove hexane then at 56° C. and 26 in Hg to afford the 5-(trifluoromethyl)-picolinaldehyde product (154 g, ˜90% purity, 0.79 mol, 61%) as a pale yellow moist crystal. A solution of 5-(trifluoromethyl)picolinaldehyde (150 g, ˜90% purity, 0.77 mol) in DCM (1.5 L) was bubbled with nitrogen for 5 minutes and cooled to 9° C. (Formylmethylene)triphenylphosphorane (257 g, 0.85 mol) was added in one portion [exotherm to 19° C.], and the reaction stirred at 20° C. for 60 minutes. The mixture was filtered through a pad of magnesol (100 g) and washed with DCM (400 ml). The filtrate was evaporated at 40° C. The residue was triturated with MTBE (150 ml) then diluted with hexane (300 ml) and filtered, washing with MTBE/hexane. The filtrate was diluted with DCM to solubilize a small amount of oil that had separated and then chromatographed (3 kg silica, 20-35% MTBE in hexane) to afford the desired product (60.3 g, 0.30 mol, 39%) as a dark red solid. ¹H NMR (500 MHz, CDCl₃) δ 9.85 (d, 1H, J=8.0 Hz), 8.95 (s, 1H), 8.02 (dd, 1H, J=8.0, 2.0 Hz), 7.66 (d, 1H, J=8 Hz), 7.55 (d, 1H, J=16.0 Hz), 7.19 (dd, 1H, J=16.0, 8.0 Hz), GC 99.47%.

### Intermediate 1.18: 2-methoxy-8-methylquinoline-6-carbaldehyde

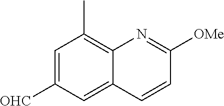

Intermediate 1.18 can be prepared by oxidizing 2-methoxy-6,8-dimethylquinoline with selenium dioxide according to procedures described by Tsotinis, et. al., in Letters in Drug Design & Discovery, 2005, 2, 189-192.

The following Examples were made in accordance with the schemes and reaction steps as described herein and also using the following scheme,

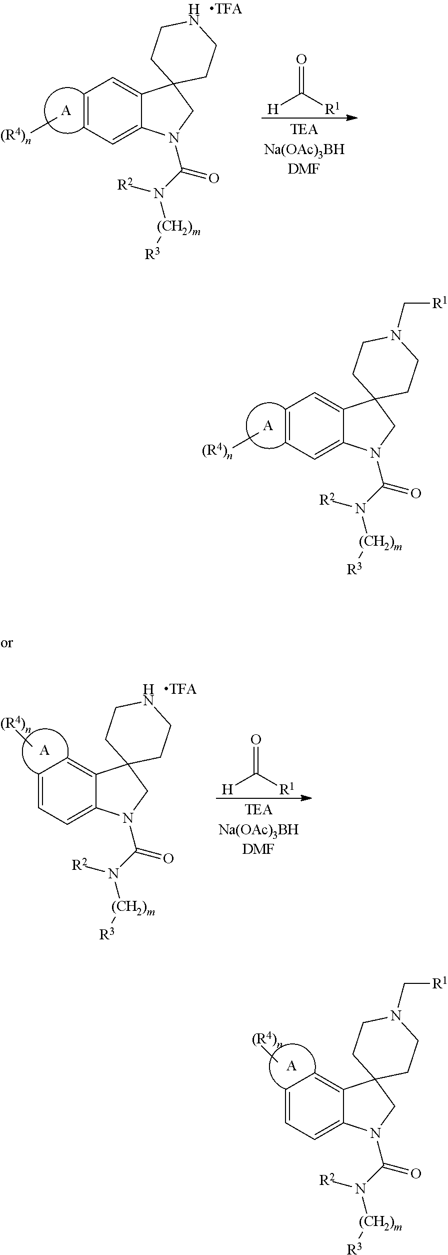

wherein A, R¹, R², R³, R⁴, n, and m are as defined herein.

Parallel reductive amination reactions were carried out with the general structure as shown in the Schemes presented herein. The stock solution of a template amine (0.05 mmol for each reaction) was prepared in dimethyl formamide along with triethylamine (0.5 mmol/reaction). Template stock solution was added to vials containing respective aldehyde monomers (0.1 mmol/reaction). The reaction was allowed to stir for 15 minutes at room temperature followed by the addition of sodium triacetoxyborohydride (21.2 mg, 0.1 mmol/reaction). All reactions were allowed to stir at room temperature for 16 hours. The crude product obtained from each reaction was analyzed by LCMS analysis. Volatiles were removed under vacuum. All compounds were further purified by Preparation HPLC. The isolated compounds were analyzed by LCMS.

### Dihydrofuran, Dioxolane, and Dioxane Analogs (Examples 1-11)

### Example 1

### (E)-N-((2-chloropyridin-4-yl)methyl)-1′-(3-(3,4-dichlorophenyl) allyl)-5,7-dihydrospiro[furo[3,4-f]indole-3,4′-piperidine]-1(2H)-carboxamide

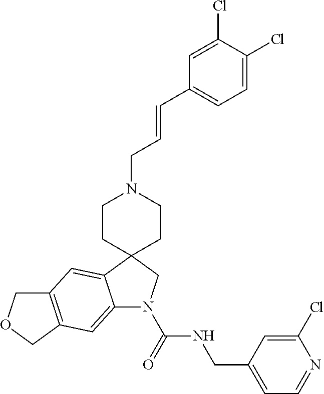 [SMILES: O=C(NCc1ccnc(Cl)c1)N1CC2(CCN(C/C=C/c3ccc(Cl)c(Cl)c3)CC2)c2cc3c(cc21)COC3]

### Step 1: Preparation of tert-butyl 1-((2-chloropyridin-4-yl)methylcarbamoyl)-1,2,5,7-tetrahydrospiro[furo[3,4-f]indole-3,4′-piperidine]-1′-carboxylate

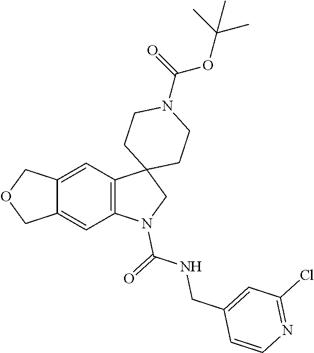 [SMILES: CC(C)(C)OC(=O)N1CCC2(CC1)CN(C(=O)NCc1ccnc(Cl)c1)c1cc3c(cc12)COC3]

In 10 mL vial, to the stirred solution of (2-chloro-pyridin-4-yl)-methylamine hydrochloride (135 mg, 0.758 mmol, 1 eq.) in dichloromethane (6 mL) was added triethylamine (0.426 mL, 3.034 mmol, 4 eq) followed by the addition of 1,1-carbonyldiimidazole (160 mg, 0.986 mmol, 1.3 eq) at 0° C. Reaction was allowed to warm slowly at room temperature and stirred for 4 hours. After consumption of starting material, reaction was cooled to 0° C. and added tert-butyl 1,2,5,7-tetrahydrospiro-[furo[3,4-f]indole-3,4′-piperidine]-1′-carboxylate (Intermediate 1.12b, 201 mg, 0.607 mmol, 0.8 eq) and stirred at room temperature for 16 hours. After completion, reaction mass was concentrated under reduced pressure to afford brown thick mass. Purification was done by column chromatography using silica gel (100-200 mesh). Desired compound was eluted in 2% methanol in DCM to afford off white solid (202 mg, 53%). ¹H NMR (400 MHz, DMSO-d₆) δ: 1.42 (s, 9H), 1.60-1.64 (m, 2H), 1.66-1.71 (m, 2H), 2.84-2.88 (m, 2H), 3.95-4.00 (m, 4H), 4.36 (d, J=5.52 Hz, 2H), 4.89 (s, 4H), 7.13 (s, 1H), 7.37 (m, 1H), 7.39-7.42 (m, 1H), 7.44 (s, 1H), 7.74 (s, 1H), 8.34 (d, J=5.12 Hz, 1H). LC-MS (m/z): 499.0 (M+H).

### Step 2: Preparation of trifluoroacetic acid salt of N-((2-chloropyridin-4-yl)methyl)-5,7-dihydrospiro [furo[3,4-f]indole-3,4′-piperidine]-1(2H)-carboxamide

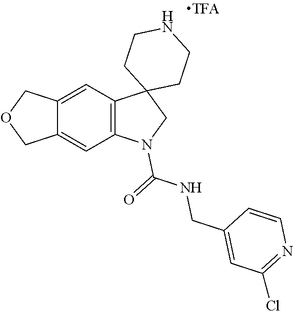 [SMILES: O=C(NCc1ccnc(Cl)c1)N1CC2(CCNCC2)c2cc3c(cc21)COC3]

In a 10 mL vial, to the stirred solution of tert-butyl 1-((2-chloropyridin-4-yl)methylcarbamoyl)-1,2,5,7-tetrahydrospiro[furo[3,4-f]indole-3,4′-piperidine]-1′-carboxylate (200 mg, 0.401 mmol, 1 eq) in 3 mL dichloromethane was added trifluoroacetic acid (0.5 mL) slowly at 0° C. Resulting reaction mass allowed to warm at room temperature and stirred for 2 hours. After complete consumption of starting material, reaction mass was concentrated under reduced pressure and stripped out with chloroform (3×3 mL) to afford brown sticky material (230 mg, crude). The crude material was used as such for next step.

### Step 3: Preparation of Example 1

In a 10 mL vial, to a stirred solution of N-((2-chloropyridin-4-yl)methyl)-5,7-dihydrospiro [furo[3,4-f]indole-3,4′-piperidine]-1(2H)-carboxamide TFA salt crude (0.230 g, 0.448 mmol, 1 eq) in dimethylformamide (5 mL) was added triethylamine (0.312 mL, 2.24 mmol, 5 eq) and (E)-3-(3,4-dichloro-phenyl)-propenal (0.117 g, 0.583 mmol, 1.3 eq). Reaction was stirred at room temperature for 15 minutes. Sodium triacetoxyborohydride (0.19 g, 0.897 mmol, 2 eq) was added to the reaction mixture and reaction was allowed to stir at room temperature for 16 hours. After completion, reaction mass was concentrated in a speed vacuum to afford a brown sticky mass. Crude material (0.270 g) was purified by preparative HPLC to afford off white solid (88.82 mg, 34%). ¹H NMR (400 MHz, DMSO-d₆) δ: 1.60-1.63 (m, 2H), 1.82-1.88 (m, 2H), 2.05-2.11 (m, 2H), 2.88-2.91 (m, 2H), 3.13-3.17 (m, 2H), 3.88 (s, 2H), 4.35 (d, J=5.56 Hz, 2H), 4.89-4.91 (m, 4H), 6.47-6.58 (m, 2H), 7.10 (s, 1H), 7.36 (d, J=5.2 Hz, 1H), 7.43-7.48 (m, 3H), 7.57 (d, J=8.36 Hz, 1H), 7.74-7.76 (m, 2H), 8.34 (d, J=5.04 Hz, 1H). LC-MS: (m/z): 583.0 (M+H), HPLC: 98.91%

### Example 2

### (E)-1′-(3-(4-chlorophenyl)allyl)-N-((2-fluoropyridin-4-yl)methyl)-5,7-dihydrospiro[furo[3,4-f]indole-3,4′-piperidine]-1(2H)-carboxamide

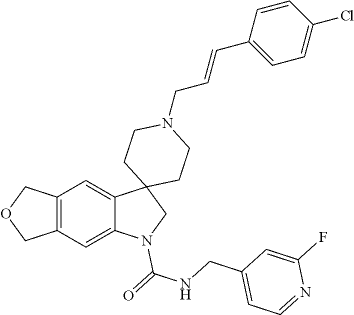 [SMILES: O=C(NCc1ccnc(F)c1)N1CC2(CCN(C/C=C/c3ccc(Cl)cc3)CC2)c2cc3c(cc21)COC3]

Example 2 was prepared similarly to Example 1 except that (2-fluoro-pyridin-4-yl)-methylamine hydrochloride was used in Step 1 rather than (2-chloro-pyridin-4-yl)-methylamine hydrochloride and (E)-3-(3,4-dichloro-phenyl)-propenal was replaced with (E)-3-(4-chloro-phenyl)-propenal. 1H NMR (400 MHz, DMSO) δ: 1.62 (d, J=1.216 Hz, 2H), 1.82-1.85 (m, 2H), 2.08 (t, J=11.24 Hz, 2H), 2.90 (d, J=11.44 Hz, 2H), 3.14 (d, J=6.4 Hz, 2H), 3.89 (s, 2H), 4.38 (d, J=5.64 Hz, 2H), 4.90 (d, J=4.4 Hz, 4H) 6.34-6.41 (m, 1H), 6.56 (d, J=15.88 Hz, 1H), 7.10 (d, J=5.64 Hz, 2H), 7.30 (d, J=4.76 Hz, 1H), 7.37 (d, J=8.46 Hz, 2H), 7.45-7.50 (m, 3H), 7.74 (s, 1H), 8.16 (d, J=5.08 Hz, 1H). LC-MS (m/z): 532.8 (M+H).

### Example 3

### (E)-1′-(3-(4-chlorophenyl)allyl)-N-((2-chlorothiazol-5-yl)methyl)-5,7-dihydrospiro[furo[3,4-f]indole-3,4′-piperidine]-1(2H)-carboxamide

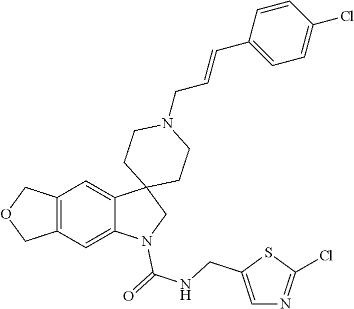 [SMILES: O=C(NCc1cnc(Cl)s1)N1CC2(CCN(C/C=C/c3ccc(Cl)cc3)CC2)c2cc3c(cc21)COC3]

Example 3 was prepared similarly to Example 2 except that (2-chlorothiazol-5-yl)methylamine hydrochloride was used in place of (2-chloro-pyridin-4-yl)-methylamine hydrochloride. 1H NMR (400 MHz, DMSO) δ: 1.56 (d, J=12.48 Hz, 2H), 1.82-1.86 (m, 2H), 2.03 (t, J=11.92 Hz, 2H), 2.87 (d, J=11.04 Hz, 2H), 3.12 (d, J=6.32 Hz, 2H), 3.77 (s, 2H), 4.41 (d, J=5.44 Hz, 2H), 4.91 (s, 4H) 6.33-6.40 (m, 1H), 6.55 (d, J=15.84 Hz, 1H), 7.10 (s, 1H), 7.37 (d, J=8.52 Hz, 2H), 7.48 (d, J=8.56 Hz, 2H), 7.54 (t, J=5.6 Hz, 1H), 7.58 (s, 1H), 7.78 (s, 1H). LC-MS (m/z): 554.6 (M+H).

### Example 4

### (E)-N-((2-chloropyridin-4-yl)methyl)-1′-(3-(4-fluorophenyl)allyl)-5,7-dihydrospiro[furo[3,4-f]indole-3,4′-piperidine]-1(2H)-carboxamide

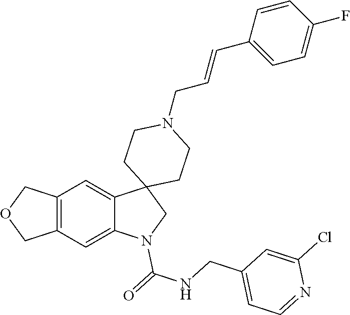 [SMILES: O=C(NCc1ccnc(Cl)c1)N1CC2(CCN(C/C=C/c3ccc(F)cc3)CC2)c2cc3c(cc21)COC3]

Example 4 was prepared similarly to Example 1, except that (E)-3-(3,4-dichloro-phenyl)-propenal was replaced with (E)-3-(4-fluoro-phenyl)-propenal. 1H NMR (400 MHz, DMSO) δ: 1.61 (d, J=12.2 Hz, 2H), 1.84-1.88 (m, 2H), 2.08 (t, J=9.84 Hz, 2H), 2.91 (d, J=11.56 Hz, 2H), 3.13 (d, J=6.4 Hz, 2H), 3.88 (s, 2H), 4.35 (d, J=5.64 Hz, 2H), 4.90 (d, J=3.76 Hz, 4H) 6.26-6.33 (m, 1H), 6.55 (d, J=15.96 Hz, 1H), 7.11-7.17 (m, 3H), 7.36 (d, J=5.12 Hz, 1H), 7.43-7.52 (m, 4H), 7.74 (s, 1H), 8.34 (d, J=5.04 Hz, 1H). LC-MS (m/z): 532.6 (M+H).

### Example 5

### (E)-N-((2-chloropyridin-4-yl)methyl)-1′-(3-(4-cyanophenyl)allyl)-5,7-dihydrospiro[furo[3,4-f]indole-3,4′-piperidine]-1(2H)-carboxamide

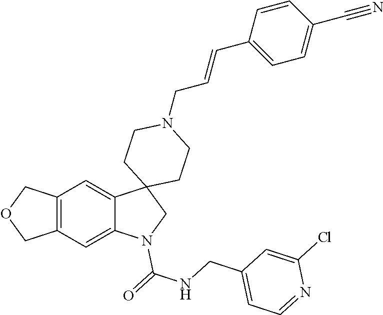 [SMILES: N#Cc1ccc(/C=C/CN2CCC3(CC2)CN(C(=O)NCc2ccnc(Cl)c2)c2cc4c(cc23)COC4)cc1]

Example 5 was prepared similarly to Example 1, except that (E)-3-(3,4-dichloro-phenyl)-propenal was replaced with (E)-3-(4-cyano-phenyl)-propenal. 1H NMR (400 MHz, DMSO) δ: 1.62 (d, J=11.32 Hz, 2H), 1.83-1.89 (m, 2H), 2.08-2.09 (m, 2H), 2.90 (brs, 2H), 3.18 (brs, 2H), 3.89 (s, 2H), 4.35 (d, J=5.56 Hz, 2H), 4.90 (d, J=4.2 Hz, 4H), 6.59-6.64 (m, 2H), 7.10 (s, 1H), 7.36 (d, J=5.08 Hz, 1H), 7.43-7.45 (m, 2H), 7.66 (d, J=8.32 Hz, 2H), 7.73 (s, 1H) 7.79 (d, J=8.2 Hz, 2H), 8.34 (d, J=5 Hz, 1H). LC-MS (m/z): 540.3 (M+H).

### Example 6

### (E)-1-(3-(4-chlorophenyl)allyl)-N-((2-chloropyridin-4-yl)methyl)spiro[piperidine-4,7′-[1,3]dioxolo[4,5-f]indole]-5′(6′H)-carboxamide

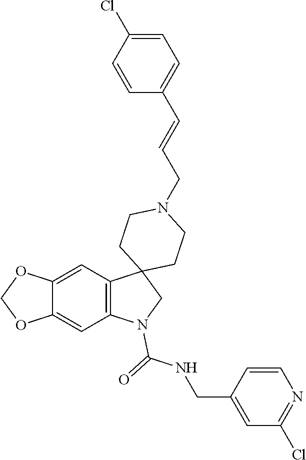 [SMILES: O=C(NCc1ccnc(Cl)c1)N1CC2(CCN(C/C=C/c3ccc(Cl)cc3)CC2)c2cc3c(cc21)OCO3]

Example 6 was prepared similarly to Example 1 except that (E)-3-(3,4-dichloro-phenyl)-propenal was replaced with (E)-3-(4-chloro-phenyl)-propenal. LC-MS (m/z): 550.2 (M+H).

### Example 7

### (E)-N-((2-chloropyridin-4-yl)methyl)-1-(3-(3,4-dichlorophenyl)allyl)-2′,2′-dimethylspiro[piperidine-4,7′-[1,3]dioxolo[4,5-f]indole]-5′(6′H)-carboxamide

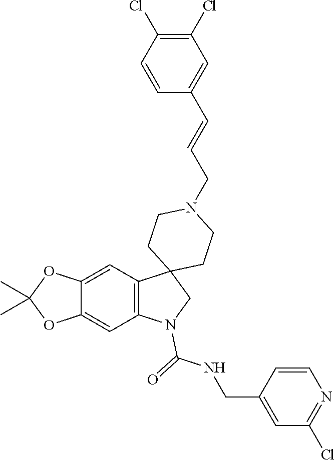 [SMILES: CC1(C)Oc2cc3c(cc2O1)C1(CCN(C/C=C/c2ccc(Cl)c(Cl)c2)CC1)CN3C(=O)NCc1ccnc(Cl)c1]

Example 7 was prepared similarly to Example 1 except that in step 1 and the synthesis of intermediate 1.11 1,3-dihydroisobenzofuran-5-amine was replaced with 2,2-dimethylbenzo[d][1,3]dioxol-5-amine. LC-MS (m/z): 584.1 (M+H).

### Example 8

### (E)-1-(3-(4-chlorophenyl)allyl)-N-((2-chloropyridin-4-yl)methyl)-2′,2′-dimethylspiro[piperidine-4,7′-[1,3]dioxolo[4,5-f]indole]-5′(6′H)-carboxamide

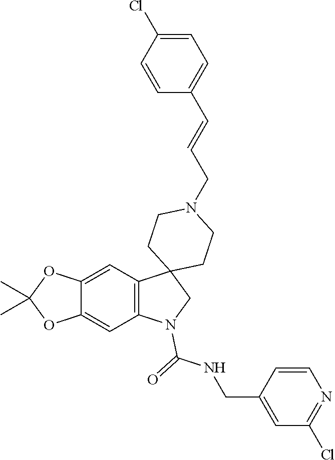 [SMILES: CC1(C)Oc2cc3c(cc2O1)C1(CCN(C/C=C/c2ccc(Cl)cc2)CC1)CN3C(=O)NCc1ccnc(Cl)c1]

Example 8 was prepared similarly to Example 1 except that in step 1 and the synthesis of intermediate 1.11 1,3-dihydroisobenzofuran-5-amine was replaced with 2,2-dimethylbenzo[d][1,3]dioxol-5-amine and that (E)-3-(3,4-dichloro-phenyl)-propenal was replaced with (E)-3-(4-chloro-phenyl)-propenal. LC-MS (m/z): 578.2 (M+H).

### Example 9

### (E)-N-((2-chloropyridin-4-yl)methyl)-1-(3-(3,4-dichlorophenyl)allyl)-2′,3′-dihydrospiro[piperidine-4,8′-[1,4]dioxino[2,3-f]indole]-6′(7′H)-carboxamide

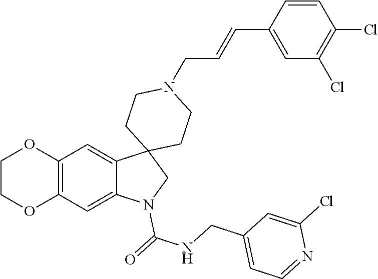 [SMILES: O=C(NCc1ccnc(Cl)c1)N1CC2(CCN(C/C=C/c3ccc(Cl)c(Cl)c3)CC2)c2cc3c(cc21)OCCO3]

Example 9 was prepared similarly to Example 1, except that Intermediate 1.12b was replaced with Intermediate 1.16b. 1H NMR (400 MHz, CHLOROFORM-d) δ: 1.73 (d, J=12.88 Hz, 2H) 1.91-2.28 (m, 4H) 3.04 (d, J=8.34 Hz, 2H) 3.24 (br. s., 2H) 3.79 (s, 2H) 4.23 (s, 4H) 4.53 (d, J=5.81 Hz, 2H) 5.25 (br. s., 1H) 6.28-6.39 (m, 1H) 6.42-6.52 (m, 1H) 6.71 (s, 1H) 7.18-7.25 (m, 2H) 7.31 (s, 1H) 7.34-7.42 (m, 2H) 7.47 (d, J=1.77 Hz, 1H) 8.34 (d, J=5.05 Hz, 1H). LC-MS (m/z): 599.0 (M+H).

### Example 10

### (E)-N-((2-chloropyridin-4-yl)methyl)-1′-(3-(3,4-dichlorophenyl)allyl)-6,8-dihydrospiro[furo[3,4-g]indole-3,4′-piperidine]-1(2H)-carboxamide

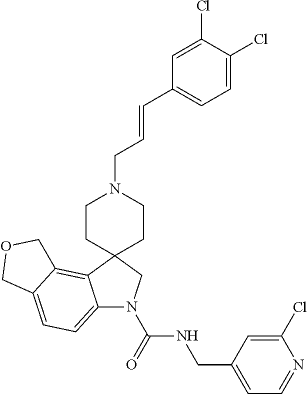 [SMILES: O=C(NCc1ccnc(Cl)c1)N1CC2(CCN(C/C=C/c3ccc(Cl)c(Cl)c3)CC2)c2c1ccc1c2COC1]

### Step 1: Preparation of tert-butyl 6-(((2-chloropyridin-4-yl)methyl)carbamoyl)-1,3,6,7-tetrahydrospiro[furo[3,4-e]indole-8,4′-piperidine]-1′-carboxylate

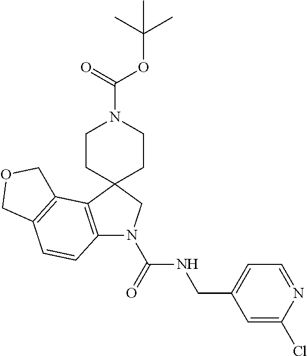 [SMILES: CC(C)(C)OC(=O)N1CCC2(CC1)CN(C(=O)NCc1ccnc(Cl)c1)c1ccc3c(c12)COC3]

To the stirred solution of (2-chloropyridin-4-yl)methanamine (100 mg, 0.562 mmol, 1 eq) in dichloromethane (6 mL) was added triethylamine (0.315 mL, 2.247 mmol, 4 eq) followed by addition of 1,1′-carbonyldiimidazole (118.31 mg, 0.73 mmol, 1.3 eq) at 0° C. Reaction was allowed to warm slowly at room temperature and stirred for 4 h. Reaction mass was cooled to 0° C. and tert-butyl 1,3,6,7-tetrahydro-spiro[furo[3,4-e]indole-8,4′-piperidine]-1′-carboxylate (148.31 mg, 0.449 mmol, 0.8 eq) was added. The reaction mass was stirred at room temperature for 16 h. Progress of reaction was monitored by TLC. After complete consumption of starting material, reaction mixture was concentrated under reduced pressure to get brown thick mass. Purification was done by column chromatography over silica gel (100-200 mesh) using 2% methanol: dichloromethane as an eluent to afford product as an off white solid (128 mg, 45.66%). ¹H NMR (400 MHz, DMSO-d₆): δ 1.42 (s, 9H), 1.59-1.62 (m, 2H), 1.65-1.73 (m, 2H), 2.82 (bs, 2H), 3.96 (m, 4H), 4.37 (d, J=5.52 Hz, 2H), 4.89 (s, 2H), 5.04 (s, 2H), 7.06 (d, J=8.2 Hz, 1H), 7.37-7.40 (m, 2H), 7.45 (s, 1H), 7.84 (d, J=8.16 Hz, 1H), 8.35 (d, J=5.0 Hz, 1H). LC-MS (m/z): 499.0 (M+H).

### Step 2: Preparation of trifluoroacetic acid salt of N-((2-chloropyridin-4-yl)methyl)-3,7-dihydrospiro[furo[3,4-e]indole-8,4′-piperidine]-6(1H)-carboxamide

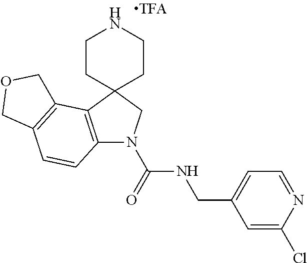 [SMILES: O=C(NCc1ccnc(Cl)c1)N1CC2(CCNCC2)c2c1ccc1c2COC1]

To the stirred solution of tert-butyl 6-((2-chloropyridin-4-yl)methylcarbamoyl)-1,3,6,7-tetrahydrospiro[furo[3,4-e]indole-8,4′-piperidine]-1′-carboxylate (125 mg, 0.251 mmol, 1 eq) in dichloromethane (3 mL) was added trifluoroacetic acid (0.5 mL) slowly at 0° C. Resulting reaction mass allowed to warm at room temperature and stirred it for 2 h. Progress of reaction was monitored by TLC. After complete consumption of starting material, reaction was concentrated under reduced pressure and stripped out with chloroform (3×3 mL) to afford brown sticky material (150 mg, crude). The crude material was used as such for next step.

### Step 3: Preparation of Example 10

In 10 mL vial, N-((2-chloropyridin-4-yl)methyl)-3,7-dihydrospiro[furo[3,4-e]indole-8,4′-piperidine]-6(1H)-carboxamide trifluoroacetic acid salt (crude, 150 mg, 0.292 mmol, 1 eq) was dissolved in dimethylformamide (5 mL), to this triethylamine (0.2 mL, 1.462 mmol, 5 eq) and (E)-3-(3,4-dichlorophenyl)-acrylaldehyde (76 mg, 0.38 mmol, 1.3 eq) were added subsequently. Reaction was stirred at room temperature for 15 minutes and sodium triacetoxy borohydride (124 mg, 0.585 mmol, 2 eq) was added to the reaction mixture and reaction was allowed to stir at room temperature for 16 h. Progress of reaction was monitored by TLC. After consumption of starting material, reaction mass was concentrated under reduced pressure to afford brown sticky mass (190 mg), which was purified by preparative HPLC to afford off white solid (66 mg, 39%). ¹H NMR (400 MHz, DMSO-d₆): δ 1.57-1.60 (m, 2H), 1.82-1.88 (m, 2H), 2.04-2.10 (m, 2H), 2.88-2.91 (m, 2H), 3.14 (d, J=5.48 Hz, 2H), 3.88 (s, 2H), 4.36 (d, J=5.56 Hz, 2H), 4.88 (s, 2H), 5.09 (s, 2H), 6.47-6.58 (m, 2H), 7.04 (d, J=8.16 Hz, 1H), 7.37 (d, J=5.04 Hz, 1H), 7.44-7.49 (m, 3H), 7.57 (d, J=8.36 Hz, 1H), 7.77 (d, J=1.88 Hz, 1H), 7.84 (d, J=8.2 Hz, 1H), 8.34 (d, J=5.08 Hz, 1H). LC-MS: (m/z): 583.0 (M+H), HPLC: 98.34%

### Example 11

### (E)-N-((2-chloropyridin-4-yl)methyl)-1-(3-(3,4-dichlorophenyl)allyl)-2′,3′-dihydrospiro[piperidine-4,9′-[1,4]dioxino[2,3-e]indole]-7′(8′H)-carboxamide

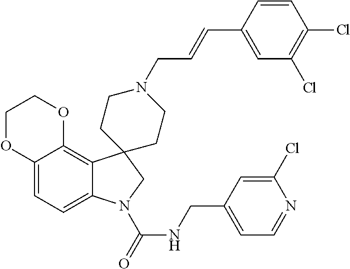 [SMILES: O=C(NCc1ccnc(Cl)c1)N1CC2(CCN(C/C=C/c3ccc(Cl)c(Cl)c3)CC2)c2c1ccc1c2OCCO1]

Example 11 was prepared similarly to Example 9, except that Intermediate 1.12a was replaced with Intermediate 1.16a. 1H NMR (400 MHz, CHLOROFORM-d) δ: 1.61 (d, J=13.39 Hz, 2H) 1.97-2.14 (m, 2H) 2.54-2.69 (m, 2H) 3.00 (d, J=9.85 Hz, 2H) 3.20 (d, J=4.55 Hz, 2H) 3.79 (s, 2H) 4.17-4.32 (m, 4H) 4.51 (d, J=6.06 Hz, 2H) 5.16 (br. s., 1H) 6.25-6.39 (m, 1H) 6.39-6.49 (m, 1H) 6.72 (d, J=8.59 Hz, 1H) 7.17-7.23 (m, 2H) 7.28-7.34 (m, 2H) 7.38 (d, J=8.34 Hz, 1H) 7.45 (d, J=1.77 Hz, 1H) 8.33 (d, J=5.05 Hz, 1H). LC-MS (m/z): 599.0 (M+H).

### Cyclopentyl Analog (Example 12)

The following examples were prepared in accordance to the schemes and similarly to procedures described in Examples 1 and 2, except that Intermediates 1.13, 1.14(a) and 1.14(b) were used in place of Intermediates 1.11, 1.12(a) and 1.12(b).

### Example 12

### (E)-N-((2-chloropyridin-4-yl)methyl)-1′-(3-(3,4-dichloro phenyl)allyl)-2,5,6,7-tetrahydro-1H-spiro[cyclopenta[f]indole-3,4′-piperidine]-1-carboxamide

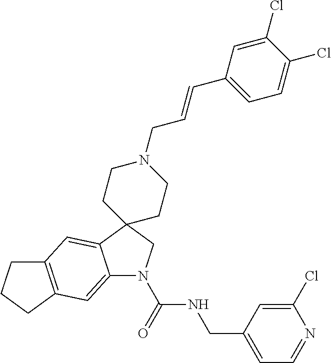 [SMILES: O=C(NCc1ccnc(Cl)c1)N1CC2(CCN(C/C=C/c3ccc(Cl)c(Cl)c3)CC2)c2cc3c(cc21)CCC3]

### Step 1: tert-butyl 1-((2-chloropyridin-4-yl)carbamoyl)-2,5,6,7-tetrahydro-1H-spiro[cyclopenta[f]indole-3,4′-piperidine]-1′-carboxylate

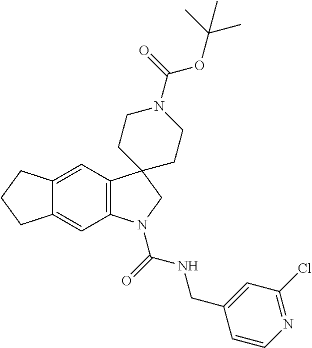 [SMILES: CC(C)(C)OC(=O)N1CCC2(CC1)CN(C(=O)NCc1ccnc(Cl)c1)c1cc3c(cc12)CCC3]

In a 10 mL vial, to the stirred solution of (2-chloropyridin-4-yl)methanamine hydrochloride (135 mg, 0.758mmoL, 1 eq) in dichloromethane (6 mL) was added triethylamine (0.42 mL, 3.034mmoL, 4 eq) followed by 1,1′-carbonyldiimidazole (159.72 mg, 0.986 mmol, 1.3 eq) at 0° C. Reaction was allowed to warm slowly at room temperature and stirred for 4 hours. Progress of the reaction was monitored by TLC using ethyl acetate as mobile phase. After completion, reaction was cooled to 0° C. and tert-butyl 2,5,6,7-tetrahydro-1H-spiro[cyclopenta[f]indole-3,4′-piperidine]-1′-carboxylate (Intermediate 1.14b, 199 mg, 0.607mmoL, 0.8 eq) was added and stirred at room temperature for 16 hours. After consumption of starting material, reaction mass was concentrated under reduced pressure to get brown thick mass which was purified by column chromatography using silica gel (100-200 mesh size) and product was eluted with 2% methanol: dichloromethane to afford off white solid (284 mg, 75%). ¹H NMR (400 MHz, DMSO-d₆) δ: 1.42 (s, 9H), 1.56-1.59 (m, 2H), 1.64-1.72 (m, 2H), 1.95-2.0 (m, 2H), 2.74-2.76 (m, 4H), 2.82-2.84 (m, 2H), 3.90 (s, 2H), 3.96-3.99 (m, 2H), 4.35 (d, J=5.56 Hz, 2H), 7.02 (s, 1H), 7.31-7.33 (m, 1H), 7.36 (d, J=4.92 Hz, 1H), 7.43 (s, 1H), 7.68 (s, 1H), 8.34 (d, J=5.04 Hz, 1H), LC-MS (m/z): 497.2 (M+H).

### Step 2: Preparation of trifluoroacetic acid salt of N-((2-chloropyridin-4-yl)methyl)-2,5,6,7-tetrahydro-1H-spiro[cyclopenta[f]indole-3,4′-piperidine]-1-carboxamide

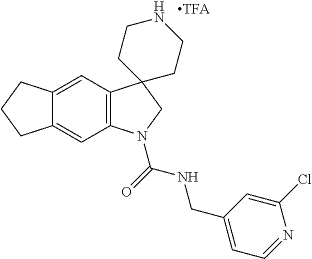 [SMILES: O=C(NCc1ccnc(Cl)c1)N1CC2(CCNCC2)c2cc3c(cc21)CCC3]

In a 10 mL vial, to the stirred solution of tert-butyl 1-((2-chloropyridin-4-yl)carbamoyl)-2,5,6,7-tetrahydro-1H-spiro[cyclopenta[f]indole-3,4′-piperidine]-1′-carboxylate (200 mg, 0.402mmoL, 1 eq) in dichloromethane (3 mL) was added trifluoroacetic acid (0.5 mL) slowly at 0° C. Resulting reaction mass was allowed to warm to room temperature and stirred for 2 hours. Progress of reaction was monitored by TLC. After consumption of starting material, reaction was concentrated under reduced pressure and stripped out with chloroform (3×3 mL) to afford brown sticky material (220 mg, crude). The crude material was used as such for next step.

### Step 3: Preparation of Example 12

In 10 mL vial, N-((2-chloropyridin-4-yl)methyl)-2,5,6,7-tetrahydro-1H-spiro[cyclopenta[f]indole-3,4′-piperidine]-1-carboxamide trifluoroacetic acid salt as crude (0.220 g, 0.431 mmoL, 1 eq) was dissolved in dimethylformamide (5 mL) and to the reaction mixture was added triethylamine (0.3 mL, 2.153mmoL, 5 eq) and (E)-3-(3,4-dichlorophenyl) acrylaldehyde (0.112 g, 0.56mmoL, 1.3 eq). Reaction was stirred at room temperature for 15 minutes. Sodium triacetoxy borohydride (0.183 g, 0.861 mmoL, 2 eq) was added to the reaction mixture and reaction was allowed to stir at room temperature for 16 hours. After consumption of starting material, reaction mass was concentrated in speed vac to afford brown sticky mass which was purified by preparative HPLC to afford off white solid (168 mg, 67%). ¹H NMR (400 MHz, DMSO-d₆) δ: 1.56-1.59 (m, 2H), 1.81-1.84 (m, 2H), 1.90-1.99 (m, 2H), 2.00-2.10 (m, 2H), 2.72-2.77 (m, 4H), 2.87-2.90 (m, 2H), 3.14 (d, J=5.72 Hz, 2H), 3.83 (s, 2H), 4.34 (d, J=5.6 Hz, 2H), 6.48-6.52 (m, 1H), 6.56 (d, J=16.0 Hz, 1H), 7.00 (s, 1H), 7.35-7.38 (m, 2H), 7.42 (s, 1H), 7.47 (dd, J₁=1.92 Hz, J₂=8.4 Hz, 1H), 7.57 (d, J=7.96 Hz, 1H), 7.69 (s, 1H), 7.76 (d, J=1.88 Hz, 1H), 8.34 (d, J=5.08 Hz, 1H), LC-MS: (m/z): 581.0 (M+H). HPLC: 99.67%

### Thiazole Analogs

The following thiazole analogs (Example 13-47) were prepared in accordance to the schemes described herein and similarly to procedures described in Examples 1 and 2, except that the thiazole intermediates were used and alternate commercially available amines were used to provide an alternate urea.

### Example 13

### (E)-1-(3-(3,4-dichlorophenyl)allyl)-N-(pyridin-4-ylmethyl)spiro[piperidine-4,8′-thiazolo[4,5-e]indole]-6′(7′H)-carboxamide

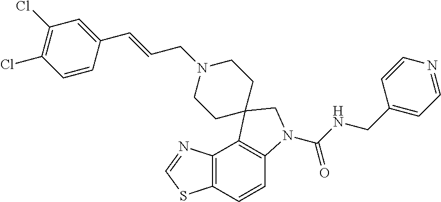 [SMILES: O=C(NCc1ccncc1)N1CC2(CCN(C/C=C/c3ccc(Cl)c(Cl)c3)CC2)c2c1ccc1scnc21]

Example 13 was prepared similarly to Example 1 and 2 except that Intermediates 1.3 and 1.4 were used and commercially available pyridin-4-ylmethanamine was substituted for (2-chloropyridin-4-yl)methanamine.

### Example 14

### (E)-1-(3-(3,4-dichlorophenyl)allyl)-N-(pyrimidin-2-ylmethyl)spiro[piperidine-4,8′-thiazolo[4,5-e]indole]-6′(7′H)-carboxamide

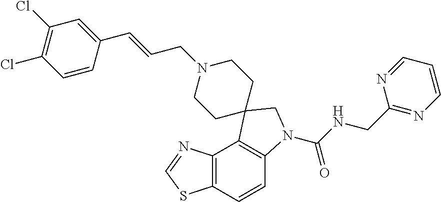 [SMILES: O=C(NCc1ncccn1)N1CC2(CCN(C/C=C/c3ccc(Cl)c(Cl)c3)CC2)c2c1ccc1scnc21]

Example 14 was prepared similarly to Example 1 and 2 except that Intermediates 1.3 and 1.4 were used and commercially available pyrimidin-2-ylmethanamine was substituted for (2-chloropyridin-4-yl)methanamine.

### Example 15

### (E)-N-benzyl-1-(3-(3,4-dichlorophenyl)allyl)spiro[piperidine-4,8′-thiazolo[4,5-e]indole]-6′(7′H)-carboxamide

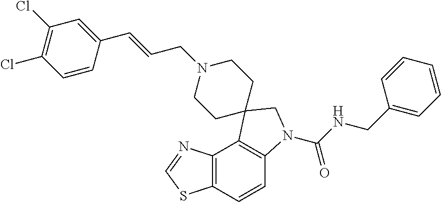 [SMILES: O=C(NCc1ccccc1)N1CC2(CCN(C/C=C/c3ccc(Cl)c(Cl)c3)CC2)c2c1ccc1scnc21]

Example 15 was prepared similarly to Example 1 and 2 except that Intermediates 1.3 and 1.4 were used and commercially available benzylamine was substituted for (2-chloropyridin-4-yl)methanamine.

### Example 16

### (E)-1-(3-(3,4-dichlorophenyl)allyl)-N-(4-fluorobenzyl)spiro[piperidine-4,8′-thiazolo[4,5-e]indole]-6′(7′H)-carboxamide

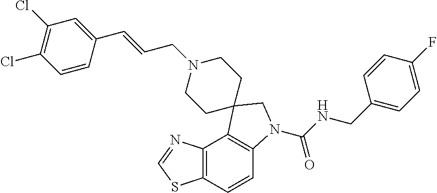 [SMILES: O=C(NCc1ccc(F)cc1)N1CC2(CCN(C/C=C/c3ccc(Cl)c(Cl)c3)CC2)c2c1ccc1scnc21]

Example 16 was prepared similarly to Example 1 and 2 except that Intermediates 1.3 and 1.4 were used and commercially available (4-fluorophenyl)methanamine was substituted for (2-chloropyridin-4-yl)methanamine.

### Example 17

### (E)-1-(3-(3,4-dichlorophenyl)allyl)-N-(4-nitrobenzyl)spiro[piperidine-4,8′-thiazolo[4,5-e]indole]-6′(7′H)-carboxamide

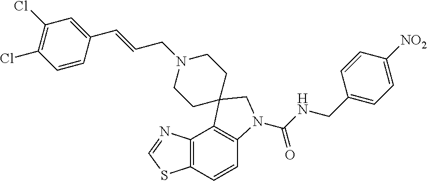 [SMILES: O=C(NCc1ccc([N+](=O)[O-])cc1)N1CC2(CCN(C/C=C/c3ccc(Cl)c(Cl)c3)CC2)c2c1ccc1scnc21]

Example 17 was prepared similarly to Example 1 and 2 except that Intermediates 1.3 and 1.4 were used and commercially available (4-nitrophenyl)methanamine was substituted for (2-chloropyridin-4-yl)methanamine.

### Example 18

### (E)-1-(3-(3,4-dichlorophenyl)allyl)-N-(4-hydroxybenzyl)spiro[piperidine-4,8′-thiazolo[4,5-e]indole]-6′(7′H)-carboxamide

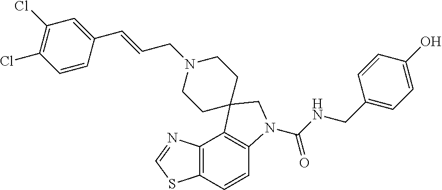 [SMILES: O=C(NCc1ccc(O)cc1)N1CC2(CCN(C/C=C/c3ccc(Cl)c(Cl)c3)CC2)c2c1ccc1scnc21]

Example 18 was prepared similarly to Example 1 and 2 except that Intermediates 1.3 and 1.4 were used and commercially available 4-(aminomethyl)phenol was substituted for (2-chloropyridin-4-yl)methanamine.

### Example 19

### (E)-1-(3-(3,4-dichlorophenyl)allyl)-N-(pyrazin-2-ylmethyl)spiro[piperidine-4,8′-thiazolo[4,5-e]indole]-6′(7′H)-carboxamide

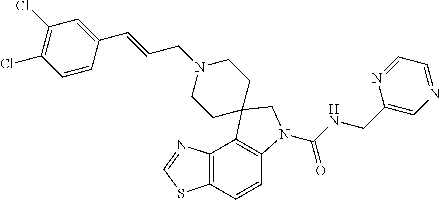 [SMILES: O=C(NCc1cnccn1)N1CC2(CCN(C/C=C/c3ccc(Cl)c(Cl)c3)CC2)c2c1ccc1scnc21]

Example 19 was prepared similarly to Example 1 and 2 except that Intermediates 1.3 and 1.4 were used and commercially available pyrazin-2-ylmethanamine was substituted for (2-chloropyridin-4-yl)methanamine.

### Example 20

### (E)-N-(4-chlorobenzyl)-1-(3-(3,4-dichlorophenyl)allyl)spiro[piperidine-4,8′-thiazolo[4,5-e]indole]-6′(7′H)-carboxamide

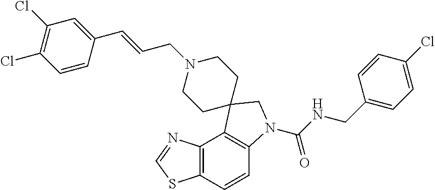 [SMILES: O=C(NCc1ccc(Cl)cc1)N1CC2(CCN(C/C=C/c3ccc(Cl)c(Cl)c3)CC2)c2c1ccc1scnc21]

Example 20 was prepared similarly to Example 1 and 2 except that Intermediates 1.3 and 1.4 were used and commercially available (4-chlorophenyl)methanamine was substituted for (2-chloropyridin-4-yl)methanamine.

### Example 21

### (E)-N-((2-chlorothiazol-5-yl)methyl)-1-(3-(3,4-dichlorophenyl)allyl)spiro[piperidine-4,8′-thiazolo[4,5-e]indole]-6′(7′H)-carboxamide

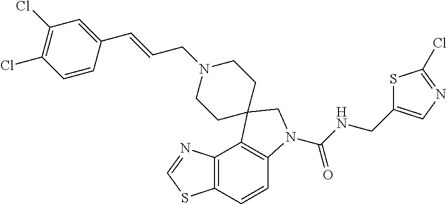 [SMILES: O=C(NCc1cnc(Cl)s1)N1CC2(CCN(C/C=C/c3ccc(Cl)c(Cl)c3)CC2)c2c1ccc1scnc21]

Example 21 was prepared similarly to Example 1 and 2 except that Intermediates 1.3 and 1.4 were used and commercially available (2-chlorothiazol-5-yl)methanamine was substituted for (2-chloropyridin-4-yl)methanamine.

### Example 22

### (E)-1-(3-(3,4-dichlorophenyl)allyl)-N-(thiazol-5-ylmethyl)spiro[piperidine-4,8′-thiazolo[4,5-e]indole]-6′(7′H)-carboxamide

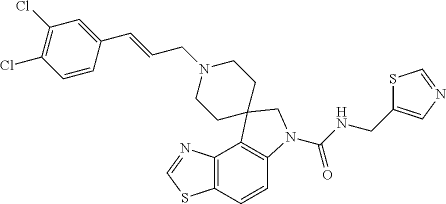 [SMILES: O=C(NCc1cncs1)N1CC2(CCN(C/C=C/c3ccc(Cl)c(Cl)c3)CC2)c2c1ccc1scnc21]

Example 22 was prepared similarly to Example 1 and 2 except that Intermediates 1.3 and 1.4 were used and commercially available thiazol-5-ylmethanamine was substituted for (2-chloropyridin-4-yl)methanamine.

### Example 23

### (E)-1-(3-(3,4-dichlorophenyl)allyl)-N-(thiazol-2-ylmethyl)spiro[piperidine-4,8′-thiazolo[4,5-e]indole]-6′(7′H)-carboxamide

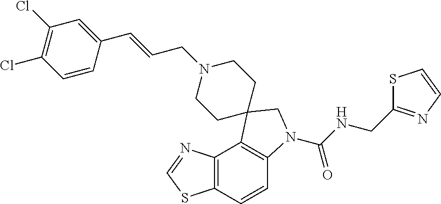 [SMILES: O=C(NCc1nccs1)N1CC2(CCN(C/C=C/c3ccc(Cl)c(Cl)c3)CC2)c2c1ccc1scnc21]

Example 23 was prepared similarly to Example 1 and 2 except that Intermediates 1.3 and 1.4 were used and commercially available thiazol-2-ylmethanamine was substituted for (2-chloropyridin-4-yl)methanamine.

### Example 24

### (E)-1-(3-(3,4-dichlorophenyl)allyl)-N-(thiazol-4-ylmethyl)spiro[piperidine-4,8′-thiazolo[4,5-e]indole]-6′(7′H)-carboxamide

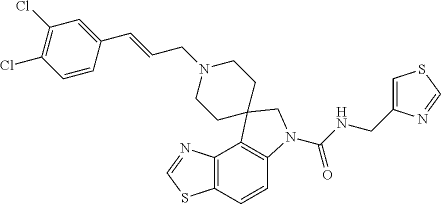 [SMILES: O=C(NCc1cscn1)N1CC2(CCN(C/C=C/c3ccc(Cl)c(Cl)c3)CC2)c2c1ccc1scnc21]

Example 24 was prepared similarly to Examples 1 and 2 except that Intermediates 1.3 and 1.4 were used and commercially available thiazol-2-ylmethanamine was substituted for (2-chloropyridin-4-yl)methanamine.

### Example 25

### (E)-N-(cyclohexylmethyl)-1-(3-(3,4-dichlorophenyl)allyl)spiro[piperidine-4,8′-thiazolo[4,5-e]indole]-6′(7′H)-carboxamide

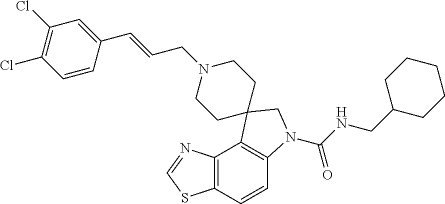 [SMILES: O=C(NCC1CCCCC1)N1CC2(CCN(C/C=C/c3ccc(Cl)c(Cl)c3)CC2)c2c1ccc1scnc21]

Example 25 was prepared similarly to Example 1 and 2 except that Intermediates 1.3 and 1.4 were used and commercially available cyclohexylmethanamine was substituted for (2-chloropyridin-4-yl)methanamine.

### Example 26

### (E)-1-(3-(3,4-dichlorophenyl)allyl)-N-((1-methyl-1H-pyrazol-4-yl)methyl)spiro[piperidine-4,8′-thiazolo[4,5-e]indole]-6′(7′H)-carboxamide

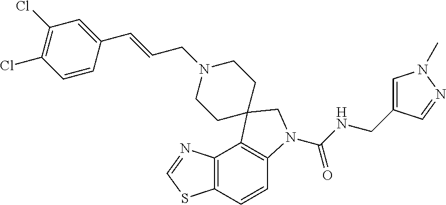 [SMILES: Cn1cc(CNC(=O)N2CC3(CCN(C/C=C/c4ccc(Cl)c(Cl)c4)CC3)c3c2ccc2scnc32)cn1]

Example 26 was prepared similarly to Example 1 and 2 except that Intermediates 1.3 and 1.4 were used and commercially available (1-methyl-1H-pyrazol-4-yl)methanamine was substituted for (2-chloropyridin-4-yl)methanamine.

### Example 27

### (E)-1-(3-(3,4-dichlorophenyl)allyl)-N-((1-methyl-1H-pyrazol-5-yl)methyl)spiro[piperidine-4,8′-thiazolo[4,5-e]indole]-6′(7′H)-carboxamide

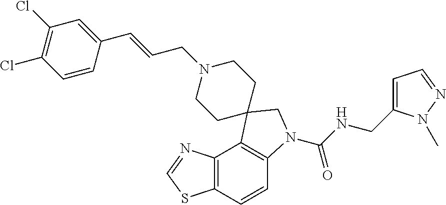 [SMILES: Cn1nccc1CNC(=O)N1CC2(CCN(C/C=C/c3ccc(Cl)c(Cl)c3)CC2)c2c1ccc1scnc21]

Example 27 was prepared similarly to Example 1 and 2 except that Intermediates 1.3 and 1.4 were used and commercially available (1-methyl-1H-pyrazol-5-yl)methanamine was substituted for (2-chloropyridin-4-yl)methanamine.

### Example 28

### (E)-1-(3-(3,4-dichlorophenyl)allyl)-N-((1-methyl-1H-pyrazol-3-yl)methyl)spiro[piperidine-4,8′-thiazolo[4,5-e]indole]-6′(7′H)-carboxamide

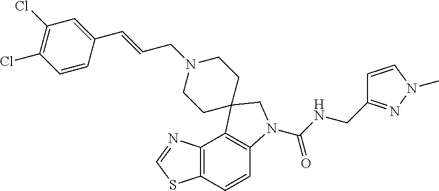 [SMILES: Cn1ccc(CNC(=O)N2CC3(CCN(C/C=C/c4ccc(Cl)c(Cl)c4)CC3)c3c2ccc2scnc32)n1]

Example 28 was prepared similarly to Example 1 and 2 except that Intermediates 1.3 and 1.4 were used and commercially available (1-methyl-1H-pyrazol-3-yl)methanamine was substituted for (2-chloropyridin-4-yl)methanamine.

### Example 29

### (E)-1-(3-(3,4-dichlorophenyl)allyl)-N-(oxazol-4-ylmethyl)spiro[piperidine-4,8′-thiazolo[4,5-e]indole]-6′(7′H)-carboxamide

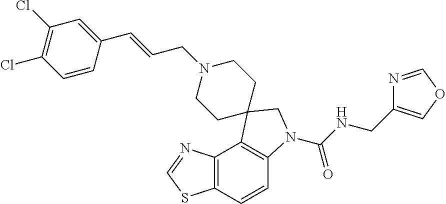 [SMILES: O=C(NCc1cocn1)N1CC2(CCN(C/C=C/c3ccc(Cl)c(Cl)c3)CC2)c2c1ccc1scnc21]

Example 29 was prepared similarly to Example 1 and 2 except that Intermediates 1.3 and 1.4 were used and commercially available (1-methyl-1H-pyrazol-3-yl)methanamine was substituted for (2-chloropyridin-4-yl)methanamine.

### Example 30

### (E)-1-(3-(3,4-dichlorophenyl)allyl)-N-(oxazol-5-ylmethyl)spiro[piperidine-4,8′-thiazolo[4,5-e]indole]-6′(7′H)-carboxamide

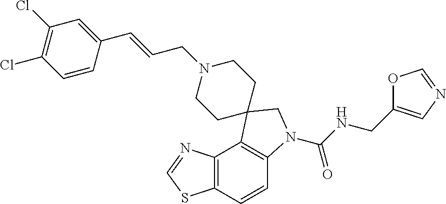 [SMILES: O=C(NCc1cnco1)N1CC2(CCN(C/C=C/c3ccc(Cl)c(Cl)c3)CC2)c2c1ccc1scnc21]

Example 30 was prepared similarly to Example 1 and 2 except that Intermediates 1.3 and 1.4 were used and commercially available oxazol-5-ylmethanamine was substituted for (2-chloropyridin-4-yl)methanamine.

### Example 31

### (E)-1-(3-(3,4-dichlorophenyl)allyl)-N-isobutylspiro[piperidine-4,8′-thiazolo[4,5-e]indole]-6′(7′H)-carboxamide

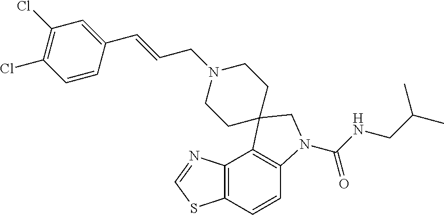 [SMILES: CC(C)CNC(=O)N1CC2(CCN(C/C=C/c3ccc(Cl)c(Cl)c3)CC2)c2c1ccc1scnc21]

Example 31 was prepared similarly to Example 1 and 2 except that Intermediates 1.3 and 1.4 were used and commercially available 2-methylpropan-1-amine was substituted for (2-chloropyridin-4-yl)methanamine.

### Example 32

### (E)-N-(cyclopropylmethyl)-1-(3-(3,4-dichlorophenyl)allyl)spiro[piperidine-4,8′-thiazolo[4,5-e]indole]-6′(7′H)-carboxamide

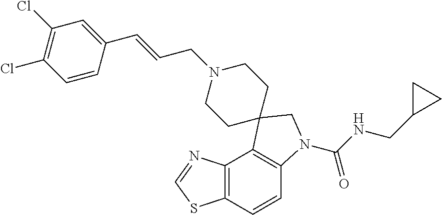 [SMILES: O=C(NCC1CC1)N1CC2(CCN(C/C=C/c3ccc(Cl)c(Cl)c3)CC2)c2c1ccc1scnc21]

Example 32 was prepared similarly to Example 1 and 2 except that Intermediates 1.3 and 1.4 were used and commercially available cyclopropylmethanamine was substituted for (2-chloropyridin-4-yl)methanamine.

### Example 33

### (E)-N-(cyclopentylmethyl)-1-(3-(3,4-dichlorophenyl)allyl)spiro[piperidine-4,8′-thiazolo[4,5-e]indole]-6′(7′H)-carboxamide

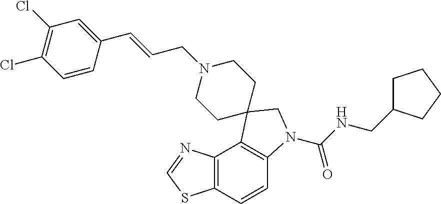 [SMILES: O=C(NCC1CCCC1)N1CC2(CCN(C/C=C/c3ccc(Cl)c(Cl)c3)CC2)c2c1ccc1scnc21]

Example 33 was prepared similarly to Example 1 and 2 except that Intermediates 1.3 and 1.4 were used and commercially available cyclopentylmethanamine was substituted for (2-chloropyridin-4-yl)methanamine.

### Example 34

### (+/−)(E)-1-(3-(3,4-dichlorophenyl)allyl)-N-((tetrahydrofuran-2-yl)methyl)spiro[piperidine-4,8′-thiazolo[4,5-e]indole]-6′(7′H)-carboxamide

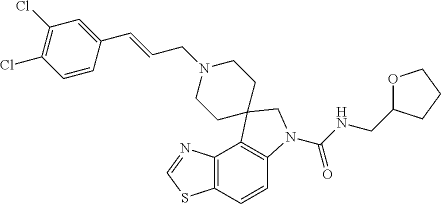 [SMILES: O=C(NCC1CCCO1)N1CC2(CCN(C/C=C/c3ccc(Cl)c(Cl)c3)CC2)c2c1ccc1scnc21]

Example 34 was prepared similarly to Example 1 and 2 except that Intermediates 1.3 and 1.4 were used and commercially available (tetrahydrofuran-2-yl)methanamine was substituted for (2-chloropyridin-4-yl)methanamine.

### Example 35

### (+/−)(E)-1-(3-(3,4-dichlorophenyl)allyl)-N-((tetrahydrofuran-3-yl)methyl)spiro[piperidine-4,8′-thiazolo[4,5-e]indole]-6′(7′H)-carboxamide

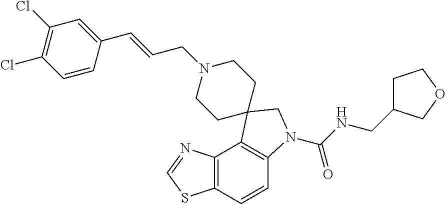 [SMILES: O=C(NCC1CCOC1)N1CC2(CCN(C/C=C/c3ccc(Cl)c(Cl)c3)CC2)c2c1ccc1scnc21]

Example 35 was prepared similarly to Example 1 and 2 except that Intermediates 1.3 and 1.4 were used and commercially available (tetrahydrofuran-3-yl)methanamine was substituted for (2-chloropyridin-4-yl)methanamine.

### Example 36

### (E)-1-(3-(3,4-dichlorophenyl)allyl)-N-(pyridin-3-methyl)spiro[piperidine-4,8′-thiazolo[4,5-e]indole]-6′(7′H)-carboxamide

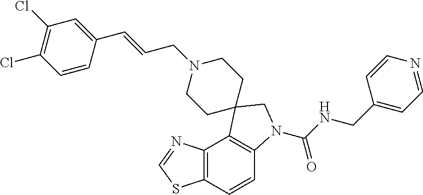 [SMILES: O=C(NCc1ccncc1)N1CC2(CCN(C/C=C/c3ccc(Cl)c(Cl)c3)CC2)c2c1ccc1scnc21]

Example 36 was prepared similarly to Example 1 and 2 except that Intermediates 1.3 and 1.4 were used and commercially available pyridin-3-ylmethanamine was substituted for (2-chloropyridin-4-yl)methanamine.

### Example 37

### (E)-1-(3-(3,4-dichlorophenyl)allyl)-N-(pyridazin-4-ylmethyl)spiro[piperidine-4,8′-thiazolo[4,5-e]indole]-6′(7′H)-carboxamide

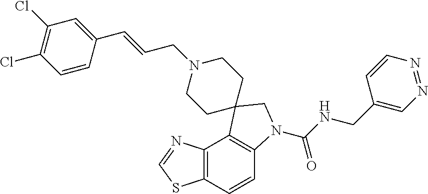 [SMILES: O=C(NCc1ccnnc1)N1CC2(CCN(C/C=C/c3ccc(Cl)c(Cl)c3)CC2)c2c1ccc1scnc21]

Example 37 was prepared similarly to Example 1 and 2 except that Intermediates 1.3 and 1.4 were used and commercially available pyridazin-4-ylmethanamine was substituted for (2-chloropyridin-4-yl)methanamine.

### Example 38

### (E)-N-((2-chloropyridin-4-yl)methyl)-1-(3-(4-fluorophenyl)allyl)spiro[piperidine-4,8′-thiazolo[4,5-e]indole]-6′(7′H)-carboxamide

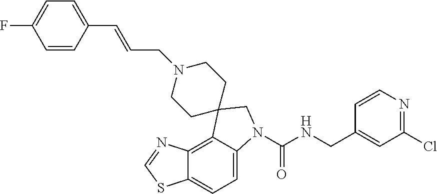 [SMILES: O=C(NCc1ccnc(Cl)c1)N1CC2(CCN(C/C=C/c3ccc(F)cc3)CC2)c2c1ccc1scnc21]

Example 38 was prepared similarly to Example 1 and 2 except that Intermediates 1.3 and 1.4 were used and commercially available (E)-3-(4-fluorophenyl)acrylaldehyde was substituted for (E)-3-(3,4-dichlorophenyl)acrylaldehyde.

### Example 39

### (E)-1-(3-(4-chlorophenyl)allyl)-N-((2-chloropyridin-4-yl)methyl)spiro[piperidine-4,8′-thiazolo[4,5-e]indole]-6′(7′H)-carboxamide

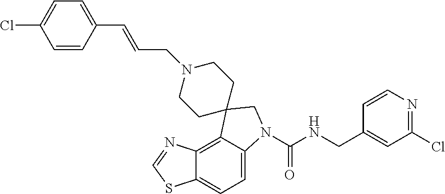 [SMILES: O=C(NCc1ccnc(Cl)c1)N1CC2(CCN(C/C=C/c3ccc(Cl)cc3)CC2)c2c1ccc1scnc21]

Example 39 was prepared similarly to Example 1 and 2 except that Intermediates 1.3 and 1.4 were used and commercially available (E)-3-(4-chlorophenyl)acrylaldehyde was substituted for (E)-3-(3,4-dichlorophenyl)acrylaldehyde.

### Example 40

### (E)-N-((2-chloropyridin-4-yl)methyl)-1-(3-(3,4-dichlorophenyl)allyl)spiro[piperidine-4,8′-thiazolo[4,5-e]indole]-6′(7′H)-carboxamide

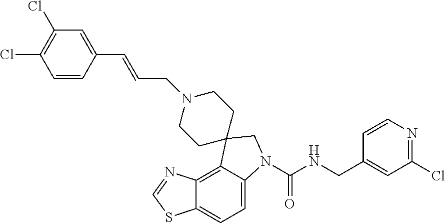 [SMILES: O=C(NCc1ccnc(Cl)c1)N1CC2(CCN(C/C=C/c3ccc(Cl)c(Cl)c3)CC2)c2c1ccc1scnc21]

Example 40 was prepared similarly to Example 1 and 2 except that Intermediates 1.3 and 1.4 were used and commercially available (E)-3-(4-chlorophenyl)acrylaldehyde was substituted for (E)-3-(3,4-dichlorophenyl)acrylaldehyde.

### Example 41

### (E)-N-((2-chloropyridin-4-yl)methyl)-1-(3-(5-(trifluoromethyl)pyridin-2-yl)allyl)spiro[piperidine-4,8′-thiazolo[4,5-e]indole]-6′(7′H)-carboxamide

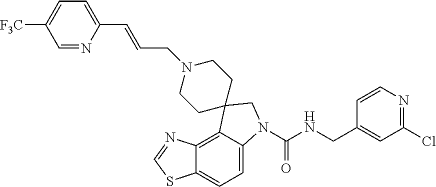 [SMILES: O=C(NCc1ccnc(Cl)c1)N1CC2(CCN(C/C=C/c3ccc(C(F)(F)F)cn3)CC2)c2c1ccc1scnc21]

Example 41 was prepared similarly to Example 1 and 2 except that Intermediates 1.3 and 1.4 and 1.17 were used.

### Example 42

### (E)-N-((2-chloropyridin-4-yl)methyl)-1-(3-(4-methoxyphenyl)allyl)spiro[piperidine-4,8′-thiazolo[4,5-e]indole]-6′(7′H)-carboxamide

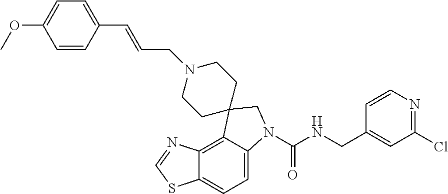 [SMILES: COc1ccc(/C=C/CN2CCC3(CC2)CN(C(=O)NCc2ccnc(Cl)c2)c2ccc4scnc4c23)cc1]

Example 42 was prepared similarly to Example 1 and 2 except that Intermediates 1.3 and 1.4 were used and commercially available (E)-3-(4-methoxyphenyl)acrylaldehyde was substituted for (E)-3-(3,4-dichlorophenyl)acrylaldehyde.

### Example 43

### (E)-1-(3-(4-chlorophenyl)allyl)-N-((2-chloropyridin-4-yl)methyl)-2′-methylspiro[piperidine-4,8′-thiazolo[4,5-e]indole]-6′(7′H)-carboxamide

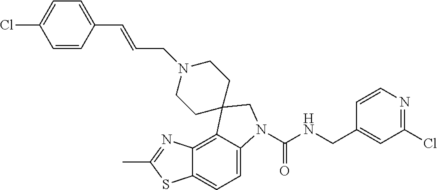 [SMILES: Cc1nc2c3c(ccc2s1)N(C(=O)NCc1ccnc(Cl)c1)CC31CCN(C/C=C/c2ccc(Cl)cc2)CC1]

Example 43 was prepared similarly to Example 1 and 2 except that Intermediates 1.5 and 1.6 were used and commercially available (E)-3-(4-chlorophenyl)acrylaldehyde was substituted for (E)-3-(3,4-dichlorophenyl)acrylaldehyde.

### Example 44

### (E)-N-((2-chloropyridin-4-yl)methyl)-1-(3-(4-fluorophenyl)allyl)-2′-methylspiro[piperidine-4,8′-thiazolo[4,5-e]indole]-6′(7′H)-carboxamide

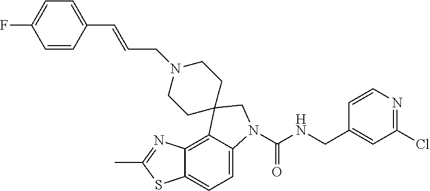 [SMILES: Cc1nc2c3c(ccc2s1)N(C(=O)NCc1ccnc(Cl)c1)CC31CCN(C/C=C/c2ccc(F)cc2)CC1]

Example 44 was prepared similarly to Example 1 and 2 except that Intermediates 1.5 and 1.6 were used and commercially available (E)-3-(4-fluorophenyl)acrylaldehyde was substituted for (E)-3-(3,4-dichlorophenyl)acrylaldehyde.

### Example 45

### (E)-N-((2-chloropyridin-4-yl)methyl)-2′-methyl-1-(3-(5-(trifluoromethyl)pyridin-2-yl)allyl)spiro[piperidine-4,8′-thiazolo[4,5-e]indole]-6′(7′H)-carboxamide

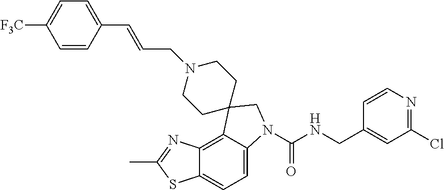 [SMILES: Cc1nc2c3c(ccc2s1)N(C(=O)NCc1ccnc(Cl)c1)CC31CCN(C/C=C/c2ccc(C(F)(F)F)cc2)CC1]

Example 45 was prepared similarly to Example 1 and 2 except that Intermediates 1.5 and 1.6 were used and Intermediate 1.17 was substituted for (E)-3-(3,4-dichlorophenyl)acrylaldehyde.

### Example 46

### (E)-N-((2-chloropyridin-4-yl)methyl)-1-(3-(4-methoxyphenyl)allyl)-2′-methylspiro[piperidine-4,8′-thiazolo[4,5-e]indole]-6′(7′H)-carboxamide

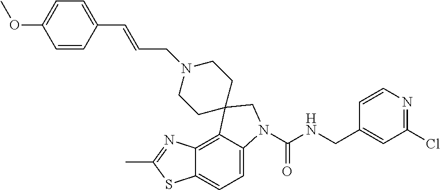 [SMILES: COc1ccc(/C=C/CN2CCC3(CC2)CN(C(=O)NCc2ccnc(Cl)c2)c2ccc4sc(C)nc4c23)cc1]

Example 46 was prepared similarly to Example 1 and 2 except that Intermediates 1.5 and 1.6 were used and commercially available (E)-3-(4-methoxyphenyl)acrylaldehyde was substituted for (E)-3-(3,4-dichlorophenyl)acrylaldehyde.

### Example 47

### (E)-N-((2-bromopyridin-4-yl)methyl)-1-(3-(3,4-dichlorophenyl)allyl)spiro[piperidine-4,8′-thiazolo[4,5-e]indole]-6′(7′H)-carboxamide

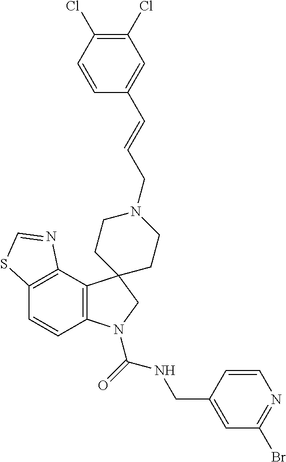 [SMILES: O=C(NCc1ccnc(Br)c1)N1CC2(CCN(C/C=C/c3ccc(Cl)c(Cl)c3)CC2)c2c1ccc1scnc21]

Example 47 was prepared similarly to Example 1 and 2 except that Intermediates 1.3 and 1.4 were used and commercially available (2-bromopyridin-4-yl)methanamine was substituted for (2-chloropyridin-4-yl)methanamine.

### Isothiazole Analogs

The isothiazole analogs (Examples 48-50) were prepared similarly to the schemes as described herein, and similarly to earlier examples, except that an alternate commercially available aldehyde was used to provide the example with an alternate R¹ substitution.

### Example 48

### (E)-N-((2-chloropyridin-4-yl)methyl)-1′-(3-(4-fluorophenyl)allyl)spiro[isothiazolo-[4,5-e]indole-8,4′-piperidine]-6(7H)-carboxamide

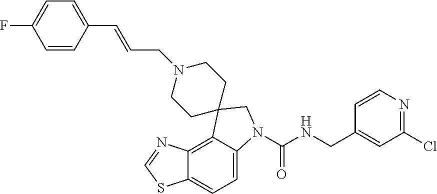 [SMILES: O=C(NCc1ccnc(Cl)c1)N1CC2(CCN(C/C=C/c3ccc(F)cc3)CC2)c2c1ccc1scnc21]

Example 48 was prepared similarly to Example 1 and 2 except that Intermediates 1.7 and 1.8 were used and commercially available (E)-3-(4-fluorophenyl)acrylaldehyde was substituted for (E)-3-(3,4-dichlorophenyl)acrylaldehyde.

### Example 49

### (E)-N-((2-chloropyridin-4-yl)methyl)-1′-(3-(5-(trifluoromethyl)pyridin-2-yl)allyl)spiro[isothiazolo[4,5-e]indole-8,4′-piperidine]-6(7H)-carboxamide

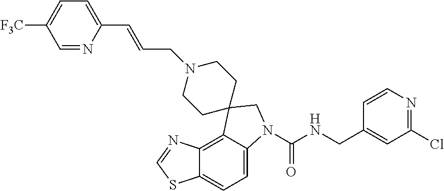 [SMILES: O=C(NCc1ccnc(Cl)c1)N1CC2(CCN(C/C=C/c3ccc(C(F)(F)F)cn3)CC2)c2c1ccc1scnc21]

Example 49 was prepared similarly to Example 1 and 2 except that Intermediates 1.7 and 1.8 were used and Intermediate 1.17 was substituted for (E)-3-(3,4-dichlorophenyl)acrylaldehyde.

### Example 50

### N-((2-chloropyridin-4-yl)methyl)-1′-((6-fluoroquinolin-2-yl)methyl)spiro[isothiazolo[4,5-e]indole-8,4′-piperidine]-6(7H)-carboxamide

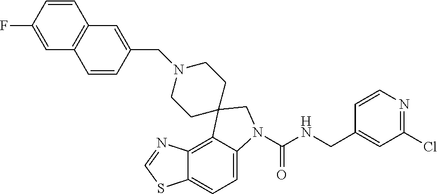 [SMILES: O=C(NCc1ccnc(Cl)c1)N1CC2(CCN(Cc3ccc4cc(F)ccc4c3)CC2)c2c1ccc1scnc21]

Example 50 was prepared similarly to Example 1 and 2 except that Intermediates 1.7 and 1.8 were used and commercially available 6-fluoroquinoline-2-carbaldehyde was substituted for (E)-3-(3,4-dichlorophenyl)acrylaldehyde.

### Indazole Analogs

The indazole analogs (Examples 51-54) were prepared similarly to the schemes as described herein, and similarly to earlier examples, except that an alternate commercially available aldehyde was used to provide the example with an alternate R¹ substitution.

### Example 51

### (E)-1-(3-(4-chlorophenyl)allyl)-N-((2-chloropyridin-4-yl)methyl)-3′-methyl-3′H-spiro[piperidine-4,8′-pyrrolo[3,2-e]indazole]-6′(7′H)-carboxamide

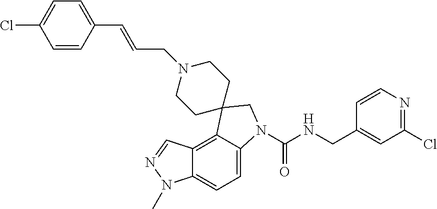 [SMILES: Cn1ncc2c3c(ccc21)N(C(=O)NCc1ccnc(Cl)c1)CC31CCN(C/C=C/c2ccc(Cl)cc2)CC1]

Example 51 was prepared similarly to Example 1 and 2 except that Intermediates 1.9 and 1.10 were used and commercially available (E)-3-(4-chlorophenyl)acrylaldehyde was substituted for (E)-3-(3,4-dichlorophenyl)acrylaldehyde.

### Example 52

### (E)-N-((2-chloropyridin-4-yl)methyl)-1-(3-(4-fluorophenyl)allyl)-3′-methyl-3′H-spiro[piperidine-4,8′-pyrrolo[3,2-e]indazole]-6′(7′H)-carboxamide

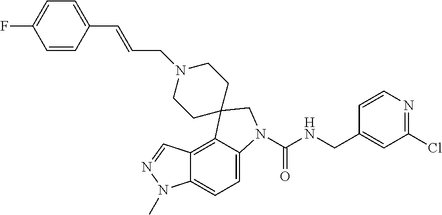 [SMILES: Cn1ncc2c3c(ccc21)N(C(=O)NCc1ccnc(Cl)c1)CC31CCN(C/C=C/c2ccc(F)cc2)CC1]

Example 52 was prepared similarly to Example 1 and 2 except that Intermediates 1.9 and 1.10 were used and commercially available (E)-3-(4-fluorophenyl)acrylaldehyde was substituted for (E)-3-(3,4-dichlorophenyl)acrylaldehyde.

### Example 53

### (E)-N-((2-chloropyridin-4-yl)methyl)-3′-methyl-1-(3-(5-(trifluoromethyl)pyridin-2-yl)allyl)-3′H-spiro[piperidine-4,8′-pyrrolo[3,2-e]indazole]-6′(7′H)-carboxamide

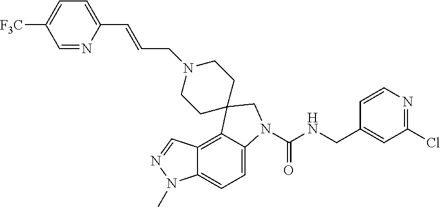 [SMILES: Cn1ncc2c3c(ccc21)N(C(=O)NCc1ccnc(Cl)c1)CC31CCN(C/C=C/c2ccc(C(F)(F)F)cn2)CC1]

Example 53 was prepared similarly to Example 1 and 2 except that Intermediates 1.9 and 1.10 were used and Intermediate 1.17 was substituted for (E)-3-(3,4-dichlorophenyl)acrylaldehyde.

### Example 54

### (E)-N-((2-chloropyridin-4-yl)methyl)-1-(3-(4-methoxyphenyl)allyl)-3′-methyl-3′H-spiro[piperidine-4,8′-pyrrolo[3,2-e]indazole]-6′(7′H)-carboxamide

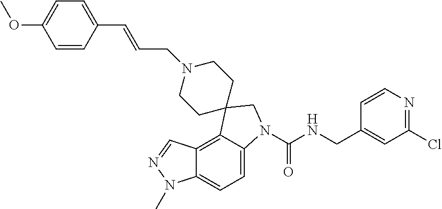 [SMILES: COc1ccc(/C=C/CN2CCC3(CC2)CN(C(=O)NCc2ccnc(Cl)c2)c2ccc4c(cnn4C)c23)cc1]

Example 54 was prepared similarly to Example 1 and 2 except that Intermediates 1.9 and 1.10 were used and commercially available (E)-3-(4-methoxyphenyl)acrylaldehyde was substituted for (E)-3-(3,4-dichlorophenyl)acrylaldehyde.

The analytical procedures used to obtain the physicochemical properties of each Formula (1A) or Formula (1B) example is described below:

### LCMS Method

Solvent A: 0.05% formic acid in water; Solvent B: acetonitrile; Gradient and Flow rate (1.5 mL/minute):

| Time | | |
|---|---|---|
| (minutes) | % A | % B |
| 0 | 98 | 2 |
| 0.75 | 98 | 2 |
| 1 | 90 | 10 |
| 2 | 2 | 98 |
| 2.25 | 2 | 98 |
| 2.9 | 98 | 2 |
| 3 | 98 | 2 |

Column: RESTEK C18, 30×2.1 mm×3μ with a 3 μL injection volume. Mass Spectrometer ionization source: ESI; with positive/negative polarity; scan range of 180-800; source temperature of 130° C.; desolvation temperature of 400° C.; cone potential (V): 25/40. Gas flow: desolvation 750 L/hr; Cone 50 L/hr, extractor voltage 3, and column temperature of 50° C. EL SD: gain of 1.5; temperature 50° C., and nitrogen pressure of 3.5 barr. UV Detector: 215 nm.

### HPLC Method 1

Solvent A: 10 mM ammonium, Solvent B: acetonitrile, gradient and flow rate (1.5 mL/minute):

| Time | | |
|---|---|---|
| (minutes) | % A | % B |
| 0 | 95 | 5 |
| 0.75 | 95 | 5 |
| 1.5 | 85 | 15 |
| 3 | 10 | 90 |
| 4 | 10 | 90 |
| 5 | 95 | 5 |
| 5.1 | 95 | 5 |

Column: Zorbax Extend C18 (50×4.6), 5μ with a 3 μL injection volume. Mass Spectrometer ionization source: ESI; with positive/negative polarity; scan range of 180-800; source temperature of 130° C.; desolvation temperature of 400° C.; cone potential (V): 40. Gas flow: desolvation 750 L/hr; Cone 50 L/hr, extractor voltage 3, and column temperature of 50° C. EL SD: gain of 1.5; temperature 50° C., and nitrogen pressure of 3.5 barr. UV Detector: 215 nm.

### HPLC Method 2.

Compounds were purified on a Gemini RP18 reversed phased HPLC column, fraction collection was triggered by a PDA trace (Unipoint settings: flow 24 ml/min, peak threshold 20000 AU, peak width 1.3, lambda 1-220 nm, lambda 2-260). Acetonitrile and 0.05% ammonia in water were used as eluent. This standard method was used for most of the samples with slight modification in the gradient as per the need. Fractions and consolidated fractions were analysed by LCMS. Column: Gemini C-18 (50×21.2 mm); Mobile phase: A) 0.05% ammonia in water B) Acetonitrile; General Gradient: Started with 10% of acetonitrile from 0 minutes and gradually increased to 100% in 8 minutes then hold 100% acetonitrile until 9 minutes and came to starting gradient at 10 minutes.

TABLE 1
Analytical Data: Formula (A) and Formula (B) Compounds

| | | Exact | Observed | Retention | HPLC- |
|---|---|---|---|---|---|
| Example | MH+ | Mass | (M + 1/M − 1) | time | uv Purity |
| 13 | 565 | 563 | 564.2 | 1.39 | 100 |
| 14 | 566 | 564 | 565.21 | 1.52 | 100 |
| 15 | 564 | 562 | 563.21 | 1.6 | 100 |
| 16 | 582 | 580 | 581.21 | 1.6 | 100 |
| 17 | 609 | 607 | 608.17 | 1.61 | 100 |
| 18 | 580 | 578 | 579.21 | 1.54 | 100 |
| 19 | 566 | 564 | | | |
| 20 | 598 | 596 | 597.15 | 1.63 | 100 |
| 21 | 605 | 603 | 604.09 | 1.57 | 100 |
| 22 | 571 | 569 | 570.14 | 1.53 | 95.6 |
| 23 | 571 | 569 | 570.14 | 1.53 | 96 |
| 24 | 571 | 569 | 570.14 | 1.54 | 100 |
| 25 | 570 | 568 | 569.25 | 1.65 | 100 |
| 26 | 568 | 566 | 567.18 | 1.52 | 100 |
| 27 | 568 | 566 | 567.21 | 1.52 | 100 |
| 28 | 568 | 566 | 567.2 | 1.53 | 100 |
| 29 | 554 | 553 | 554.17 | 1.52 | 100 |
| 30 | 554 | 553 | | | |
| 31 | 530 | 528 | 529.19 | 1.58 | 100 |
| 32 | 528 | 526 | 527.21 | 1.56 | 100 |
| 33 | 556 | 554 | 555.22 | 1.63 | 100 |
| 34 | 558 | 556 | 557.22 | 1.55 | 100 |
| 35 | 558 | 556 | 557.22 | 1.53 | 100 |
| 36 | 564 | 563 | 563.8 | 2.33 | 99.69 |
| 37 | 565 | 564 | 564.7 | 1.83 | 99.19 |
| 38 | 548 | 547 | 548.2 | 1.51 | 100 |
| 39 | 564 | 563 | 564.17 | 1.54 | 100 |
| 40 | 598 | 597 | 598.12 | 1.58 | 100 |
| 41 | 599 | 598 | 598.9 | 2.63 | 86.6 |
| 42 | 560 | 559 | 560.24 | 1.51 | 98.4 |
| 43 | 578 | 577 | 578.17 | 1.57 | 100 |
| 44 | 562 | 561 | 562.21 | 1.55 | 59.8 |
| 45 | 613 | 612 | 613 | 2.67 | 97.9 |
| 46 | 574 | 573 | 574.22 | 1.54 | 97.8 |
| 48 | 548 | 547 | 548.21 | 1.5 | 100 |
| 49 | 599 | 598 | 598.9 | 2.6 | 97.5 |
| 50 | 573 | 572 | 573.19 | 1.5 | 100 |
| 51 | 561 | 560 | 561.21 | 1.5 | 100 |
| 52 | 545 | 544 | 545.25 | 1.47 | 100 |
| 53 | 596 | 595 | 596.26 | 1.47 | 96.1 |
| 54 | 557 | 556 | 557.25 | 1.46 | 100 |

The following Formula (1C) examples Examples 55-189 were prepared in accordance with methods described herein.

### Example 55

### Radical Template Synthesis

### (E)-N-((2-chloropyridin-4-yl)methyl)-1′-(3-(3,4-dichlorophenyl)allyl)-5-(trifluoromethoxy)spiro[indoline-3,4′-piperidine]-1-carboxamide

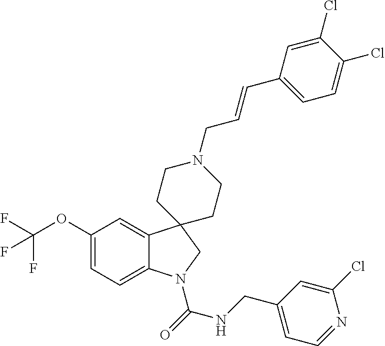 [SMILES: O=C(NCc1ccnc(Cl)c1)N1CC2(CCN(C/C=C/c3ccc(Cl)c(Cl)c3)CC2)c2cc(OC(F)(F)F)ccc21]

### Step 1: 2-bromo-4-(trifluoromethoxy)aniline

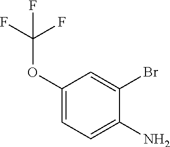 [SMILES: Nc1ccc(OC(F)(F)F)cc1Br]

N-Bromosuccinamide (3.62 g, 20.34 mmol) was added to a solution of commercially available 4-(trifluoromethoxy)aniline (3.00 g, 16.95 mmol) in acetonitrile (30 mL) at 0° C. The resulting mixture was allowed to warm to room temperature and stirred for 4 hours. The solvent was removed under reduced pressure to give the crude product which was purified by column chromatography eluting in 10% ethyl acetate/hexane to afford the title compound as brown oil (3.6 gm, 83%): 1H NMR (400 MHz, CDCl₃) δ: 4.11 (bs, 2H), 6.73 (d, 1H), 6.99 (dd, 1H), 7.31 (d, 1H). LC-Ms (m/z): [M−H]=253.1.

### Step 2: tert-butyl 4-((2-bromo-4-(trifluoromethoxy)phenylamino)methyl)-5,6-dihydropyridine-1(2H)-carboxylate

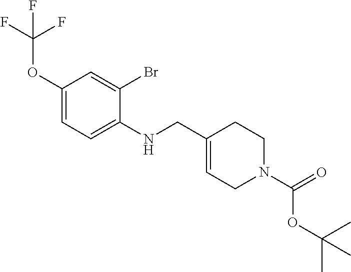 [SMILES: CC(C)(C)OC(=O)N1CC=C(CNc2ccc(OC(F)(F)F)cc2Br)CC1]

A solution of commercially available 2-bromo-4-(trifluoromethoxy)aniline (the product of step 1, Example 55, 4.00 g, 15.63 mmol) in DMF (10 mL) was added slowly to a suspension of NaH (60% in mineral oil, 1.13 g, 46.88 mmol) in DMF (20 mL) at 0° C. After warming to room temperature the reaction mixture was stirred for 30 minutes. Commercially available tert-butyl 4-(chloromethyl)-5,6-dihydropyridine-1(2H)-carboxylate (3.62 g, 15.63 mmol) in DMF (10 mL) was added and the mixture stirred at room temperature for 1 hour. Cold water (100 mL) was added and the mixture extracted with ethyl acetate (3×100 mL). The combined organic layers were washed with cold water (2×50 mL) and brine (50 mL), dried over anhydrous sodium sulphate and concentrated. The crude material was purified by flash chromatography eluting with 20% ethyl acetate/hexane to afford the title compound as grey solid (2.5 g, 36%): 1H NMR (400 MHz, DMSO-d₆) δ: 1.38 (s, 9H), 1.99 (bs, 2H), 3.39 (t, 2H), 3.75 (bs, 4H), 5.50 (s, 1H), 5.79 (t, 1H), 6.60 (d, 1H), 7.17 (d, 1H), 7.47 (d, 1H). LC-Ms (m/z): [M−H]=448.8.

### Step 3: tert-butyl 5-(trifluoromethoxy)spiro[indoline-3,4′-piperidine]-1′-carboxylate

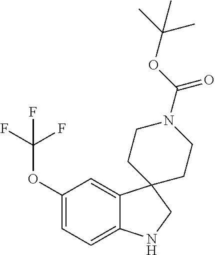 [SMILES: CC(C)(C)OC(=O)N1CCC2(CC1)CNc1ccc(OC(F)(F)F)cc12]

A solution of AIBN (55 mg, 0.33 mmol) and tributyl tinhydride (1.34 mL, 4.99 mmol) in toluene (2 mL) was added slowly to a stirred solution of the product of step 2 example 55 (1.5 g, 3.32 mmol) in toluene (48 mL) 90° C. After stirring for 16 hours at 100° C., the mixture was cooled to room temperature. A saturated solution of KF (50 mL) was added and the mixture extracted with ethyl acetate (3×50 mL). The combined extracts were washed with water followed by brine, dried over sodium sulphate and concentrated. The crude compound was purified by column chromatography eluting in 10% ethyl acetate/hexane to afford the title compound as off white solid (500 mg, 40%): 1H NMR (400 MHz, DMSO-d₆) δ: 1.41 (s, 9H), 1.56-1.68 (m, 4H), 2.85-2.89 (m, 2H), 3.40 (s, 2H), 3.89-3.90 (m, 2H), 5.80 (s, 1H), 6.48 (d, 1H), 6.88 (d, 1H), 7.02 (d, 1H). LC-MS (m/z): [M+H]=373.0.

### Step 4: tert-butyl 1-((2-chloropyridin-4-yl)methylcarbamoyl)-5-(trifluoromethoxy)spiro[indoline-3,4′-piperidine]-1′-carboxylate

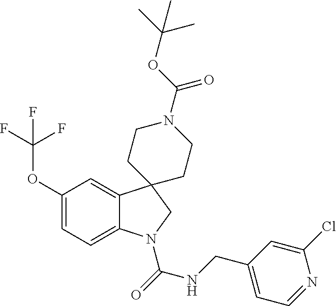 [SMILES: CC(C)(C)OC(=O)N1CCC2(CC1)CN(C(=O)NCc1ccnc(Cl)c1)c1ccc(OC(F)(F)F)cc12]

Potassium carbonate (2.30 g, 16.85 mmol) in water (2.5 mL) was added to a solution of triphosgene (1.2 g, 4.05 mmol) in DCM (7.5 mL) at 0° C. After stirring at 0° C. for 10 minutes, commercially available (2-chloro-pyridin-4-yl)-methylamine hydrochloride (600 mg, 3.37 mmol, 1 eq.) was added. After stirring at 0° C. for 2 hours the organic layer was separated, dried over sodium sulphate, filtered and evaporated. The product of step 3, Example 55 (300 mg, 0.84 mmol, 1 eq.) was added to the aforementioned isocyanate in THF (2 mL). After stirring at room temperature for 16 hours the crude material was purified by column chromatography on silica gel eluting in 2% methanol/DCM to afford the title compound as off white solid (350 mg, 77%): 1 NMR (400 MHz, DMSO-d₆) δ: 1.42 (s, 9H), 1.66 (d, 2H), 1.72-1.80 (m, 2H), 2.82 (bs, 2H), 3.98 (s, 3H), 4.02-4.03 (m, 1H), 4.37 (d, 2H), 7.10 (d, 1H), 7.30 (d, 1H), 7.37 (d, 1H), 7.45 (s, 1H), 7.48 (t, 1H), 7.87 (d, 1H), 8.34 (d, 1H). LC-Ms (m/z): [M−H]=539.1.

### Step 5: N-((2-chloropyridin-4-yl)methyl)-5-(trifluoromethoxy)spiro[indoline-3,4′-piperidine]-1-carboxamide

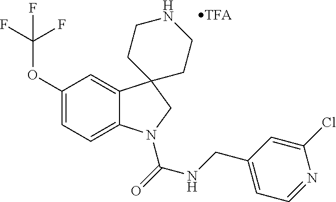 [SMILES: O=C(NCc1ccnc(Cl)c1)N1CC2(CCNCC2)c2cc(OC(F)(F)F)ccc21]

Trifluoroacetic acid (0.5 mL) was added to a solution of the product of step 4 Example 55 (350 mg, 0.648 mmol, 1 eq.) in DCM (5 mL). After stirring at room temperature for 2 hours the reaction mixture was evaporated to afford the title compounds 360 mg, which was used as is in the next step: LC-Ms (m/z): [M+H]=441.2.

### Step 6: Preparation of Example 55

(E)-N-((2-chloropyridin-4-yl)methyl)-1′-(3-(3,4-dichlorophenyl)allyl)-5-(trifluoromethoxy)spiro[indoline-3,4′-piperidine]-1-carboxamide. Commercially available (E)-3-(3,4-Dichloro-phenyl)-propenal (190 mg, 0.95 mmol) was added to a solution of the product of step 5 (Example 55) (350 mg, 0.63 mmol) and triethylamine (0.44 mL, 3.15 mmol, 5 eq) in DMF (2 mL). After stirring at room temperature for 15 minutes sodium triacetoxy borohydride (268 mg, 1.26 mmol) was added and reaction mixture was stirred at room temperature for 16 hours. The reaction mixture was concentrated under reduced and the crude material purified by preparative HPLC using ammonia as a buffer to afford title compound (92 mg, 24%) as a off white solid: 1H NMR (400 MHz, DMSO-d6) δ: 1.67 (d, J=12.96 Hz, 2H), 1.85-1.90 (m, 2H), 2.07 (t, J=11.64 Hz, 2H), 2.91 (d, J=11.16 Hz, 2H), 3.14 (d, J=5.72 Hz, 2H), 3.91 (s, 2H), 4.36 (d, J=5.56 Hz, 2H), 6.44-6.51 (m, 1H), 6.57 (d, J=15.96 Hz, 1H), 7.09 (d, J=8.6 Hz, 1H), 7.21 (s, 1H), 7.37 (d, J=5.08 Hz, 1H), 7.44-7.48 (m, 2H), 7.52-7.59 (m, 2H), 7.76 (d, J=1.76 Hz, 1H), 7.87 (d, J=8.8 Hz, 1H), 8.34 (d, J=5.08 Hz, 1H). LC-MS (m/z): 625.10 (M+H) r.t. 6.32 min. HPLC: 99.90%.

### Example 56

### Fischer Indole Template Synthesis

### (E)-N-((2-chloropyridin-4-yl)methyl)-1′-(3-(3,4-dichlorophenyl)allyl)-4-(trifluoromethyl)spiro[indoline-3,4′-piperidine]-1-carboxamide

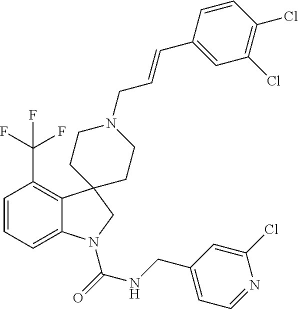 [SMILES: O=C(NCc1ccnc(Cl)c1)N1CC2(CCN(C/C=C/c3ccc(Cl)c(Cl)c3)CC2)c2c1cccc2C(F)(F)F]

### Step 1: (3-(trifluoromethyl)phenyl)hydrazine

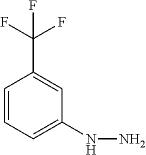 [SMILES: NNc1cccc(C(F)(F)F)c1]

Using procedures outlined in Tetrahedron, 8, 67-72; 1960, the title compound was obtained. A solution of sodium nitrite (82 g) in water (160 mL) was added to a vigorously stirred suspension of commercially available 3-(trifluoromethyl)-aniline hydrochloride (147 g) in a mixture of concentrated HCl (400 mL) and water (270 mL) at 5° C. After 15 minutes, the diazo-solution was poured slowly with stirring into a solution of stannous chloride dehydrate (530 g) in concentrated HCl (530 mL). After being stirred for a further 10 minutes, the solution was diluted with water, filtered and then rendered alkaline with 4 N NaOH. After working up by partitioning between DCM and water, then separating and drying the organic layers over MgSO₄, the title compound was obtained as a pale-yellow liquid (10 g, 69%) 1H NMR (400 mHz, CDCl3) 2H 3.50 (2H, brs), 5.40 (1H, br s), 6.90-7.30 (3H, m).

### Step 2: tert-butyl 4-(trifluoromethyl)spiro[indole-3,4′-piperidine]-1′-carboxylate

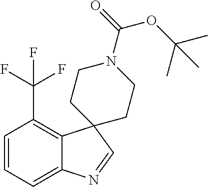 [SMILES: CC(C)(C)OC(=O)N1CCC2(C=Nc3cccc(C(F)(F)F)c32)CC1]

N-Boc-piperidine carbaldehyde (10.12 g, 47.529 mmol) was added to a stirred solution of (3-trifluoromethyl-phenyl)-hydrazine hydrochloride/the product of step 1, Example 56 (10 g, 47.06 mmol) in chloroform (500 mL) 0° C., followed by ethanol (1.37 mL, 23.53 mmol) and trifluoroacetic acid (23.06 mL, 301.17 mmol). After heating to 65° C. for 100 hours, the reaction mixture was quenched with ice cold water (100 mL) and ammonia solution (500 mL). The mixture was extracted with chloroform (3×150 mL) and the combined organic layers dried over sodium sulphate, filtered and evaporated under reduced pressure to afford brown solid (14 g, crude), which was used in the next step as is. LC-MS (m/z): 355.1 (M+H).

### Step 3: tert-butyl 4-(trifluoromethyl)spiro[indoline-3,4′-piperidine]-1′-carboxylate

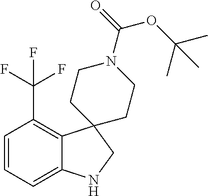 [SMILES: CC(C)(C)OC(=O)N1CCC2(CC1)CNc1cccc(C(F)(F)F)c12]

Sodium borohydride (5.984 gm, 158.192 mmol, 4 eq) was added to a solution of the product of Step 2 Example 56 (14 g, 39.55 mmol) in ethanol (140 mL) at 0° C. After stirring at room temperature for 16 hours, the reaction mixture was quenched with water (50 mL). Ethanol was evaporated under reduced pressure and the resulting residue diluted with water (250 mL). The reaction mixture was extracted with chloroform (3×200 mL), and the combined organic layers washed with brine (50 mL), dried over sodium sulphate, filtered and evaporated under reduced pressure. The crude material was purified by column chromatography on silica gel eluting with 15% ethyl acetate/hexane to afford the title compound as brown liquid (4.00 g, 28%): 1H NMR (400 MHz, DMSO) δ: 1.40 (s, 9H), 1.56 (d, 2H), 2.81-2.89 (m, 2H), 3.46 (s, 1H), 3.89 (bs, 1H), 6.13 (s, 1H), 6.78-6.88 (m, 2H), 7.13 (t, 1H). LC-MS (m/z): 357.2 (M+H). 5.00 g of tert-butyl 6-(trifluoromethyl)spiro[indoline-3,4′-piperidine]-1′-carboxylate was also isolated 1H NMR (400 MHz, DMSO) δ: 1.41 (s, 9H), 1.57-1.69 (m, 4H), 2.88 (bs, 2H), 3.43 (s, 2H), 3.89 (d, 2H), 6.06 (s, 1H), 6.68 (s, 1H), 6.81 (d, 1H), 7.18 (d, 1H). LC-MS (m/z): 357.3 (M+H).

### Step 4: tert-butyl 1-((2-chloropyridin-4-yl)methylcarbamoyl)-4-(trifluoromethyl) spiro[indoline-3,4′-piperidine]-1′-carboxylate

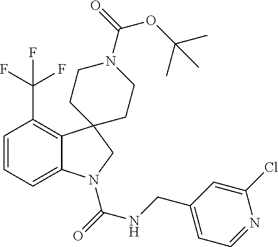 [SMILES: CC(C)(C)OC(=O)N1CCC2(CC1)CN(C(=O)NCc1ccnc(Cl)c1)c1cccc(C(F)(F)F)c12]

Potassium carbonate (23.26 g, 168.54 mmol) was added to a solution of triphosgene (12.00 g, 40.45 mmol) in DCM (250 mL) and water (100 ml) at 0° C. After stirring for 15 minutes at 0° C., commercially available (2-Chloro-pyridin-4-yl)-methylamine hydrochloride (6.00 g, 33.71 mmol) was added. After stirring at 0° C. for 1.5 hours, the organic layer was separated, dried over sodium sulphate, filtered and evaporated to afford light yellow semisolid intermediate isocyanate (˜8 g crude). The isocyanate intermediate was dissolved in THF (100 mL) and the product of Step 3 Example 56 (tert-butyl 4-(trifluoromethyl)spiro[indoline-3,4′-piperidine]-1′-carboxylate, 6 g, 16.854 mmol) added. After stirring for 1 hours at room temperature the mixture was purified by column chromatography on silica gel eluting with 2% methanol/DCM to afford the title compound as an off white solid (5.8 g, 66%): 1H NMR (400 MHz, DMSO) δ: 1.42 (s, 9H), 1.61 (d, 2H), 1.98-2.06 (2, 4 H), 2.87 (bs, 2H), 3.97 (d, 2H), 4.06 (s, 2H), 4.39 (d, 2H), 7.24 (d, 1H), 7.34-7.38 (d, 2H), 7.46 (s, 1H), 7.44 (s, 1H), 7.60 (t, 1H), 7.28 (d, 1H), 7.35 (d, 1H). LC-MS (m/z): 524.9 (M+H).

### Step 5: N-((2-chloropyridin-4-yl)methyl)-4-(trifluoromethyl)spiro[indoline-3,4′-piperidine]-1-carboxamide

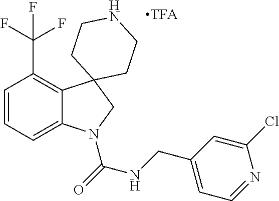 [SMILES: O=C(NCc1ccnc(Cl)c1)N1CC2(CCNCC2)c2c1cccc2C(F)(F)F]

Trifluoroacetic acid (11.943 g, 104.766 mmol) was added to a solution of the product of Step 4 Example 56 (11.00 g, 20.95 mmol) in DCM (200 mL). After stirring at room temperature for 3 hours, the solvent was removed under reduced pressure to afford a yellow semi-solid. The resulting salt was dissolved in water (500 mL) and basified to pH˜8 using a saturated aqueous solution of NaHCO₃. The aqueous layer was extracted with 5% methanol/DCM (3×200 mL). The combined organic layers were dried over sodium sulphate, filtered, and evaporated to afford title compound as a yellow solid (8.90 g, 100%): 1H NMR (400 MHz, DMSO) δ: 1.62 (d, 2H), 2.08-2.16 (2, 4 H), 2.77 (t, 2H), 3.07-3.16 (d, 2H), 4.03 (s, 2H), 4.39 (d, 2H), 7.24 (d, 1H), 7.34-7.38 (d, 2H), 7.45 (s, 1H), 7.64 (t, 1H), 7.28 (d, 1H), 7.35 (d, 1H). LC-MS (m/z): 425.0 (M+H).

### Step 6: Preparation of Example 56

(E)-N-((2-chloropyridin-4-yl)methyl)-1′-(3-(3,4-dichlorophenyl)allyl)-4-(trifluoromethyl)spiro[indoline-3,4′-piperidine]-1-carboxamide. Sodium triacetoxy borohydride (6.38 g, 30.07 mmol) was added to a solution of the product from Step 5 Example 56 (8.50 g, 20.05 mmol) and (E)-3-(3,4-Dichloro-phenyl)-propenal (4.84 g, 24.06 mmol) in DCM (200 mL). After stirring for 1 hour at room temperature the reaction mixture was quenched with water (200 mL). The aqueous layer was extracted with DCM (3×200 mL). The combined organic layers were dried over sodium sulphate, filtered, and evaporated to afford yellow solid, which was purified by preparative HPLC using ammonium acetate: water buffer to afford title compound as a off white solid (7.9 g, 65%): 1H NMR (400 MHz, DMSO-d6) δ: 1.59 (d, J=11.8 Hz, 2H), 2.10-2.24 (m, 4H), 2.89 (d, J=10.56 Hz, 2H), 3.14 (d, J=4.84 Hz, 2H), 3.96 (s, 2H), 4.37 (d, J=5.64 Hz, 2H), 6.47-6.56 (m, 2H), 7.24 (d, J=7.72 Hz, 1H), 7.33 (d, J=8.12 Hz, 1H), 7.36 (d, J=4.84 Hz, 1H), 7.44 (s, 1H), 7.46-7.49 (m, 1H), 7.56 (d, J=8.36 Hz, 1H), 7.67 (t, J=5.74 Hz, 1H), 7.77 (d, J=1.88 Hz, 1H), 8.28 (d, J=8.12 Hz, 1H), 8.34 (d, J=5.08 Hz, 1H). LC-MS (m/z): 608.90 (M+H) r.t. 11.10 min HPLC: 99.89%.

### Example 57

### (E)-7-chloro-1′-(3-(4-chlorophenyl)allyl)-N-((2-chlorothiazol-5-yl)methyl)-4-(trifluoromethoxy)spiro[indoline-3,4′-piperidine]-1-carboxamide

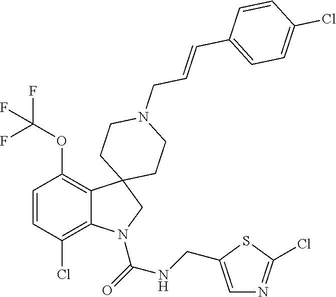 [SMILES: O=C(NCc1cnc(Cl)s1)N1CC2(CCN(C/C=C/c3ccc(Cl)cc3)CC2)c2c(OC(F)(F)F)ccc(Cl)c21]

Using the same procedures as Step 1 through 6 of Example 55 and replacing 4-(trifluoromethoxy)aniline with commercially available 2-chloro-5-(trifluoromethoxy)aniline and (2-Chloro-pyridin-4-yl)-methylamine hydrochloride with commercially available 2-chloro-5-(aminomethyl)thiazole and (E)-3-(3,4-Dichloro-phenyl)-propenal with commercially available (E)-3-(4-chlorophenyl)acrylaldehyde the title compound is obtained: 1H NMR (400 MHz, DMSO-d6) δ: 1.52 (d, J=12.56 Hz, 2H), 1.92 (t, J=11.88 Hz, 2H), 2.07-2.12 (m, 2H), 2.85 (d, J=11.52 Hz, 2H), 3.11 (d, J=6.48 Hz, 2H), 3.88 (s, 2H), 4.42 (d, J=5.68 Hz, 2H), 6.33-6.40 (m, 1H), 6.53 (d, J=15.88 Hz, 1H), 7.01-7.04 (m, 1H), 7.37 (d, J=8.76 Hz, 3H), 7.48 (d, J=8.52 Hz, 2H), 7.56 (s, 1H), 8.11 (t, J=5.86 Hz, 1H). LC-MS (m/z): 630.6 (M+H) r.t. 5.40 min. HPLC: 98.45%.

### Example 58

### (E)-7-chloro-N-((2-chloropyridin-4-yl)methyl)-1′-(3-(4-fluorophenyl)allyl)-4-(trifluoromethoxy)spiro[indoline-3,4′-piperidine]-1-carboxamide

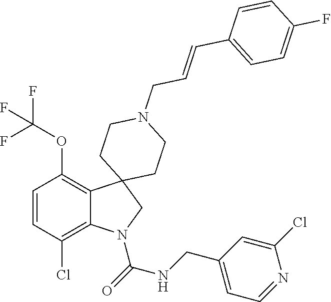 [SMILES: O=C(NCc1ccnc(Cl)c1)N1CC2(CCN(C/C=C/c3ccc(F)cc3)CC2)c2c(OC(F)(F)F)ccc(Cl)c21]

Using the same procedures as Step 1 through 6 of Example 55 and replacing 4-(trifluoromethoxy)aniline with commercially available 2-chloro-5-(trifluoromethoxy)aniline and (E)-3-(3,4-Dichloro-phenyl)-propenal with commercially available (E)-3-(4-fluorophenyl)acrylaldehyde the title compound is obtained: 1H NMR (400 MHz, DMSO-d6) δ: 1.54 (d, J=12.44 Hz, 2H), 1.99 (t, J=11.78 Hz, 2H), 2.09-2.16 (m, 2H), 2.91 (d, J=11.08 Hz, 2H), 3.13 (d, J=6.44 Hz, 2H), 3.98 (s, 2H), 4.34 (d, J=5.84 Hz, 2H), 6.26-6.34 (m, 1H), 6.53 (d, J=15.84 Hz, 1H), 7.01-7.03 (m, 1H), 7.15 (t, J=8.84 Hz, 2H), 7.31 (d, J=5.04 Hz, 1H), 7.37 (d, J=8.88 Hz, 2H), 7.48-7.52 (m, 2H), 8.06 (t, J=5.9 Hz, 1H), 8.34 (d, J=5.08 Hz, 1H), LC-MS (m/z): 608.9 (M+H) r.t. 5.76 min.

### Example 59

### (E)-4,6-dichloro-1′-(3-(4-chlorophenyl)allyl)-N-((2-chloropyridin-4-yl)methyl)spiro[indoline-3,4′-piperidine]-1-carboxamide

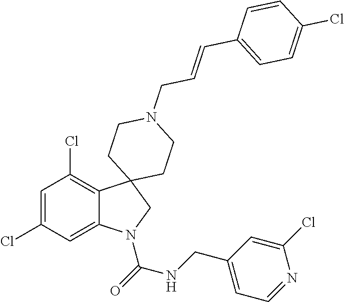 [SMILES: O=C(NCc1ccnc(Cl)c1)N1CC2(CCN(C/C=C/c3ccc(Cl)cc3)CC2)c2c(Cl)cc(Cl)cc21]

Using the same procedures as Step 2 through 6 of Example 56 and replacing (3-Trifluoromethyl-phenyl)-hydrazine with commercially available 2-chloro-5-(trifluoromethoxy)aniline and (E)-3-(3,4-Dichloro-phenyl)-propenal with commercially available (E)-3-(4-chlorophenyl)acrylaldehyde the title compound is obtained: 1H NMR (400 MHz, DMSO-d6) δ: 1.58 (d, J=13.08 Hz, 2H), 2.02-2.08 (m, 2H), 2.49-2.51 (m, 2H), 2.91 (d, J=11.4 Hz, 2H), 3.12 (d, J=6.32 Hz, 2H), 3.94 (s, 2H), 4.36 (d, J=5.52 Hz, 2H), 6.37-6.43 (m, 1H), 6.54 (d, J=15.96 Hz, 1H), 7.00 (d, J=1.92 Hz, 1H), 7.36-7.38 (m, 3H), 7.46-7.50 (m, 3H), 7.67 (t, J=5.9 Hz, 1H), 7.94 (d, J=1.96 Hz, 1H), 8.35 (d, J=5.16 Hz, 1H). LC-MS (m/z): 574.6 (M+H) r.t. 15.4 min. HPLC: 99.22%.

### Example 60

### (E)-4,6-dichloro-1′-(3-(4-chlorophenyl)allyl)-N-((2-fluoropyridin-4-yl)methyl)spiro[indoline-3,4′-piperidine]-1-carboxamide

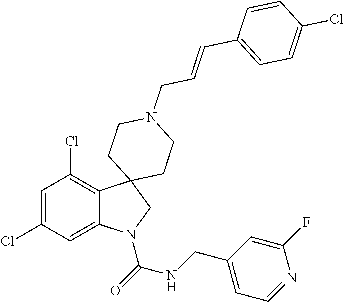 [SMILES: O=C(NCc1ccnc(F)c1)N1CC2(CCN(C/C=C/c3ccc(Cl)cc3)CC2)c2c(Cl)cc(Cl)cc21]

Using the same procedures as Step 2 through 6 of Example 56 and replacing (3-Trifluoromethyl-phenyl)-hydrazine with commercially available (3,5-dichlorophenyl)hydrazine and (2-Chloro-pyridin-4-yl)-methylamine hydrochloride with commercially available (2-fluoropyridin-4-yl)methanamine and (E)-3-(3,4-Dichloro-phenyl)-propenal with commercially available (E)-3-(4-chlorophenyl)acrylaldehyde the title compound is obtained: 1H NMR (400 MHz, DMSO-d6) δ: 1.55 (d, J=12.56 Hz, 2H), 2.03 (t, J=11.76 Hz, 2H), 2.45-2.48 (m, 2H), 2.88 (d, J=11.72 Hz, 2H), 3.10 (d, J=6.36 Hz, 2H), 3.92 (s, 2H), 4.37 (d, J=5.6 Hz, 2H), 6.34-6.40 (m, 1H), 6.52 (d, J=16.0 Hz, 1H), 6.97 (d, J=1.96 Hz, 1H), 7.09 (s, 1H), 7.29 (d, J=5.12 Hz, 1H), 7.34 (d, J=8.52 Hz, 2H), 7.47 (d, J=8.56 Hz, 2H), 7.66 (t, J=5.74 Hz, 1H), 7.91 (d, J=1.96 Hz, 1H), 8.15 (d, J=5.12 Hz, 1H). LC-MS (m/z): 558.6 (M+H) r.t. 4.95 min. HPLC: 99.48%.

### Example 61

### (E)-4,6-dichloro-1′-(3-(4-chlorophenyl)allyl)-5-fluoro-N-((2-fluoropyridin-4-yl)methyl)spiro[indoline-3,4′-piperidine]-1-carboxamide

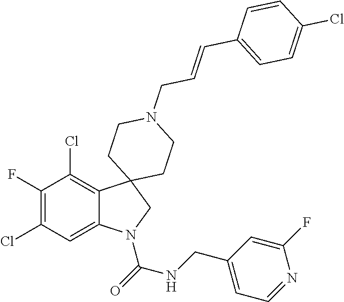 [SMILES: O=C(NCc1ccnc(F)c1)N1CC2(CCN(C/C=C/c3ccc(Cl)cc3)CC2)c2c1cc(Cl)c(F)c2Cl]

Using the same procedures as Step 1 through 6 of Example 55 and replacing 4-(trifluoromethoxy)aniline with commercially available 3,5-dichloro-4-fluoroaniline and (2-Chloro-pyridin-4-yl)-methylamine hydrochloride with commercially available (2-fluoropyridin-4-yl)methanamine and (E)-3-(3,4-Dichloro-phenyl)-propenal with commercially available (E)-3-(4-chlorophenyl)acrylaldehyde the title compound is obtained: 1H NMR (400 MHz, DMSO-d6) δ: 1.63 (d, J=13.04 Hz, 2H), 2.05 (t, J=11.66 Hz, 2H), 2.44-2.46 (m, 2H), 2.92 (d, J=11.52 Hz, 2H), 3.13 (d, J=6.32 Hz, 2H), 3.96 (s, 2H), 4.39 (d, J=5.48 Hz, 2H), 6.35-6.43 (m, 1H), 6.55 (d, J=16.0 Hz, 1H), 7.12 (s, 1H), 7.31 (d, J=5.0 Hz, 1H), 7.37 (d, J=8.48 Hz, 2H), 7.49 (d, J=8.52 Hz, 2H), 7.68 (t, J=5.68 Hz, 1H), 8.03 (d, J=6.48 Hz, 1H), 8.17 (d, J=5.16 Hz, 1H). LC-MS (m/z): 577.1 (M+H) r.t. 11.2 min. HPLC: 99.78%.

### Example 62

### (E)-4,5-dichloro-1′-(3-(4-chlorophenyl)allyl)-N-((2-fluoropyridin-4-yl)methyl)spiro[indoline-3,4′-piperidine]-1-carboxamide

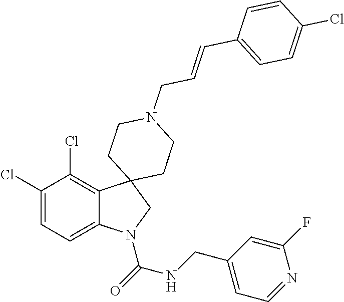 [SMILES: O=C(NCc1ccnc(F)c1)N1CC2(CCN(C/C=C/c3ccc(Cl)cc3)CC2)c2c1ccc(Cl)c2Cl]

Using the same procedures as Step 2 through 6 of Example 56 and replacing (3-Trifluoromethyl-phenyl)-hydrazine with commercially available (3,4-dichlorophenyl)hydrazine and (2-Chloro-pyridin-4-yl)-methylamine hydrochloride with commercially available (2-fluoropyridin-4-yl)methanamine and (E)-3-(3,4-Dichloro-phenyl)-propenal with commercially available (E)-3-(4-chlorophenyl)acrylaldehyde the title compound is obtained: 1H NMR (400 MHz, DMSO-d6) δ: 1.58 (d, J=12.44 Hz, 2H), 2.07 (t, J=11.62 Hz, 2H), 2.53-2.60 (m, 2H), 2.92 (d, J=11.48 Hz, 2H), 3.13 (d, J=6.44 Hz, 2H), 3.95 (s, 2H), 4.40 (d, J=5.6 Hz, 2H), 6.38-6.44 (m, 1H), 6.55 (d, J=15.92 Hz, 1H), 7.12 (s, 1H), 7.31 (d, J=5.24 Hz, 1H), 7.36-7.40 (m, 3H), 7.50 (d, J=8.52 Hz, 2H), 7.62 (t, J=5.86 Hz, 1H), 7.91 (d, J=8.76 Hz, 1H), 8.17 (d, J=5.12 Hz, 1H). LC-MS (m/z): 558.7 (M+H) r.t. 4.66 min. HPLC: 99.75%.

### Example 63

### (E)-4-chloro-1′-(3-(4-chlorophenyl)allyl)-5-fluoro-N-((2-fluoropyridin-4-yl)methyl)spiro[indoline-3,4′-piperidine]-1-carboxamide

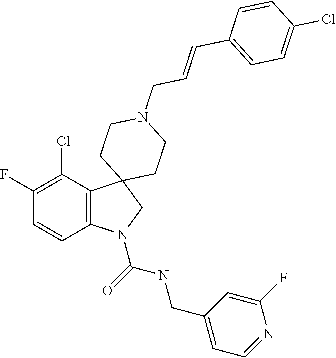 [SMILES: O=C(NCc1ccnc(F)c1)N1CC2(CCN(C/C=C/c3ccc(Cl)cc3)CC2)c2c1ccc(F)c2Cl]

Using the same procedures as Step 2 through 6 of Example 56 and replacing (3-Trifluoromethyl-phenyl)-hydrazine with commercially available (3-chloro-4-fluorophenyl)hydrazine and (2-Chloro-pyridin-4-yl)-methylamine hydrochloride with commercially available (2-fluoropyridin-4-yl)methanamine and (E)-3-(3,4-Dichloro-phenyl)-propenal with commercially available (E)-3-(4-chlorophenyl)acrylaldehyde the title compound is obtained. 1H NMR (400 MHz, DMSO-d6) δ: 1.60 (d, J=12.48 Hz, 2H), 2.04-22.10 (m, 2H), 2.29-2.66 (m, 2H), 2.92 (d, J=11.52 Hz, 2H), 3.13 (d, J=6.44 Hz, 2H), 3.95 (s, 2H), 4.39 (d, J=5.68 Hz, 2H), 6.36-6.43 (m, 1H), 6.55 (d, J=15.88 Hz, 1H), 7.11 (s, 1H), 7.16 (t, J=9.12 Hz, 1H), 7.31 (d, J=5.2 Hz, 1H), 7.37 (d, J=8.52 Hz, 2H), 7.49 (d, J=9.72 Hz, 2H), 7.56 (t, J=5.8 Hz, 1H), 7.87-7.90 (m, 1H), 8.17 (d, J=5.12 Hz, 1H). LC-MS (m/z): 543 (M+H) r.t. 5.86 min. HPLC: 99.67%.

### Example 64

### (E)-4,5-dichloro-1′-(3-(4-chlorophenyl)allyl)-N-((2-chlorothiazol-5-yl)methyl)spiro[indoline-3,4′-piperidine]-1-carboxamide

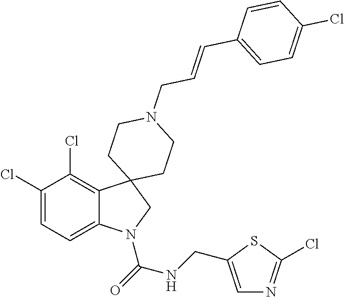 [SMILES: O=C(NCc1cnc(Cl)s1)N1CC2(CCN(C/C=C/c3ccc(Cl)cc3)CC2)c2c1ccc(Cl)c2Cl]

Using the same procedures as Step 2 through 6 of Example 56 and replacing (3-Trifluoromethyl-phenyl)-hydrazine with commercially available (3,4-dichlorophenyl)hydrazine and (2-Chloro-pyridin-4-yl)-methylamine hydrochloride with commercially available (2-chlorothiazol-5-yl)methanamine and (E)-3-(3,4-Dichloro-phenyl)-propenal with commercially available (E)-3-(4-chlorophenyl)acrylaldehyde the title compound is obtained: 1H NMR (400 MHz, DMSO-d6) δ: 1.53 (d, J=13.12 Hz, 2H), 2.02 (t, J=11.18 Hz, 2H), 2.49-2.50 (m, 2H), 2.89 (d, J=10.8 Hz, 2H), 3.12 (d, J=6.2 Hz, 2H), 3.83 (s, 2H), 4.43 (d, J=5.4 Hz, 2H), 6.37-6.42 (m, 1H), 6.54 (d, J=15.84 Hz, 1H), 7.37 (d, J=8.52 Hz, 2H), 7.42 (d, J=8.72 Hz, 1H), 7.49 (d, J=8.52 Hz, 2H), 7.60 (s, 1H), 7.71 (t, J=5.66 Hz, 1H), 7.95 (d, J=8.72 Hz, 1H). LC-MS (m/z): 580.6 (M+H) r.t. 5.42 min. HPLC: 99.49%.

### Example 65

### (E)-N-((2-chloropyridin-4-yl)methyl)-1-(3-(3,4-dichlorophenyl)allyl)-6′-methoxyspiro[piperidine-4,3′-pyrrolo[3,2-c]pyridine]-1′(2′H)-carboxamide

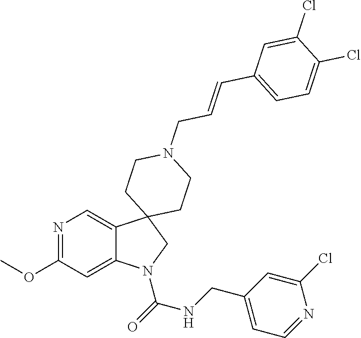 [SMILES: COc1cc2c(cn1)C1(CCN(C/C=C/c3ccc(Cl)c(Cl)c3)CC1)CN2C(=O)NCc1ccnc(Cl)c1]

Using the same procedures as Step 1 through 6 of Example 55 and replacing 4-(trifluoromethoxy)aniline with commercially available 2-methoxypyridin-4-amine the title compound is obtained: 1H NMR (400 MHz, DMSO-d6) δ: 1.68 (d, J=12.76 Hz, 2H), 1.88 (t, J=11.26 Hz, 2H), 2.06 (t, J=11.84 Hz, 2H), 2.90 (d, J=11.56 Hz, 2H), 3.14 (d, J=5.72 Hz, 2H), 3.76 (s, 3H), 3.89 (s, 2H), 4.36 (d, J=5.56 Hz, 2H), 6.44-6.58 (m, 2H), 7.02 (s, 1H), 7.37 (d, J=4.48 Hz, 1H), 7.45-7.48 (m, 2H), 7.57 (d, J=8.36 Hz, 1H), 7.68 (t, J=5.68 Hz, 1H), 7.75 (d, J=1.56 Hz, 1H), 7.94 (s, 1H), 8.34 (d, J=5.12 Hz, 1H). LC-MS (m/z): 571.6 (M+H) r.t. 3.74 min.

### Example 66

### (E)-N-((2-chloropyridin-4-yl)methyl)-5′-cyano-1-(3-(3,4-dichlorophenyl)allyl)spiro[piperidine-4,3′-pyrrolo[2,3-c]pyridine]-1′(2′H)-carboxamide

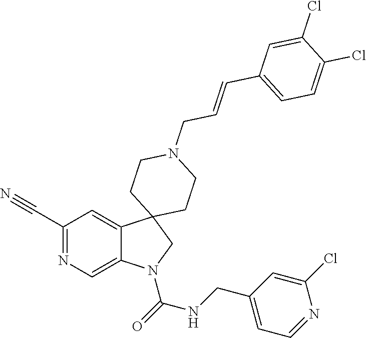 [SMILES: N#Cc1cc2c(cn1)N(C(=O)NCc1ccnc(Cl)c1)CC21CCN(C/C=C/c2ccc(Cl)c(Cl)c2)CC1]

Using the same procedures as Step 1 through 6 of Example 55 and replacing 4-(trifluoromethoxy)aniline with commercially available 5-aminopicolinonitrile the title compound is obtained: 1H NMR (400 MHz, DMSO-d6) δ: 1.71 (d, J=12.72 Hz, 2H), 1.88-1.94 (m, 2H), 2.06 (t, J=11.6 Hz, 2H), 2.92 (d, J=11.76 Hz, 2H), 3.15 (d, J=6.0 Hz, 2H), 3.98 (s, 2H), 4.39 (d, J=5.52 Hz, 2H), 6.44-6.51 (m, 1H), 6.57 (d, J=16.04 Hz, 1H), 7.39 (d, J=5.04 Hz, 1H), 7.46-7.48 (m, 2H), 7.58 (d, J=8.36 Hz, 1H), 7.75 (d, J=1.92 Hz, 1H), 7.84 (t, J=5.82 Hz, 1H), 8.00 (s, 1H), 8.35 (d, J=5.08 Hz, 1H), 9.07 (s, 1H). LC-MS (m/z): 567.3 (M+H) r.t. HPLC: 98.73%.

### Example 67

### (E)-N-((2-chloropyridin-4-yl)methyl)-5′-cyano-1-(3-(3,4-dichlorophenyl)allyl)spiro[piperidine-4,3′-pyrrolo[3,2-b]pyridine]-1′(2′H)-carboxamide

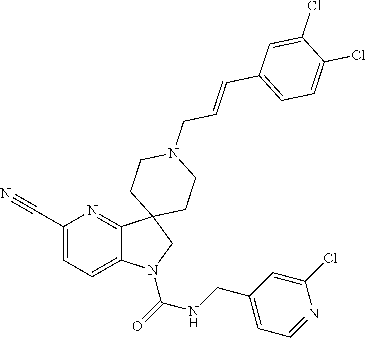 [SMILES: N#Cc1ccc2c(n1)C1(CCN(C/C=C/c3ccc(Cl)c(Cl)c3)CC1)CN2C(=O)NCc1ccnc(Cl)c1]

Using the same procedures as Step 1 through 6 of Example 55 and replacing 4-(trifluoromethoxy)aniline with commercially available 5-hydrazinylpicolinonitrile the title compound is obtained: 1H NMR (400 MHz, DMSO-d6) δ: 1.71 (d, J=11.48 Hz, 2H), 1.88-1.94 (m, 2H), 2.06 (t, J=11.6 Hz, 2H), 2.94 (d, J=11.6 Hz, 2H), 3.15-3.17 (m, 2H), 3.99 (s, 2H), 4.39 (d, J=5.64 Hz, 2H), 6.46-6.51 (m, 1H), 6.58 (d, J=14.42 Hz, 1H), 7.39 (d, J=5.08 Hz, 1H), 7.47-7.49 (m, 2H), 7.58 (d, J=8.4 Hz, 1H), 7.74-7.77 (m, 2H), 7.88 (t, J=6.14 Hz, 1H), 8.07 (d, J=8.4 Hz, 1H), 8.35 (d, J=5.0 Hz, 1H). LC-MS (m/z): 566.7 (M+H) r.t. 3.68 min. HPLC: 99.08%.

### Example 68

### (E)-N-((2-chloropyridin-4-yl)methyl)-1-(3-(3,4-dichlorophenyl)allyl)-4′-methoxyspiro[piperidine-4,3′-pyrrolo[3,2-c]pyridine]-1′(2′H)-carboxamide

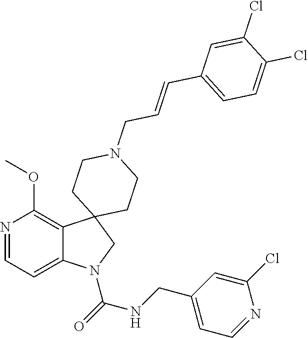 [SMILES: COc1nccc2c1C1(CCN(C/C=C/c3ccc(Cl)c(Cl)c3)CC1)CN2C(=O)NCc1ccnc(Cl)c1]

Using the same procedures as Step 1 through 6 of Example 55 and replacing 4-(trifluoromethoxy)aniline with commercially available 2-methoxypyridin-4-amine and (E)-3-(3,4-Dichloro-phenyl)-propenal with commercially available (E)-3-(4-chlorophenyl)acrylaldehyde the title compound is obtained: LC-MS (m/z): 571.9 (M+H) r.t. 3.78 min.

### Example 69

### (E)-6-chloro-1′-(3-(4-chlorophenyl)allyl)-5-fluoro-N-((2-fluoropyridin-4-yl)methyl)spiro[indoline-3,4′-piperidine]-1-carboxamide

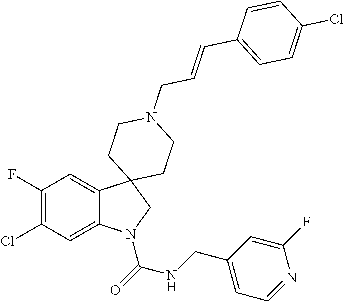 [SMILES: O=C(NCc1ccnc(F)c1)N1CC2(CCN(C/C=C/c3ccc(Cl)cc3)CC2)c2cc(F)c(Cl)cc21]

Using the same procedures as Step 2 through 6 of Example 56 and replacing (3-Trifluoromethyl-phenyl)-hydrazine with commercially available (3-chloro-4-fluorophenyl)hydrazine and (2-Chloro-pyridin-4-yl)-methylamine hydrochloride with commercially available (2-fluoropyridin-4-yl)methanamine and (E)-3-(3,4-Dichloro-phenyl)-propenal with commercially available (E)-3-(4-chlorophenyl)acrylaldehyde the title compound is obtained: 1H NMR (400 MHz, DMSO) δ: 1.64 (d, J=12.62, 2H), 1.84-1.90 (m, 2H), 2.05 (t, J=11.36 Hz, 2H), 2.90 (d, J=11.4 Hz, 2H), 3.13 (d, J=6.28 Hz, 2H), 3.90 (s, 2H), 4.38 (d, J=5.64 Hz, 2H), 6.32-6.40 (m, 1H), 6.56 (d, J=15.88 Hz, 1H), 7.10 (s, 1H), 7.30 (d, J=5.32 Hz, 1H), 7.36-7.39 (m, 3H), 7.48 (d, J=8.56 Hz, 2H), 7.58 (t, J=5.64 Hz, 1H), 7.91 (d, J=6.84 Hz, 1H), 8.17 (d, J=5.16 Hz, 1H). LC-MS (m/z): 542.9 (M+H) r.t. 5.86 min. HPLC: 99.62%.

### Example 70

### (E)-5,6-dichloro-1′-(3-(4-chlorophenyl)allyl)-N-((2-fluoropyridin-4-yl)methyl)spiro[indoline-3,4′-piperidine]-1-carboxamide

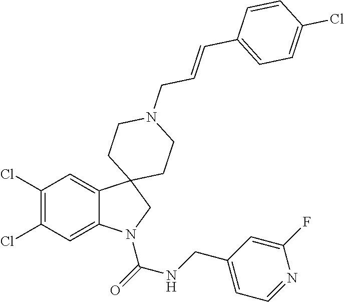 [SMILES: O=C(NCc1ccnc(F)c1)N1CC2(CCN(C/C=C/c3ccc(Cl)cc3)CC2)c2cc(Cl)c(Cl)cc21]

Using the same procedures as Step 2 through 6 of Example 56 and replacing (3-Trifluoromethyl-phenyl)-hydrazine with commercially (3,4-dichlorophenyl)hydrazine and (2-Chloro-pyridin-4-yl)-methylamine hydrochloride with commercially available (2-fluoropyridin-4-yl)methanamine and (E)-3-(3,4-Dichloro-phenyl)-propenal with commercially available (E)-3-(4-chlorophenyl)acrylaldehyde the title compound is obtained: 1H NMR (400 MHz, DMSO-d6) δ: 1.65 (d, J=11.84 Hz, 2H), 1.85-1.92 (m, 2H), 2.05 (t, J=11.14 Hz, 2H), 2.90 (d, J=11.96 Hz, 2H), 3.14 (d, J=6.4 Hz, 2H), 3.91 (s, 2H), 4.39 (d, J=5.68 Hz, 2H), 6.32-6.40 (m, 1H), 6.56 (d, J=15.84 Hz, 1H), 7.11 (s, 1H), 7.31 (d, J=5.16 Hz, 1H), 7.38 (d, J=8.52 Hz, 2H), 7.47-7.49 (m, 3H), 7.64 (t, J=5.8 Hz, 1H), 7.98 (s, 1H), 8.17 (d, J=5.16 Hz, 1H). LC-MS (m/z): 558.9, r.t. 10.78 min. (M+H). HPLC: 99.88%.

### Example 71

### (E)-5-chloro-1′-(3-(4-chlorophenyl)allyl)-6-fluoro-N-((2-fluoropyridin-4-yl)methyl)spiro[indoline-3,4′-piperidine]-1-carboxamide

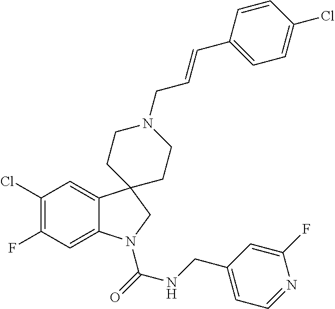 [SMILES: O=C(NCc1ccnc(F)c1)N1CC2(CCN(C/C=C/c3ccc(Cl)cc3)CC2)c2cc(Cl)c(F)cc21]

Using the same procedures as Step 2 through 6 of Example 56 and replacing (3-Trifluoromethyl-phenyl)-hydrazine with (4-chloro-3-fluorophenyl)hydrazine and (2-Chloro-pyridin-4-yl)-methylamine hydrochloride with commercially available (2-fluoropyridin-4-yl)methanamine and (E)-3-(3,4-Dichloro-phenyl)-propenal with commercially available (E)-3-(4-chlorophenyl)acrylaldehyde the title compound is obtained: 1H NMR (400 MHz, DMSO-d6) δ: 1.64 (d, J=12.12 Hz, 2H), 1.87 (t, J=11.98 Hz, 2H), 2.05 (t, J=11.8 Hz, 2H), 2.90 (d, J=11.56 Hz, 2H), 3.13 (d, J=6.2 Hz, 2H), 3.91 (s, 2H), 4.39 (d, J=5.28 Hz, 2H), 6.33-6.40 (m, 1H), 6.56 (d, J=15.96 Hz, 1H), 7.11 (s, 1H), 7.31 (d, J=4.0 Hz, 1H), 7.37-7.42 (m, 3H), 7.48 (d, J=8.0 Hz, 2H), 7.64 (t, J=5.52 Hz, 1H), 7.73 (d, J=11.36 Hz, 1H), 8.17 (d, J=5.04 Hz, 1H). LC-MS (m/z): 542.8 (M+H) r.t. 8.14 min. HPLC: 97.08%.

### Example 72

### (E)-5-chloro-N-((2-chloropyridin-4-yl)methyl)-1′-(3-(4-cyanophenyl)allyl)-6-(trifluoromethyl)spiro[indoline-3,4′-piperidine]-1-carboxamide

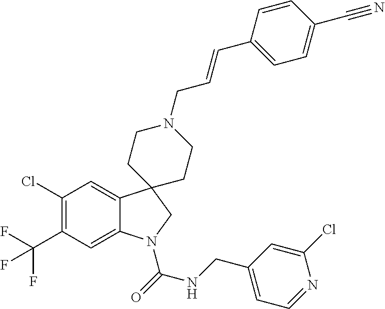 [SMILES: N#Cc1ccc(/C=C/CN2CCC3(CC2)CN(C(=O)NCc2ccnc(Cl)c2)c2cc(C(F)(F)F)c(Cl)cc23)cc1]

Using the same procedures as Step 1 through 6 of Example 55 and replacing 4-(trifluoromethoxy)aniline with commercially available 2-chloro-5-(trifluoromethoxy)aniline and (E)-3-(3,4-Dichloro-phenyl)-propenal with commercially available (E)-3-(4-chlorophenyl)acrylaldehyde the title compound is obtained. 1H NMR (400 MHz, DMSO) δ: 1.69 (d, J=10.84 Hz, 2H), 1.90-1.95 (m, 2H), 2.08 (t, J=12.08 Hz, 2H), 2.92 (d, J=10.28 Hz, 2H), 3.19 (d, J=6.12 Hz, 2H), 3.95 (s, 2H), 4.36 (d, J=5.68 Hz, 2H), 6.55-6.60 (m, 1H), 6.67 (d, J=15.96 Hz, 1H), 7.38 (d, J=5.04 Hz, 1H), 7.46 (s, 1H), 7.59 (s, 1H), 7.65-7.70 (m, 3H), 7.79 (d, J=8.36 Hz, 2H), 8.25 (s, 1H), 8.34 (d, J=5 Hz, 1H). LC-MS (m/z): 600.1 (M+H) r.t. 5.76 min. HPLC: 99.81%.

### Example 73

### (E)-1′-(3-(4-chlorophenyl)allyl)-N-((2-fluoropyridin-4-yl)methyl)-6-(2-(trifluoromethoxy)phenyl)spiro[indoline-3,4′-piperidine]-1-carboxamide

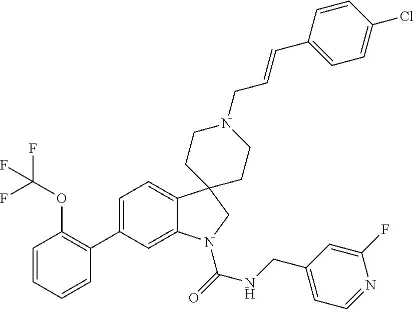 [SMILES: O=C(NCc1ccnc(F)c1)N1CC2(CCN(C/C=C/c3ccc(Cl)cc3)CC2)c2ccc(-c3ccccc3OC(F)(F)F)cc21]

Using the same procedures as Step 1 through 6 of Example 56 and replacing 4-(trifluoromethoxy)aniline with commercially available 2′-(trifluoromethoxy)-[1,1′-biphenyl]-3-amine and (2-Chloro-pyridin-4-yl)-methylamine hydrochloride with commercially (2-fluoropyridin-4-yl)methanamine and (E)-3-(3,4-Dichloro-phenyl)-propenal with commercially available (E)-3-(4-chlorophenyl)acrylaldehyde the title compound is obtained: 1H NMR (400 MHz, DMSO-d6) δ: 1.69 (d, J=12.52 Hz, 2H), 1.91 (t, J=11.12 Hz, 2H), 2.10 (t, J=11.64 Hz, 2H), 2.93 (d, J=11.04 Hz, 2H), 3.16 (d, J=6.24 Hz, 2H), 3.92 (s, 2H), 4.39 (d, J=5.48 Hz, 2H), 6.36-6.43 (m, 1H), 6.57 (d, J=16.0 Hz, 1H), 6.98 (d, J=8.48 Hz, 1H), 7.10 (s, 1H), 7.28-7.31 (m, 2H), 7.38 (d, J=8.4 Hz, 2H), 7.42-7.53 (m, 7H), 7.96 (s, 1H), 8.16 (d, J=5.16 Hz, 1H). LC-MS (m/z): 651.0 (M+H) r.t. 6.41 min.

### Example 74

### (E)-1′-(3-(4-chlorophenyl)allyl)-N-((2-chloropyridin-4-yl)methyl)-5-(2-oxopyridin-1(2H)-yl)spiro[indoline-3,4′-piperidine]-1-carboxamide

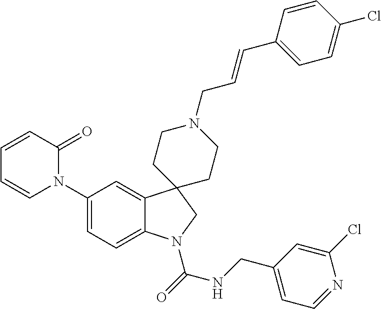 [SMILES: O=C(NCc1ccnc(Cl)c1)N1CC2(CCN(C/C=C/c3ccc(Cl)cc3)CC2)c2cc(-n3ccccc3=O)ccc21]

Using the same procedures as Step 1 through 6 of Example 56 and replacing 4-(trifluoromethoxy)aniline with commercially available 1-(4-aminophenyl)pyridin-2(1H)-one and (E)-3-(3,4-Dichloro-phenyl)-propenal with commercially available (E)-3-(4-chlorophenyl)acrylaldehyde the title compound is obtained: 1H NMR (400 MHz, DMSO-d6) δ: 1.67 (d, J=12.32 Hz, 2H), 1.86-1.92 (m, 2H), 2.07-2.12 (m, 2H), 2.91 (d, J=10.68 Hz, 2H), 3.14 (d, J=6.52 Hz, 2H), 3.92 (s, 2H), 4.38 (d, J=5.6 Hz, 2H), 6.27 (dt, J=1.36 Hz, J=6.7 Hz, 1H), 6.34-6.40 (m, 1H), 6.44 (d, J=8.88 Hz, 1H), 6.56 (d, J=15.8 Hz, 1H), 7.10 (dd, J=2.2 Hz, J=8.52 Hz, 1H), 7.23 (d, J=2.2 Hz, 1H), 7.36-7.38 (m, 3H), 7.45-7.49 (m, 4H), 7.55-7.58 (m, 2H), 7.88 (d, J=8.56 Hz, 1H), 8.34 (d, J=5.12 Hz, 1H). LC-MS (m/z): 600.0M+H) r.t. 9.14 min. HPLC: 98.14%.

### Example 75

### (E)-1′-(3-(4-chlorophenyl)allyl)-N-((2-fluoropyridin-4-yl)methyl)-5-(2-oxopyridin-1(2H)-yl)spiro[indoline-3,4′-piperidine]-1-carboxamide

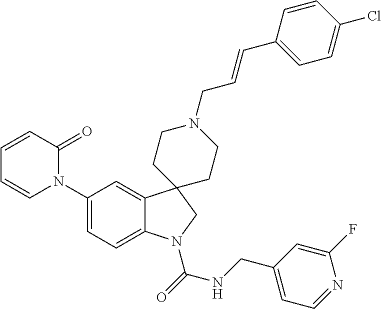 [SMILES: O=C(NCc1ccnc(F)c1)N1CC2(CCN(C/C=C/c3ccc(Cl)cc3)CC2)c2cc(-n3ccccc3=O)ccc21]

Using the same procedures as Step 1 through 6 of Example 56 and replacing 4-(trifluoromethoxy)aniline with commercially available 1-(4-aminophenyl)pyridin-2(1H)-one and (2-Chloro-pyridin-4-yl)-methylamine hydrochloride with commercially available (2-fluoropyridin-4-yl)methanamine and (E)-3-(3,4-Dichloro-phenyl)-propenal with commercially available (E)-3-(4-chlorophenyl)acrylaldehyde the title compound is obtained: 1H NMR (400 MHz, DMSO-d6) δ: 1.67 (d, J=12.6 Hz, 2H), 1.86-1.92 (m, 2H), 2.07-2.13 (m, 2H), 2.90 (d, J=11.56 Hz, 2H), 3.14 (d, J=6.48 Hz, 2H), 3.93 (s, 2H), 4.41 (d, J=5.6 Hz, 2H), 6.27-6.29 (m, 1H), 6.34-6.38 (m, 1H), 6.44 (d, J=9.16 Hz, 1H), 6.56 (d, J=15.96 Hz, 1H), 7.09-7.11 (m, 2H), 7.23 (d, J=2.8 Hz, 1H), 7.31 (d, J=5.04 Hz, 1H), 7.37 (d, J=8.48 Hz, 2H), 7.45-7.49 (m, 3H), 7.56-7.58 (m, 2H), 7.89 (d, J=8.56 Hz, 1H), 8.17 (d, J=5.16 Hz, 1H). LC-MS (m/z): 584 (M+H) r.t. 7.23 min. HPLC: 98.60%.

### Example 76

### (E)-1′-(3-(4-chlorophenyl)allyl)-N-((2-chloropyridin-4-yl)methyl)-5-(pyrrolidin-1-yl)spiro[indoline-3,4′-piperidine]-1-carboxamide

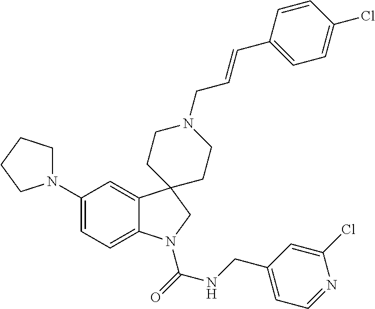 [SMILES: O=C(NCc1ccnc(Cl)c1)N1CC2(CCN(C/C=C/c3ccc(Cl)cc3)CC2)c2cc(N3CCCC3)ccc21]

Using the same procedures as Step 1 through 6 of Example 56 and replacing 4-(trifluoromethoxy)aniline with commercially available 4-(pyrrolidin-1-yl)aniline and (E)-3-(3,4-Dichloro-phenyl)-propenal with commercially available (E)-3-(4-chlorophenyl)acrylaldehyde the title compound is obtained: LC-MS (m/z): 575.6 (M+H) r.t. 6.06 min.

### Example 77

### (E)-1′-(3-(4-chlorophenyl)allyl)-N-((2-fluoropyridin-4-yl)methyl)-5-(trifluoromethoxy)spiro[indoline-3,4′-piperidine]-1-carboxamide

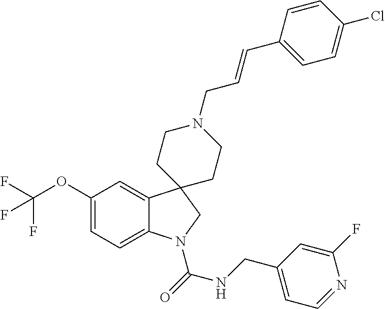 [SMILES: O=C(NCc1ccnc(F)c1)N1CC2(CCN(C/C=C/c3ccc(Cl)cc3)CC2)c2cc(OC(F)(F)F)ccc21]

Using the same procedures as Step 1 through 6 of Example 55 and replacing (2-Chloro-pyridin-4-yl)-methylamine hydrochloride with commercially available (2-fluoropyridin-4-yl)methanamine and (E)-3-(3,4-Dichloro-phenyl)-propenal with commercially available (E)-3-(4-chlorophenyl)acrylaldehyde the title compound is obtained: 1H NMR (400 MHz, DMSO-d6) δ: 1.67 (d, J=12.4 Hz, 2H), 1.85-1.91 (m, 2H), 2.05-2.10 (m, 2H), 2.90 (d, J=11.44 Hz, 2H), 3.14 (d, J=6.36 Hz, 2H), 3.92 (s, 2H), 4.40 (d, J=5.6 Hz, 2H), 6.33-6.40 (m, 1H), 6.56 (d, J=15.92 Hz, 1H), 7.07-7.10 (m, 2H), 7.22 (s, 1H), 7.31 (d, J=5 Hz, 1H), 7.37 (d, J=8.44 Hz, 2H), 7.48 (d, J=8.48 Hz, 2H), 7.55 (t, J=5.8 Hz, 1H), 7.87 (d, J=8.76 Hz, 1H), 8.17 (d, J=5.12 Hz, 1H). LC-MS (m/z): 574.8 (M+H) r.t. 5.91 min. HPLC: 99.54%.

### Example 78

### (E)-1′-(3-(4-chlorophenyl)allyl)-N-((2-chloropyridin-4-yl)methyl)-5-(trifluoromethylsulfonyl)spiro[indoline-3,4′-piperidine]-1-carboxamide

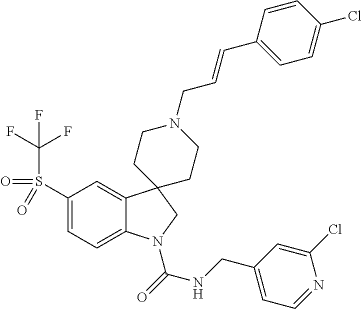 [SMILES: O=C(NCc1ccnc(Cl)c1)N1CC2(CCN(C/C=C/c3ccc(Cl)cc3)CC2)c2cc(S(=O)(=O)C(F)(F)F)ccc21]

Using the same procedures as Step 1 through 6 of Example 55 and replacing 4-(trifluoromethoxy)aniline with commercially available 4-((trifluoromethyl)-sulfonyl)aniline and (E)-3-(3,4-Dichloro-phenyl)-propenal with commercially available (E)-3-(4-chlorophenyl)acrylaldehyde the title compound is obtained: 1H NMR (400 MHz, DMSO-d6) δ: 1.74 (d, J=12.4 Hz, 2H), 1.88-1.94 (m, 2H), 2.06-2.11 (m, 2H), 2.92 (d, J=11.08 Hz, 2H), 3.15 (d, J=6.16 Hz, 2H), 4.03 (s, 2H), 4.40 (d, J=5.64 Hz, 2H), 6.32-6.39 (m, 1H), 6.58 (d, J=15.96 Hz, 1H), 7.32-7.39 (m, 3H), 7.47-7.49 (m, 3H), 7.78 (s, 1H), 7.88-7.90 (m, 2H), 8.16 (d, J=8.76 Hz, 1H), 8.34 (d, J=5.08 Hz, 1H). LC-MS (m/z): 638.9 (M+H) r.t. 5.99 min. HPLC: 99.74%.

### Example 79

### (E)-1′-(3-(4-chlorophenyl)allyl)-N-((2-chloropyridin-4-yl)methyl)-5-(methylsulfonyl)spiro[indoline-3,4′-piperidine]-1-carboxamide

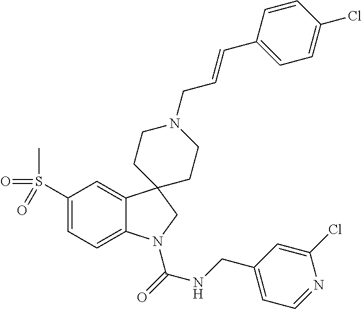 [SMILES: CS(=O)(=O)c1ccc2c(c1)C1(CCN(C/C=C/c3ccc(Cl)cc3)CC1)CN2C(=O)NCc1ccnc(Cl)c1]

Using the same procedures as Step 1 through 6 of Example 55 and replacing 4-(trifluoromethoxy)aniline with commercially available 4-(methylsulfonyl)aniline and (E)-3-(3,4-Dichloro-phenyl)-propenal with commercially available (E)-3-(4-chlorophenyl)acrylaldehyde the title compound is obtained: 1H NMR (400 MHz, DMSO-d6) δ: 1.70 (d, J=12.74 Hz, 2H), 1.89-1.95 (m, 2H), 2.09 (t, J=11.87 Hz, 2H), 2.93 (d, J=11.32 Hz, 2H), 3.15-3.16 (m, 5H), 3.96 (s, 2H), 4.38 (d, J=5.56 Hz, 2H), 6.33-6.40 (m, 1H), 6.58 (d, J=5.15 Hz, 1H), 7.38-7.39 (m, 3H), 7.46-7.49 (m, 3H), 7.65-7.71 (m, 3H), 7.99 (d, J=8.36 Hz, 1H), 8.35 (d, J=5.04 Hz, 1H). LC-MS (m/z): 584.7 (M+H) r.t. 2.92 min. HPLC: 99.90%.

### Example 80

### (E)-1′-(3-(4-chlorophenyl)allyl)-N-((2-chloropyridin-4-yl)methyl)-5-(trifluoromethylthio)spiro[indoline-3,4′-piperidine]-1-carboxamide

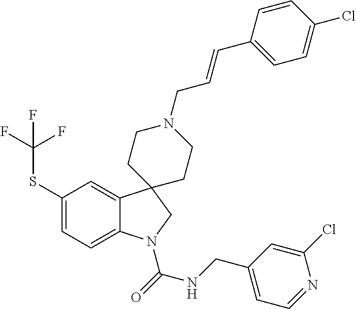 [SMILES: O=C(NCc1ccnc(Cl)c1)N1CC2(CCN(C/C=C/c3ccc(Cl)cc3)CC2)c2cc(SC(F)(F)F)ccc21]

Using the same procedures as Step 1 through 6 of Example 55 and replacing 4-(trifluoromethoxy)aniline with commercially available 4-((trifluoromethyl)thio)-aniline and (E)-3-(3,4-Dichloro-phenyl)-propenal with commercially available (E)-3-(4-chlorophenyl)acrylaldehyde the title compound is obtained: 1H NMR (400 MHz, DMSO-d6) δ: 1.69 (d, J=11.84 Hz, 2H), 1.84-1.91 (m, 2H), 2.08 (t, J=11.24 Hz, 2H), 2.91 (d, J=11.76 Hz, 2H), 3.15 (d, J=6.6 Hz, 2H), 3.93 (s, 2H), 4.37 (d, J=5.68 Hz, 2H), 6.33-6.40 (m, 1H), 6.57 (d, J=15.88 Hz, 1H), 7.45-7.47 (m, 3H), 7.47-7.49 (m, 5H), 7.64 (t, J=5.78 Hz, 1H), 7.93 (d, J=8.4 Hz, 1H), 8.34 (d, J=5.0 Hz, 1H). (LC-MS (m/z): 606.6 (M+H) r.t. 4.97 min. HPLC: 99.88%.

### Example 81

### (E)-1′-(3-(4-chlorophenyl)allyl)-N-((2-chloropyridin-4-yl)methyl)-5-(thiazol-2-yl)spiro[indoline-3,4′-piperidine]-1-carboxamide

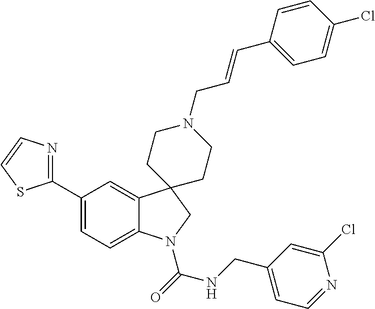 [SMILES: O=C(NCc1ccnc(Cl)c1)N1CC2(CCN(C/C=C/c3ccc(Cl)cc3)CC2)c2cc(-c3nccs3)ccc21]

Using the same procedures as Step 1 through 6 of Example 56 and replacing 4-(trifluoromethoxy)aniline with commercially available 4-(thiazol-2-yl)aniline and (E)-3-(3,4-Dichloro-phenyl)-propenal with commercially available (E)-3-(4-chlorophenyl)acrylaldehyde the title compound is obtained: 1H NMR (400 MHz, DMSO-d6) δ: 1.71 (d, J=12.28 Hz, 2H), 1.90-1.96 (m, 2H), 2.10 (t, J=12.26 Hz, 2H), 2.94 (d, J=11.16 Hz, 2H), 3.16 (d, J=6.44 Hz, 2H), 3.93 (s, 2H), 4.37 (d, J=5.52 Hz, 2H), 6.35-6.42 (m, 1H), 6.58 (d, J=15.88 Hz, 1H), 7.37-7.39 (m, 3H), 7.46-7.50 (m, 3H), 7.59 (t, J=5.82 Hz, 1H), 7.67 (d, J=3.24 Hz, 1H), 7.71-7.73 (m, 2H), 7.84 (d, J=3.28 Hz, 1H), 7.91 (d, J=8.9 Hz, 1H), 8.35 (d, J=5.08 Hz, 1H), LC-MS (m/z): 589.6 (M+H) r.t. 3.79 min. HPLC: 99.62%.

### Example 82

### (E)-1′-(3-(4-chlorophenyl)allyl)-N-((2-fluoropyridin-4-yl)methyl)-5-(trifluoromethyl)spiro[indoline-3,4′-piperidine]-1-carboxamide

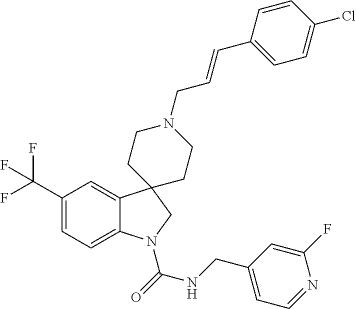 [SMILES: O=C(NCc1ccnc(F)c1)N1CC2(CCN(C/C=C/c3ccc(Cl)cc3)CC2)c2cc(C(F)(F)F)ccc21]

Using the same procedures as Step 1 through 6 of Example 55 and replacing 4-(trifluoromethoxy)aniline with commercially available 4-(trifluoromethyl)aniline and (2-Chloro-pyridin-4-yl)-methylamine hydrochloride with commercially available (2-fluoropyridin-4-yl)methanamine and (E)-3-(3,4-Dichloro-phenyl)-propenal with commercially available (E)-3-(4-chlorophenyl)acrylaldehyde the title compound is obtained: 1H NMR (400 MHz, DMSO-d6) δ: 1.68 (d, J=12.2 Hz, 2H), 1.89-1.95 (m, 2H), 2.08 (t, J=11.12 Hz, 2H), 2.92 (d, J=11.4 Hz, 2H), 3.15 (d, J=6.28 Hz, 2H), 3.95 (s, 2H), 4.40 (d, J=5.68 Hz, 2H), 6.33-6.40 (m, 1H), 6.57 (d, J=15.96 Hz, 1H), 7.12 (s, 1H), 7.32 (d, J=5.12 Hz, 1H), 7.38 (d, J=8.52 Hz, 2H), 7.40-7.52 (m, 4H), 7.65 (t, J=5.7 Hz, 1H), 7.97 (d, J=8.44 Hz, 1H), 8.17 (d, J=5.12 Hz, 1H). LC-MS (m/z): 558.9 (M+H) r.t. 5.83 min. HPLC: 99.09%.

### Example 83

### (E)-1′-(3-(4-chlorophenyl)allyl)-N-((2-fluoropyridin-4-yl)methyl)-5-(2-oxo-1,2-dihydropyridin-4-yl)spiro[indoline-3,4′-piperidine]-1-carboxamide

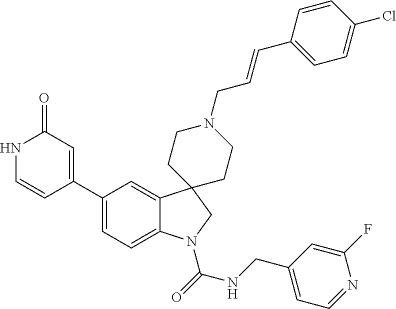 [SMILES: O=C(NCc1ccnc(F)c1)N1CC2(CCN(C/C=C/c3ccc(Cl)cc3)CC2)c2cc(-c3cc[nH]c(=O)c3)ccc21]

Using the same procedures as Step 1 through 6 of Example 56 and replacing 4-(trifluoromethoxy)aniline with commercially available 4-(4-aminophenyl)pyridin-2(1H)-one and (2-Chloro-pyridin-4-yl)-methylamine hydrochloride with commercially available (2-fluoropyridin-4-yl)methanamine and (E)-3-(3,4-Dichloro-phenyl)-propenal with commercially available (E)-3-(4-chlorophenyl)acrylaldehyde the title compound is obtained. 1H NMR (400 MHz, DMSO-d6) δ: 1.65 (d, J=12.16 Hz, 2H), 1.98-2.13 (m, 4H), 2.93 (d, J=11.12 Hz, 2H), 3.14 (d, J=6.36 Hz, 2H), 3.92 (s, 2H), 4.41 (d, J=5.6, 2H), 6.37-6.41 (m, 1H), 6.53-6.60 (m, 3H), 7.11 (s, 1H), 7.32 (d, J=5.16 Hz, 1H), 7.37-7.39 (m, 3H), 7.47-7.50 (m, 3H), 7.55-7.58 (m, 2H), 7.88 (d, J=8.48 Hz, 1H), 8.18 (d, J=5.16 Hz, 1H), 11.44 (s, 1H). LC-MS (m/z): 584.2 (M+H) r.t. 4.15 min. HPLC: 99.68%.

### Example 84

### (E)-1′-(3-(4-chlorophenyl)allyl)-5-cyclopropyl-N-((2-fluoropyridin-4-yl)methyl)spiro[indoline-3,4′-piperidine]-1-carboxamide

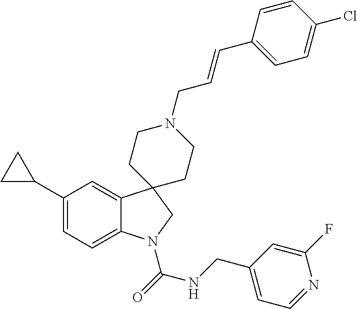 [SMILES: O=C(NCc1ccnc(F)c1)N1CC2(CCN(C/C=C/c3ccc(Cl)cc3)CC2)c2cc(C3CC3)ccc21]

Using the same procedures as Step 1 through 6 of Example 56 and replacing 4-(trifluoromethoxy)aniline with commercially available 4-cyclopropylaniline and (2-Chloro-pyridin-4-yl)-methylamine hydrochloride with commercially available (2-fluoropyridin-4-yl)methanamine and (E)-3-(3,4-Dichloro-phenyl)-propenal with commercially available (E)-3-(4-chlorophenyl)acrylaldehyde the title compound is obtained: 1H NMR (400 MHz, DMSO-d6) δ: 0.58-0.60 (m, 2H), 0.83-0.86 (m, 2H), 1.58 (d, J=12.84 Hz, 2H), 1.81-1.90 (m, 3H), 2.07 (t, J=10.96 Hz, 2H), 2.90 (d, J=10.32 Hz, 2H), 3.13 (d, J=6.24 Hz, 2H), 3.83 (s, 2H), 4.37 (d, J=5.6, 2H), 6.33-6.40 (m, 1H), 6.56 (d, J=15.76 Hz, 1H), 6.78-6.80 (m, 1H), 6.89 (s, 1H), 7.0 (s, 1H), 7.29 (d, J=4.84 Hz, 1H), 7.37-7.39 (m, 3H), 7.48 (d, J=8.48 Hz, 2H), 7.66 (d, J=8.24 Hz, 1H), 8.16 (d, J=5.16 Hz, 1H). LC-MS (m/z): 531.2 (M+H) r.t. 5.77 min. HPLC: 99.56%.

### Example 85

### (E)-1′-(3-(4-chlorophenyl)allyl)-N-((2-chlorothiazol-5-yl)methyl)-5-(trifluoromethyl)spiro[indoline-3,4′-piperidine]-1-carboxamide

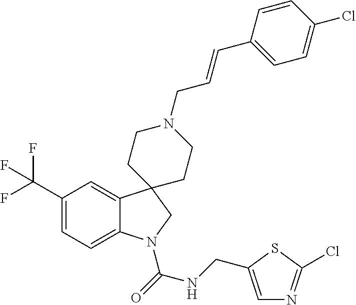 [SMILES: O=C(NCc1cnc(Cl)s1)N1CC2(CCN(C/C=C/c3ccc(Cl)cc3)CC2)c2cc(C(F)(F)F)ccc21]

Using the same procedures as Step 1 through 6 of Example 55 and replacing 4-(trifluoromethoxy)aniline with commercially available 4-(trifluoromethyl)aniline and (2-Chloro-pyridin-4-yl)-methylamine hydrochloride with commercially available 2-chloro-5-(aminomethyl)thiazole and (E)-3-(3,4-Dichloro-phenyl)-propenal with commercially available (E)-3-(4-chlorophenyl)acrylaldehyde the title compound is obtained: 1H NMR (400 MHz, DMSO) δ: 1.62 (d, J=12.6 Hz, 2H), 1.87-1.92 (m, 2H), 2.01-2.07 (m, 2H), 2.89 (d, J=11.2 Hz, 2H), 3.13 (d, J=6.4 Hz, 2H), 3.83 (s, 2H), 4.44 (s, 2H), 6.32-6.39 (m, 1H), 6.56 (d, J=15.88 Hz, 1H), 7.38 (d, J=8.48 Hz, 2H), 7.47-7.51 (m, 4H), 7.60 (s, 1H), 7.75 (brs, 1H), 8.01 (d, J=8.32 Hz, 1H). LC-MS (m/z): 581.0 (M+H) r.t. 6.27 min. HPLC: 99.89%.

### Example 86

### (E)-1-(3-(4-chlorophenyl)allyl)-N-((2-chloropyridin-4-yl)methyl)-5′-cyanospiro[piperidine-4,3′-pyrrolo[2,3-c]pyridine]-1′(2′H)-carboxamide

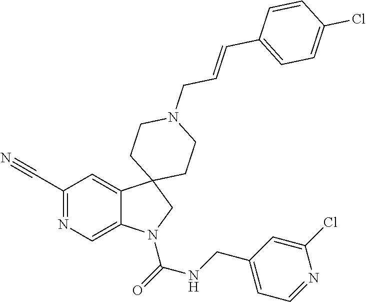 [SMILES: N#Cc1cc2c(cn1)N(C(=O)NCc1ccnc(Cl)c1)CC21CCN(C/C=C/c2ccc(Cl)cc2)CC1]

Using the same procedures as Step 1 through 6 of Example 55 and replacing 4-(trifluoromethoxy)aniline with commercially available 6-aminonicotinonitrile and (E)-3-(3,4-Dichloro-phenyl)-propenal with commercially available (E)-3-(4-chlorophenyl)acrylaldehyde the title compound is obtained: 1H NMR (400 MHz, DMSO-d6) δ: 1.71 (d, J=11.92 Hz, 2H), 1.88-1.94 (m, 2H), 2.05 (t, J=13 Hz, 2H), 2.92 (d, J=12.04 Hz, 2H), 3.14 (d, J=6.68 Hz, 2H), 3.97 (s, 2H), 4.39 (d, J=5.64 Hz, 2H), 6.33-6.40 (m, 1H), 6.56 (d, J=15.96 Hz, 1H), 7.37-7.39 (m, 3H), 7.47-7.49 (m, 3H), 7.84 (t, J=6.12, 1H), 8.01 (s, 1H), 8.35 (d, J=5.06 Hz, 1H), 9.06 (s, 1H), LC-MS (m/z): 532.9 (M+H) r.t. 3.17 min. HPLC: 99.23%.

### Example 87

### (E)-1-(3-(4-chlorophenyl)allyl)-N-((2-chloropyridin-4-yl)methyl)-5′-cyanospiro[piperidine-4,3′-pyrrolo[3,2-b]pyridine]-1′(2′H)-carboxamide

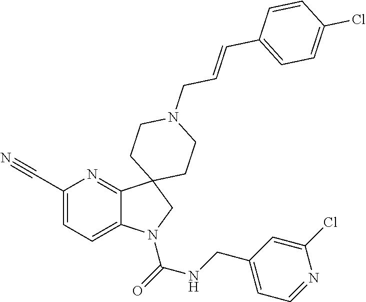 [SMILES: N#Cc1ccc2c(n1)C1(CCN(C/C=C/c3ccc(Cl)cc3)CC1)CN2C(=O)NCc1ccnc(Cl)c1]

Using the same procedures as Step 1 through 6 of Example 55 and replacing 4-(trifluoromethoxy)aniline with commercially available and (E)-3-(3,4-Dichloro-phenyl)-propenal with commercially available (E)-3-(4-chlorophenyl)-acrylaldehyde the title compound is obtained: 1H NMR (400 MHz, DMSO-d6) δ: 1.71 (d, J=11.72 Hz, 2H), 1.90-1.97 (m, 2H), 2.07 (t, J=12.44 Hz, 2H), 2.94 (d, J=13.8 Hz, 2H), 3.15 (d, J=6.24 Hz, 2H), 3.99 (s, 2H), 4.39 (d, J=5.64 Hz, 2H), 6.34-6.41 (m, 1H), 6.57 (d, J=15.86 Hz, 1H), 7.36-7.39 (m, 3H), 7.47-7.50 (m, 3H), 7.74 (d, J=8.36, 1H), 7.88 (t, J=6.88 Hz, 1H), 8.07 (d, J=8.36 Hz, 1H), 8.34 (d, J=5.12 Hz, 1H), LC-MS (m/z): 533.2 (M+H) r.t. 5.06 min.

### Example 88

### (E)-1′-(3-(4-chlorophenyl)allyl)-N-((2-chloropyridin-4-yl)methyl)-4-(trifluoromethoxy)spiro[indoline-3,4′-piperidine]-1-carboxamide

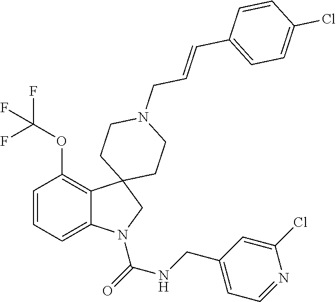 [SMILES: O=C(NCc1ccnc(Cl)c1)N1CC2(CCN(C/C=C/c3ccc(Cl)cc3)CC2)c2c(OC(F)(F)F)cccc21]

Using the same procedures as Step 1 through 6 of Example 55 and replacing 4-(trifluoromethoxy)aniline with commercially available 3-(trifluoromethoxy)aniline and (E)-3-(3,4-Dichloro-phenyl)-propenal with commercially available (E)-3-(4-chlorophenyl)acrylaldehyde the title compound is obtained: 1H NMR (400 MHz, DMSO-d6) δ: 1.66 (d, J=11.92 Hz, 2H), 2.03-2.09 (m, 2H), 2.13-2.19 (m, 2H), 2.90 (d, J=11.2 Hz, 2H), 3.14 (d, J=6.2 Hz, 2H), 3.92 (s, 2H), 4.36 (d, J=5.68 Hz, 2H), 6.36-6.43 (m, 1H), 6.54 (d, J=15.88 Hz, 1H), 6.80-6.82 (m, 1H), 7.22 (t, J=8.28 Hz, 1H), 7.36-7.38 (m, 3H), 7.45-7.50 (m, 3H), 7.57 (t, J=5.64, 1H), 7.86 (d, J=8.2 Hz, 1H), 8.34 (d, J=5.16 Hz, 1H), LC-MS (m/z): 590.8 (M+H) r.t. 6.22 min. HPLC: 99.83%.

### Example 89

### (E)-1′-(3-(4-chlorophenyl)allyl)-N-((2-chloropyridin-4-yl)methyl)-4-(trifluoromethylthio)spiro[indoline-3,4′-piperidine]-1-carboxamide

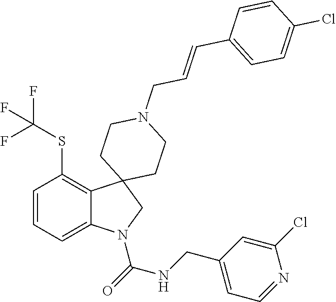 [SMILES: O=C(NCc1ccnc(Cl)c1)N1CC2(CCN(C/C=C/c3ccc(Cl)cc3)CC2)c2c(SC(F)(F)F)cccc21]

Using the same procedures as Step 1 through 6 of Example 55 and replacing 4-(trifluoromethoxy)aniline with commercially available 3-((trifluoromethyl)thio)aniline and (E)-3-(3,4-Dichloro-phenyl)-propenal with commercially available (E)-3-(4-chlorophenyl)acrylaldehyde the title compound is obtained: 1H NMR (400 MHz, DMSO-d6) δ: 1.65 (d, J=12.4 Hz, 2H), 2.09 (t, J=11.68 Hz, 2H), 2.43-2.50 (m, 2H), 2.92 (d, J=11.32 Hz, 2H), 3.14 (d, J=6.32 Hz, 2H), 3.92 (s, 2H), 4.37 (d, J=5.64 Hz, 2H), 6.41-6.45 (m, 1H), 6.54 (d, J=15.92 Hz, 1H), 7.13 (d, J=7.8 Hz, 1H), 7.27 (t, J=7.96 Hz, 1H), 7.36-7.38 (m, 3H), 7.46 (s, 1H), 7.50 (d, J=8.52 Hz, 2H), 7.60 (t, J=5.8 Hz, 1H), 8.13-8.15 (dd, J=0.8 Hz, J=8.12 Hz, 1H), 8.34 (d, J=5.12 Hz, 1H), LC-MS (m/z): 606.6 (M+H) r.t. 5.07 min. HPLC: 99.68%.

### Example 90

### (E)-1′-(3-(4-chlorophenyl)allyl)-4-fluoro-N-((2-fluoropyridin-4-yl)methyl)-6-(trifluoromethyl)spiro[indoline-3,4′-piperidine]-1-carboxamide

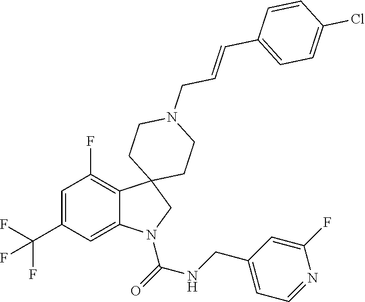 [SMILES: O=C(NCc1ccnc(F)c1)N1CC2(CCN(C/C=C/c3ccc(Cl)cc3)CC2)c2c(F)cc(C(F)(F)F)cc21]

Using the same procedures as Step 1 through 6 of Example 55 and replacing 4-(trifluoromethoxy)aniline with commercially available 3-fluoro-5-(trifluoromethyl)aniline and (2-Chloro-pyridin-4-yl)-methylamine hydrochloride with commercially available (2-fluoropyridin-4-yl)methanamine and (E)-3-(3,4-Dichloro-phenyl)-propenal with commercially available (E)-3-(4-chlorophenyl)acrylaldehyde the title compound is obtained: 1H NMR (400 MHz, DMSO-d6) δ: 1.76 (d, J=12 Hz, 2H), 2.01-2.08 (m, 2H), 2.12-2.17 (m, 2H), 2.92 (d, J=11.1 Hz, 2H), 3.13 (d, J=6.28 Hz, 2H), 3.99 (s, 2H), 4.40 (d, J=5.6 Hz, 2H), 6.38-6.42 (m, 1H), 6.55 (d, J=15.96 Hz, 1H), 7.11-7.13 (m, 2H), 7.32 (d, J=4.6 Hz, 1H), 7.36-7.38 (m, 2H), 7.49 (d, J=8.56 Hz, 2H), 7.71 (t, J=5.68 Hz, 1H), 8.03 (d, J=0.96 Hz, 1H), 8.17 (d, J=5.16 Hz, 1H), LC-MS (m/z): 576.8. (M+H) r.t. 6.24 min. HPLC: 99.67%.

### Example 91

### (E)-1′-(3-(4-chlorophenyl)allyl)-N-((2-fluoropyridin-4-yl)methyl)-4-phenylspiro[indoline-3,4′-piperidine]-1-carboxamide

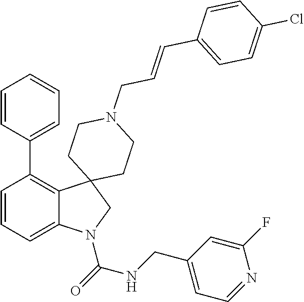 [SMILES: O=C(NCc1ccnc(F)c1)N1CC2(CCN(C/C=C/c3ccc(Cl)cc3)CC2)c2c(-c3ccccc3)cccc21]

Using the same procedures as Step 1 through 6 of Example 56 and replacing 4-(trifluoromethoxy)aniline with commercially available [1,1′-biphenyl]-3-amine and (2-Chloro-pyridin-4-yl)-methylamine hydrochloride with commercially available (2-fluoropyridin-4-yl)methanamine and (E)-3-(3,4-Dichloro-phenyl)-propenal with commercially available (E)-3-(4-chlorophenyl)acrylaldehyde the title compound is obtained: 1H NMR (400 MHz, DMSO) δ: 1.47 (d, J=12.2 Hz, 2H), 1.66-1.73 (m, 2H), 1.97-2.03 (m, 2H), 2.64 (d, J=12.48 Hz, 2H), 2.97 (d, J=6.36 Hz, 2H), 3.84 (s, 2H), 4.41 (d, J=5.64 Hz, 2H), 6.21-6.26 (m, 1H), 6.41 (d, J=15.88 Hz, 1H), 6.53 (dd, J=0.86 Hz, J=7.56 Hz, 1H), 7.08-7.12 (m, 2H), 7.24-7.26 (m, 2H), 7.31 (d, J=5.04 Hz, 1H), 7.35 (d, J=8.56 Hz, 2H), 7.40-7.44 (m, 5H), 7.52 (m, 1H), 7.97 (dd, J=0.96 Hz, J=8.12 Hz, 1H), 8.17 (d, J=5.16 Hz, 1H). LC-MS (m/z): 567.4 (M+H) r.t. 13.48 min. HPLC: 95.39%.

### Example 92

### (E)-1′-(3-(4-chlorophenyl)allyl)-N-((2-fluoropyridin-4-yl)methyl)-4-(trifluoromethyl)spiro[indoline-3,4′-piperidine]-1-carboxamide

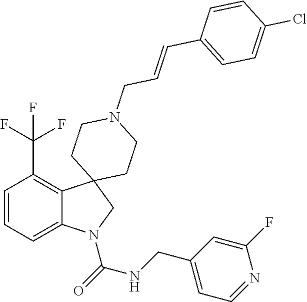 [SMILES: O=C(NCc1ccnc(F)c1)N1CC2(CCN(C/C=C/c3ccc(Cl)cc3)CC2)c2c1cccc2C(F)(F)F]

Using the same procedures as Step 1 through 6 of Example 56 and replacing 4-(trifluoromethoxy)aniline with commercially available (2-Chloro-pyridin-4-yl)-methylamine hydrochloride with commercially available 2-chloro-5-(aminomethyl)thiazole and (E)-3-(3,4-Dichloro-phenyl)-propenal with commercially available (E)-3-(4-chlorophenyl)acrylaldehyde the title compound is obtained: 1H NMR (400 MHz, DMSO) δ: 1.60 (d, J=11.52 Hz, 2H), 2.08-2.21 (m, 4H), 2.88 (d, J=10.44 Hz, 2H), 3.14 (d, J=6.36 Hz, 2H), 3.97 (s, 2H), 4.41 (d, J=5.64 Hz, 2H), 6.35-6.43 (m, 1H), 6.54 (d, J=15.96 Hz, 1H), 7.11 (s, 1H), 7.23 (d, J=7.32 Hz, 1H), 7.30-7.38 (m, 4H), 7.49 (d, J=8.56 Hz, 2H), 7.69 (t, J=5.72 Hz, 1H), 8.17 (d, J=5.16 Hz, 1H), 8.28 (d, J=8 Hz, 1H). LC-MS (m/z): 558.9 (M+H) r.t. 11.09 min. HPLC: 99.49%.

### Example 93

### (E)-1′-(3-(4-chlorophenyl)allyl)-4-cyclopropyl-N-((2-fluoropyridin-4-yl)methyl)spiro[indoline-3,4′-piperidine]-1-carboxamide

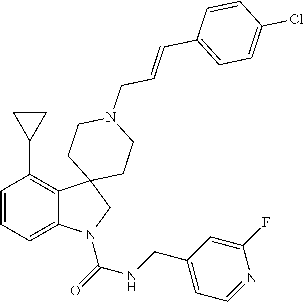 [SMILES: O=C(NCc1ccnc(F)c1)N1CC2(CCN(C/C=C/c3ccc(Cl)cc3)CC2)c2c(C3CC3)cccc21]

Using the same procedures as Step 1 through 6 of Example 56 and replacing 4-(trifluoromethoxy)aniline with commercially available 3-cyclopropylaniline and (2-Chloro-pyridin-4-yl)-methylamine hydrochloride with commercially available (2-fluoropyridin-4-yl)methanamine and (E)-3-(3,4-Dichloro-phenyl)-propenal with commercially available (E)-3-(4-chlorophenyl)acrylaldehyde the title compound is obtained: 1H NMR (400 MHz, DMSO-d6) δ: 0.69-0.73 (m, 2H), 0.96-1.00 (m, 2H), 1.63 (d, J=16 Hz, 2H), 2.08-2.15 (m, 3H), 2.49-2.53 (m, 2H), 2.92 (d, J=10.92 Hz, 2H), 3.14 (d, J=6.4 Hz, 2H), 3.86 (s, 2H), 4.39 (d, J=5.56 Hz, 2H), 6.35-6.43 (m, 2H), 6.55 (d, J=15.96 Hz, 1H), 6.98 (t, J=7.98 Hz, 1H) 7.09 (s, 1H), 7.30 (d, J=5.12 Hz, 1H), 7.37 (d, J=8.52 Hz, 2H), 7.43-7.50 (m, 3H), 7.75 (d, J=8.08 Hz, 1H), 8.17 (d, J=5.16 Hz, 1H) (LC-MS (m/z): 530.8 (M+H) r.t. 5.78 min. HPLC: 99.09%.

### Example 94

### (E)-1′-(3-(4-chlorophenyl)allyl)-N-((2-fluoropyridin-4-yl)methyl)-4-(trifluoromethoxy)spiro[indoline-3,4′-piperidine]-1-carboxamide

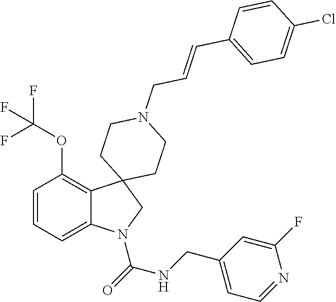 [SMILES: O=C(NCc1ccnc(F)c1)N1CC2(CCN(C/C=C/c3ccc(Cl)cc3)CC2)c2c(OC(F)(F)F)cccc21]

Using the same procedures as Step 1 through 6 of Example 55 and replacing 4-(trifluoromethoxy)aniline with commercially available 3-(trifluoromethoxy)aniline and (2-Chloro-pyridin-4-yl)-methylamine hydrochloride with commercially available (2-fluoropyridin-4-yl)methanamine and (E)-3-(3,4-Dichloro-phenyl)-propenal with commercially available (E)-3-(4-chlorophenyl)acrylaldehyde the title compound is obtained: 1H NMR (400 MHz, DMSO-d6) δ: 1.67 (d, J=11.96 Hz, 2H), 2.06 (t, J=11.78 Hz, 2H), 2.14-2.19 (m, 2H), 2.90 (d, J=11.36 Hz, 2H), 3.14 (d, J=6.36 Hz, 2H), 3.93 (s, 2H), 4.40 (d, J=5.6 Hz, 2H), 6.36-6.43 (m, 1H), 6.54 (d, J=15.96 Hz, 1H), 6.80-6.82 (m, 1H), 7.11 (s, 1H), 7.22 (t, J=8.26 Hz, 1H), 7.31 (d, J=5.16 Hz, 1H), 7.37 (d, J=8.52 Hz, 2H), 7.49 (d, J=8.56 Hz, 2H), 7.59 (t, J=5.76 Hz, 1H), 7.87 (d, J=8.16 Hz, 1H), 8.17 (d, J=5.16 Hz, 1H). (LC-MS (m/z): 575.2 (M+H) r.t. 16.4 min. HPLC: 99.07%.

### Example 95

### (E)-1′-(3-(4-chlorophenyl)allyl)-N-((2-chlorothiazol-5-yl)methyl)-4-(trifluoromethoxy)spiro[indoline-3,4′-piperidine]-1-carboxamide

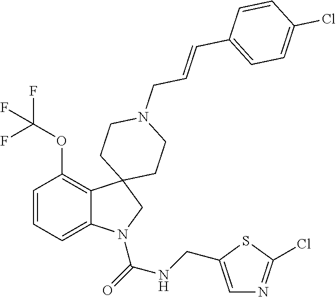 [SMILES: O=C(NCc1cnc(Cl)s1)N1CC2(CCN(C/C=C/c3ccc(Cl)cc3)CC2)c2c(OC(F)(F)F)cccc21]

Using the same procedures as Step 1 through 6 of Example 55 and replacing 4-(trifluoromethoxy)aniline with commercially available 3-(trifluoromethoxy)aniline and (2-Chloro-pyridin-4-yl)-methylamine hydrochloride with commercially available 2-chloro-5-(aminomethyl)thiazole and (E)-3-(3,4-Dichloro-phenyl)-propenal with commercially available (E)-3-(4-chlorophenyl)acrylaldehyde the title compound is obtained: 1H NMR (400 MHz, DMSO-d6) δ: 1.61 (d, J=12.2 Hz, 2H), 2.01 (t, J=11.88 Hz, 2H), 2.11-2.14 (m, 2H), 2.87 (d, J=11.08 Hz, 2H), 3.12 (d, J=6.32 Hz, 2H), 3.81 (s, 2H), 4.43 (d, J=5.44 Hz, 2H), 6.38-6.42 (m, 1H), 6.53 (d, J=15.92 Hz, 1H), 6.81-6.83 (m, 1H), 7.25 (t, J=8.26 Hz, 1H), 7.37 (d, J=8.52 Hz, 2H), 7.49 (d, J=8.56 Hz, 2H), 7.60 (s, 1H), 7.67 (t, J=5.62 Hz, 1H), 7.90 (d, J=7.84 Hz, 1H). (LC-MS (m/z): 596.6 (M+H) r.t. 8.88 min. HPLC: 99.63%.

### Example 96

### (E)-N-((2-chloropyridin-4-yl)methyl)-1′-(3-(4-cyanophenyl)allyl)-6-(2-(trifluoromethyl)phenyl)spiro[indoline-3,4′-piperidine]-1-carboxamide

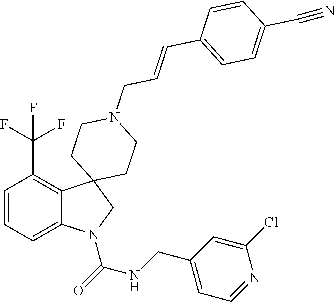 [SMILES: N#Cc1ccc(/C=C/CN2CCC3(CC2)CN(C(=O)NCc2ccnc(Cl)c2)c2cccc(C(F)(F)F)c23)cc1]

Using the same procedures as Step 1 through 6 of Example 56 and replacing 4-(trifluoromethoxy)aniline with commercially available 2′-(trifluoromethyl)-[1,1′-biphenyl]-3-amine and (E)-3-(3,4-Dichloro-phenyl)-propenal with commercially available (E)-4-(3-oxoprop-1-en-1-yl)benzonitrile the title compound is obtained: 1H NMR (400 MHz, DMSO-d6) δ: 1.70 (d, J=12.12 Hz, 2H), 1.92 (t, J=11.06 Hz, 2H), 2.12 (t, J=12.0 Hz, 2H), 2.94 (d, J=10.76 Hz, 2H), 3.20 (d, J=5.48 Hz, 2H), 3.92 (s, 2H), 4.34 (d, J=5.56 Hz, 2H), 6.56-6.70 (m, 2H), 6.83 (d, J=7.72 Hz, 1H), 7.25 (d, J=7.72 Hz, 1H), 6.34-6.36 (m, 2H), 7.43 (s, 1H), 7.49 (t, J=5.64 Hz, 1H), 7.55-7.59 (m, 1H), 7.65-7.68 (m, 3H), 7.78-7.81 (m, 4H), 8.32 (d, J=5.68 Hz, 1H). LC-MS (m/z): 642.1 (M+H) r.t. 8.44 min.

### Example 97

### (E)-N-((2-chloropyridin-4-yl)methyl)-1′-(3-(4-cyanophenyl)allyl)-5-nitrospiro[indoline-3,4′-piperidine]-1-carboxamide

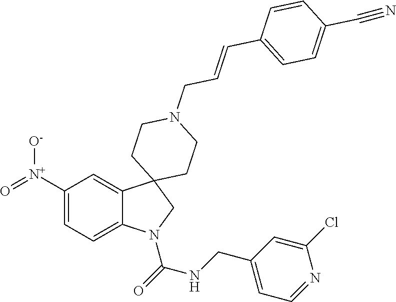 [SMILES: N#Cc1ccc(/C=C/CN2CCC3(CC2)CN(C(=O)NCc2ccnc(Cl)c2)c2ccc([N+](=O)[O-])cc23)cc1]

Using the same procedures as Step 2 through 6 of Example 55 and replacing (3-Trifluoromethyl-phenyl)-hydrazine with commercially (4-nitrophenyl)hydrazine and (E)-3-(3,4-Dichloro-phenyl)-propenal with commercially available (E)-4-(3-oxoprop-1-en-1-yl)benzonitrile the title compound is obtained: 1H NMR (400 MHz, DMSO) δ: 1.72 (d, J=12.4 Hz, 2H), 1.91-1.97 (m, 2H), 2.07-2.13 (m, 2H), 2.93 (d, J=11.59 Hz, 2H), 3.20 (d, J=5.84 Hz, 2H), 4.01 (s, 2H), 4.39 (d, J=5.64 Hz, 2H), 6.56-6.61 (m, 1H), 6.68 (d, J=15.96 Hz, 1H), 7.38-7.39 (m, 1H), 7.48 (s, 1H), 7.66 (d, J=8.4 Hz, 2H), 7.78-7.83 (m, 3H), 7.97-8.03 (m, 2H), 8.07-8.10 (m, 1H), 8.33 (d, J=5.08 Hz, 1H). LC-MS (m/z): 543.2 (M+H) r.t. 5.00 min.

### Example 98

### (E)-N-((2-chloropyridin-4-yl)methyl)-1′-(3-(4-cyanophenyl)allyl)-7-fluoro-4-(trifluoromethyl)spiro[indoline-3,4′-piperidine]-1-carboxamide

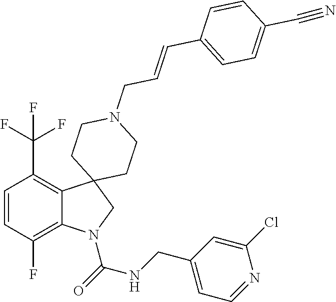 [SMILES: N#Cc1ccc(/C=C/CN2CCC3(CC2)CN(C(=O)NCc2ccnc(Cl)c2)c2c(F)ccc(C(F)(F)F)c23)cc1]

Using the same procedures as Step 1 through 6 of Example 55 and replacing 4-(trifluoromethoxy)aniline with commercially available 2-fluoro-5-(trifluoromethyl)aniline and (E)-3-(3,4-Dichloro-phenyl)-propenal with commercially available (E)-4-(3-oxoprop-1-en-1-yl)benzonitrile the title compound is obtained: 1H NMR (400 MHz, DMSO-d6) δ: 1.48 (d, J=12.28 Hz, 2H), 2.07-2.10 (m, 2H), 2.17 (t, J=11.06 Hz, 2H), 2.91 (d, J=11.04 Hz, 2H), 3.18 (d, J=5.48 Hz, 2H), 4.04 (s, 2H), 4.36 (d, J=5.68 Hz, 2H), 6.54-6.66 (m, 2H), 7.28-7.32 (m, 3H), 7.45 (dd, J₁=4.36 Hz, J₂=8.84 Hz, 1H), 7.66 (d, J=8.36 Hz, 2H), 7.78 (d, J=8.32 Hz, 2H), 8.01 (t, J=5.68 Hz, 1H), 8.34 (d, J=5.04 Hz, 1H). LC-MS (m/z): 583.9 (M+H) r.t. 5.29 min. HPLC: 99.89%.

### Example 99

### (E)-N-((2-chloropyridin-4-yl)methyl)-1′-(3-(4-cyanophenyl)allyl)-4-(trifluoromethyl)spiro[indoline-3,4′-piperidine]-1-carboxamide

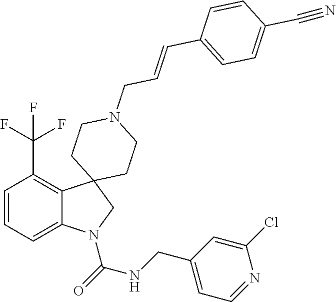 [SMILES: N#Cc1ccc(/C=C/CN2CCC3(CC2)CN(C(=O)NCc2ccnc(Cl)c2)c2cccc(C(F)(F)F)c23)cc1]

Using the same procedures as Step 2 through 6 of Example 56 and replacing (3-Trifluoromethyl-phenyl)-hydrazine with commercially available (3-(trifluoromethyl)phenyl)hydrazine and (E)-3-(3,4-Dichloro-phenyl)-propenal with commercially available (E)-4-(3-oxoprop-1-en-1-yl)benzonitrile the title compound is obtained: 1H NMR (400 MHz, DMSO) δ: 1.60 (d, J=11.12 Hz, 2H), 2.12-2.21 (m, 4H), 2.89 (d, J=9.88 Hz, 2H), 3.18 (d, J=4.98 Hz, 2H), 3.96 (s, 2H), 4.38 (d, J=5.64 Hz, 2H), 6.60-6.62 (m, 2H), 7.24 (d, J=7.32 Hz, 1H), 7.32-7.37 (m, 2H), 7.45 (s, 1H), 7.67 (d, J=8.36 Hz, 3H), 7.78 (d, J=8.4 Hz, 2H), 8.28 (d, J=8.04 Hz, 1H), 8.34 (d, J=5.08 Hz, 1H). LC-MS (m/z): 566.0 (M+H) r.t. 5.52 min

### Example 100

### (E)-1′-(3-(4-fluorophenyl)allyl)-N-((2-fluoropyridin-4-yl)methyl)-6-(trifluoromethoxy)spiro[indoline-3,4′-piperidine]-1-carboxamide

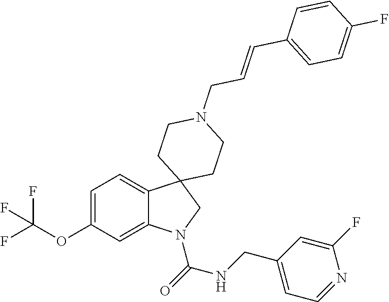 [SMILES: O=C(NCc1ccnc(F)c1)N1CC2(CCN(C/C=C/c3ccc(F)cc3)CC2)c2ccc(OC(F)(F)F)cc21]

Using the same procedures as Step 1 through 6 of Example 55 and replacing 4-(trifluoromethoxy)aniline with commercially available 3-(trifluoromethoxy)aniline and (2-Chloro-pyridin-4-yl)-methylamine hydrochloride with commercially available (2-fluoropyridin-4-yl)methanamine and (E)-3-(3,4-Dichloro-phenyl)-propenal with commercially available (E)-3-(4-fluorophenyl)acrylaldehyde the title compound is obtained: 1H-NMR (400 MHz, DMSO-d6) δ: 1.66 (d, J=12.48 Hz, 2H), 1.83-1.89 (m, 2H), 2.03-2.09 (m, 2H), 2.91 (d, J=11.56 Hz, 2H), 3.13 (d, J=6.40 Hz, 2H), 3.92 (s, 2H), 4.40 (d, J=5.60 Hz, 2H), 6.26-6.33 (m, 1H), 6.55 (d, J=15.92 Hz, 1H), 6.83-6.84 (d, J=6.60 Hz, 1H), 7.12-7.17 (m, 3H), 7.28-7.32 (m, 2H), 7.48-7.52 (m, 2H), 7.62 (t, J=5.76 Hz, 1H), 7.78 (s, 1H), 8.16 (d, J=5.12 Hz, 1H). LC-MS (m/z): 558.8 (M+H) r.t. 6.92 min. HPLC: 99.73%.

### Example 101

### (E)-1′-(3-(4-fluorophenyl)allyl)-N-((2-fluoropyridin-4-yl)methyl)-6-(trifluoromethyl)spiro[indoline-3,4′-piperidine]-1-carboxamide

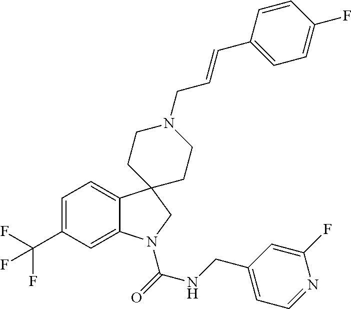 [SMILES: O=C(NCc1ccnc(F)c1)N1CC2(CCN(C/C=C/c3ccc(F)cc3)CC2)c2ccc(C(F)(F)F)cc21]

Using the same procedures as Step 1 through 6 of Example 55 and replacing 4-(trifluoromethoxy)aniline with commercially available 3-(trifluoromethyl)aniline and (2-Chloro-pyridin-4-yl)-methylamine hydrochloride with commercially available (2-fluoropyridin-4-yl)methanamine and (E)-3-(3,4-Dichloro-phenyl)-propenal with commercially available (E)-3-(4-fluorophenyl)acrylaldehyde the title compound is obtained: 1H NMR (400 MHz, DMSO) δ: 1.68 (d, J=12.24 Hz, 2H), 1.86-1.91 (m, 2H), 2.08 (t, J=11.36 Hz, 2H), 2.92 (d, J=11.44 Hz, 2H), 3.14 (d, J=6.44 Hz, 2H), 3.94 (s, 2H), 4.40 (d, J=5.64 Hz, 2H), 6.26-6.34 (m, 1H), 6.56 (d, J=15.96 Hz, 1H), 7.12-7.17 (m, 3H), 7.23 (d, J=7.12 Hz, 1H), 7.32 (d, J=4.6 Hz, 1H), 7.42 (d, J=7.76 Hz, 1H), 7.48-7.52 (m, 2H), 7.64 (t, J=5.72 Hz, 1H), 8.13 (d, J=1.08 Hz, 1H), 8.17 (d, J=5.12 Hz, 1H). LC-MS (m/z): 542.8 (M+H) r.t. 5.53 min. HPLC: 99.95%.

### Example 102

### (E)-4-bromo-N-((2-chloropyridin-4-yl)methyl)-1′-(3-(3,4-dichlorophenyl)allyl)spiro[indoline-3,4′-piperidine]-1-carboxamide

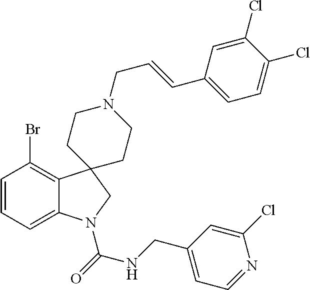 [SMILES: O=C(NCc1ccnc(Cl)c1)N1CC2(CCN(C/C=C/c3ccc(Cl)c(Cl)c3)CC2)c2c(Br)cccc21]

Using the same procedures as Step 2 through 6 of Example 56 and replacing (3-Trifluoromethyl-phenyl)-hydrazine with commercially available (3-bromophenyl)hydrazine the title compound is obtained: ¹H NMR (400 MHz, Chloroform-d) δ ppm 8.30 (dd, J=5.1, 0.5 Hz, 1H), 8.02 (dd, J=7.8, 1.2 Hz, 1H), 7.47 (d, J=2.0 Hz, 1H), 7.39 (d, J=8.3 Hz, 1H), 7.31 (d, J=0.7 Hz, 1H), 7.19-7.25 (m, 2H), 7.04-7.15 (m, 2H), 6.53 (d, J=15.9 Hz, 1H), 6.33 (dt, J=15.9, 6.8 Hz, 1H), 6.14 (br. s., 1H), 4.50 (d, J=5.6 Hz, 2H), 3.94 (s, 2H), 3.53 (d, J=6.8 Hz, 2H), 3.14 (d, J=12.5 Hz, 2H), 2.99 (td, J=13.8, 4.0 Hz, 2H), 2.69-2.82 (m, 2H), 1.59 (d, J=13.9 Hz, 2H). LC-MS (m/z): 619.0 (M+H) r.t. 2.72 min.

### Example 103

### 4-bromo-1′-[(E)-3-(3,4-dichlorophenyl)allyl]-N-[(2-fluoro-4-pyridyl)methyl]spiro[indoline-3,4′-piperidine]-1-carboxamide

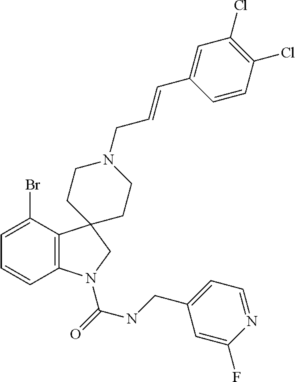 [SMILES: O=C(NCc1ccnc(F)c1)N1CC2(CCN(C/C=C/c3ccc(Cl)c(Cl)c3)CC2)c2c(Br)cccc21]

Using the same procedures as Step 2 through 6 of Example 56 and replacing (3-Trifluoromethyl-phenyl)-hydrazine with commercially available (3-bromophenyl)hydrazine and (2-Chloro-pyridin-4-yl)-methylamine hydrochloride with commercially available (2-fluoropyridin-4-yl)methanamine the title compound is obtained. 1H NMR (400 MHz, DMSO-d6) δ: 1.53 (d, 2H), 2.10 (t, 2H), 2.67 (t, 2H), 2.93 (d, 2H), 3.14 (d, 2H), 3.92 (s, 2H), 4.40 (d, 2H), 6.53-6.57 (m, 2H), 7.03-7.12 (m, 3H), 7.32 (m, 1H), 7.49-7.56 (m, 3H), 7.77 (s, 1H), 7.96 (m, 1H), 8.17 (d, 1H), LC-MS (m/z): 605 (M+H), r.t. 3.35 min.

### Example 104

### 5-bromo-1′-[(E)-3-(3,4-dichlorophenyl)allyl]-N-[(2-fluoro-4-pyridyl)methyl]spiro[indoline-3,4′-piperidine]-1-carboxamide

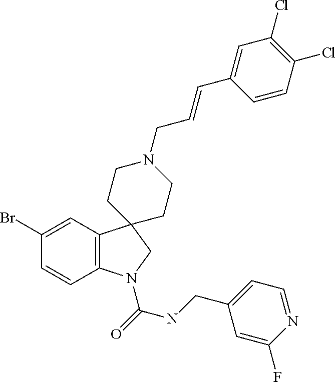 [SMILES: O=C(NCc1ccnc(F)c1)N1CC2(CCN(C/C=C/c3ccc(Cl)c(Cl)c3)CC2)c2cc(Br)ccc21]

Using the same procedures as Step 2 through 6 of Example 56 and replacing (3-Trifluoromethyl-phenyl)-hydrazine with commercially available (4-bromophenyl)hydrazine and (2-Chloro-pyridin-4-yl)-methylamine hydrochloride with commercially available (2-fluoropyridin-4-yl)methanamine the title compound is obtained: 1H NMR (400 MHz, CDCl₃) δ: 1.81 (d, 2H), 2.20 (t, 2H), 2.37 (t, 2H), 3.15 (d, 2H), 3.37 (d, 2H), 3.85 (s, 2H), 4.58 (d, 2H), 5.32 (m, 1H), 6.34-6.38 (m, 1H), 6.53 (d, 1H), 6.94 (s, 1H), 7.18-7.26 (m, 2H), 7.29-7.35 (m, 2H), 7.42 (d, 1H), 7.48 (d, 1H), 7.79 (m, 1H), 8.18 (d, 1H), LC-MS (m/z): 605 (M+H), r.t. 2.98 min.

### Example 105

### 5-bromo-1′-[(E)-3-(4-chlorophenyl)allyl]-N-[(2-fluoro-4-pyridyl)methyl]spiro[indoline-3,4′-piperidine]-1-carboxamide

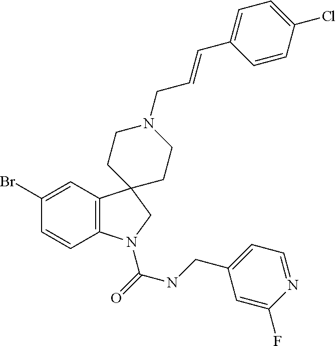 [SMILES: O=C(NCc1ccnc(F)c1)N1CC2(CCN(C/C=C/c3ccc(Cl)cc3)CC2)c2cc(Br)ccc21]

Using the same procedures as Step 2 through 6 of Example 56 and replacing (3-Trifluoromethyl-phenyl)-hydrazine with commercially available (4-bromophenyl)hydrazine and (2-Chloro-pyridin-4-yl)-methylamine hydrochloride with commercially available (2-fluoropyridin-4-yl)methanamine and (E)-3-(3,4-Dichloro-phenyl)-propenal with commercially available (E)-3-(4-chlorophenyl)acrylaldehyde the title compound is obtained: 1H NMR (400 MHz, CDCl₃) δ: 1.82 (d, 2H), 2.21 (t, 2H), 2.37 (t, 2H), 3.16 (d, 2H), 3.37 (d, 2H), 3.84 (s, 2H), 4.58 (d, 2H), 5.33 (m, 1H), 6.31-6.35 (m, 1H), 6.57 (d, 1H), 6.94 (s, 1H), 7.19 (m, 1H), 7.28-7.36 (m, 6H), 7.79 (m, 1H), 8.19 (d, 1H), LC-MS (m/z): 571 (M+H), r.t. 2.93 min.

### Example 106

### 5-bromo-1′-[(E)-3-(4-fluorophenyl)allyl]-N-[(2-fluoro-4-pyridyl)methyl]spiro[indoline-3,4′-piperidine]-1-carboxamide

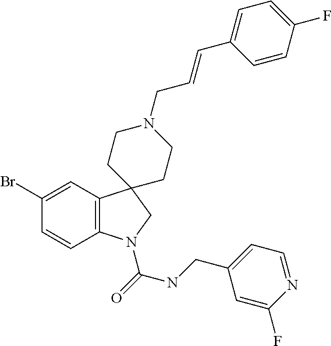 [SMILES: O=C(NCc1ccnc(F)c1)N1CC2(CCN(C/C=C/c3ccc(F)cc3)CC2)c2cc(Br)ccc21]

Using the same procedures as Step 2 through 6 of Example 56 and replacing (3-Trifluoromethyl-phenyl)-hydrazine with commercially available (4-bromophenyl)hydrazine and (2-Chloro-pyridin-4-yl)-methylamine hydrochloride with commercially available (2-fluoropyridin-4-yl)methanamine and (E)-3-(3,4-Dichloro-phenyl)-propenal with commercially available (E)-3-(4-fluorophenyl)acrylaldehyde the title compound is obtained: 1H NMR (400 MHz, CDCl₃) δ: 1.81 (d, 2H), 2.22 (t, 2H), 2.37 (t, 2H), 3.17 (d, 2H), 3.37 (d, 2H), 3.84 (s, 2H), 4.58 (d, 2H), 5.33 (m, 1H), 6.25-6.31 (m, 1H), 6.58 (d, 1H), 6.94 (s, 1H), 7.20 (m, 1H), 7.29-7.40 (m, 6H), 7.79 (m, 1H), 8.19 (d, 1H), LC-MS (m/z): 555 (M+H), r.t. 2.80 min.

### Example 107

### 5-bromo-N-[(2-fluoro-4-pyridyl)methyl]-1′-[(E)-3-[4-(trifluoromethyl)phenyl]allyl]spiro[indoline-3,4′-piperidine]-1-carboxamide

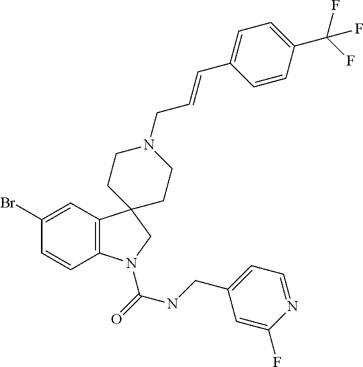 [SMILES: O=C(NCc1ccnc(F)c1)N1CC2(CCN(C/C=C/c3ccc(C(F)(F)F)cc3)CC2)c2cc(Br)ccc21]

Using the same procedures as Step 2 through 6 of Example 56 and replacing (3-Trifluoromethyl-phenyl)-hydrazine with commercially available (4-bromophenyl)hydrazine and (2-Chloro-pyridin-4-yl)-methylamine hydrochloride with commercially available (2-fluoropyridin-4-yl)methanamine and (E)-3-(3,4-Dichloro-phenyl)-propenal with commercially available (E)-3-(4-(trifluoromethyl)phenyl)acrylaldehyde the title compound is obtained: 1H NMR (400 MHz, CDCl₃) δ: 1.83 (d, 2H), 2.22 (t, 2H), 2.37 (t, 2H), 3.17 (d, 2H), 3.38 (d, 2H), 3.85 (s, 2H), 4.58 (d, 2H), 5.30 (m, 1H), 6.43-6.51 (m, 1H), 6.66 (d, 1H), 6.94 (s, 1H), 7.19 (m, 1H), 7.29-7.40 (m, 2H), 7.51 (d, 2H), 7.61 (d, 2H), 7.78 (m, 1H), 8.20 (d, 1H), LC-MS (m/z): 605 (M+H), r.t. 2.99 min.

### Example 108

### 5-(3-cyanophenyl)-1′-[(E)-3-(3,4-dichlorophenyl)allyl]-N-[(2-fluoro-4-pyridyl)methyl]spiro[indoline-3,4′-piperidine]-1-carboxamide

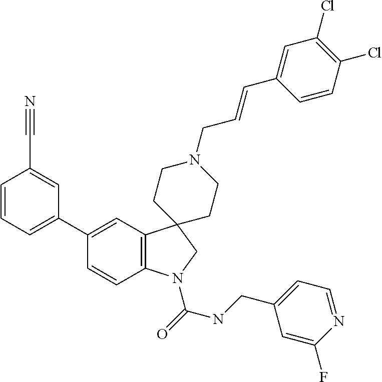 [SMILES: N#Cc1cccc(-c2ccc3c(c2)C2(CCN(C/C=C/c4ccc(Cl)c(Cl)c4)CC2)CN3C(=O)NCc2ccnc(F)c2)c1]

Using the same procedures as Step 1 through 6 of Example 56 and replacing 4-(trifluoromethoxy)aniline with commercially available 4′-amino-[1,1′-biphenyl]-3-carbonitrile and (2-Chloro-pyridin-4-yl)-methylamine hydrochloride with commercially available (2-fluoropyridin-4-yl)methanamine the title compound is obtained: 1H NMR (400 MHz, DMSO-d6) δ: 1.67 (d, 2H), 2.05-2.12 (m, 4H), 2.95 (d, 2H), 3.17 (d, 2H), 3.93 (s, 2H), 4.42 (d, 2H), 6.46-6.52 (m, 1H), 6.59 (d, 1H), 7.12 (s, 1H), 7.17 (m, 1H), 7.49-7.57 (m, 5H), 7.60-7.75 (m, 3H), 7.91 (d, 1H), 8.03 (d, 1H), 8.18-8.21 (m, 2H), LC-MS (m/z): 626 (M+H), r.t. 3.09 min.

### Example 109

### 1′-[(E)-3-(4-chlorophenyl)allyl]-5-(6-cyano-3-pyridyl)-N-[(2-fluoro-4-pyridyl)methyl]spiro[indoline-3,4′-piperidine]-1-carboxamide

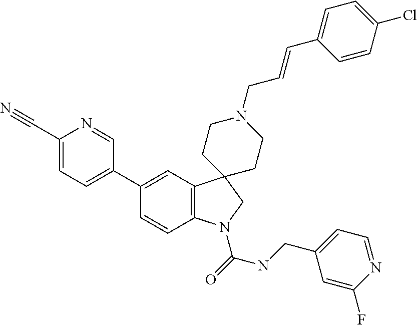 [SMILES: N#Cc1ccc(-c2ccc3c(c2)C2(CCN(C/C=C/c4ccc(Cl)cc4)CC2)CN3C(=O)NCc2ccnc(F)c2)cn1]

Step 1: 1′-[(E)-3-(4-chlorophenyl)allyl]-N-[(2-fluoro-4-pyridyl)methyl]-5-(4,4,5,5-tetramethyl-1,3,2-dioxaborolan-2-yl)spiro[indoline-3,4′-piperidine]-1-carboxamide

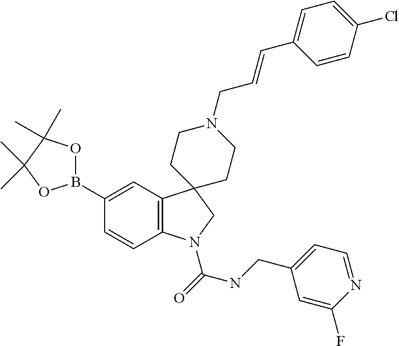 [SMILES: CC1(C)OB(c2ccc3c(c2)C2(CCN(C/C=C/c4ccc(Cl)cc4)CC2)CN3C(=O)NCc2ccnc(F)c2)OC1(C)C]

To a solution of 5-bromo-1′-[(E)-3-(4-chlorophenyl)allyl]-N-[(2-fluoro-4-pyridyl)methyl]-spiro[indoline-3,4′-piperidine]-1-carboxamide (Example 105) (1000 mg, 1.8 mmol) in dioxane (100 mL) was added bis(pinacolato)diboron (934 mg, 3.7 mmol), PdCl₂(dppf)₂ (66 mg, 0.09 mmol), and KOAc (950 mg, 9.2 mmol). The reaction mixture was heated to 95° C. for 18 h. Next, the reaction mixture was cooled, filtered through a celite plug, washed with additional dioxane, and concentrated under vacuum. The crude material was chromatographed (80 g Redi-Sep column) eluting from 100% hexanes to 90:10EtOAc:MeOH to afford the intermediate (435 mg, 40%) as a solid. LC-MS (m/z): 617 (M+H).

### Step 2: Preparation of Example 109

1′-[(E)-3-(4-chlorophenyl)allyl]-5-(6-cyano-3-pyridyl)-N-[(2-fluoro-4-pyridyl)methyl]spiro[indoline-3,4′-piperidine]-1-carboxamide. To a microwave vial containing 1′-[(E)-3-(4-chlorophenyl)allyl]-N-[(2-fluoro-4-pyridyl)methyl]-5-(4,4,5,5-tetramethyl-1,3,2-dioxaborolan-2-yl)spiro[indoline-3,4′-piperidine]-1-carboxamide (the product of step 2, Example 109) (100 mg, 0.16 mmol) and 3.5 mL dioxane was added 5-bromo-2-cyanopyridine (30 mg, 0.16 mmol), saturated NaHCO₃ (1 mL), and PdCl₂(dppf)₂ (10 mg, 0.014 mmol). The reaction was heated under microwave irradiation at 115° C. for 15 min. Next, the reaction was cooled, diluted with water and extracted with EtOAc (75 mL). The organic phase was dried (Na₂SO₄), concentrated under vacuum and chromatographed (24 g Redi-Sep column) eluting from 100% hexanes to 90:10 EtOAc:MeOH to afford the product (50 mg, 52%) as a glass. 1H NMR (400 MHz, DMSO-d6) δ: 1.67 (d, 2H), 2.04-2.13 (m, 4H), 2.95 (d, 2H), 3.17 (d, 2H), 3.95 (s, 2H), 4.42 (d, 2H), 6.34-6.40 (m, 1H), 6.57 (d, 1H), 7.13 (s, 1H), 7.32 (m, 1H), 7.39 (d, 2H), 7.49 (d, 2H), 7.58 (t, 1H), 7.66 (m, 1H), 7.78 (m, 1H), 7.96 (d, 1H), 8.05 (d, 1H), 8.18 (d, 1H), 8.35 (m, 1H), 9.12 (m, 1H), LC-MS (m/z): 593 (M+H), r.t. 3.25 min.

### Example 110

### 1′-[(E)-3-(3,4-dichlorophenyl)allyl]-N-[(2-fluoro-4-pyridyl)methyl]-5-(3-pyridyl)spiro[indoline-3,4′-piperidine]-1-carboxamide

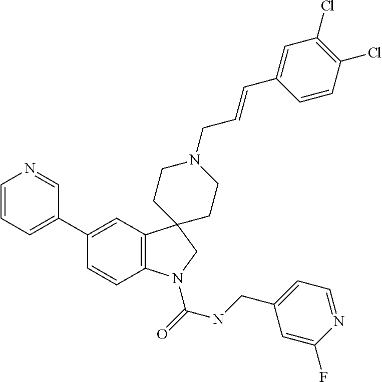 [SMILES: O=C(NCc1ccnc(F)c1)N1CC2(CCN(C/C=C/c3ccc(Cl)c(Cl)c3)CC2)c2cc(-c3cccnc3)ccc21]

Using the same procedures as Step 1 through 2 of Example 109 and replacing 5-bromo-2-cyanopyridine with commercially available 3-bromopyridine the title compound is obtained: 1H NMR (400 MHz, DMSO-d6) δ: 1.67 (d, 2H), 2.03-2.13 (m, 4H), 2.95 (d, 2H), 3.17 (d, 2H), 3.94 (s, 2H), 4.42 (d, 2H), 6.43-6.61 (m, 2H), 7.12 (s, 1H), 7.33 (m, 1H), 7.43-7.62 (m, 6H), 7.76 (m, 1H), 7.93 (d, 1H), 8.06 (m, 1H), 8.18 (d, 1H), 8.49 (m, 1H), 8.88 (m, 1H), LC-MS (m/z): 602 (M+H), r.t. 2.57 min.

### Example 111

### 1′-[(E)-3-(3,4-dichlorophenyl)allyl]-N-[(2-fluoro-4-pyridyl)methyl]-5-(1H-pyrazol-4-yl)spiro[indoline-3,4′-piperidine]-1-carboxamide

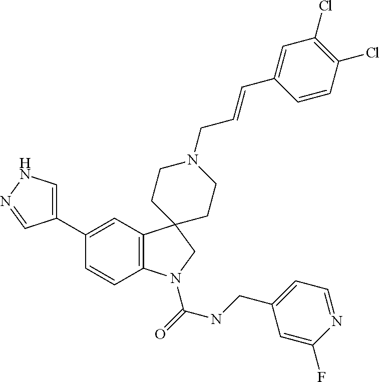 [SMILES: O=C(NCc1ccnc(F)c1)N1CC2(CCN(C/C=C/c3ccc(Cl)c(Cl)c3)CC2)c2cc(-c3cn[nH]c3)ccc21]

Using the same procedures as Step 1 through 2 of Example 109 and replacing 5-bromo-2-cyanopyridine with commercially available 4-bromo-1H-pyrazole the title compound is obtained: 1H NMR (400 MHz, DMSO-d6) δ: 1.65 (d, 2H), 196-2.14 (m, 4H), 2.94 (d, 2H), 3.16 (d, 2H), 3.88 (s, 2H), 4.40 (d, 2H), 6.45-6.60 (m, 2H), 7.11 (s, 1H), 7.31-7.49 (m, 5H), 7.58 (d, 1H), 7.76-7.79 (m, 2H), 7.87 (brs, 1H), 8.10 (brs, 1H), 8.17 (d, 1H), LC-MS (m/z): 591 (M+H), r.t. 2.72 min.

### Example 112

### 1′-[(E)-3-(3,4-dichlorophenyl)allyl]-N-[(2-fluoro-4-pyridyl)methyl]-5-pyrimidin-5-yl-spiro[indoline-3,4′-piperidine]-1-carboxamide

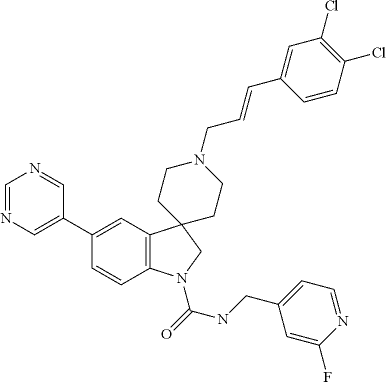 [SMILES: O=C(NCc1ccnc(F)c1)N1CC2(CCN(C/C=C/c3ccc(Cl)c(Cl)c3)CC2)c2cc(-c3cncnc3)ccc21]

Using the same procedures as Step 1 through 2 of Example 109 and replacing 5-bromo-2-cyanopyridine with commercially available 5-bromopyrimidine the title compound is obtained: 1H NMR (400 MHz, DMSO-d6) δ: 1.67 (d, 2H), 2.03-2.12 (m, 4H), 2.95 (d, 2H), 3.17 (d, 2H), 3.94 (s, 2H), 4.42 (d, 2H), 6.41-6.60 (m, 2H), 7.13 (s, 1H), 7.33 (m, 1H), 7.47 (d, 1H), 7.52-7.62 (m, 3H), 7.73 (m, 2H), 7.95 (d, 1H), 8.18 (d, 1H), 9.10 (s, 1H), 9.13 (s, 2H), LC-MS (m/z): 603 (M+H), r.t. 2.76 min.

### Example 113

### 5-cyano-1′-[(E)-3-(3, 4-dichlorophenyl)allyl]-N-[(2-fluoro-4-pyridyl)methyl]spiro[indoline-3,4′-piperidine]-1-carboxamide

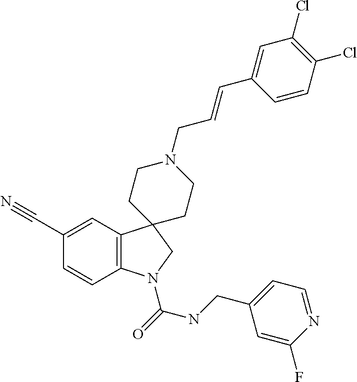 [SMILES: N#Cc1ccc2c(c1)C1(CCN(C/C=C/c3ccc(Cl)c(Cl)c3)CC1)CN2C(=O)NCc1ccnc(F)c1]

### Step 1: tert-butyl 5-cyanospiro[indoline-3,4′-piperidine]-1′-carboxylate

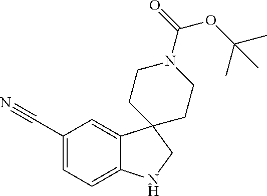 [SMILES: CC(C)(C)OC(=O)N1CCC2(CC1)CNc1ccc(C#N)cc12]

To a solution of commercially available tert-butyl 5-bromospiro[indoline-3,4′-piperidine]-1′-carboxylate (5000 mg, 14 mmol) in DMF (100 mL) was added zinc cyanide (1100 mg, 9.5 mmol), zinc acetate (500 mg, 2.7 mmol), PdCL₂(dppf)₂ (300 mg, 0.41 mmol), and zinc powder (360 mg, 5.4 mmol). The reaction mixture was purged with N₂ and heated to 100° C. for 18 hours. Next, the reaction mixture was cooled and poured onto ice water (300 mL). The resulting precipitate was filtered and washed with additional water. The precipitate was dissolved in DCM (300 mL), dried (Na₂SO₄), and concentrated under vacuum to afford the title compound as a solid (4.1 g, 96%): LC-MS (m/z): 258 (M+H-tbutyl), r.t. 3.43 min.

### Steps 2-4: 5-cyano-1′-[(E)-3-(3, 4-dichlorophenyl)allyl]-N-[(2-fluoro-4-pyridyl)methyl]spiro[indoline-3,4′-piperidine]-1-carboxamide (Example 113)

Using the same procedures as Step 4 through 6 of Example 56 and replacing tert-butyl 4-(trifluoromethyl)spiro[indoline-3,4′-piperidine]-1′-carboxylate with tert-butyl 5-cyanospiro[indoline-3,4′-piperidine]-1′-carboxylate (with the product of step 1, example 67) and (2-Chloro-pyridin-4-yl)-methylamine with (2-fluoropyridin-4-yl)methanamine the title compound is obtained: 1H NMR (400 MHz, DMSO-d6) δ: 1.66 (d, 2H), 1.91 (t, 2H), 2.09 (t, 2H), 2.93 (d, 2H), 3.15 (d, 2H), 3.95 (s, 2H), 4.41 (d, 2H), 6.41-6.58 (m, 2H), 7.13 (s, 1H), 7.32 (m, 1H), 7.48 (d, 1H), 7.53-7.61 (m, 2H), 7.68-7.76 (m, 3H), 7.95 (d, 1H), 8.18 (d, 1H), LC-MS (m/z): 550 (7M+H), r.t. 3.24 min.

### Example 114

### 1′-[(E)-3-(3, 4-dichlorophenyl)allyl]-N-[(2-fluoro-4-pyridyl)methyl]-4-methyl-spiro[indoline-3,4′-piperidine]-1-carboxamide

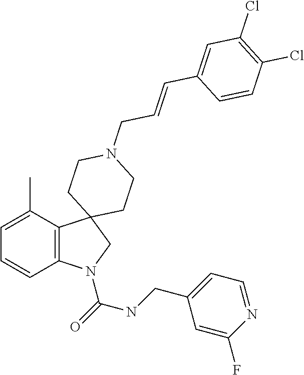 [SMILES: Cc1cccc2c1C1(CCN(C/C=C/c3ccc(Cl)c(Cl)c3)CC1)CN2C(=O)NCc1ccnc(F)c1]

To a microwave vial containing 4-bromo-1′-[(E)-3-(3,4-dichlorophenyl)allyl]-N-[(2-fluoro-4-pyridyl)methyl]spiro[indoline-3,4′-piperidine]-1-carboxamide (85 mg, 0.14 mmol) in dioxane (4 mL) was added methylboronic acid (25 mg, 0.42 mmol), PdCl₂(dppf)₂ (10 mg, 0.01 mmol), and saturated NaHCO₃(1 mL). The reaction mixture was heated under microwave irradiation for 15 min at 110° C. Next, the reaction mixture was cooled, diluted with dichloromethane (50 mL), washed with water, and concentrated under vacuum. The crude material was purified using reverse phase chromatography eluting from 95:5 water:acetonitrile with 0.1% TFA to 95:5 MeCN:water with 0.1% TFA to afford a glassy material. The glassy material was dissolved in DCM (3 mL) and washed with NaHCO₃, dried over MgSO₄, filtered and evaporated to give a glass (31 mg, 41%). 1H NMR (400 MHz, DMSO-d6) δ: 1.55 (d, 2H), 2.11 (t, 2H), 2.29 (t, 2H), 2.39 (s, 3H), 2.91 (d, 2H), 3.15 (d, 2H), 3.85 (s, 2H), 4.39 (d, 2H), 6.48-6.55 (m, 2H), 6.64 (d, 1H), 6.98 (t, 1H), 7.10 (s, 1H), 7.30 (m, 1H), 7.42 (t, 1H), 7.48 (d, 1H), 7.57 (d, 1H), 7.76-7.80 (m, 2H), 8.17 (d, 1H), LC-MS (m/z): 539 (M+H), r.t. 3.29 min.

### Example 115

### 1′-[(E)-3-(4-chlorophenyl)allyl]-5-(5-cyano-3-pyridyl)-N-[(2-fluoro-4-pyridyl)methyl]spiro[indoline-3,4′-piperidine]-1-carboxamide

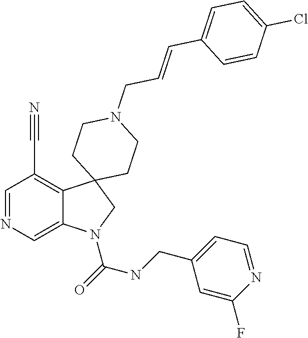 [SMILES: N#Cc1cncc2c1C1(CCN(C/C=C/c3ccc(Cl)cc3)CC1)CN2C(=O)NCc1ccnc(F)c1]

Using the same procedures as Step 1 through 2 of Example 109 and replacing 5-bromo-2-cyanopyridine with commercially available 5-bromonicotinonitrile the title compound is obtained: 1H NMR (400 MHz, DMSO-d6) δ: 1.67 (d, 2H), 2.05-2.14 (m, 4H), 2.95 (d, 2H), 3.17 (d, 2H), 3.94 (s, 2H), 4.42 (d, 2H), 6.33-641 (m, 1H), 6.59 (d, 1H), 7.13 (s, 1H), 7.32 (m, 1H), 7.39 (d, 2H), 7.49 (d, 2H), 7.57 (t, 1H), 7.64 (m, 1H), 7.81 (m, 1H), 7.93 (m, 1H), 8.18 (d, 1H), 8.69 (m, 1H), 8.90 (m, 1H) 9.20 (m, 1H), LC-MS (m/z): 593 (M+H), r.t. 3.19 min.

### Example 116

### 5-(6-cyano-3-pyridyl)-1′-[(E)-3-(3,4-dichlorophenyl)allyl]-N-[(2-fluoro-4-pyridyl)methyl]spiro[indoline-3,4′-piperidine]-1-carboxamide

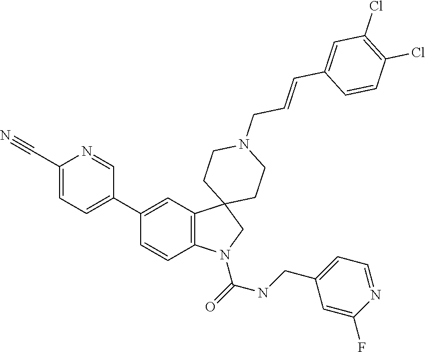 [SMILES: N#Cc1ccc(-c2ccc3c(c2)C2(CCN(C/C=C/c4ccc(Cl)c(Cl)c4)CC2)CN3C(=O)NCc2ccnc(F)c2)cn1]

Using the same procedures as Step 1 through 2 of Example 109 and replacing 5-bromo-2-cyanopyridine with commercially available 5-bromopicolinonitrile the title compound is obtained: 1H NMR (400 MHz, DMSO-d6) δ: 1.67 (d, 2H), 2.04-2.13 (m, 4H), 2.95 (d, 2H), 3.17 (d, 2H), 3.95 (s, 2H), 4.42 (d, 2H), 6.42-6.61 (m, 2H), 7.13 (s, 1H), 7.31 (m, 1H), 7.48 (d, 1H), 7.57-7.67 (m, 3H), 7.76 (m, 2H), 7.96 (d, 1H), 8.06 (m, 1H), 8.18 (d, 1H), 8.35 (m, 1H), 9.11 (m, 1H), LC-MS (m/z): 627 (M+H), r.t. 2.93 min.

### Example 117

### 5-cyano-N-[(2-fluoro-4-pyridyl)methyl]-1′-[(2-methoxy-8-methyl-7-quinolyl)methyl]spiro[indoline-3,4′-piperidine]-1-carboxamide

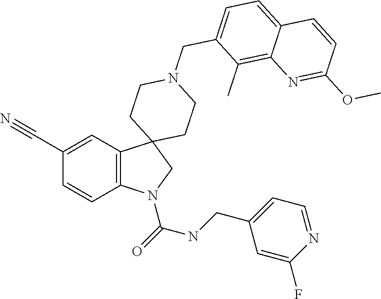 [SMILES: COc1ccc2ccc(CN3CCC4(CC3)CN(C(=O)NCc3ccnc(F)c3)c3ccc(C#N)cc34)c(C)c2n1]

Using the same procedures as Step 4 through 6 of Example 56 and replacing tert-butyl 4-(trifluoromethyl)spiro[indoline-3,4′-piperidine]-1′-carboxylate with tert-butyl 5-cyanospiro[indoline-3,4′-piperidine]-1′-carboxylate (with the product of step 1, example 113) and (2-Chloro-pyridin-4-yl)-methylamine with (2-fluoropyridin-4-yl)methanamine and (E)-3-(3,4-dichlorophenyl)acrylaldehyde with 2-methoxy-8-methylquinoline-7-carbaldehyde the title compound is obtained: 1H NMR (400 MHz, DMSO-d6) δ: 1.65 (d, 2H), 1.92 (t, 2H), 2.11 (t, 2H), 2.65 (s, 3H), 2.88 (d, 2H), 3.62 (s, 2H), 3.95 (s, 2H), 4.00 (s, 3H), 4.41 (d, 2H), 7.00 (d, 1H), 7.12 (s, 1H), 7.32 (m, 1H), 7.55-7.58 (m, 2H), 7.63 (s, 1H), 7.71-7.73 (m, 2H), 7.94 (d, 1H), 8.17-8.20 (m, 2H), LC-MS (m/z): 551 (M+H), r.t. 3.19 min.

### Example 118

### N-[(2-chloro-4-pyridyl)methyl]-5-cyano-1′-[(2-methoxy-8-methyl-7-quinolyl)methyl]spiro[indoline-3,4′-piperidine]-1-carboxamide

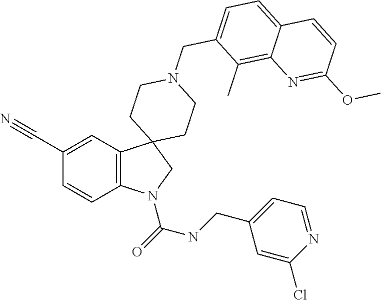 [SMILES: COc1ccc2ccc(CN3CCC4(CC3)CN(C(=O)NCc3ccnc(Cl)c3)c3ccc(C#N)cc34)c(C)c2n1]

Using the same procedures as Step 4 through 6 of Example 56 and replacing tert-butyl 4-(trifluoromethyl)spiro[indoline-3,4′-piperidine]-1′-carboxylate with tert-butyl 5-cyanospiro[indoline-3,4′-piperidine]-1′-carboxylate (with the product of step 1, Example 113) and (E)-3-(3,4-dichlorophenyl)acrylaldehyde with 2-methoxy-8-methylquinoline-7-carbaldehyde the title compound is obtained: 1H NMR (400 MHz, DMSO-d6) δ: 1.65 (d, J=12.0 Hz, 2H), 1.88-1.92 (m, 2H), 2.11 (t, J=12.0 Hz, 2H), 2.65 (s, 3H), 2.88 (d, J=8.0 Hz, 2H), 3.62 (s, 2H), 3.94 (s, 2H), 4.00 (s, 3H), 4.37 (d, J=4.0 Hz, 2H), 7.00 (d, J=12.0 Hz, 1H), 7.37 (d, J=4.0 Hz, 1H), 7.46 (s, 1H), 7.55-7.58 (m, 2H), 7.62 (s, 1H), 7.69-7.72 (m, 2H), 7.94 (d, J=8.0 Hz, 1H), 8.19 (d, J=8.0 Hz, 1H), 8.34 (d, J=4.0 Hz, 1H), LC-MS (m/z): 567 (M+H), r.t. 3.23 min.

### Example 119

### 1′-[(E)-3-(4-chlorophenyl)allyl]-N-[(2-chloro-4-pyridyl)methyl]-5-(6-methoxy-3-pyridyl)spiro[indoline-3,4′-piperidine]-1-carboxamide

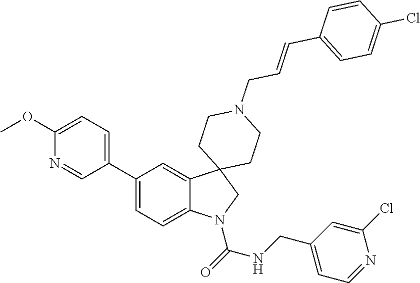 [SMILES: COc1ccc(-c2ccc3c(c2)C2(CCN(C/C=C/c4ccc(Cl)cc4)CC2)CN3C(=O)NCc2ccnc(Cl)c2)cn1]

### Step 1: tert-butyl 5-(6-methoxy-3-pyridyl)spiro[indoline-3,4′-piperidine]-1′-carboxylate

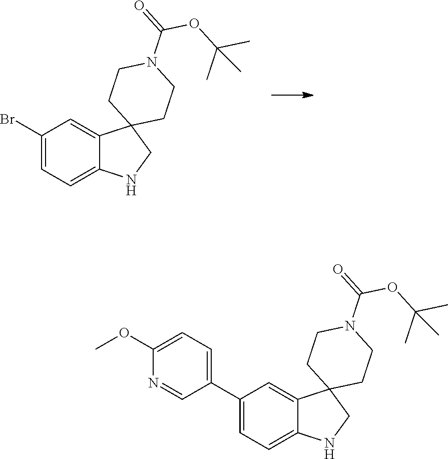 [SMILES: CC(C)(C)OC(=O)N1CCC2(CC1)CNc1ccc(Br)cc12 ; COc1ccc(-c2ccc3c(c2)C2(CCN(C(=O)OC(C)(C)C)CC2)CN3)cn1]

Tert-butyl-5-bromospiro[indoline-3,4′-piperidine]-1′-carboxylate (500 mg, 1.36 mmol) in 7 mL dioxane is treated with (6-methoxy-3-pyridyl)boronic acid (415 mg, 2.7 mmol), potassium carbonate (1.06 g, 6.8 mmol), and Pd tetrakis (80 mg, 0.07 mmol). Water (3 mL) is added and the reaction is heated at 120° C. for 1 hour in microwave then washed with water and brine, dried, filtered, conc and adsorbed on silica, MPLC to give (540 mg, 41%) of title compound: 1H NMR (400 MHz, CHLOROFORM-d) δ: 1.44-1.58 (m, 9H) 2.96 (b s, 2H) 3.58 (s, 2H) 3.99 (s, 3H) 4.12 (bs, 2H) 6.79 (t, J=8.44 Hz, 2H) 7.21 (d, J=1.47 Hz, 1H) 7.28 (s, 1H) 7.74 (d, J=11.25 Hz, 1H) 8.33 (d, J=2.20 Hz, 1H); LC-MS (m/z): 396 (M+H). HPLC: >95%.

### Step 2: tert-butyl 1-[(2-chloro-4-pyridyl)methylcarbamoyl]-5-(6-methoxy-3-pyridyl)spiro[indoline-3,4′-piperidine]-1′-carboxylate

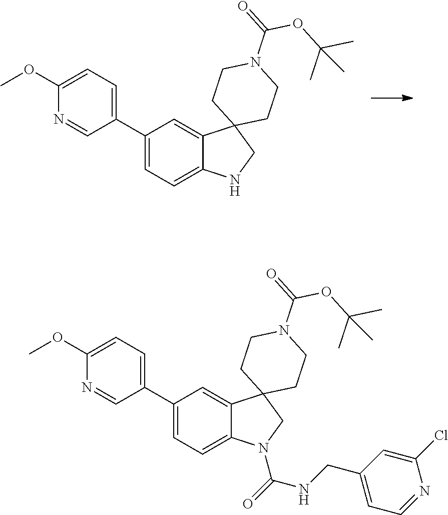

Round bottom flask equipped with stir bar is charged with carbonyl diimidazole (105 mg, 0.64 mmol) followed by DCE (8 mL) and TEA (0.27 mL, 1.95 mmol). (2-chloropyridin-4-yl)methanamine hydrochloride (87 mg, 0.62 mmol) is added in one portion and the mixture warmed to 50° C. and stirred for 1 hour then cooled to room temperature, added tert-butyl 5-(6-methoxy-3-pyridyl)spiro[indoline-3,4′-piperidine]-1′-carboxylate and heated at 50° C. overnite, washed with water, dried, filtered, concentrated to give 314 mg of urea, M+H=564. Urea in DCM (5 mL) is treated with 3 ml TFA, stirred at ambient temp for 1 h, concentrated with toluene, dissolved in DCM, washed with aq sodium bicarbonate solution, dried, filtered, and conc to give title compound (255 mg, 78%): 1H NMR (400 MHz, DMSO-d6) δ: 1.44 (s, 9H) 1.66 (d, J=12.96 Hz, 2H) 1.87 (d, J=4.16 Hz, 2H) 3.88 (s, 3H) 3.98 (s, 2H) 4.04 (d, J=11.00 Hz, 2H) 4.39 (d, J=5.38 Hz, 2H) 6.86 (d, J=8.56 Hz, 1H) 7.26-7.52 (m, 4H) 7.55 (d, J=1.71 Hz, 1H) 7.88 (d, J=8.56 Hz, 1H) 8.00 (dd, J=8.68, 2.57 Hz, 1H) 8.35 (d, J=5.13 Hz, 1H) 8.45 (d, J=2.20 Hz, 1H) LC-MS (m/z): 465 (M+H). HPLC: >90% used as is.

### Step 3, Preparation of Example 119: 1′-[(E)-3-(4-chlorophenyl)allyl]-N-[(2-chloro-4-pyridyl)methyl]-5-(6-methoxy-3-pyridyl)spiro[indoline-3,4′-piperidine]-1-carboxamide

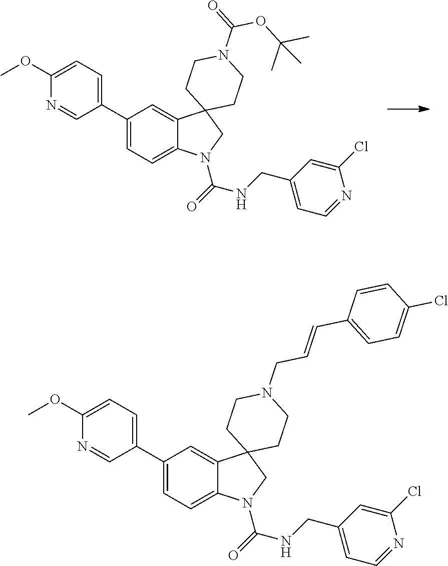 [SMILES: COc1ccc(-c2ccc3c(c2)C2(CCN(C(=O)OC(C)(C)C)CC2)CN3C(=O)NCc2ccnc(Cl)c2)cn1 ; COc1ccc(-c2ccc3c(c2)C2(CCN(C/C=C/c4ccc(Cl)cc4)CC2)CN3C(=O)NCc2ccnc(Cl)c2)cn1]

A 35 mL vial equipped with stirbar is charged with tert-butyl 1-[(2-chloro-4-pyridyl)methylcarbamoyl]-5-(6-methoxy-3-pyridyl)spiro[indoline-3,4′-piperidine]-1′-carboxylate (200 mg, 0.35 mmol), (E)-3-(4-chlorophenyl)prop-2-enal (115 mg, 0.70 mmol), and Si—CBH (800 mg of 0.88 mmol/g, 2 eq). Added 10:1 THF/AcOH (14 mL) and the resulting yellow mixture was heated in a microwave at about 120° C. for 15 minutes then chromatographed to give the title compound (213 mg, 75%): 1H NMR (400 MHz, DMSO-d6) δ: ppm 2.01 (d, J=13.69 Hz, 2H) 2.14-2.37 (m, 2H) 3.65 (br. s., 1H) 3.89 (s, 4H) 3.97 (br. s., 1H) 4.00-4.14 (m, 3H) 4.40 (d, J=5.14 Hz, 2H) 6.40-6.54 (m, 1H) 6.89 (d, J=8.80 Hz, 2H) 7.34 (br. s., 1H) 7.36-7.54 (m, 8H) 7.58 (d, J=8.31 Hz, 2H) 7.84-8.00 (m, 2H) 8.29-8.46 (m, 2H); LC-MS (m/z): 615 (M+H), r.t. 2.88 min. HPLC: >95%.

### Example 120

### N-[(2-chloro-4-pyridyl)methyl]-5-(6-cyano-3-pyridyl)-1′-[(E)-3-[4-(trifluoromethyl)phenyl]allyl]spiro[indoline-3,4′-piperidine]-1-carboxamide

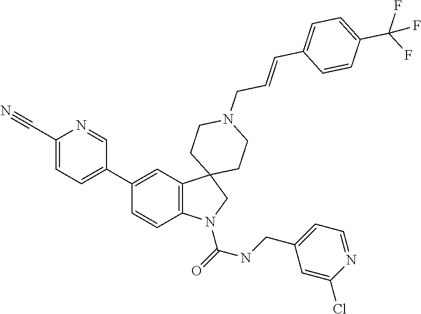 [SMILES: N#Cc1ccc(-c2ccc3c(c2)C2(CCN(C/C=C/c4ccc(C(F)(F)F)cc4)CC2)CN3C(=O)NCc2ccnc(Cl)c2)cn1]

Using the same procedures as Step 1 through 3 of Example 119 and replacing (E)-3-(4-chlorophenyl)acrylaldehyde with (E)-3-(4-(trifluoromethyl)phenyl)-acrylaldehyde the title compound is obtained. 1H NMR (400 MHz, DMSO-d6) δ: 1.69 (d, 2H), 2.05-2.15 (m, 4H), 2.96 (d, 2H), 3.21 (d, 2H), 3.95 (s, 2H), 4.39 (d, 2H), 6.48-6.56 (m, 1H), 6.69 (d, 1H), 7.39 (m, 1H), 7.47 (s, 1H), 7.58 (t, 1H), 7.64-7.68 (m, 5H), 7.79 (m, 1H), 7.95 (d, 1H), 8.06 (d, 1H), 8.36 (m, 2H), 9.12 (m, 1H), LC-MS (m/z): 643 (M+H), r.t. 2.96 min.

### Example 121

### (E)-7-chloro-1′-(3-(4-chlorophenyl)allyl)-N-((2-chloropyridin-4-yl)methyl)-4-(trifluoromethoxy)spiro[indoline-3,4′-piperidine]-1-carboxamide

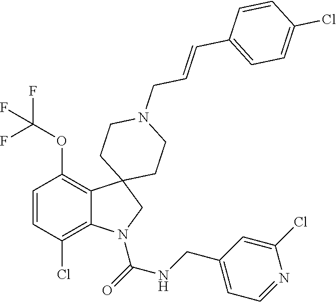 [SMILES: O=C(NCc1ccnc(Cl)c1)N1CC2(CCN(C/C=C/c3ccc(Cl)cc3)CC2)c2c(OC(F)(F)F)ccc(Cl)c21]

Using the same procedures as Step 1 through 6 of Example 55 and replacing 4-(trifluoromethoxy)aniline with commercially 2-chloro-5-(trifluoromethoxy)aniline and (E)-3-(3,4-Dichloro-phenyl)-propenal with commercially available (E)-3-(4-chlorophenyl)acrylaldehyde the title compound is obtained: 1H NMR (400 MHz, DMSO-d6) δ: 1.53 (d, J=12.52 Hz, 2H), 1.99 (t, J=11.72 Hz, 2H), 2.07-2.16 (m, 2H), 2.91 (d, J=11.24 Hz, 2H), 3.14 (d, J=6.44 Hz, 2H), 3.98 (s, 2H), 4.34 (d, J=5.8 Hz, 2H), 6.34-6.41 (m, 1H), 6.54 (d, J=15.96 Hz, 1H), 7.01-7.03 (m, 1H), 7.31 (d, J=5.04 Hz, 1H), 7.35-7.39 (m, 4H), 7.49 (d, J=8.56 Hz, 2H), 8.05 (t, J=5.86 Hz, 1H), 8.34 (d, J=5.12 Hz, 1H). LC-MS (m/z): 624.6 (M+H) r.t. 4.66 min. HPLC: 99.47%.

### Example 122

### (E)-1′-(3-(4-chlorophenyl)allyl)-N-((2-chloropyridin-4-yl)methyl)-4-fluoro-6-(trifluoromethyl)spiro[indoline-3,4′-piperidine]-1-carboxamide

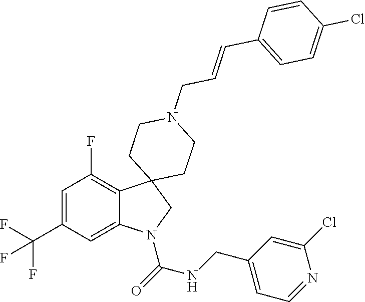 [SMILES: O=C(NCc1ccnc(Cl)c1)N1CC2(CCN(C/C=C/c3ccc(Cl)cc3)CC2)c2c(F)cc(C(F)(F)F)cc21]

Using the same procedures as Step 1 through 6 of Example 55 and replacing 4-(trifluoromethoxy)aniline with commercially available 3-fluoro-5-(trifluoromethyl)aniline and (E)-3-(3,4-Dichloro-phenyl)-propenal with commercially available (E)-3-(4-chlorophenyl)acrylaldehyde the title compound is obtained: 1H NMR (400 MHz, DMSO-d6) δ: 1.76 (d, J=11.9 Hz, 2H), 2.01-2.17 (m, 4H), 2.91 (d, J=11.3 Hz, 2H), 3.13 (d, J=6.28 Hz, 2H), 3.98 (s, 2H), 4.36 (d, J=5.6 Hz, 2H), 6.37-6.41 (m, 1H), 6.55 (d, J=15.9 Hz, 1H), 7.12 (d, J=9.5 Hz, 1H), 7.36-7.38 (m, 3H), 7.48 (t, J=8.6 Hz, 3H), 7.69 (t, J=5.4 Hz, 1H), 8.02 (d, J=0.8 Hz, 1H), 8.34 (d, J=5.0 Hz, 1H). LC-MS (m/z): 592.9 (M+H), r.t. 6.34 min. HPLC: 99.31%.

### Example 123

### (E)-N-((2-chloropyridin-4-yl)methyl)-1′-(3-(3,4-dichlorophenyl)allyl)-5-methylspiro[indoline-3,4′-piperidine]-1-carboxamide

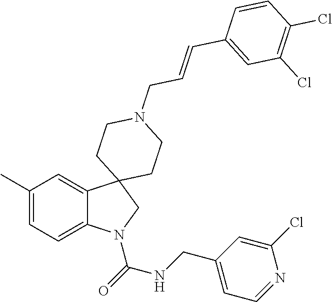 [SMILES: Cc1ccc2c(c1)C1(CCN(C/C=C/c3ccc(Cl)c(Cl)c3)CC1)CN2C(=O)NCc1ccnc(Cl)c1]

Using the same procedures as Step 2 through 6 of Example 56 and replacing (3-Trifluoromethyl-phenyl)-hydrazine with commercially p-tolylhydrazine the title compound is obtained: LC-MS (m/z): 554.8 (M+H), r.t. 5.24 min.

### Example 124

### (E)-N-((2-chloropyridin-4-yl)methyl)-6-(trifluoromethyl)-1′-(3-(5-(trifluoromethyl)pyridin-2-yl)allyl)spiro[indoline-3,4′-piperidine]-1-carboxamide

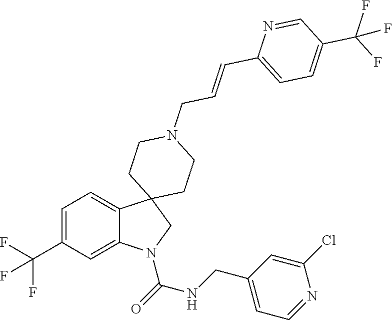 [SMILES: O=C(NCc1ccnc(Cl)c1)N1CC2(CCN(C/C=C/c3ccc(C(F)(F)F)cn3)CC2)c2ccc(C(F)(F)F)cc21]

### Step 1: (E)-3-(5-(trifluoromethyl)pyridin-2-yl)acrylaldehyde

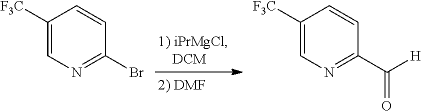 [SMILES: FC(F)(F)c1ccc(Br)nc1 ; O=Cc1ccc(C(F)(F)F)cn1]

iPrMgCl (2M in Et₂O, 750 ml, 1.5 mol) was added to 2-bromo-5-trifluoromethylpyridine (295 g, 1.3 mol) in DCM (4 L) at −2° C. over 3 minutes.

After stirring at 0-6° C. for 40 minutes the mixture was cooled to −20° C. and DMF (200 ml, 2.6 mol) added in one portion. The mixture was cooled to 0° C. over 20 minutes then quenched by addition of 1.5 L saturated NaHCO₃ in one portion. The mixture was stirred at 12° C. for 15 minutes then filtered through a celite pad. The layers were separated. The filtered solids were washed with 1 L DCM and this was then used to re-extract the aqueous layer. The combined organics were dried over MgSO₄, filtered through a pad of 1 kg silica, washed with 5 L DCM and evaporated (bath temp 35° C.). The brown oil was dissolved in 2 L hexane and washed with 2×1 L 12% brine to remove DMF. The organics were filtered through a pad of MgSO₄ and concentrated to low volume. This oil was distilled at 35° C. and 20° C. in Hg to remove hexane then at 56° C. and 26° C. in Hg to afford the title compound as a pale yellow moist crystal (154 g, ˜90% purity, 0.79 mol, 61%): 1H NMR (400 MHz, CDCl3) 7.60 (1H, s), 7.83 (1H, d), 8.85 (1H, d), 9.86 (1H, d).

### Step 2: (E)-3-(5-(trifluoromethyl)pyridin-2-yl)acrylaldehyde

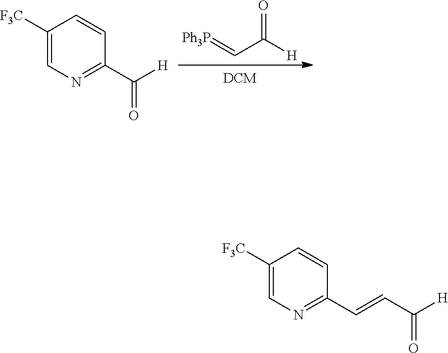

Step 2: A solution of Step 1 (150 g, 0.77 mol) in DCM (1.5 L) was bubbled with nitrogen for 5 minutes and cooled to 9° C. (Formylmethylene)-triphenylphosphorane (257 g, 0.85 mol) was added in one portion, and the reaction stirred at 20° C. for 60 minutes. The mixture was filtered through a pad of magnesol (100 g) and washed with DCM (400 ml). The filtrate was evaporated at 40° C. The residue was triturated with MTBE (150 ml) then diluted with hexane (300 ml) and filtered, washing with MTBE/hexane. The filtrate was diluted with DCM to solubilize a small amount of oil that had separated and then chromatographed (3 kg silica, 20-35% MTBE in hexane) to afford the desired product as a dark red solid (60.3 g, 0.30 mol, 39%): 1H (400 MHz, CDCl3) 7.19 (1H, dd), 7.56 (1H, d), 7.66 (1H, dd), 8.02 (1H, dd), 8.95 (1H, d), 9.85 (1H, d).

### Step 3: Preparation of Example 124

(E)-N-((2-chloropyridin-4-yl)methyl)-6-(trifluoromethyl)-1′-(3-(5-(trifluoromethyl)pyridin-2-yl)allyl)spiro[indoline-3,4′-piperidine]-1-carboxamide. Using the same procedures as Step 2 through 6 of Example 56 and replacing (3-Trifluoromethyl-phenyl)-hydrazine with commercially available (3-(trifluoromethyl)phenyl)hydrazine and (E)-3-(3,4-Dichloro-phenyl)-propenal with (E)-3-(5-(trifluoromethyl)pyridin-2-yl)acrylaldehyde (the product from step 2) the title compound is obtained: 1H NMR (400 MHz, DMSO-d6) δ: 1.68 (d, J=12.4 Hz, 2H), 1.89-1.96 (m, 2H), 2.13 (t, J=13.4 Hz, 2H), 2.93 (d, J=11.6 Hz, 2H), 3.25 (d, J=6.32 Hz, 2H), 3.94 (s, 2H), 4.37 (d, J=5.4 Hz, 2H), 6.77-6.82 (m, 1H), 7.93-7.97 (m, 1H), 7.23 (d, J=8.2 Hz, 1H), 7.38 (d, J=5.0 Hz, 1H), 7.43-7.48 (m, 2H), 7.54-7.63 (m, 1H), 7.69-7.71 (m, 1H), 8.13-8.17 (m, 2H), 8.34 (d, J=5.0 Hz, 1H), 8.89 (s, 1H). LC-MS (m/z): 609.7 (M+H) r.t. 3.89 min.

### Example 125

### (E)-N-((2-chloropyridin-4-yl)methyl)-1′-(3-(4-methoxyphenyl)allyl)-6-(trifluoromethyl)spiro[indoline-3,4′-piperidine]-1-carboxamide

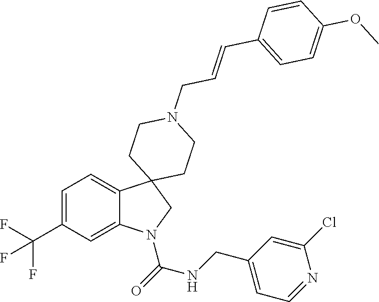 [SMILES: COc1ccc(/C=C/CN2CCC3(CC2)CN(C(=O)NCc2ccnc(Cl)c2)c2cc(C(F)(F)F)ccc23)cc1]

Using the same procedures as Step 2 through 6 of Example 56 and replacing (3-Trifluoromethyl-phenyl)-hydrazine with commercially available (3-(trifluoromethyl)phenyl)hydrazine and (E)-3-(3,4-Dichloro-phenyl)-propenal with commercially (E)-3-(4-methoxyphenyl)acrylaldehyde the title compound is obtained: 1H NMR (400 MHz, DMSO-d6) δ: 1.67 (d, J=12.24 Hz, 2H), 1.89 (t, J=9.4 Hz, 2H), 2.07 (t, J=11.7 Hz, 2H), 2.92 (d, J=11.04 Hz, 2H), 3.12 (d, J=6.6 Hz, 2H), 3.74 (s, 3H), 3.93 (s, 2H), 4.36 (d, J=5.44 Hz, 2H), 6.14-6.21 (m, 1H), 6.49 (d, J=16.0 Hz, 1H), 6.88 (d, J=8.6 Hz, 2H), 7.23 (d, J=7.92 Hz, 1H), 7.37-7.43 (m, 4H), 7.46 (s, 1H), 7.63 (t, J=5.2 Hz, 1H), 8.12 (s, 1H), 8.34 (d, J=5.04 Hz, 1H). LC-MS (m/z): 571.2 (M+H). HPLC purity: 99.72%.

### Example 126

### (E)-1′-(3-(4-chlorophenyl)allyl)-N-((2-chloropyridin-4-yl)methyl)-5-fluoro-6-(trifluoromethyl)spiro[indoline-3,4′-piperidine]-1-carboxamide

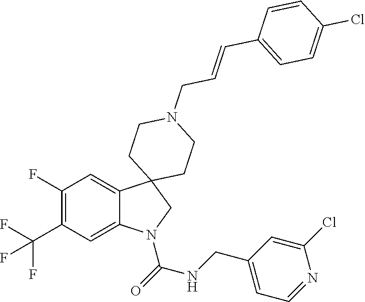 [SMILES: O=C(NCc1ccnc(Cl)c1)N1CC2(CCN(C/C=C/c3ccc(Cl)cc3)CC2)c2cc(F)c(C(F)(F)F)cc21]

Using the same procedures as Step 1 through 6 of Example 55 and replacing 4-(trifluoromethoxy)aniline with commercially available 4-fluoro-3-(trifluoromethyl)aniline and (E)-3-(3,4-Dichloro-phenyl)-propenal with commercially available (E)-3-(4-chlorophenyl)acrylaldehyde the title compound is obtained: 1H NMR (400 MHz, DMSO-d6) δ: 1.68 (d, J=12.28 Hz, 2H), 1.88-1.95 (m, 2H), 2.08 (t, J=5.32 Hz, 2H), 2.92 (d, J=11.52 Hz, 2H), 3.14 (d, J=6.32 Hz, 2H), 3.94 (s, 2H), 4.36 (d, J=5.6 Hz, 2H), 6.33-6.40 (m, 1H), 6.56 (d, J=15.96 Hz, 1H), 7.36-7.39 (m, 3H), 7.47 (t, J=8.76 Hz, 4H), 7.62 (t, J=5.7 Hz, 1H), 8.11 (d, J=6.48 Hz, 1H), 8.34 (d, J=5.08 Hz, 1H). LC-MS (m/z): 592.8 (M+H) r.t. 4.61 min. HPLC purity: 99.27%.

### Example 127

### (E)-1′-(3-(4-chlorophenyl)allyl)-N-((2-fluoropyridin-4-yl)methyl)-6-(trifluoromethoxy)spiro[indoline-3,4′-piperidine]-1-carboxamide

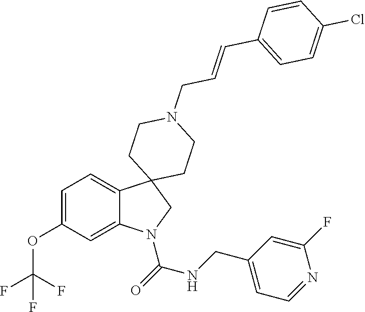 [SMILES: O=C(NCc1ccnc(F)c1)N1CC2(CCN(C/C=C/c3ccc(Cl)cc3)CC2)c2ccc(OC(F)(F)F)cc21]

Using the same procedures as Step 2 through 6 of Example 56 and replacing (3-Trifluoromethyl-phenyl)-hydrazine with commercially available (3-(trifluoromethoxy)phenyl)hydrazine and (2-Chloro-pyridin-4-yl)-methylamine hydrochloride with commercially available (2-fluoropyridin-4-yl)methanamine and (E)-3-(3,4-Dichloro-phenyl)-propenal with commercially available (E)-3-(4-chlorophenyl)acrylaldehyde the title compound is obtained: 1H NMR (400 MHz, DMSO-d6) δ: 1.66 (d, J=12.4 Hz, 2H), 1.83-1.90 (m, 2H), 2.06 (t, J=11.6 Hz, 2H), 2.91 (d, J=11.36 Hz, 2H), 3.14 (d, J=6.32 Hz, 2H), 3.92 (s, 2H), 4.39 (d, J=5.6 Hz, 2H), 6.34-6.41 (m, 1H), 6.56 (d, J=15.96 Hz, 1H), 6.82-6.85 (m, 1H), 7.12 (s, 1H), 7.28-7.32 (m, 2H), 7.37 (d, J=8.52 Hz, 2H), 7.48 (d, J=8.56 Hz, 2H), 7.61 (t, J=5.76 Hz, 1H), 7.78 (d, J=1.08 Hz, 1H), 8.17 (d, J=5.16 Hz, 1H), LC-MS (m/z): 575.0 (M+H) r.t. 16.15 min. HPLC: 98.72%.

### Example 128

### (E)-5-chloro-N-((2-chloropyridin-4-yl)methyl)-6-fluoro-1′-(3-(4-(trifluoromethyl)phenyl)allyl)spiro[indoline-3,4′-piperidine]-1-carboxamide

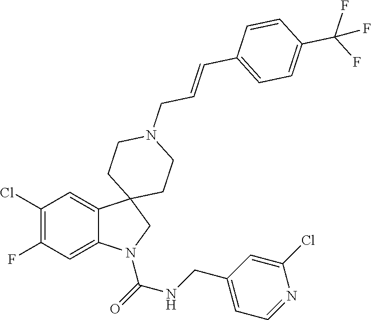 [SMILES: O=C(NCc1ccnc(Cl)c1)N1CC2(CCN(C/C=C/c3ccc(C(F)(F)F)cc3)CC2)c2cc(Cl)c(F)cc21]

Using the same procedures as Step 2 through 6 of Example 56 and replacing (3-Trifluoromethyl-phenyl)-hydrazine with commercially available (4-chloro-3-fluorophenyl)hydrazine and (E)-3-(3,4-Dichloro-phenyl)-propenal with commercially available (E)-3-(4-(trifluoromethyl)phenyl)acrylaldehyde the title compound is obtained: 1H NMR (400 MHz, DMSO-d6) δ: 1.64 (d, J=12.4 Hz, 2H), 1.86-1.91 (m, 2H), 2.07 (t, J=11.1 Hz, 2H), 2.91 (d, J=11.4 Hz, 2H), 3.17 (d, J=6.2 Hz, 2H), 3.91 (s, 2H), 4.35 (d, J=5.6 Hz, 2H), 6.48-6.55 (m, 1H), 6.67 (d, J=16.0 Hz, 1H), 7.36 (d, J=5.0 Hz, 1H), 7.42 (d, J=7.5 Hz, 1H), 7.45 (s, 1H), 7.63 (t, J=5.7 Hz, 1H), 7.67 (s, 4H), 7.72 (d, J=11.36 Hz, 1H), 8.34 (d, J=5.1 Hz, 1H). LC-MS (m/z): 592.9 (M+H) r.t. 12.54 min.

### Example 129

### (E)-N-((2-chloropyridin-4-yl)methyl)-1′-(3-(3,4-dichlorophenyl)allyl)-5-fluoro-6-(trifluoromethyl)spiro[indoline-3,4′-piperidine]-1-carboxamide

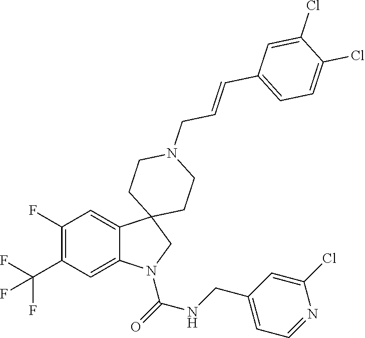 [SMILES: O=C(NCc1ccnc(Cl)c1)N1CC2(CCN(C/C=C/c3ccc(Cl)c(Cl)c3)CC2)c2cc(F)c(C(F)(F)F)cc21]

Using the same procedures as Step 1 through 6 of Example 56 and replacing 4-(trifluoromethoxy)aniline with commercially available 4-fluoro-3-(trifluoromethyl)aniline the title compound is obtained: LC-MS (m/z): 627 (M+H) r.t. 6.40 min.

### Example 130

### (E)-N-((2-chloropyridin-4-yl)methyl)-1′-(3-(4-(trifluoromethoxy)phenyl)allyl)-6-(trifluoromethyl)spiro[indoline-3,4′-piperidine]-1-carboxamide

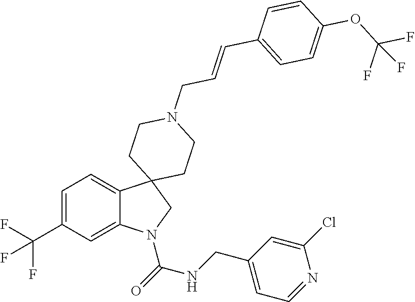 [SMILES: O=C(NCc1ccnc(Cl)c1)N1CC2(CCN(C/C=C/c3ccc(OC(F)(F)F)cc3)CC2)c2ccc(C(F)(F)F)cc21]

Using the same procedures as Step 2 through 6 of Example 56 and replacing (3-Trifluoromethyl-phenyl)-hydrazine with commercially available (3-(trifluoromethyl)phenyl)hydrazine and (E)-3-(3,4-Dichloro-phenyl)-propenal with commercially available (E)-3-(4-(trifluoromethoxy)phenyl)acrylaldehyde the title compound is obtained: 1H NMR (400 MHz, DMSO-d6) δ: 1.68 (d, J=12.36 Hz, 2H), 1.86-1.92 (m, 2H), 2.08 (t, J=11.44 Hz, 2H), 2.92 (d, J=11.28 Hz, 2H), 3.16 (d, J=6.32 Hz, 2H), 3.93 (s, 2H), 4.36 (d, J=5.64 Hz, 2H), 6.37-6.43 (m, 1H), 6.60 (d, J=15.92 Hz, 1H), 7.23 (d, J=7.76 Hz, 1H), 7.32 (d, J=8.2 Hz, 2H), 7.38 (d, J=5.12 Hz, 1H), 7.42 (d, J=7.76 Hz, 1H), 7.46 (s, 1H), 7.59 (d, J=8.72 Hz, 2H), 7.63 (t, J=5.6 Hz, 1H), 8.12 (d, J=1.16 Hz, 1H), 8.34 (d, J=5.12 Hz, 1H). LC-MS (m/z): 625 (M+H) r.t. 10.07 min. HPLC purity: 99.75%.

### Example 131

### (E)-4-chloro-N-((2-chloropyridin-4-yl)methyl)-1′-(3-(3,4-dichlorophenyl)allyl) spiro[indoline-3,4′-piperidine]-1-carboxamide

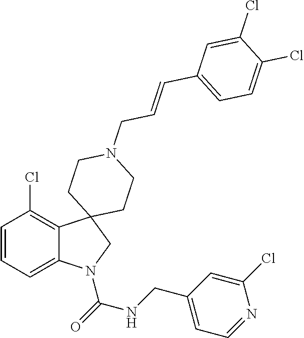 [SMILES: O=C(NCc1ccnc(Cl)c1)N1CC2(CCN(C/C=C/c3ccc(Cl)c(Cl)c3)CC2)c2c(Cl)cccc21]

Using the same procedures as Step 2 through 6 of Example 56 and replacing (3-Trifluoromethyl-phenyl)-hydrazine with commercially available (3-chlorophenyl)hydrazine the title compound is obtained: 1H NMR (400 MHz, DMSO-d6) δ: 1.53 (d, J=12.48 Hz, 2H), 2.04 (t, J=11.7 Hz, 2H), 2.46-2.53 (m, 2H), 2.89 (d, J=10.92 Hz, 2H), 3.11 (d, J=4.72 Hz, 2H), 3.88 (s, 2H), 4.33 (d, J=5.68 Hz, 2H), 6.45-6.54 (m, 2H), 6.84 (d, J=7.96 Hz, 1H), 7.09 (t, J=8.08 Hz, 1H), 7.35 (d, J=5.12 Hz, 1H), 7.42-7.46 (m, 2H), 7.50-7.55 (m, 2H), 7.75 (d, J=1.92 Hz, 1H), 7.87 (d, J=8.08 Hz, 1H), 8.32 (d, J=5.08 Hz, 1H). LC-MS (m/z): 574.9 (M+H) 6.22 min. HPLC: 99.88%.

### Example 132

### (E)-N-((2-chloropyridin-4-yl)methyl)-5-cyano-1′-(3-(3,4-dichlorophenyl)allyl)-6-(trifluoromethyl)spiro[indoline-3,4′-piperidine]-1-carboxamide

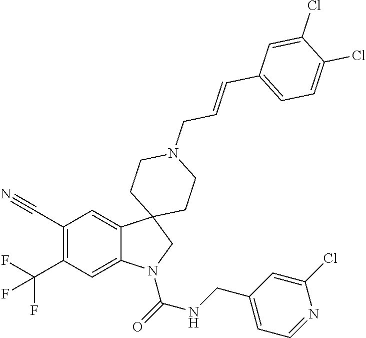 [SMILES: N#Cc1cc2c(cc1C(F)(F)F)N(C(=O)NCc1ccnc(Cl)c1)CC21CCN(C/C=C/c2ccc(Cl)c(Cl)c2)CC1]

Using the same procedures as Step 1 through 6 of Example 55 and replacing 4-(trifluoromethoxy)aniline with commercially available 4-amino-2-(trifluoromethyl)benzonitrile the title compound is obtained: LC-MS (m/z): 633.9 (M+H).

### Example 133

### (E)-N-((2-chloropyridin-4-yl)methyl)-1′-(3-(3,4-dichlorophenyl)allyl)-5-(trifluoromethyl)spiro[indoline-3,4′-piperidine]-1-carboxamide

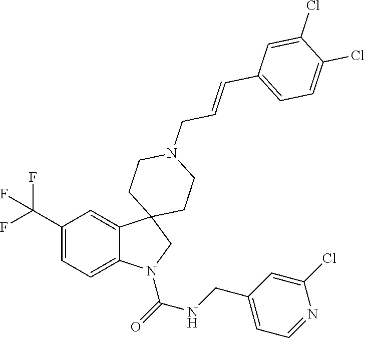 [SMILES: O=C(NCc1ccnc(Cl)c1)N1CC2(CCN(C/C=C/c3ccc(Cl)c(Cl)c3)CC2)c2cc(C(F)(F)F)ccc21]

Using the same procedures as Step 1 through 6 of Example 55 and replacing 4-(trifluoromethoxy)aniline with commercially available 4-(trifluoromethyl)aniline the title compound is obtained: 1H NMR (400 MHz, DMSO-d6) δ: 1.64 (d, J=12.32 Hz, 2H), 1.90 (t, J=10.86 Hz, 2H), 2.07 (t, J=11.44 Hz, 2H), 2.89 (d, J=11.44 Hz, 2H), 3.12 (d, J=5.96 Hz, 2H), 3.91 (s, 2H), 4.30-4.35 (m, 2H), 6.39-6.48 (m, 1H), 6.54 (d, J=16.04 Hz, 1H), 7.35 (d, J=4.88 Hz, 1H), 7.43 (s, 2H), 7.45-7.48 (m, 2H), 7.52-7.56 (m, 1H), 7.61 (t, J=5.82 Hz, 1H), 7.72 (d, J=1.68 Hz, 1H), 7.94 (d, J=8.4 Hz, 1H), 8.32 (d, J=5.08 Hz, 1H). LC-MS (m/z): 609.0 (M+H) 4.86 min. HPLC: 99.85%.

### Example 134

### 5-chloro-N-((2-chloropyridin-4-yl)methyl)-1′-((2-methoxyquinolin-3-yl)methyl)spiro[indoline-3,4′-piperidine]-1-carboxamide

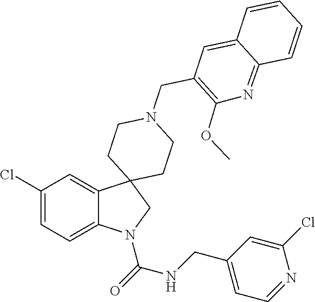 [SMILES: COc1nc2ccccc2cc1CN1CCC2(CC1)CN(C(=O)NCc1ccnc(Cl)c1)c1ccc(Cl)cc12]

Using the same procedures as Step 2 through 6 of Example 56 and replacing (3-Trifluoromethyl-phenyl)-hydrazine with commercially available (4-chlorophenyl) and (E)-3-(3,4-Dichloro-phenyl)-propenal with commercially available 2-methoxyquinoline-3-carbaldehyde the title compound is obtained: 1H NMR (400 MHz, DMSO-d6) δ: 1.65 (d, J=12.28 Hz, 2H), 1.93-1.98 (m, 2H), 2.19 (t, J=12.08 Hz, 2H), 2.93 (d, J=11.8 Hz, 2H), 3.64 (s, 2H), 3.89 (s, 2H), 4.02 (s, 3H), 4.35 (d, J=5.56 Hz, 2H), 7.12-7.15 (dd, J=2.24 Hz J=8.64 Hz, 1H), 7.31 (d, J=2.2 Hz, 1H), 7.36 (d, J=5.16 Hz, 1H), 7.41-7.45 (m, 2H), 7.51 (t, J=5.6 Hz, 1H), 7.61-7.65 (m, 1H), 7.77 (d, J=8.68 Hz, 1H), 7.80 (d, J=8.6 Hz, 1H), 7.92 (d, J=8.0 Hz, 1H), 8.23 (s, 1H), 8.31-8.34 (m, 1H), LC-MS (m/z): 561.80 (M+H) r.t. 5.97 min. HPLC: 99.66%.

### Example 135

### (E)-5-chloro-N-((2-chloropyridin-4-yl)methyl)-1′-(3-(3,4-dichlorophenyl)allyl)-6-(trifluoromethyl)spiro[indoline-3,4′-piperidine]-1-carboxamide

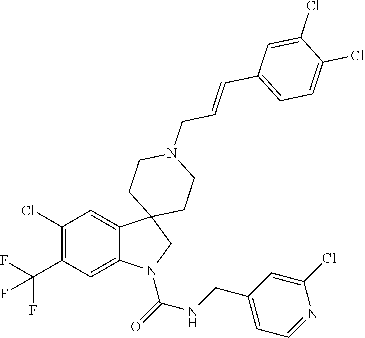 [SMILES: O=C(NCc1ccnc(Cl)c1)N1CC2(CCN(C/C=C/c3ccc(Cl)c(Cl)c3)CC2)c2cc(Cl)c(C(F)(F)F)cc21]

Using the same procedures as Step 1 through 6 of Example 55 and replacing 4-(trifluoromethoxy)aniline with commercially available 4-chloro-3-(trifluoromethyl)aniline the title compound is obtained: 1H NMR (400 MHz, DMSO-d6) δ: 1.69 (d, J=12.44 Hz, 2H), 1.93 (t, J=13.08 Hz, 2H), 2.07 (t, J=12.86 Hz, 2H), 2.93 (d, J=11.2 Hz, 2H), 3.15 (d, J=5.96 Hz, 2H), 3.95 (s, 2H), 4.37 (d, J=5.52 Hz, 2H), 6.44-6.48 (m, 1H), 6.52 (d, J=16.04 Hz, 1H), 7.38 (d, J=5.16 Hz, 1H), 7.46-7.49 (m, 2H), 7.57-7.59 (m, 2H), 7.68 (t, J=4.78 Hz, 1H), 7.75 (d, J=1.6 Hz, 1H), 8.26 (s, 1H), 8.35 (d, J=5.04 Hz, 1H). LC-MS (m/z): 643.1 (M+H) r.t. 6.51 min.

### Example 136

### (E)-4,5-dichloro-N-((2-chloropyridin-4-yl)methyl)-1′-(3-(3,4-dichlorophenyl)allyl)spiro[indoline-3,4′-piperidine]-1-carboxamide

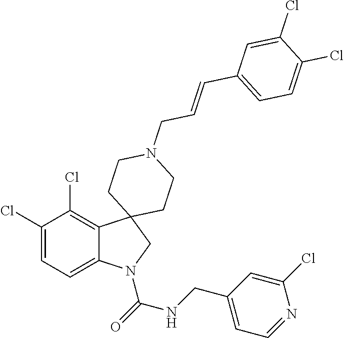 [SMILES: O=C(NCc1ccnc(Cl)c1)N1CC2(CCN(C/C=C/c3ccc(Cl)c(Cl)c3)CC2)c2c1ccc(Cl)c2Cl]

Using the same procedures as Step 2 through 6 of Example 56 and replacing (3-Trifluoromethyl-phenyl)-hydrazine with commercially available (3,4-dichlorophenyl)hydrazine the title compound is obtained: 1H NMR (400 MHz, DMSO-d6) δ: 1.58 (d, J=12.76 Hz, 2H), 2.06 (t, J=12.6 Hz, 2H), 2.54-2.57 (m, 2H), 2.92 (d, J=11.2 Hz, 2H), 3.13 (d, J=4.8 Hz, 2H), 3.94 (s, 2H), 4.36 (d, J=5.6 Hz, 2H), 6.52-6.53 (m, 2H), 7.37-7.40 (m, 2H), 7.46-7.49 (m, 2H), 7.57 (d, J=8.36 Hz, 1H), 7.58 (t, J=9.34 Hz, 1H), 7.78 (d, J=1.92 Hz, 1H), 7.91 (d, J=8.76 Hz, 1H), 8.34 (d, J=5.16 Hz, 1H). LC-MS (m/z): 608.7 (M+H) 3.56 min. HPLC: 95.67%.

### Example 137

### (E)-N-((2-chloropyridin-4-yl)methyl)-1′-(3-(3,4-dichlorophenyl)allyl)-5-(dimethylamino)spiro[indoline-3,4′-piperidine]-1-carboxamide

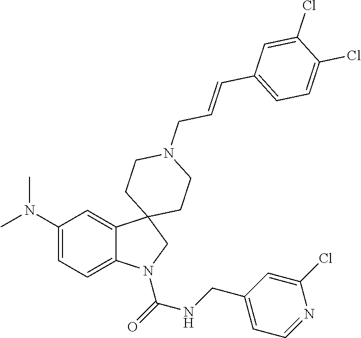 [SMILES: CN(C)c1ccc2c(c1)C1(CCN(C/C=C/c3ccc(Cl)c(Cl)c3)CC1)CN2C(=O)NCc1ccnc(Cl)c1]

Using the same procedures as Step 2 through 6 of Example 56 and replacing (3-Trifluoromethyl-phenyl)-hydrazine with commercially available 4-hydrazinyl-N,N-dimethylaniline the title compound is obtained: 1H NMR (400 MHz, DMSO-d6) δ: 1.58 (d, J=12.6 Hz, 2H), 1.88-1.94 (m, 2H), 2.04 (t, J=11.0 Hz, 2H), 2.80 (s, 6H), 2.90 (d, J=11.2 Hz, 2H), 3.13 (d, J=6.0 Hz, 2H), 3.79 (s, 2H), 3.33 (d, J=5.7 Hz, 2H), 6.48-6.51 (m, 2H), 6.57 (d, J=16.0 Hz, 1H), 6.63 (d, J=2.5 Hz, 1H), 7.25 (t, J=5.8 Hz, 1H), 7.35 (d, J=5.0 Hz, 1H), 7.42 (s, 1H), 7.47 (dd, J₁=8.4 Hz, J₂=1.9 Hz, 1H), 7.57 (d, J=8.4 Hz, 1H), 7.62 (d, J=8.7 Hz, 1H), 7.75 (d, J=1.9 Hz, 1H), 8.33 (d, J=5.0 Hz, 1H). LC-MS (m/z): 586.1 (M+H) r.t. 5.70 min.

### Example 138

### (E)-N-((2-chloropyridin-4-yl)methyl)-6-(trifluoromethyl)-1′-(3-(4-(trifluoromethyl)phenyl)allyl)spiro[indoline-3,4′-piperidine]-1-carboxamide

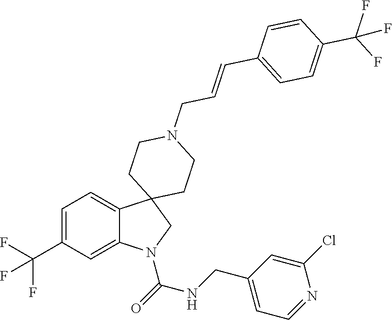 [SMILES: O=C(NCc1ccnc(Cl)c1)N1CC2(CCN(C/C=C/c3ccc(C(F)(F)F)cc3)CC2)c2ccc(C(F)(F)F)cc21]

Using the same procedures as Step 2 through 6 of Example 56 and replacing (3-Trifluoromethyl-phenyl)-hydrazine with commercially (3-(trifluoromethyl)phenyl)-hydrazine and (E)-3-(3,4-Dichloro-phenyl)-propenal with commercially available (E)-3-(4-(trifluoromethyl)phenyl)acrylaldehyde the title compound is obtained: 1H NMR (400 MHz, DMSO-d6) δ: 1.68 (d, J=12.4 Hz, 2H), 1.87-1.93 (m, 2H), 2.10 (t, J=11.26 Hz, 2H), 2.93 (d, J=11.8 Hz, 2H), 3.19 (d, J=6.08 Hz, 2H), 3.94 (s, 2H), 4.37 (d, J=5.56 Hz, 2H), 6.50-6.57 (m, 1H), 6.68 (d, J=15.96 Hz, 1H), 7.23 (d, J=7.76 Hz, 1H), 7.38 (d, J=5.12 Hz, 1H), 7.43 (d, J=7.8 Hz, 1H), 7.46 (s, 1H), 7.63 (t, J=5.68 Hz, 1H), 7.68 (s, 4H), 8.13 (d, J=1.2 Hz, 1H), 8.34 (d, J=5.08 Hz, 1H). LC-MS (m/z): 609.1 (M+H) r.t. 6.06 min. HPLC: 99.31%.

### Example 139

### 5-chloro-N-((2-chloropyridin-4-yl)methyl)-1′-((6-fluoronaphthalen-2-yl)methyl)spiro[indoline-3,4′-piperidine]-1-carboxamide

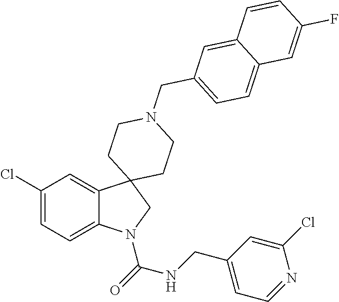 [SMILES: O=C(NCc1ccnc(Cl)c1)N1CC2(CCN(Cc3ccc4cc(F)ccc4c3)CC2)c2cc(Cl)ccc21]

Using the same procedures as Step 2 through 6 of Example 56 and replacing (3-Trifluoromethyl-phenyl)-hydrazine with commercially (4-chlorophenyl)hydrazine and (E)-3-(3,4-Dichloro-phenyl)-propenal with commercially available 6-fluoro-2-naphthaldehyde the title compound is obtained: 1H NMR (400 MHz, DMSO-d6) δ: 1.62 (d, J=12.64 Hz, 2H), 1.86-1.91 (m, 2H), 2.08-2.15 (m, 2H), 2.86 (d, J=11.46 Hz, 2H), 3.67 (s, 2H), 3.88 (s, 2H), 4.34 (d, J=5.68 Hz, 2H), 7.12 (dd, J₁=2.2 Hz, J₂=8.56 Hz, 1H), 7.28 (d, J=2.12 Hz, 1H), 7.36 (d, J=5.04 Hz, 1H), 7.40-7.42 (m, 1H), 7.44 (s, 1H), 7.52 (t, J=5.7 Hz, 1H), 7.57 (d, J=8.4 Hz, 1H), 7.69 (dd, J₁=2.44, J₂=10.35 Hz, 1H), 7.79 (d, J=8.6 Hz, 1H), 7.87-7.90 (m, 2H), 7.97-8.01 (m, 1H), 8.33 (d, J=5.12 Hz, 1H). LC-MS (m/z): 549.0 (M+H) r.t. 10.59 min HPLC: 99.47%.

### Example 140

### (E)-4-chloro-1′-(3-(4-chlorophenyl)allyl)-N-((2-chloropyridin-4-yl)methyl)-5-fluorospiro[indoline-3,4′-piperidine]-1-carboxamide

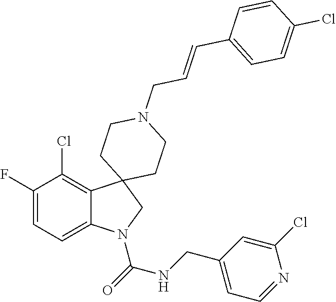 [SMILES: O=C(NCc1ccnc(Cl)c1)N1CC2(CCN(C/C=C/c3ccc(Cl)cc3)CC2)c2c1ccc(F)c2Cl]

Using the same procedures as Step 2 through 6 of Example 56 and replacing (3-Trifluoromethyl-phenyl)-hydrazine with commercially available (3-chloro-4-fluorophenyl)hydrazine and (E)-3-(3,4-Dichloro-phenyl)-propenal with commercially available (E)-3-(4-chlorophenyl)acrylaldehyde the title compound is obtained: 1H NMR (400 MHz, DMSO-d6) δ: 1.60 (d, J=12.92 Hz, 2H), 2.06 (t, J=11.78 Hz, 2H), 2.49-2.55 (m, 2H), 2.92 (d, J=10.72 Hz, 2H), 3.13 (d, J=6.4 Hz, 2H), 3.94 (s, 2H), 4.35 (d, J=5.56 Hz, 2H), 6.36-6.43 (m, 1H), 6.55 (d, J=15.96 Hz, 1H), 7.17 (t, J=9.12 Hz, 1H), 7.36-7.38 (m, 3H), 7.45 (s, 1H), 7.49 (d, J=8.48 Hz, 2H), 7.54 (t, J=5.74 Hz, 1H), 7.86-7.90 (m, 1H), 8.34 (d, J=5.04 Hz, 1H). LC-MS (m/z): 558.9 (M+H) r.t. 10.36 min HPLC: 99.04%.

### Example 141

### (E)-5,6-dichloro-N-((2-chloropyridin-4-yl)methyl)-1′-(3-(4-fluorophenyl)allyl)spiro[indoline-3,4′-piperidine]-1-carboxamide

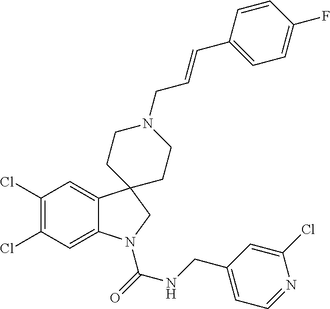 [SMILES: O=C(NCc1ccnc(Cl)c1)N1CC2(CCN(C/C=C/c3ccc(F)cc3)CC2)c2cc(Cl)c(Cl)cc21]

Using the same procedures as Step 2 through 6 of Example 56 and replacing (3-Trifluoromethyl-phenyl)-hydrazine with commercially available (3,4-dichlorophenyl)hydrazine and (E)-3-(3,4-Dichloro-phenyl)-propenal with commercially available (E)-3-(4-fluorophenyl)acrylaldehyde the title compound is obtained: 1H NMR (400 MHz, DMSO-d6) δ: 1.65 (d, J=12.6 Hz, 2H), 1.85-1.91 (m, 2H), 2.05 (t, J=11.04 Hz, 2H), 2.90 (d, J=11.8 Hz, 2H), 3.13 (d, J=6.6 Hz, 2H), 3.90 (s, 2H), 4.35 (d, J=5.64 Hz, 2H), 6.28-6.32 (m, 1H), 6.55 (d, J=15.88 Hz, 1H), 7.15 (t, J=8.8 Hz, 2H), 7.37 (d, 4.1 Hz, 1H), 7.45-7.51 (m, 4H), 7.63 (t, J=5.8 Hz, 1H), 7.98 (s, 1H), 8.34 (d, J=5.12 Hz, 1H), LC-MS (m/z): 558.9 (M+H) r.t 10.28 min HPLC: 99.14%.

### Example 142

### (E)-5,6-dichloro-1′-(3-(4-chlorophenyl)allyl)-N-((2-chloropyridin-4-yl)methyl)spiro[indoline-3,4′-piperidine]-1-carboxamide

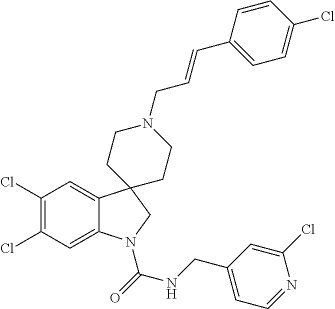 [SMILES: O=C(NCc1ccnc(Cl)c1)N1CC2(CCN(C/C=C/c3ccc(Cl)cc3)CC2)c2cc(Cl)c(Cl)cc21]

Using the same procedures as Step 2 through 6 of Example 56 and replacing (3-Trifluoromethyl-phenyl)-hydrazine with commercially available (3,4-dichlorophenyl)hydrazine and (E)-3-(3,4-Dichloro-phenyl)-propenal with commercially available (E)-3-(4-chlorophenyl)acrylaldehyde the title compound is obtained: 1H NMR (400 MHz, DMSO-d6) δ: 1.65 (d, J=12.76 Hz, 2H), 1.88 (t, J=9.52 Hz, 2H), 2.05 (t, J=11.6 Hz, 2H), 2.90 (d, J=11.8 Hz, 2H), 3.13 (t, J=6.2 Hz, 2H), 3.90 (s, 2H), 4.35 (d, J=5.56 Hz, 2H), 6.32-6.40 (m, 1H), 6.56 (d, J=15.84 Hz, 1H), 7.36-7.39 (m, 3H), 7.45-7.49 (m, 4H), 7.63 (t, J=5.74 Hz, 1H), 7.98 (s, 1H), 8.34 (d, J=5.12 Hz, 1H), LC-MS (m/z): 574.9 (M+H) r.t. 11.12 min HPLC: 99.78%.

### Example 143

### (E)-4,5-dichloro-N-((2-chloropyridin-4-yl)methyl)-1′-(3-(4-fluorophenyl)allyl)spiro[indoline-3,4′-piperidine]-1-carboxamide

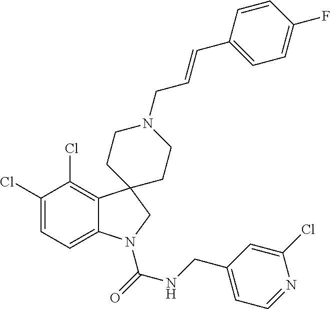 [SMILES: O=C(NCc1ccnc(Cl)c1)N1CC2(CCN(C/C=C/c3ccc(F)cc3)CC2)c2c1ccc(Cl)c2Cl]

Using the same procedures as Step 2 through 6 of Example 56 and replacing (3-Trifluoromethyl-phenyl)-hydrazine with commercially available (3,4-dichlorophenyl)hydrazine and (E)-3-(3,4-Dichloro-phenyl)-propenal with commercially available (E)-3-(4-fluorophenyl)acrylaldehyde the title compound is obtained: 1H NMR (400 MHz, DMSO-d6) δ: 1.57 (d, J=13.32 Hz, 2H), 2.03-2.09 (m, 2H), 2.54-2.60 (m, 2H), 2.92 (d, J=11.48 Hz, 2H), 3.12 (d, J=6.28 Hz, 2H), 3.94 (s, 2H), 4.36 (d, J=5.52, 2H), 6.28-6.36 (m, 1H), 6.54 (d, J=15.84 Hz, 1H), 7.15 (t, J=8.88 Hz, 2H), 7.37-7.40 (m, 2H), 7.45 (s, 1H), 7.49-7.53 (m, 2H), 7.61 (t, J=5.88 Hz, 1H), 7.91 (d, J=8.76 Hz, 1H), 8.34 (d, J=5.08 Hz, 1H). LC-MS (m/z): 558.7 (M+H) r.t. 4.31 min HPLC: 98.39%.

### Example 144

### (E)-4,5-dichloro-1′-(3-(4-chlorophenyl)allyl)-N-((2-chloropyridin-4-yl)methyl)spiro[indoline-3,4′-piperidine]-1-carboxamide

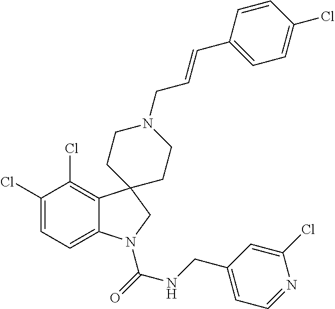 [SMILES: O=C(NCc1ccnc(Cl)c1)N1CC2(CCN(C/C=C/c3ccc(Cl)cc3)CC2)c2c1ccc(Cl)c2Cl]

Using the same procedures as Step 2 through 6 of Example 56 and replacing (3-Trifluoromethyl-phenyl)-hydrazine with commercially available (3,4-dichlorophenyl)hydrazine and (E)-3-(3,4-Dichloro-phenyl)-propenal with commercially available (E)-3-(4-chlorophenyl)acrylaldehyde the title compound is obtained. 1H NMR (400 MHz, DMSO-d6) δ: 1.57 (d, J=13.2 Hz, 2H), 2.06 (t, J=11.04 Hz, 2H), 2.57 (d, J=4.2 Hz, 2H), 2.91 (d, J=11.72 Hz, 2H), 3.13 (d, J=6.28 Hz, 2H), 3.94 (s, 2H), 4.36 (d, J=5.56 Hz, 2H), 6.38-6.43 (m, 1H), 6.55 (d, J=16.04 Hz, 1H), 7.36-7.40 (m, 4H), 7.45 (s, 1H), 7.49 (d, J=8.56 Hz, 2H), 7.61 (t, J=5.72 Hz, 1H), 7.91 (d, J=8.76 Hz, 1H), 8.34 (d, J=5.04 Hz, 1H), LC-MS (m/z): 574.7 (M+H) 4.84 min HPLC: 97.73%.

### Example 145

### (E)-6-chloro-N-((2-chloropyridin-4-yl)methyl)-5-fluoro-1′-(3-(4-(trifluoromethyl)phenyl)allyl)spiro[indoline-3,4′-piperidine]-1-carboxamide

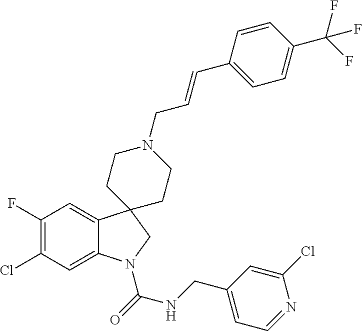 [SMILES: O=C(NCc1ccnc(Cl)c1)N1CC2(CCN(C/C=C/c3ccc(C(F)(F)F)cc3)CC2)c2cc(F)c(Cl)cc21]

Using the same procedures as Step 2 through 6 of Example 56 and replacing (3-Trifluoromethyl-phenyl)-hydrazine with commercially available (3-chloro-4-fluorophenyl)hydrazine and (E)-3-(3,4-Dichloro-phenyl)-propenal with commercially available (E)-3-(4-(trifluoromethyl)phenyl)acrylaldehyde the title compound is obtained: 1H NMR (400 MHz, DMSO-d6) δ: 1.64 (d, J=12.32 Hz, 2H), 1.85-1.91 (m, 2H), 2.04-2.09 (m, 2H), 2.91 (d, J=11.28 Hz, 2H), 3.16 (d, J=6.2 Hz, 2H), 3.90 (s, 2H), 4.35 (d, J=5.56 Hz, 2H), 6.50-6.55 (m, 1H), 6.67 (d, J=15.96 Hz, 1H), 7.36-7.39 (m, 2H), 7.44 (s, 1H), 7.57 (t, J=5.52 Hz, 1H), 7.67 (s, 4H), 7.91 (d, J=6.84 Hz, 1H), 8.33 (d, J=5.08 Hz, 1H). LC-MS (m/z): 593.2 r.t. 8.59 min HPLC: 98.59%.

### Example 146

### (E)-4-chloro-N-((2-chloropyridin-4-yl)methyl)-1′-(3-(3,4-dichlorophenyl)allyl)-5-fluorospiro[indoline-3,4′-piperidine]-1-carboxamide

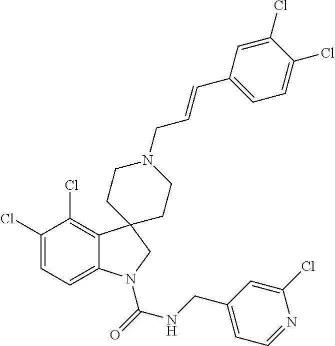 [SMILES: O=C(NCc1ccnc(Cl)c1)N1CC2(CCN(C/C=C/c3ccc(Cl)c(Cl)c3)CC2)c2c1ccc(Cl)c2Cl]

Using the same procedures as Step 2 through 6 of Example 56 and replacing (3-Trifluoromethyl-phenyl)-hydrazine with commercially available (3,4-dichlorophenyl)hydrazine the title compound is obtained: 1H NMR (400 MHz, DMSO-d6) δ: 1.60 (d, J=12.6 Hz, 2H), 2.06 (t, J=11.44 Hz, 2H), 2.53-2.56 (m, 2H), 2.92 (d, J=11.36 Hz, 2H), 3.14 (d, J=4.92 Hz, 2H), 3.94 (s, 2H), 4.36 (d, J=5.60 Hz, 2H), 6.47-6.57 (m, 2H), 7.16 (t, J=9.14 Hz, 1H), 7.36-7.37 (m, 1H), 7.45 (s, 1H), 7.47-7.49 (dd, J₁=8.44 Hz, J₂=2.0 Hz, 1H), 7.53-7.54 (m, 1H), 7.57 (d, J=8.36 Hz, 1H), 7.78 (d, J=1.92 Hz, 1H), 7.86-7.90 (m, 1H), 8.34 (d, J=5.08 Hz, 1H). LC-MS (m/z): 592.9 (M+H) r.t. 6.40 min HPLC: 99.02%.

### Example 147

### (E)-1′-(3-(4-chlorophenyl)allyl)-N-((2-chloropyridin-4-yl)methyl)-5-(trifluoromethoxy)spiro[indoline-3,4′-piperidine]-1-carboxamide

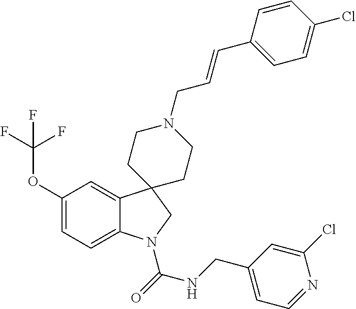 [SMILES: O=C(NCc1ccnc(Cl)c1)N1CC2(CCN(C/C=C/c3ccc(Cl)cc3)CC2)c2cc(OC(F)(F)F)ccc21]

Using the same procedures as Step 1 through 6 of Example 55 and replacing 4-(trifluoromethoxy)aniline with commercially available 4-(trifluoromethoxy)aniline and (E)-3-(3,4-Dichloro-phenyl)-propenal with commercially available (E)-3-(4-chlorophenyl)acrylaldehyde the title compound is obtained: 1H NMR (400 MHz, DMSO-d6) δ: 1.66 (d, J=12.36 Hz, 2H), 1.85-1.90 (m, 2H), 2.07 (t, J=11.72 Hz, 2H), 2.91 (d, J=11.2 Hz, 2H), 3.14 (d, J=6.28 Hz, 2H), 3.91 (s, 2H), 4.36 (d, J=5.6, 2H), 6.33-6.40 (m, 1H), 6.56 (d, J=15.96 Hz, 1H), 7.07-7.10 (m, 1H), 7.23 (d, J=1.6 Hz, 1H), 7.36-7.39 (m, 3H), 7.44-7.49 (m, 3H), 7.54 (t, J=5.76 Hz, 1H), 7.87 (d, J=8.8 Hz, 1H), 8.34 (d, J=5.04 Hz, 1H). LC-MS (m/z): 589.1 (M−H) r.t. 6.04 min HPLC: 99.81%.

### Example 148

### (E)-7-chloro-1′-(3-(4-chlorophenyl)allyl)-N-((2-fluoropyridin-4-yl)methyl)-4-(trifluoromethoxy)spiro[indoline-3,4′-piperidine]-1-carboxamide

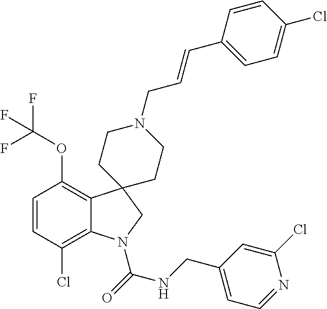 [SMILES: O=C(NCc1ccnc(Cl)c1)N1CC2(CCN(C/C=C/c3ccc(Cl)cc3)CC2)c2c(OC(F)(F)F)ccc(Cl)c21]

Using the same procedures as Step 1 through 6 of Example 55 and replacing 4-(trifluoromethoxy)aniline with commercially available 2-chloro-5-(trifluoromethoxy)aniline and (2-Chloro-pyridin-4-yl)-methylamine hydrochloride with commercially available (2-fluoropyridin-4-yl)methanamine and (E)-3-(3,4-Dichloro-phenyl)-propenal with commercially available (E)-3-(4-chlorophenyl)acrylaldehyde the title compound is obtained: 1H NMR (400 MHz, DMSO-d6) δ: 1.54 (d, J=12.48 Hz, 2H), 1.99 (t, J=11.98 Hz, 2H), 2.09-2.15 (m, 2H), 2.89 (d, J=10.4 Hz, 2H), 3.13 (d, J=6.36 Hz, 2H), 3.97 (s, 2H), 4.38 (d, J=5.76 Hz, 2H), 6.34-6.41 (m, 1H), 6.54 (d, J=15.92 Hz, 1H), 7.02-7.03 (m, 2H), 7.25 (d, J=4.8 Hz, 1H), 7.35-7.39 (m, 3H), 7.49 (d, J=8.44 Hz, 2H), 8.05 (t, J=5.84 Hz, 1H), 8.18 (d, J=5.12 Hz, 1H). LC-MS (m/z): 608.9 (M+H) r.t. 6.00 min. HPLC: 99.90%.

### Example 149

### (E)-1′-(3-(4-chlorophenyl)allyl)-N-((2-chloropyridin-4-yl)methyl)-4-(trifluoromethyl)spiro[indoline-3,4′-piperidine]-1-carboxamide

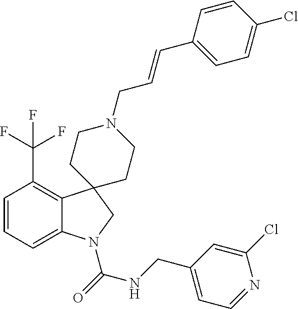 [SMILES: O=C(NCc1ccnc(Cl)c1)N1CC2(CCN(C/C=C/c3ccc(Cl)cc3)CC2)c2c1cccc2C(F)(F)F]

Using the same procedures as Step 2 through 6 of Example 56 and replacing (3-Trifluoromethyl-phenyl)-hydrazine with commercially available 3-(trifluoromethyl)aniline and (E)-3-(3,4-Dichloro-phenyl)-propenal with commercially available (E)-3-(4-chlorophenyl)acrylaldehyde the title compound is obtained: 1H NMR (400 MHz, DMSO-d6) δ: 1.60 (d, J=11.48 Hz, 2H), 2.08-2.23 (m, 4H), 2.89 (d, J=10.4 Hz, 2H), 3.14 (d, J=6.28 Hz, 2H), 3.96 (s, 2H), 4.38 (d, J=5.68 Hz, 2H), 6.35-6.42 (m, 1H), 6.54 (d, J=15.96 Hz, 1H), 7.24 (d, J=7.48 Hz, 1H), 7.32-7.38 (m, 4H), 7.44 (s, 1H), 7.49 (d, J=8.52 Hz, 2H), 7.68 (t, J=5.82 Hz, 1H), 8.28 (d, J=8.08 Hz, 1H), 8.34 (d, J=5.04 Hz, 1H). LC-MS (m/z): 575.1 (M+H) r.t. 6.12 min HPLC: 99.48%.

### Example 150

### (E)-6-chloro-1′-(3-(4-chlorophenyl)allyl)-N-((2-chloropyridin-4-yl)methyl)-5-(trifluoromethoxy)spiro[indoline-3,4′-piperidine]-1-carboxamide

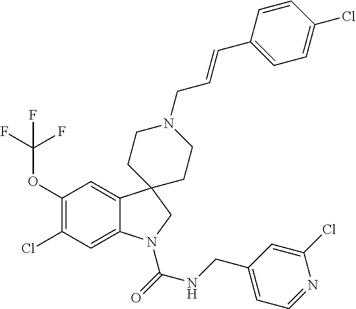 [SMILES: O=C(NCc1ccnc(Cl)c1)N1CC2(CCN(C/C=C/c3ccc(Cl)cc3)CC2)c2cc(OC(F)(F)F)c(Cl)cc21]

Using the same procedures as Step 1 through 6 of Example 55 and replacing 4-(trifluoromethoxy)aniline with commercially available 3-chloro-4-(trifluoromethoxy)aniline and (E)-3-(3,4-Dichloro-phenyl)-propenal with commercially available (E)-3-(4-chlorophenyl)acrylaldehyde the title compound is obtained 1H NMR (400 MHz, DMSO-d6) δ: 1.68 (d, J=12.48 Hz, 2H), 1.86-1.91 (m, 2H), 2.06 (t, J=11.8 Hz, 2H), 2.90 (d, J=11.36 Hz, 2H), 3.14 (d, J=6.4 Hz, 2H), 3.92 (s, 2H), 4.36 (d, J=5.56 Hz, 2H), 6.31-6.39 (m, 1H), 6.56 (d, J=16.04 Hz, 1H), 7.36-7.39 (m, 3H), 7.43-7.49 (m, 4H), 7.65 (t, J=5.88 Hz, 1H), 7.99 (s, 1H), 8.34 (d, J=5.12 Hz, 1H), LC-MS (m/z): 624.9 (M+H) r.t. 5.08 min HPLC: 99.88%.

### Example 151

### (E)-N-((2-chloropyridin-4-yl)methyl)-5-(trifluoromethyl)-1′-(3-(5-(trifluoromethyl)pyridin-2-yl)allyl)spiro[indoline-3,4′-piperidine]-1-carboxamide

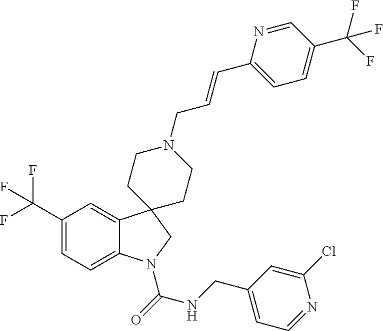 [SMILES: O=C(NCc1ccnc(Cl)c1)N1CC2(CCN(C/C=C/c3ccc(C(F)(F)F)cn3)CC2)c2cc(C(F)(F)F)ccc21]

Using the same procedures as Step 1 through 6 of Example 55 and replacing 4-(trifluoromethoxy)aniline with commercially available 4-(trifluoromethyl)aniline and (E)-3-(3,4-Dichloro-phenyl)-propenal with (E)-3-(5-(trifluoromethyl)pyridin-2-yl)acrylaldehyde (the product of step 2, Example 124) the title compound is obtained: 1H NMR (400 MHz, DMSO-d6) δ: 1.68 (d, J=12.48 Hz, 2H), 1.88-1.95 (m, 2H), 2.13 (t, J=12.08 Hz, 2H), 2.93 (d, J=11.12 Hz, 2H), 3.26 (d, J=6.08 Hz, 2H), 3.94 (s, 2H), 4.37 (d, J=5.44 Hz, 2H), 6.77-6.81 (m, 1H), 6.94-6.99 (m, 1H), 7.23 (d, J=7.84 Hz, 1H), 7.38 (d, J=5.08 Hz, 1H), 7.42-7.53 (m, 2H), 7.62-7.65 (m, 1H), 7.69-7.71 (m, 1H), 8.13-8.17 (m, 2H), 8.35 (d, J=5.12 Hz, 1H), 8.89 (s, 1H), LC-MS (m/z): 609.6 (M+H) r.t. 3.88 min.

### Example 152

### (E)-N-((2-chloropyridin-4-yl)methyl)-4-(trifluoromethyl)-1′-(3-(4-(trifluoromethyl)phenyl)allyl)spiro[indoline-3,4′-piperidine]-1-carboxamide

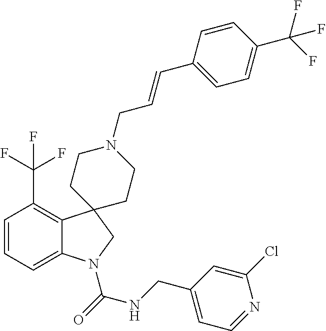 [SMILES: O=C(NCc1ccnc(Cl)c1)N1CC2(CCN(C/C=C/c3ccc(C(F)(F)F)cc3)CC2)c2c1cccc2C(F)(F)F]

Using the same procedures as Step 2 through 6 of Example 56 and replacing (3-Trifluoromethyl-phenyl)-hydrazine with commercially (3-(trifluoromethyl)phenyl)hydrazine and (E)-3-(3,4-Dichloro-phenyl)-propenal with commercially available (E)-3-(4-(trifluoromethyl)phenyl)acrylaldehyde the title compound is obtained: 1H NMR (400 MHz, DMSO-d6) δ: 1.60 (d, J=11.36 Hz, 2H), 2.12-2.25 (m, 4H), 2.90 (d, J=10.04 Hz, 2H), 3.18 (d, J=6.1 Hz, 2H), 3.97 (s, 2H), 4.38 (d, J=5.6 Hz, 2H), 6.53-6.58 (m, 1H), 6.65 (d, J=16.0 Hz, 1H), 7.24 (d, J=7.2 Hz, 1H), 7.32-7.37 (m, 2H), 7.45 (s, 1H), 7.65-7.70 (m, 5H), 8.28 (d, J=8.0 Hz, 1H), 8.34 (d, J=5.1 Hz, 1H). LC-MS (m/z): 608.9 (M+H) r.t. 6.20 min. HPLC: 99.84%.

### Example 153

### (E)-N-((2-chloropyridin-4-yl)methyl)-1′-(3-(4-fluorophenyl)allyl)-4-(trifluoromethyl)spiro[indoline-3,4′-piperidine]-1-carboxamide

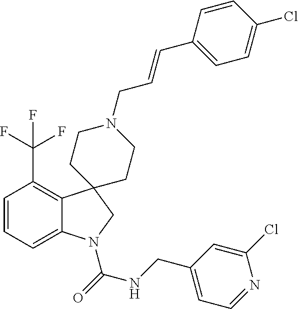 [SMILES: O=C(NCc1ccnc(Cl)c1)N1CC2(CCN(C/C=C/c3ccc(Cl)cc3)CC2)c2c1cccc2C(F)(F)F]

Using the same procedures as Step 2 through 6 of Example 56 and replacing (3-Trifluoromethyl-phenyl)-hydrazine with commercially (3-(trifluoromethyl)phenyl)-hydrazine and (E)-3-(3,4-Dichloro-phenyl)-propenal with commercially available (E)-3-(4-fluorophenyl)acrylaldehyde the title compound is obtained: 1H NMR (400 MHz, DMSO-d6) δ: 1.60 (d, J=11.8 Hz, 2H), 2.13-2.21 (m, 4H), 2.89 (d, J=10.4 Hz, 2H), 3.13 (d, J=6.4 Hz, 2H), 3.96 (s, 2H), 4.38 (d, J=5.6 Hz, 2H), 6.29-6.35 (m, 1H), 6.53 (d, J=15.9 Hz, 1H), 7.14 (t, J=8.8 Hz, 2H), 7.24 (d, J=7.2 Hz, 1H), 7.32-7.37 (m, 2H), 7.44 (s, 1H), 7.49-7.53 (m, 2H), 7.67 (t, J=5.7 Hz, 1H), 8.28 (d, J=8.0 Hz, 1H), 8.34 (d, J=5.0 Hz, 1H). LC-MS (m/z): 558.9 (M+H) r.t. 14.36 min. HPLC: 99.00%.

### Example 154

### (E)-N-((2-chloropyridin-4-yl)methyl)-1′-(3-(4-fluorophenyl)allyl)-5-(trifluoromethyl)spiro[indoline-3,4′-piperidine]-1-carboxamide

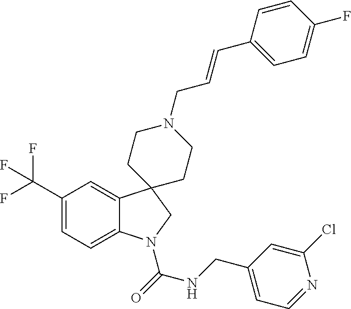 [SMILES: O=C(NCc1ccnc(Cl)c1)N1CC2(CCN(C/C=C/c3ccc(F)cc3)CC2)c2cc(C(F)(F)F)ccc21]

Using the same procedures as Step 1 through 6 of Example 55 and replacing 4-(trifluoromethoxy)aniline with commercially available 4-(trifluoromethyl)aniline and (E)-3-(3,4-Dichloro-phenyl)-propenal with commercially available (E)-3-(4-fluorophenyl)acrylaldehyde the title compound is obtained: 1H NMR (400 MHz, DMSO-d6) δ: 1.67 (d, J=12.16 Hz, 2H), 1.89-1.95 (m, 2H), 2.07 (t, J=5.76 Hz, 2H), 2.92 (d, J=11.44 Hz, 2H), 3.14 (d, J=6.16 Hz, 2H), 3.94 (s, 2H), 4.37 (d, J=5.64 2H), 6.25-6.32 (m, 1H), 6.57 (d, J=16.06 Hz, 1H), 7.15 (t, J=8.84 Hz, 2H), 7.38 (d, J=4.96 Hz, 1H), 7.46-7.52 (m, 5H), 7.64 (t, J=5.6 Hz, 1H), 7.97 (d, J=8.4 Hz, 1H), 8.34 (d, J=5.08 Hz, 1H). LC-MS (m/z): 559.2 (M+H) r.t. 5.60 min HPLC: 96.17%.

### Example 155

### (E)-1′-(3-(4-chlorophenyl)allyl)-N-((2-chloropyridin-4-yl)methyl)-5-fluoro-4-methylspiro[indoline-3,4′-piperidine]-1-carboxamide

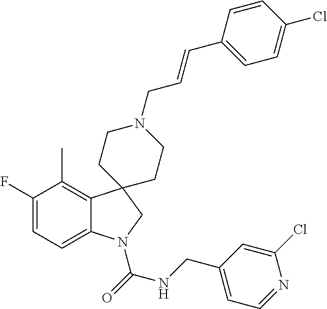 [SMILES: Cc1c(F)ccc2c1C1(CCN(C/C=C/c3ccc(Cl)cc3)CC1)CN2C(=O)NCc1ccnc(Cl)c1]

Using the same procedures as Step 1 through 6 of Example 56 and replacing 4-(trifluoromethoxy)aniline with commercially available (4-fluoro-3-methylphenyl)hydrazine and (E)-3-(3,4-Dichloro-phenyl)-propenal with commercially available (E)-3-(4-chlorophenyl)acrylaldehyde the title compound is obtained: 1H NMR (400 MHz, DMSO-d6) δ: 1.59 (d, J=13.0 Hz, 2H), 2.08-2.11 (m, 2H), 2.21-2.28 (m, 5H), 2.91 (d, J=10.36 Hz, 2H), 3.14 (d, J=6.24 Hz, 2H), 3.86 (s, 2H), 4.34 (d, J=5.6 Hz, 2H), 6.35-6.43 (m, 1H), 6.56 (d, J=16.04 Hz, 1H), 6.89 (t, J=9.66 Hz, 1H), 7.35-7.38 (m, 3H), 7.41-7.43 (m, 2H), 7.49 (d, J=8.52 Hz, 2H), 7.74-7.77 (m, 1H), 8.33 (d, J=5.08 Hz, 1H). LC-MS (m/z): 539.1 (M+H) r.t. 5.79 min HPLC: 99.85%.

### Example 156

### (E)-4-bromo-1′-(3-(4-chlorophenyl)allyl)-N-((2-chloropyridin-4-yl)methyl)spiro[indoline-3,4′-piperidine]-1-carboxamide

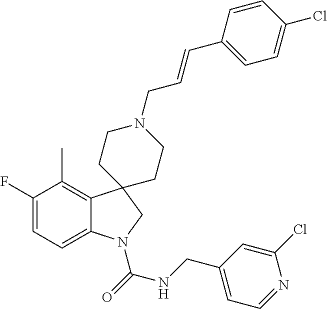 [SMILES: Cc1c(F)ccc2c1C1(CCN(C/C=C/c3ccc(Cl)cc3)CC1)CN2C(=O)NCc1ccnc(Cl)c1]

Using the same procedures as Step 2 through 6 of Example 56 and replacing (3-Trifluoromethyl-phenyl)-hydrazine with commercially available (3-bromophenyl)hydrazine and (E)-3-(3,4-Dichloro-phenyl)-propenal with commercially available (E)-3-(4-chlorophenyl)acrylaldehyde the title compound is obtained: 1H NMR (400 MHz, DMSO-d6) δ: 1.52 (d, J=12.68 Hz, 2H), 2.09 (t, J=11.42 Hz, 2H), 2.62-2.69 (m, 2H), 2.92 (d, J=11.52 Hz, 2H), 3.13 (d, J=6.4 Hz, 2H), 3.90 (s, 2H), 4.36 (d, J=5.6 Hz, 2H), 6.36-6.43 (m, 1H), 6.54 (d, J=15.96 Hz, 1H), 7.03-7.06 (m, 2H), 7.37 (d, J=8.28 Hz, 3H), 7.45 (s, 1H), 7.49 (d, J=8.52 Hz, 2H), 7.55 (t, J=5.74 Hz, 1H), 7.93-7.98 (m, 1H), 8.34 (d, J=5.08 Hz, 1H). LC-MS (m/z): 585 (M+H) r.t. 6.01 min HPLC: 98.81%.

### Example 157

### (E)-4-chloro-N-((2-chloropyridin-4-yl)methyl)-1′-(3-(4-cyanophenyl)allyl)-5-fluorospiro[indoline-3,4′-piperidine]-1-carboxamide

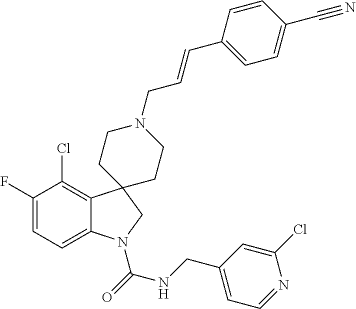 [SMILES: N#Cc1ccc(/C=C/CN2CCC3(CC2)CN(C(=O)NCc2ccnc(Cl)c2)c2ccc(F)c(Cl)c23)cc1]

Using the same procedures as Step 2 through 6 of Example 56 and replacing (3-Trifluoromethyl-phenyl)-hydrazine with commercially available (3-chloro-4-fluorophenyl)hydrazine and (E)-3-(3,4-Dichloro-phenyl)-propenal with commercially available (E)-4-(3-oxoprop-1-en-1-yl)benzonitrile the title compound is obtained: 1H NMR (400 MHz, DMSO-d6) δ: 1.60 (d, J=12.56 Hz, 2H), 2.08 (t, J=10.6 Hz, 2H), 2.91 (d, J=11.56 Hz, 2H), 3.17 (d, J=4.86 Hz, 2H), 3.94 (s, 2H), 4.35 (d, J=5.6 Hz, 2H), 6.57-6.67 (m, 2H), 7.16 (t, J=9.16 Hz, 1H), 7.36 (dd, J=5.6 Hz, 1H), 7.45 (s, 1H), 7.54 (t, J=5.72 Hz, 1H), 7.66 (d, J=8.44 Hz, 2H), 7.78 (d, J=8.4 Hz, 2H), 7.86-7.90 (m, 1H), 8.33 (d, J=5.06 Hz, 1H). LC-MS (m/z): 549.8 r.t. 3.97 min HPLC: 98.11%.

### Example 158

### (E)-4-chloro-N-((2-chloropyridin-4-yl)methyl)-5-fluoro-1′-(3-(4-fluorophenyl)allyl)spiro[indoline-3,4′-piperidine]-1-carboxamide

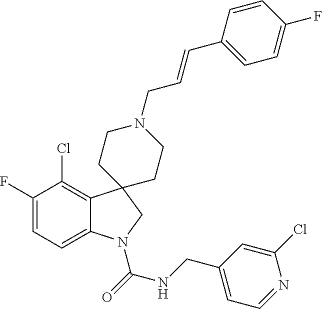 [SMILES: O=C(NCc1ccnc(Cl)c1)N1CC2(CCN(C/C=C/c3ccc(F)cc3)CC2)c2c1ccc(F)c2Cl]

Using the same procedures as Step 2 through 6 of Example 56 and replacing (3-Trifluoromethyl-phenyl)-hydrazine with commercially available (3-chloro-4-fluorophenyl)hydrazine and (E)-3-(3,4-Dichloro-phenyl)-propenal with commercially available (E)-3-(4-fluorophenyl)acrylaldehyde the title compound is obtained: 1H NMR (400 MHz, DMSO-d6) δ: 1.59 (d, J=12.76 Hz, 2H), 2.06 (t, J=11.2 Hz, 2H), 2.55 (d, J=12 Hz, 2H), 2.91 (d, J=11.76 Hz, 2H), 3.12 (d, J=6.6 Hz, 2H), 7.13-7.18 (m, 3H), 7.36 (d, J=5.16 Hz, 2H), 7.45 (s, 1H), 7.45-7.56 (m, 3H), 7.86-7.90 (m, 1H), 8.33 (d, J=4.6 Hz, 1H). LC-MS (m/z): 543.2 (M+H) 5.64 min HPLC: 99.45%.

### Example 159

### (E)-4-chloro-1′-(3-(4-chlorophenyl)allyl)-N-((2-chloropyridin-4-yl)methyl)-5-(trifluoromethoxy)spiro[indoline-3,4′-piperidine]-1-carboxamide

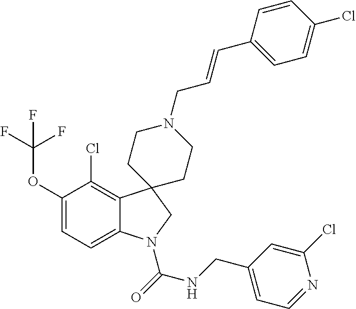 [SMILES: O=C(NCc1ccnc(Cl)c1)N1CC2(CCN(C/C=C/c3ccc(Cl)cc3)CC2)c2c1ccc(OC(F)(F)F)c2Cl]

Using the same procedures as Step 1 through 6 of Example 55 and replacing 4-(trifluoromethoxy)aniline with commercially available 3-chloro-4-(trifluoromethoxy)aniline and (E)-3-(3,4-Dichloro-phenyl)-propenal with commercially available (E)-3-(4-chlorophenyl)acrylaldehyde the title compound is obtained: 1H NMR (400 MHz, DMSO-d6) δ: 1.62 (d, J=12.8 Hz, 2H), 2.07 (t, J=11.3 Hz, 2H), 2.55-2.57 (m, 2H), 2.92 (d, J=11.36 Hz, 2H), 3.13 (d, J=6.32 Hz, 2H), 3.96 (s, 2H), 4.36 (d, J=5.64 Hz, 2H), 6.36-6.42 (m, 1H), 6.55 (d, J=16 Hz, 1H), 7.33 (dd, J=1.12, 8.9 Hz, 1H), 7.37 (d, J=8.36 Hz, 3H), 7.45 (s, 1H), 7.50 (d, J=8.44 Hz, 2H), 7.63 (t, J=5.72 Hz, 1H), 7.96 (d, J=8.92 Hz, 1H), 8.34 (d, J=5.08 Hz, 1H). LC-MS (m/z): 624.8 (M+H) r.t. 5.10 min H PLC: 99.84%.

### Example 160

### (E)-1′-(3-(4-chlorophenyl)allyl)-N-((2-chloropyridin-4-yl)methyl)-5-(trifluoromethyl)spiro[indoline-3,4′-piperidine]-1-carboxamide

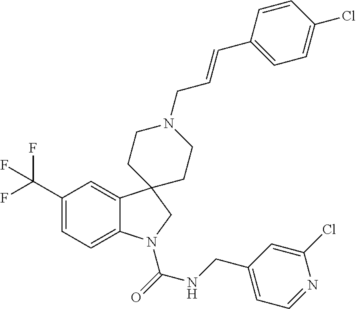 [SMILES: O=C(NCc1ccnc(Cl)c1)N1CC2(CCN(C/C=C/c3ccc(Cl)cc3)CC2)c2cc(C(F)(F)F)ccc21]

Using the same procedures as Step 1 through 6 of Example 55 and replacing 4-(trifluoromethoxy)aniline with commercially available 4-(trifluoromethyl)aniline and (E)-3-(3,4-Dichloro-phenyl)-propenal with commercially available (E)-3-(4-chlorophenyl)acrylaldehyde the title compound is obtained: 1H NMR (400 MHz, DMSO-d6) δ: 1.67 (d, J=12.40 Hz, 2H), 1.89-1.96 (m, 2H), 2.09 (t, J=11.58 Hz, 2H), 2.91 (d, J=11.36 Hz, 2H), 3.15 (d, J=6.32 Hz, 2H), 3.94 (s, 2H), 4.37 (d, J=1.14 Hz, 2H), 6.34-6.40 (m, 1H), 6.57 (d, J=15.96 Hz, 1H), 7.37-7.39 (m, 3H), 7.46-7.52 (m, 5H), 7.64 (t, J=5.78 Hz, 1H), 7.97 (d, J=8.44 Hz, 1H), 8.34 (d, J=5.08 Hz, 1H). LC-MS (m/z): 573 (M−H) 5.96 min HPLC: 99.78%.

### Example 161

### (E)-4,6-dichloro-N-((2-chloropyridin-4-yl)methyl)-1′-(3-(4-fluorophenyl)allyl)spiro[indoline-3,4′-piperidine]-1-carboxamide

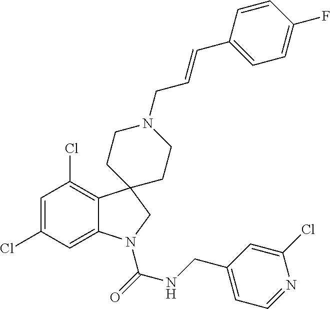 [SMILES: O=C(NCc1ccnc(Cl)c1)N1CC2(CCN(C/C=C/c3ccc(F)cc3)CC2)c2c(Cl)cc(Cl)cc21]

Using the same procedures as Step 2 through 6 of Example 56 and replacing (3-Trifluoromethyl-phenyl)-hydrazine with commercially available and (E)-3-(3,4-Dichloro-phenyl)-propenal with commercially available (E)-3-(4-fluorophenyl)acrylaldehyde the title compound is obtained: 1H NMR (400 MHz, DMSO-d6) δ: 1.58 (d, J=12.4 Hz, 2H), 2.04 (t, J=11.6 Hz, 2H), 2.46-2.54 (m, 2H), 2.91 (d, J=11.6 Hz, 2H), 3.11 (d, J=6.5 Hz, 2H), 3.9 (s.2H), 4.36 (d, J=5.6 Hz, 2H), 6.28-6.35 (m, 1H), 6.53 (d, J=15.9 Hz, 1H), 7.00 (d, J=1.9 Hz, 1H), 7.15 (t, J=8.8 Hz, 2H), 7.37 (d, J=5.0 Hz, 1H), 7.46 (s, 1H), 7.49-7.53 (m, 2H), 7.68 (t, J=5.6 Hz, 1H), 7.93 (d, J=1.9 Hz, 1H), 8.34 (d, J=5.0 Hz, 1H). LC-MS (m/z): 558.6 (M+H) 10.13 min HPLC: 99.15%.

### Example 162

### (E)-1′-(3-(4-chlorophenyl)allyl)-N-((2-chloropyridin-4-yl)methyl)-6-(methylsulfonyl)spiro[indoline-3,4′-piperidine]-1-carboxamide

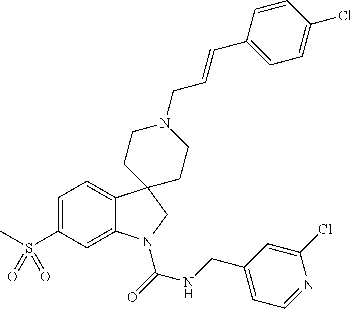 [SMILES: CS(=O)(=O)c1ccc2c(c1)N(C(=O)NCc1ccnc(Cl)c1)CC21CCN(C/C=C/c2ccc(Cl)cc2)CC1]

Using the same procedures as Step 1 through 6 of Example 55 and replacing 4-(trifluoromethoxy)aniline with commercially available 3-(methylsulfonyl)aniline and (E)-3-(3,4-Dichloro-phenyl)-propenal with commercially available (E)-3-(4-chlorophenyl)acrylaldehyde the title compound is obtained: 1H NMR (400 MHz, DMSO-d6) δ: 1.68 (d, J=15.8 Hz, 2H), 1.90 (t, J=13.14 Hz, 2H), 2.08 (t, J=10.52 Hz, 2H), 2.92 (d, J=14.44 Hz, 2H), 3.12 (s, 3H), 3.15 (d, J=5.12 Hz, 2H), 3.95 (s, 2H), 4.37 (d, J=5.56 Hz, 2H), 6.33-6.42 (m, 1H) 6.57 (d, J=17.08 Hz, 1H), 7.37-7.39 (m, 3H), 7.45-7.50 (m, 5H), 7.65-7.67 (m, 1H), 8.32 (s, 1H), 8.35 (d, J=5.08 Hz, 1H). LC-MS (m/z): 585.3 (M+H) r.t. 4.86 min. HPLC purity: 92.40%.

### Example 163

### (E)-N-((2-chloropyridin-4-yl)methyl)-1′-(3-(4-fluorophenyl)allyl)-5-(trifluoromethoxy)spiro[indoline-3,4′-piperidine]-1-carboxamide

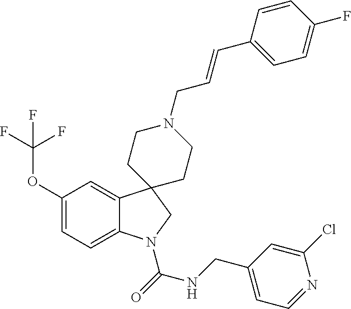 [SMILES: O=C(NCc1ccnc(Cl)c1)N1CC2(CCN(C/C=C/c3ccc(F)cc3)CC2)c2cc(OC(F)(F)F)ccc21]

Using the same procedures as Step 1 through 6 of Example 55 and replacing 4-(trifluoromethoxy)aniline with commercially available 4-(trifluoromethoxy)aniline and (E)-3-(3,4-Dichloro-phenyl)-propenal with commercially available (E)-3-(4-fluorophenyl)acrylaldehyde the title compound is obtained: 1H NMR (400 MHz, DMSO-d6) δ: 1.66 (d, J=12.36 Hz, 2H), 1.85-1.91 (m, 2H), 2.07 (t, J=11.5 Hz, 2H), 2.91 (d, J=11.16 Hz, 2H), 3.14 (d, J=6.32 Hz, 2H), 3.91 (s, 2H), 4.36 (d, J=5.6 Hz, 2H), 6.27-6.32 (m, 1H), 6.56 (d, J=15.92 Hz, 1H), 7.09 (dd, J=1.32, 8.72 Hz, 1H), 7.13 (t, J=8.86 Hz, 2H), 7.22 (d, J=1.72 Hz, 1H), 7.37 (d, J=5.08 Hz, 1H), 7.44 (s, 1H), 7.48-7.56 (m, 3H), 7.87 (d, J=8.76 Hz, 1H), 8.34 (d, J=5.08 Hz, 1H). LC-MS (m/z): [M+H]=574.9 r.t. 7.52 min. HPLC purity: 99.61%.

### Example 164

### (E)-4-chloro-N-((2-chloropyridin-4-yl)methyl)-5-fluoro-1′-(3-(4-(trifluoromethyl)phenyl)allyl)spiro[indoline-3,4′-piperidine]-1-carboxamide

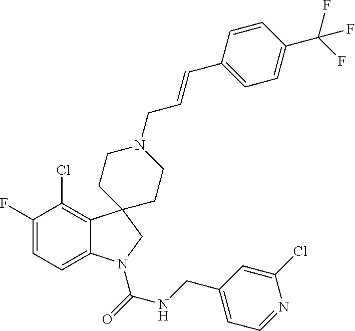 [SMILES: O=C(NCc1ccnc(Cl)c1)N1CC2(CCN(C/C=C/c3ccc(C(F)(F)F)cc3)CC2)c2c1ccc(F)c2Cl]

Using the same procedures as Step 2 through 6 of Example 56 and replacing (3-Trifluoromethyl-phenyl)-hydrazine with commercially available (3-chloro-4-fluorophenyl)hydrazine and (E)-3-(3,4-Dichloro-phenyl)-propenal with commercially available (E)-3-(4-(trifluoromethyl)phenyl)acrylaldehyde the title compound is obtained: 1H NMR (400 MHz, DMSO-d6) δ: 1.60 (d, J=12.72 Hz, 2H), 2.08 (t, J=12.04 Hz, 2H), 2.49-2.56 (m, 2H), 2.92 (d, J=11.24 Hz, 2H), 3.17 (d, J=6.06 Hz, 2H), 7.16 (t, J=9.16 Hz, 1H), 7.37 (d, J=5.6 Hz, 1H), 7.45 (s, 1H), 7.54 (t, J=5.6 Hz, 1H), 7.65-7.71 (m, 4H), 7.86-7.90 (m, 1H), 8.33 (d, J=5.16 Hz, 1H). LC-MS (m/z): 593.2 (M+H) r.t. 17.59 min. HPLC: 99.29%.

### Example 165

### (E)-1′-(3-(4-chlorophenyl)allyl)-N-((2-chloropyridin-4-yl)methyl)-4-cyanospiro[indoline-3,4′-piperidine]-1-carboxamide

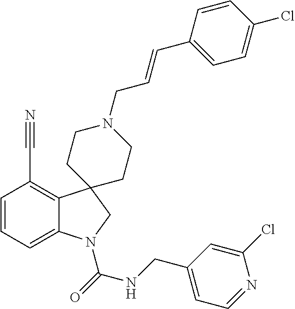 [SMILES: N#Cc1cccc2c1C1(CCN(C/C=C/c3ccc(Cl)cc3)CC1)CN2C(=O)NCc1ccnc(Cl)c1]

Using the same procedures as Step 1 through 6 of Example 55 and replacing 4-(trifluoromethoxy)aniline with commercially available 3-aminobenzonitrile and (E)-3-(3,4-Dichloro-phenyl)-propenal with commercially available (E)-3-(4-chlorophenyl)acrylaldehyde the title compound is obtained: 1H NMR (400 MHz, DMSO-d6) δ: 1.69 (d, J=12.48 Hz, 2H), 2.05-2.12 (m, 2H), 2.31-2.33 (m, 2H), 2.95 (d, J=11.72 Hz, 2H), 3.16 (d, J=6.72 Hz, 2H), 3.96 (s, 2H), 4.37 (d, J=5.76 Hz, 2H), 6.38-6.46 (m, 1H), 6.55 (d, J=16.68 Hz, 1H), 7.28-7.33 (m, 2H), 7.36-7.38 (m, 3H), 7.46 (s, 1H), 7.50 (d, J=8.52 Hz, 2H), 7.62 (t, J=6.38 Hz, 1H), 8.19 (dd, J=2.24, 7.08 Hz, 1H), 8.34 (d, J=5.12 Hz, 1H). LC-MS (m/z): 532.2 (M+H) r.t. 33.15 min. HPLC: 97.09%.

### Example 166

### (E)-1′-(3-(4-chlorophenyl)allyl)-N-((2-chloropyridin-4-yl)methyl)-4-cyano-5-fluorospiro[indoline-3,4′-piperidine]-1-carboxamide

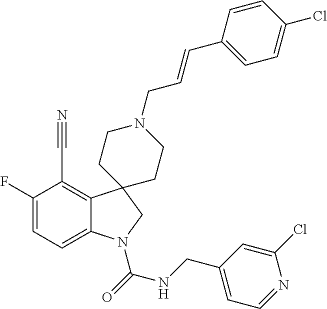 [SMILES: N#Cc1c(F)ccc2c1C1(CCN(C/C=C/c3ccc(Cl)cc3)CC1)CN2C(=O)NCc1ccnc(Cl)c1]

Using the same procedures as Step 1 through 6 of Example 55 and replacing 4-(trifluoromethoxy)aniline with commercially available 5-amino-2-fluorobenzonitrile and (E)-3-(3,4-Dichloro-phenyl)-propenal with commercially available (E)-3-(4-chlorophenyl)acrylaldehyde the title compound is obtained: 1H NMR (400 MHz, DMSO-d6) δ: 1.74 (d, J=12.4 Hz, 2H), 2.07 (t, J=12.0 Hz, 2H), 2.25 (t, J=10.6 Hz, 2H), 2.95 (d, J=11.4 Hz, 2H), 3.16 (d, J=6.2 Hz, 2H), 3.98 (s, 2H), 4.36 (d, J=5.5 Hz, 2H), 6.39-6.45 (m, 1H), 6.55 (d, J=16.5 Hz, 1H), 7.28 (t, J=7.4 Hz, 1H), 7.37 (d, J=7.4 Hz, 3H), 7.45 (s, 1H), 7.50 (d, J=8.4 Hz, 2H), 7.62 (t, J=5.6 Hz, 1H), 8.16-8.20 (m, 1H), 8.34 (d, J=5.1 Hz, 1H). LC-MS (m/z): 550.1 (M+H) r.t. 9.93 min. HPLC: 98.58%.

### Example 167

### (E)-N-((2-chloropyridin-4-yl)methyl)-1′-(3-(3,4-dichlorophenyl)allyl)-5-methoxyspiro[indoline-3,4′-piperidine]-1-carboxamide

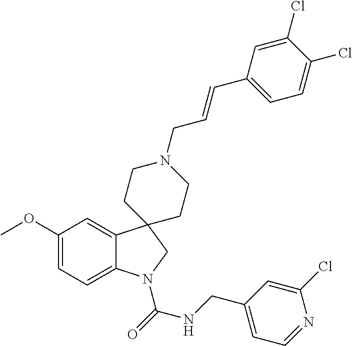 [SMILES: COc1ccc2c(c1)C1(CCN(C/C=C/c3ccc(Cl)c(Cl)c3)CC1)CN2C(=O)NCc1ccnc(Cl)c1]

Using the same procedures as Step 2 through 6 of Example 56 and replacing (3-Trifluoromethyl-phenyl)-hydrazine with commercially available (4-methoxyphenyl)hydrazine the title compound is obtained: LC-MS (m/z): 570.8 (M+H), r.t. 4.89 min.

### Example 168

### (E)-N-((2-chloropyridin-4-yl)methyl)-1′-(3-(4-methoxyphenyl)allyl)-5-(trifluoromethyl)spiro[indoline-3,4′-piperidine]-1-carboxamide

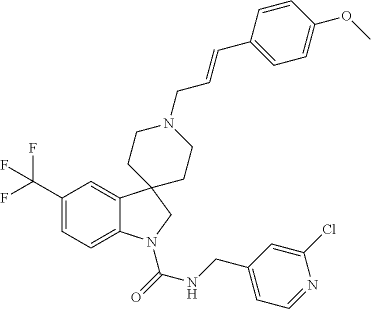 [SMILES: COc1ccc(/C=C/CN2CCC3(CC2)CN(C(=O)NCc2ccnc(Cl)c2)c2ccc(C(F)(F)F)cc23)cc1]

Using the same procedures as Step 1 through 6 of Example 55 and replacing 4-(trifluoromethoxy)aniline with commercially available 4-(trifluoromethyl)aniline and (E)-3-(3,4-Dichloro-phenyl)-propenal with commercially available (E)-3-(4-methoxyphenyl)acrylaldehyde the title compound is obtained: 1H NMR (400 MHz, DMSO-d6) δ: 1.67 (d, J=12.32 Hz, 2H), 1.89-1.91 (m, 2H), 2.07 (t, J=11.12 Hz, 2H), 2.92 (d, J=11.44 Hz, 2H), 3.12 (d, J=6.52 Hz, 2H), 3.74 (s, 3H), 3.94 (s, 2H), 4.37 (d, J=5.64 2H), 6.13-6.20 (m, 1H), 6.49 (d, J=15.76 Hz, 1H), 6.89 (d, J=8.76 Hz, 2H), 7.37-7.39 (m, 3H), 7.46-7.47 (m, H), 7.52 (s, 1H), 7.68 (t, J=12.1 Hz, 1H), 7.97 (d, J=8.4 Hz, 1H), 8.34 (d, J=5.08 Hz, 1H). LC-MS (m/z): 571.2 (M+H) r.t. 5.42 min. HPLC: 99.60%.

### Example 169

### (E)-1′-(3-(4-chlorophenyl)allyl)-N-((2-chloropyridin-4-yl)methyl)-5-(pentafluorosulfide)spiro[indoline-3,4′-piperidine]-1-carboxamide

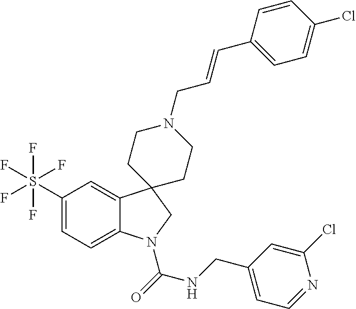 [SMILES: O=C(NCc1ccnc(Cl)c1)N1CC2(CCN(C/C=C/c3ccc(Cl)cc3)CC2)c2cc(S(F)(F)(F)(F)F)ccc21]

Using the same procedures as Step 1 through 6 of Example 55 and replacing 4-(trifluoromethoxy)aniline with commercially available (4-(pentafluoro-λ⁶-sulfanyl)phenyl)hydrazine and (E)-3-(3,4-Dichloro-phenyl)-propenal with commercially available (E)-3-(4-chlorophenyl)acrylaldehyde the title compound is obtained: 1H NMR (400 MHz, DMSO-d6) δ: 1.69 (d, J=12.44 Hz, 2H), 1.92 (t, J=10.46 Hz, 2H), 2.08 (t, J=11.38 Hz, 2H), 2.92 (d, J=11.24 Hz, 2H), 3.15 (d, J=6.24 Hz, 2H), 3.95 (s, 2H), 4.37 (d, J=5.52 Hz, 2H), 6.34-6.39 (m, 1H) 6.57 (d, J=16.04 Hz, 1H), 7.37-7.39 (m, 3H), 7.46-7.49 (m, 3H), 7.63-7.70 (m, 3H), 7.93 (d, J=9.04 Hz, 1H), 8.34 (d, J=5.08 Hz, 1H). LC-MS (m/z): 632.8 (M+H) r.t. 4.72 min. HPLC purity: 98.19%.

### Example 170

### (E)-N-((2-chloropyridin-4-yl)methyl)-5-(trifluoromethyl)-1′-(3-(4-(trifluoromethyl)phenyl)allyl)spiro[indoline-3,4′-piperidine]-1-carboxamide

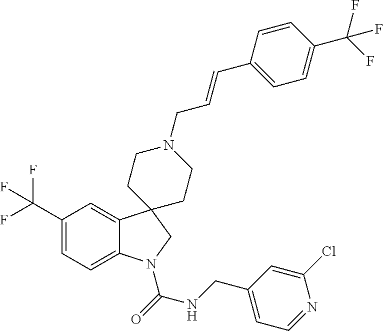 [SMILES: O=C(NCc1ccnc(Cl)c1)N1CC2(CCN(C/C=C/c3ccc(C(F)(F)F)cc3)CC2)c2cc(C(F)(F)F)ccc21]

Using the same procedures as Step 1 through 6 of Example 55 and replacing 4-(trifluoromethoxy)aniline with commercially available 4-(trifluoromethyl)aniline and (E)-3-(3,4-Dichloro-phenyl)-propenal with commercially (E)-3-(4-(trifluoromethyl)phenyl)acrylaldehyde the title compound is obtained: 1H NMR (400 MHz, DMSO-d6) δ: 1.68 (d, J=12.24 Hz, 2H), 1.90-1.98 (m, 2H), 2.10 (t, J=121.86 Hz, 2H), 2.93 (d, J=11.60 Hz, 2H), 3.19 (d, J=6.28 Hz, 2H), 3.94 (s, 2H), 4.37 (m, J=5.60 Hz, 2H), 6.49-6.56 (m, 1H), 6.68 (d, J=15.96 Hz, 1H), 7.38 (d, J=4.88 Hz, 1H), 7.47 (d, J=7.92 Hz, 2H), 7.53 (s, 1H), 7.64-7.68 (m, 5H), 7.97 (d, J=8.52 Hz, 1H), 8.34 (d, J=5.16 Hz, 1H). LC-MS (m/z): 608.9 (M+H) r.t. 9.11 min. HPLC: 99.96%.

### Example 171

### (E)-5,7-dichloro-N-((2-chloropyridin-4-yl)methyl)-1′-(3-(3,4-dichlorophenyl)allyl)spiro[indoline-3,4′-piperidine]-1-carboxamide

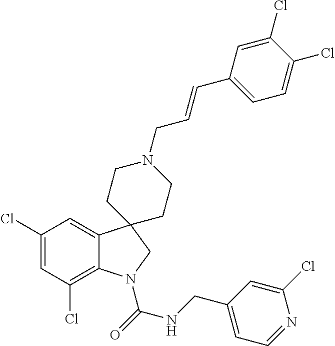 [SMILES: O=C(NCc1ccnc(Cl)c1)N1CC2(CCN(C/C=C/c3ccc(Cl)c(Cl)c3)CC2)c2cc(Cl)cc(Cl)c21]

Using the same procedures as Step 2 through 6 of Example 56 and replacing (3-Trifluoromethyl-phenyl)-hydrazine with commercially (2,4-dichlorophenyl)hydrazine the title compound is obtained: 1H NMR (400 MHz, DMSO-d6) δ: 1.48 (d, J=12.6 Hz, 2H), 1.84-1.90 (m, 2H), 2.02 (t, J=11.6 Hz, 2H), 2.89 (d, J=11.4 Hz, 2H), 3.14 (d, J=6.08 Hz, 2H), 3.93 (s, 2H), 4.33 (d, J=5.76 Hz, 2H), 6.43-6.46 (m, 1H), 6.56 (d, J=16 Hz, 1H), 7.30 (d, J=5.12 Hz, 1H), 7.34 (dd, J=1.64 Hz, 7.28 Hz, 3H), 7.46 (dd, J=1.92 Hz J=8.44 Hz, 1H), 7.58 (d, J=8.36 Hz, 1H) 7.74 (d, J=1.88 Hz, 1H), 7.95 (t, J=5.84 Hz, 1H), 8.33 (d, J=5.08 Hz, 1H), LC-MS (m/z): 608.8 (M+H) r.t. 5.97 min. HPLC: 99.31%.

### Example 172

### (E)-N-((2-chloropyridin-4-yl)methyl)-1′-(3-(4-(trifluoromethoxy)phenyl)allyl)-5-(trifluoromethyl)spiro[indoline-3,4′-piperidine]-1-carboxamide

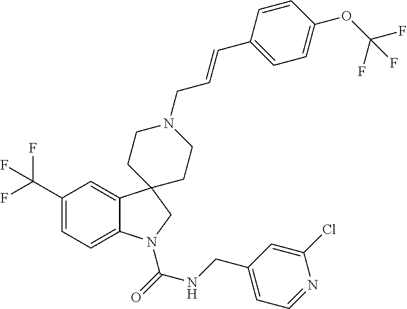 [SMILES: O=C(NCc1ccnc(Cl)c1)N1CC2(CCN(C/C=C/c3ccc(OC(F)(F)F)cc3)CC2)c2cc(C(F)(F)F)ccc21]

Using the same procedures as Step 1 through 6 of Example 55 and replacing 4-(trifluoromethoxy)aniline with commercially available 4-(trifluoromethyl)aniline and (E)-3-(3,4-Dichloro-phenyl)-propenal with commercially available (E)-3-(4-(trifluoromethoxy)phenyl)acrylaldehyde the title compound is obtained: 1H NMR (400 MHz, DMSO-d6) δ: 1.68 (d, J=12.08 Hz, 2H), 1.92 (t, J=10.9 Hz, 2H), 2.06-2.12 (m, 2H), 2.92 (d, J=11.76 Hz, 2H), 3.16 (d, J=5.92 Hz, 2H), 3.94 (s, 2H), 4.37 (d, J=5.64 2H), 6.34-6.42 (m, 1H), 6.61 (d, J=15.96 Hz, 1H), 7.32 (d, J=8.28 Hz, 2H), 7.38 (d, J=5.12 Hz, 1H), 7.46-7.48 (m, 2H), 7.52 (s, 1H), 7.58 (d, J=8.72 Hz, 2H), 7.65 (t, J=6.98 Hz, 1H), 7.97 (d, J=8.6 Hz, 1H), 8.35 (d, J=5.12 Hz, 2H) LC-MS (m/z): 625.0 (M+H) r.t. 9.55 min. HPLC: 99.90%.

### Example 173

### (E)-1′-(3-(4-chlorophenyl)allyl)-N-((2-chloropyridin-4-yl)methyl)-5-fluoro-4-(trifluoromethyl)spiro[indoline-3,4′-piperidine]-1-carboxamide

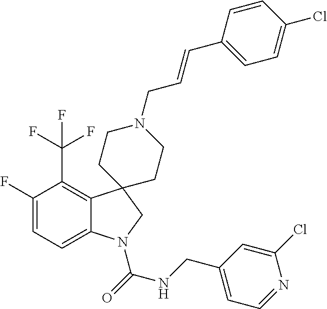 [SMILES: O=C(NCc1ccnc(Cl)c1)N1CC2(CCN(C/C=C/c3ccc(Cl)cc3)CC2)c2c1ccc(F)c2C(F)(F)F]

Using the same procedures as Step 1 through 6 of Example 55 and replacing 4-(trifluoromethoxy)aniline with commercially available 4-fluoro-3-(trifluoromethyl) and (E)-3-(3,4-Dichloro-phenyl)-propenal with commercially available (E)-3-(4-chlorophenyl)acrylaldehyde the title compound is obtained: 1H NMR (400 MHz, DMSO-d6) δ: 1.63 (d, J=12.6 Hz, 2H), 2.07-2.12 (m, 2H), 2.20-2.25 (m, 2H), 2.89 (d, J=10.56 Hz, 2H), 3.13 (d, J=6.36 Hz, 2H), 3.97 (s, 2H), 4.37 (d, J=5.52 Hz, 2H), 6.34-6.41 (m, 1H), 6.54 (d, J=15.88 Hz, 2H), 7.28 (dd, J=9.2, 11.8 Hz, 1H), 7.36-7.38 (m, 3H), 7.44 (s, 1H), 7.49 (d, J=8.52 Hz, 2H), 7.64 (t, J=5.68 Hz, 1H), 8.26-8.31 (m, 1H), 8.34 (d, J=5.08 Hz, 1H). LC-MS (m/z): 593.1 (M+H) r.t. 6.21 min. HPLC: 98.43%.

### Example 174

### (E)-N-((2-chloropyridin-4-yl)methyl)-1′-(3-(4-fluorophenyl)allyl)-4-(trifluoromethoxy)spiro[indoline-3,4′-piperidine]-1-carboxamide

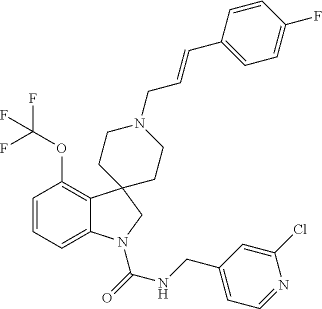 [SMILES: O=C(NCc1ccnc(Cl)c1)N1CC2(CCN(C/C=C/c3ccc(F)cc3)CC2)c2c(OC(F)(F)F)cccc21]

Using the same procedures as Step 1 through 6 of Example 55 and replacing 4-(trifluoromethoxy)aniline with commercially available 3-(trifluoromethoxy)aniline and (E)-3-(3,4-Dichloro-phenyl)-propenal with commercially available (E)-3-(4-fluorophenyl)acrylaldehyde the title compound is obtained: 1H NMR (400 MHz, DMSO-d6) δ: 1.67 (d, J=11.64 Hz, 2H), 2.03-2.09 (m, 2H), 2.14-2.19 (t, 2H), 2.91 (d, J=10.88 Hz, 2H), 3.13 (d, J=6.48 Hz, 2H), 3.92 (s, 2H), 4.37 (d, J=5.6 Hz, 2H), 6.28-6.36 (m, 1H), 6.54 (d, J=15.84 Hz, 1H), 6.82 (dd, J=1.48, 8.28 Hz, 1H), 7.15 (t, J=8.86 Hz, 2H), 7.23 (t, J=8.26 Hz, 1H), 7.37 (d, J=5.12 Hz, 1H), 7.45 (s, 1H), 7.58 (t, J=5.76 Hz, 1H), 7.85-7.87 (m, 1H), 8.34 (d, J=5.12 Hz, 1H). LC-MS (m/z): 574.8 (M+H) r.t. 4.59 min. HPLC: 99.76%.

### Example 175

### (E)-1′-(3-(4-fluorophenyl)allyl)-5-(trifluoromethoxy)-N-((2-(trifluoromethyl)pyridin-4-yl)methyl)spiro[indoline-3,4′-piperidine]-1-carboxamide

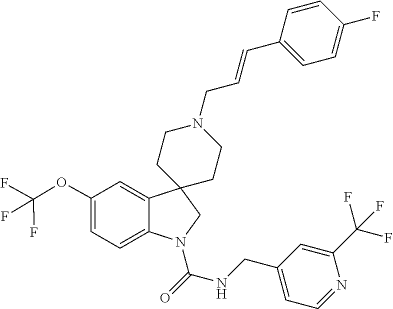 [SMILES: O=C(NCc1ccnc(C(F)(F)F)c1)N1CC2(CCN(C/C=C/c3ccc(F)cc3)CC2)c2cc(OC(F)(F)F)ccc21]

Using the same procedures as Step 2 through 6 of Example 56 and replacing (3-Trifluoromethyl-phenyl)-hydrazine with commercially available (4-(trifluoromethoxy)phenyl)hydrazine and (2-Chloro-pyridin-4-yl)-methylamine hydrochloride with commercially available (2-(trifluoromethyl)pyridin-4-yl)methanamine and (E)-3-(3,4-Dichloro-phenyl)-propenal with commercially available (E)-3-(4-fluorophenyl)acrylaldehyde the title compound is obtained: ¹H NMR (400 MHz, DMSO-d₆) δ ppm 1.99 (br. s., 5H) 3.32 (s, 4H) 4.01 (br. s., 3H) 4.47 (d, 2H) 6.37 (br. s., 1H) 7.04-7.31 (m, 3H) 7.47-7.74 (m, 3H) 7.82-7.92 (m, 2H) 8.71 (d, 1H). LC-MS (m/z): 609.0 (M+H) r.t. 2.64 min.

### Example 176

### (E)-1′-(3-(4-cyanophenyl)allyl)-5-(trifluoromethoxy)-N-((2-(trifluoromethyl)pyridin-4-yl)methyl)spiro[indoline-3,4′-piperidine]-1-carboxamide

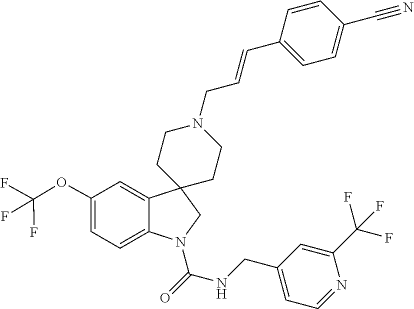 [SMILES: N#Cc1ccc(/C=C/CN2CCC3(CC2)CN(C(=O)NCc2ccnc(C(F)(F)F)c2)c2ccc(OC(F)(F)F)cc23)cc1]

Using the same procedures as Step 2 through 6 of Example 56 and replacing (3-Trifluoromethyl-phenyl)-hydrazine with commercially available (4-(trifluoromethoxy)phenyl)hydrazine and (2-Chloro-pyridin-4-yl)-methylamine hydrochloride with commercially available (2-(trifluoromethyl)pyridin-4-yl)methanamine and (E)-3-(3,4-Dichloro-phenyl)-propenal with commercially available (E)-4-(3-oxoprop-1-en-1-yl)benzonitrile the title compound is obtained: ¹H NMR (400 MHz, DMSO-d₆) δ ppm 1.52-2.44 (br. s., 5H) 3.32 (s, 4H) 3.72-4.22 (br. s., 3H) 4.47 (d, 2H) 6.51 (br. s., 1H) 7.49-7.77 (m, 3H) 7.47-7.74 (m, 3H) 7.85 (m, 2H) 8.66 (d, 1H). LC-MS (m/z): 616.0 (M+H) r.t. 2.97 min.

### Example 177

### (E)-5-(trifluoromethoxy)-1′-(3-(4-(trifluoromethyl)phenyl)allyl)-N-((2-(trifluoromethyl)pyridin-4-yl)methyl)spiro[indoline-3,4′-piperidine]-1-carboxamide

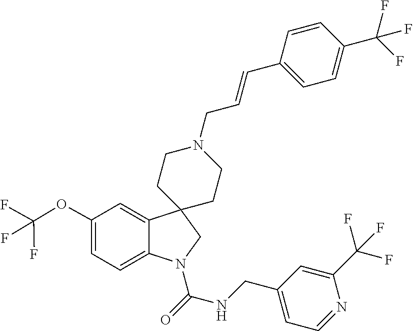 [SMILES: O=C(NCc1ccnc(C(F)(F)F)c1)N1CC2(CCN(C/C=C/c3ccc(C(F)(F)F)cc3)CC2)c2cc(OC(F)(F)F)ccc21]

Using the same procedures as Step 2 through 6 of Example 56 and replacing (3-Trifluoromethyl-phenyl)-hydrazine with commercially available (4-(trifluoromethoxy)phenyl)hydrazine and (2-Chloro-pyridin-4-yl)-methylamine hydrochloride with commercially available (2-(trifluoromethyl)pyridin-4-yl)methanamine and (E)-3-(3,4-Dichloro-phenyl)-propenal with commercially available (E)-3-(4-(trifluoromethyl)phenyl)acrylaldehyde the title compound is obtained: ¹H NMR (400 MHz, DMSO-d₆) δ ppm 1.49-2.41 (br. s., 5H) 3.32 (s, 4H) 3.83-4.17 (br. s., 3H) 4.47 (d, 2H) 6.43-6.71 (m, 1H) 7.95-7.41 (m, 3H) 7.45-7.79 (m, 3H) 7.85 (m, 2H) 8.66 (d, 1H). LC-MS (m/z): 659.1 (M+H) r.t. 2.72 min.

### Example 178

### (E)-5-(trifluoromethoxy)-1′-(3-(4-(trifluoromethoxy)phenyl)allyl)-N-((2-(trifluoromethyl)pyridin-4-yl)methyl)spiro[indoline-3,4′-piperidine]-1-carboxamide

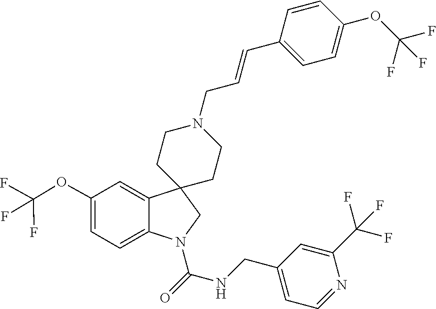 [SMILES: O=C(NCc1ccnc(C(F)(F)F)c1)N1CC2(CCN(C/C=C/c3ccc(OC(F)(F)F)cc3)CC2)c2cc(OC(F)(F)F)ccc21]

Using the same procedures as Step 2 through 6 of Example 56 and replacing (3-Trifluoromethyl-phenyl)-hydrazine with commercially available (4-(trifluoromethoxy)phenyl)hydrazine and (2-Chloro-pyridin-4-yl)-methylamine hydrochloride with commercially available (2-(trifluoromethyl)pyridin-4-yl)methanamine and (E)-3-(3,4-Dichloro-phenyl)-propenal with commercially available (E)-3-(4-(trifluoromethoxy)phenyl)acrylaldehyde the title compound is obtained: ¹H NMR (400 MHz, DMSO-d₆) δ ppm 1.50-2.37 (br. s., 5H) 3.32 (s, 4H) 3.79-4.24 (br. s., 3H) 4.47 (d, 2H) 6.30-6.59 (m, 1H) 7.37 (m, 1H) 7.42 (m, 1H) 7.51-7.74 (m, 4H) 7.85 (s, 2H) 8.66 (d, 1H). LC-MS (m/z): 675 (M+H) r.t. 3.21 min.

### Example 179

### (E)-1′-(3-(4-chlorophenyl)allyl)-5-(trifluoromethoxy)-N-((2-(trifluoromethyl)pyridin-4-yl)methyl)spiro[indoline-3,4′-piperidine]-1-carboxamide

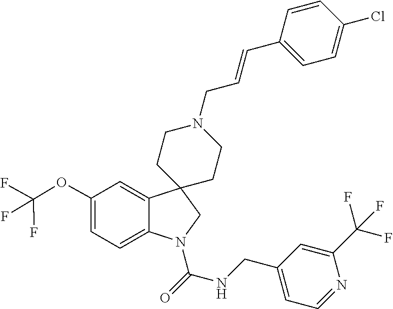 [SMILES: O=C(NCc1ccnc(C(F)(F)F)c1)N1CC2(CCN(C/C=C/c3ccc(Cl)cc3)CC2)c2cc(OC(F)(F)F)ccc21]

Using the same procedures as Step 2 through 6 of Example 56 and replacing (3-Trifluoromethyl-phenyl)-hydrazine with commercially available (4-(trifluoromethoxy)phenyl)hydrazine and (2-Chloro-pyridin-4-yl)-methylamine hydrochloride with commercially available (2-(trifluoromethyl)pyridin-4-yl)methanamine and (E)-3-(3,4-Dichloro-phenyl)-propenal with commercially available (E)-3-(4-chlorophenyl)acrylaldehyde the title compound is obtained: ¹H NMR (400 MHz, DMSO-d₆) δ ppm 1.53-2.41 (br. s., 5H) 3.32 (s, 4H) 3.71-4.22 (br. s., 3H) 4.46 (d, 2H) 6.43 (m, 1H) 7.15 (m, 2H) 7.35-7.75 (m, 4H) 7.77-7.91 (m, 2H) 8.66 (d, 1H). LC-MS (m/z): 625 (M+H) r.t. 2.69 min.

### Example 180

### (E)-N-((2-chloropyridin-4-yl)methyl)-5-(trifluoromethoxy)-1′-(3-(4-(trifluoromethoxy)phenyl)allyl)spiro[indoline-3,4′-piperidine]-1-carboxamide

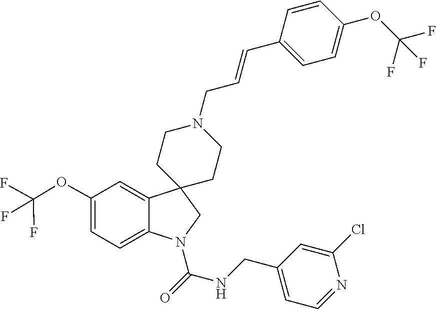 [SMILES: O=C(NCc1ccnc(Cl)c1)N1CC2(CCN(C/C=C/c3ccc(OC(F)(F)F)cc3)CC2)c2cc(OC(F)(F)F)ccc21]

Using the same procedures as Step 2 through 6 of Example 56 and replacing (3-Trifluoromethyl-phenyl)-hydrazine with commercially available (4-(trifluoromethoxy)phenyl) and (E)-3-(3,4-Dichloro-phenyl)-propenal with commercially available (E)-3-(4-(trifluoromethoxy)phenyl)acrylaldehyde the title compound is obtained: ¹H NMR (400 MHz, DMSO-d₆) δ ppm 1.57-1.74 (m, 2H) 1.80-1.96 (m, 2H) 1.98-2.18 (m, 2H) 2.85-2.97 (m, 2H) 3.08-3.19 (m, 2H) 3.93 (s, 2H) 4.32-4.46 (m, 2H) 6.08-6.27 (m, 1H) 6.44-6.56 (m, 1H) 6.85-6.94 (m, 2H) 7.06-7.13 (m, 1H) 7.19-7.24 (m, 1H) 7.35-7.43 (m, 3H) 7.44-7.48 (m, 1H) 7.50-7.62 (m, 1H) 7.82-7.98 (m, 1H) 8.27-8.42 (m, 1H) LC-MS (m/z): 587.1 (M+H) r.t. 2.95 min.

### Example 181

### (E)-N-((2-chloropyridin-4-yl)methyl)-5-(trifluoromethoxy)-1′-(3-(4-(trifluoromethyl)phenyl)allyl)spiro[indoline-3,4′-piperidine]-1-carboxamide

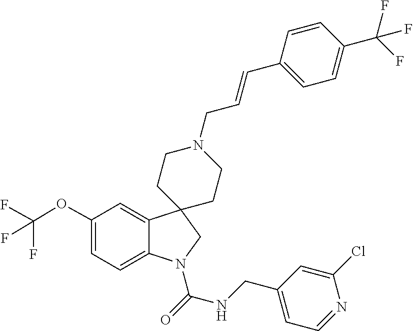 [SMILES: O=C(NCc1ccnc(Cl)c1)N1CC2(CCN(C/C=C/c3ccc(C(F)(F)F)cc3)CC2)c2cc(OC(F)(F)F)ccc21]

Using the same procedures as Step 2 through 6 of Example 56 and replacing (3-Trifluoromethyl-phenyl)-hydrazine with commercially available (4-(trifluoromethoxy)phenyl)hydrazine and (E)-3-(3,4-Dichloro-phenyl)-propenal with commercially available (E)-3-(4-(trifluoromethyl)phenyl)acrylaldehyde the title compound is obtained: ¹H NMR (400 MHz, DMSO-d₆) δ ppm 1.69 (d, 2H) 1.91 (td, 2H) 2.13 (t, 2H) 2.46-2.58 (m, 1H) 2.94 (d, 2H) 3.21 (d, 2H) 3.94 (s, 2H) 4.39 (d, 2H) 6.47-6.61 (m, 1H) 6.64-6.73 (m, 1H) 7.10 (d, 1H) 7.23 (d, 1H) 7.39 (d, 1H) 7.47 (s, 1H) 7.56 (t, 1H) 7.68 (s, 4H) 7.91 (d, 1H) 8.36 (d, 1H) LC-MS (m/z): 625.2 (M+H) r.t. 3.09 min.

### Example 182

### (E)-N-((2-chloropyridin-4-yl)methyl)-5-(trifluoromethoxy)-1′-(3-(5-(trifluoromethyl)pyridin-2-yl)allyl)spiro[indoline-3,4′-piperidine]-1-carboxamide

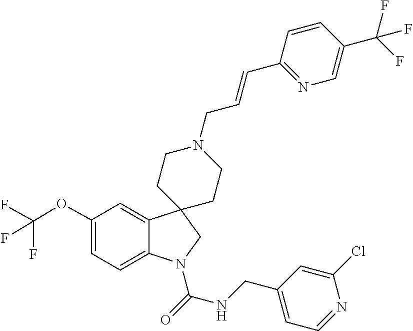 [SMILES: O=C(NCc1ccnc(Cl)c1)N1CC2(CCN(C/C=C/c3ccc(C(F)(F)F)cn3)CC2)c2cc(OC(F)(F)F)ccc21]

Using the same procedures as Step 2 through 6 of Example 2 and replacing (3-Trifluoromethyl-phenyl)-hydrazine with commercially available (4-(trifluoromethoxy)phenyl) and (E)-3-(3,4-Dichloro-phenyl)-propenal with (E)-3-(5-(trifluoromethyl)pyridin-2-yl)acrylaldehyde (Intermediate 1.17), (the product of step 2, Example 124) the title compound is obtained. ¹H NMR (400 MHz, DMSO-d₆) δ ppm 1.68 (d, 2H) 1.81-2.01 (m, 2H) 2.13 (t, 2H) 2.47-2.54 (m, 6H) 2.92 (d, 2H) 3.26 (d, 2H) 3.32 (s, 3H) 3.92 (s, 2H) 4.37 (d, 2H) 6.79 (d, 1H) 6.97 (dt, 1H) 7.10 (d, 1H) 7.19-7.29 (m, 1H) 7.37 (d, 1H) 7.45 (s, 1H) 7.54 (t, 1H) 7.64-7.76 (m, 1H) 7.88 (d, 1H) 8.16 (dd, 1H) 8.34 (d, 1H) 8.89 (s, 1H). LC-MS (m/z): 626.1 (M+H) r.t. 2.95 min.

### Example 183

### (E)-5-chloro-N-((2-methoxypyridin-4-yl)methyl)-1′-(3-(4-(trifluoromethyl)phenyl)allyl)spiro[indoline-3,4′-piperidine]-1-carboxamide

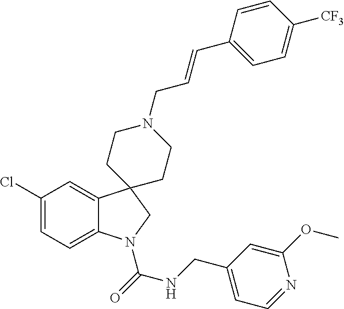 [SMILES: COc1cc(CNC(=O)N2CC3(CCN(C/C=C/c4ccc(C(F)(F)F)cc4)CC3)c3cc(Cl)ccc32)ccn1]

Using the same procedures as Step 2 through 6 of Example 56 and replacing (3-Trifluoromethyl-phenyl)-hydrazine with commercially available (4-chlorophenyl)hydrazine and (E)-3-(3,4-Dichloro-phenyl)-propenal with (E)-3-(4-(trifluoromethyl)phenyl)acrylaldehyde the title compound is obtained and (2-chloropyridin-4-yl)methanamine with (2-methoxypyridin-4-yl)methanamine, the title compound is obtained. 1H NMR (400 MHz, CDCl₃) δ: 1.79 (d, 2H), 2.07 (m, 2H), 2.18 (m, 2H), 3.08 (m, 2H), 3.31 (m, 2H), 3.81 (s, 2H), 3.96 (s, 3H), 4.51 (d, 2H), 5.05 (m, 1H), 6.40-6.49 (m, 1H), 6.62 (d, 1H), 6.73 (m, 1H), 6.87 (d, 1H), 7.14-7.19 (m, 2H), 7.51 (d, 2H), 7.60 (d, 2H), 7.84 (d, 1H), 8.15 (d, 1H), LC-MS (m/z): 571 (M+H), r.t. 2.73 min.

### Example 184

### (E)-5-chloro-N-((2-fluoropyridin-4-yl)methyl)-1′-(3-(4-(trifluoromethyl)phenyl)allyl)spiro[indoline-3,4′-piperidine]-1-carboxamide

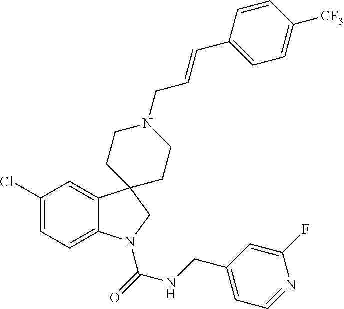 [SMILES: O=C(NCc1ccnc(F)c1)N1CC2(CCN(C/C=C/c3ccc(C(F)(F)F)cc3)CC2)c2cc(Cl)ccc21]

Using the same procedures as Step 2 through 6 of Example 56 and replacing (3-Trifluoromethyl-phenyl)-hydrazine with commercially available (4-chlorophenyl)hydrazine and (E)-3-(3,4-Dichloro-phenyl)-propenal with (E)-3-(4-(trifluoromethyl)phenyl)acrylaldehyde the title compound is obtained: 1H NMR (400 MHz, DMSO-d₆) δ: 1.65 (d, 2H), 1.92 (t, 2H), 2.10 (t, 2H), 2.92 (d, 2H), 3.19 (d, 2H), 3.90 (s, 2H), 4.40 (d, 2H), 6.49-6.56 (m, 1H), 6.68 (d, 1H), 7.12-7.15 (m, 2H), 7.26 (d, 1H), 7.32 (m, 1H), 7.52 (t, 1H), 7.68 (s, 4H), 7.82 (d, 1H), 8.17 (d, 1H), LC-MS (m/z): 559 (M+H), r.t. 2.98 min.

### Example 185

### (E)-1′-(3-(4-chlorophenyl)allyl)-N-((2-chloropyridin-4-yl)methyl)-5-(trifluoromethoxy)spiro[indoline-3,4′-piperidine]-1-carboxamide pf6763951

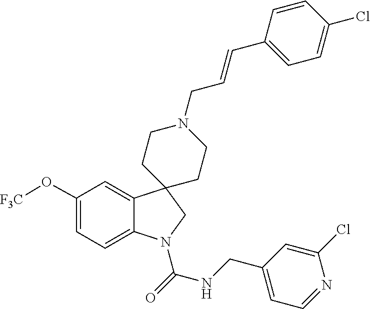 [SMILES: O=C(NCc1ccnc(Cl)c1)N1CC2(CCN(C/C=C/c3ccc(Cl)cc3)CC2)c2cc(OC(F)(F)F)ccc21]

Using the same procedures as Step 2 through 6 of Example 56 and replacing (3-Trifluoromethyl-phenyl)-hydrazine with commercially available (4-(trifluoromethoxy)phenyl)hydrazine and (E)-3-(3,4-Dichloro-phenyl)-propenal with commercially available (E)-3-(4-chlorophenyl)acrylaldehyde the title compound is obtained. LC-MS (m/z): 590.2 (M+H).

### Example 186

### (E)-1′-(3-(4-chlorophenyl)allyl)-5-(3,5-dimethylisoxazol-4-yl)-N-((2-(3,5-dimethylisoxazol-4-yl)pyridin-4-yl)methyl)spiro[indoline-3,4′-piperidine]-1-carboxamide

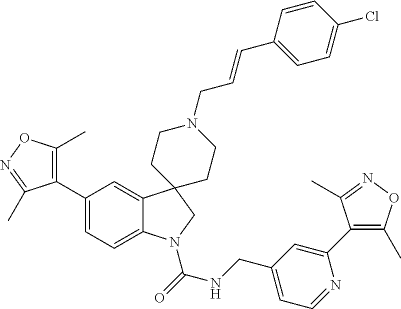 [SMILES: Cc1noc(C)c1-c1ccc2c(c1)C1(CCN(C/C=C/c3ccc(Cl)cc3)CC1)CN2C(=O)NCc1ccnc(-c2c(C)noc2C)c1]

### Step 1: 5-bromo-N-((2-chloropyridin-4-yl)methyl)spiro[indoline-3,4′-piperidine]-1-carboxamide

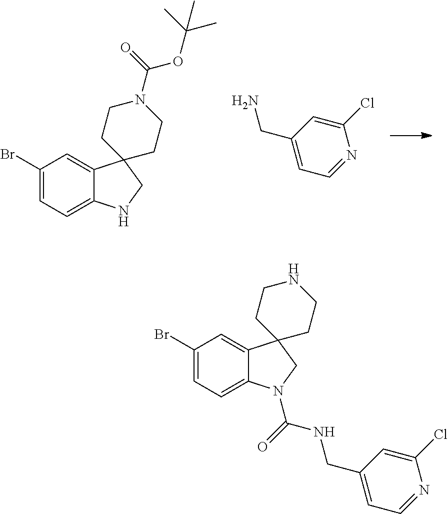 [SMILES: CC(C)(C)OC(=O)N1CCC2(CC1)CNc1ccc(Br)cc12 ; NCc1ccnc(Cl)c1 ; O=C(NCc1ccnc(Cl)c1)N1CC2(CCNCC2)c2cc(Br)ccc21]

(2-chloropyridin-4-yl)methanamine hydrochloride (487 mg, 2.72 mmol) in dichloroethane (15 mL) was treated with carbonyldiimidazole (530 mg, 3.30 mmol) and triethylamine (1.3 mL, 9.5 mmol) then heated at 50° C. for 30 minutes. Commercially available tert-butyl 5-bromospiro[indoline-3,4′-piperidine]-1′-carboxylate (1.00 g, 2.72 mmol) was added and reaction continued overnight. The reaction was cooled, washed with water, dried, filtered, concentrated and chromatographed on silica to give tert-butyl 5-bromo-1-(((2-chloropyridin-4-yl)methyl)carbamoyl)spiro[indoline-3,4′-piperidine]-1′-carboxylate. LCMS, M+H=481, Rt=3.34 minutes. The product from above in 10 mL dichloromethane was treated with 5 ml trifluoroacetic acid, stirred at ambient temperature for 1 hour then concentrated in vacuo with toluene, dissolved in DCM, washed with saturated aqueous sodium bicarbonate, dried, filtered, and concentrated to give 5-bromo-N-((2-chloropyridin-4-yl)methyl)spiro[indoline-3,4′-piperidine]-1-carboxamide, LCMS, M+H=437, Rt=2.42 min. 1H NMR (400 MHz, DMSO-d6) δ: ppm 1.73-1.93 (m, 2H) 1.93-2.12 (m, 2H) 2.88 (d, J=11.25 Hz, 2H) 3.41 (d, J=12.72 Hz, 2H), 4.01 (s, 2H) 4.38 (d, J=5.62 Hz, 2H)) 7.30-7.42 (m, 2H) 7.42-7.59 (m, 2H) 7.79 (d, J=8.56 Hz, 1H) 8.35 (d, J=5.13 Hz, 1H).

### Step 2: Preparation of Example 186

### (E)-1′-(3-(4-chlorophenyl)allyl)-5-(3,5-dimethylisoxazol-4-yl)-N-((2-(3,5-dimethylisoxazol-4-yl)pyridin-4-yl)methyl)spiro[indoline-3,4′-piperidine]-1-carboxamide

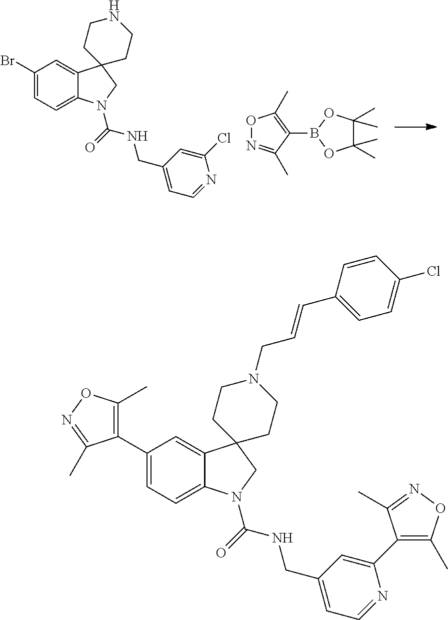

5-bromo-N-(3-chlorobenzyl)spiro[indoline-3,4′-piperidine]-1-carboxamide (100 mg, 0.23 mmol) in 4 mL dioxane and 1 mL water is treated with the 3,5-dimethyl-4-(4,4,5,5-tetramethyl-1,3,2-dioxaborolan-2-yl)isoxazole (77 mg, 0.34 mmol), potassium carbonate (108 mg, 0.69 mmol), and tetrakis(triphenylphosphine)palladium(0) (13 mg, 0.011 mmol) and heated at 120° C. for 1 hour in microwave. After cooling, the residue is diluted with ethyl acetate washed with water and brine, dried, filtered, concentrated in vacuo to give a solid, LCMS M+H=513. This solid is dissolved in 10:1 tetrahydrofuran/acetic acid (4 mL) and treated with silica impregnated sodium cyanoborohydride (500 mg of 0.88 mmol/g) and (E)-3-(4-chlorophenyl)-acrylaldehyde which is then heated in microwave at 120° C. for 15 minutes. Chromatography on silica gives the title compound. 1H NMR (400 MHz, DMSO-d6) δ: ppm 1.67 (d, J=12.47 Hz, 2H) 1.82-2.01 (m, 3H) 2.01-2.16 (m, 2H) 2.21 (s, 3H) 2.38 (d, J=3.91 Hz, 6H) 2.56 (s, 3H) 2.93 (d, J=10.76 Hz, 2H) 3.15 (d, J=6.11 Hz, 2H) 3.33 (br. s., 2H) 3.92 (s, 2H) 4.43 (d, J=5.38 Hz, 2H) 6.38 (d, J=15.89 Hz, 1H) 6.57 (d, J=16.14 Hz, 1H) 7.11 (dd, J=8.31, 1.22 Hz, 1H) 7.20 (s, 1H) 7.26-7.43 (m, 3H) 7.43-7.61 (m, 4H) 7.90 (d, J=8.31 Hz, 1H) 8.60 (d, J=4.89 Hz, 1H). LC-MS (m/z): 663 (M+H) r.t. 2.66 min. HPLC: >95%.

### Example 187

### (E)-1′-(3-(4-chlorophenyl)allyl)-N-((2-chloropyridin-4-yl)methyl)-5-(3,5-dimethylisoxazol-4-yl)spiro[indoline-3,4′-piperidine]-1-carboxamide

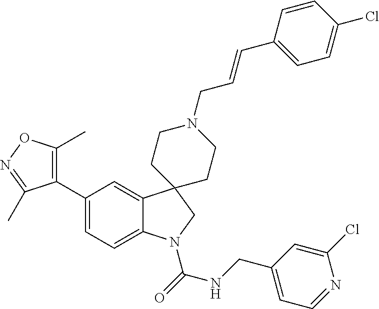 [SMILES: Cc1noc(C)c1-c1ccc2c(c1)C1(CCN(C/C=C/c3ccc(Cl)cc3)CC1)CN2C(=O)NCc1ccnc(Cl)c1]

Using the same procedures as Step 1 through 2 of Example 109 and replacing 5-bromo-2-cyanopyridine with commercially available 4-bromo-3,5-dimethylisoxazole and (2-fluoropyridin-4-yl)methanamine with (2-chloropyridin-4-yl)methanamine the title compounds is obtained. 1H NMR (400 MHz, DMSO-d6) δ: ppm 1.68 (d, J=12.23 Hz, 2H) 1.88-2.03 (m, 2H) 2.11 (t, J=11.62 Hz, 2H) 2.21 (s, 3H) 2.38 (s, 3H) 2.93 (d, J=10.76 Hz, 2H) 3.15 (d, J=5.87 Hz, 2H) 3.91 (s, 2H) 4.38 (d, J=5.38 Hz, 2H) 6.23-6.47 (m, 1H) 6.57 (d, J=15.89 Hz, 1H) 7.11 (d, J=8.31 Hz, 1H) 7.21 (s, 1H) 7.38 (d, J=7.58 Hz, 3H) 7.42-7.67 (m, 4H) 7.90 (d, J=8.07 Hz, 1H) 8.35 (d, J=5.13 Hz, 1H) LC-MS (m/z): 602 (M+H) r.t. 2.90 min, HPLC: >95%.

### Example 188

### (E)-5-chloro-1′-(3-(4-chlorophenyl)allyl)-N-((2-chloropyridin-4-yl)methyl)-N-methylspiro[indoline-3,4′-piperidine]-1-carboxamide

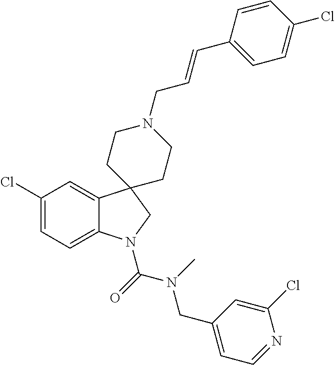 [SMILES: CN(Cc1ccnc(Cl)c1)C(=O)N1CC2(CCN(C/C=C/c3ccc(Cl)cc3)CC2)c2cc(Cl)ccc21]

Using the same procedures as Step 2 through 6 of Example 56 and replacing (3-Trifluoromethyl-phenyl)-hydrazine with commercially available (4-chlorophenyl)hydrazine and (E)-3-(3,4-Dichloro-phenyl)-propenal with 1-(2-chloropyridin-4-yl)-N-methylmethanamine the title compound is obtained. 1H NMR (400 MHz, CDCl₃) δ: 1.37-1.45 (m, 4H), 1.70-1.84 (m, 4H), 2.90 (s, 3H), 3.18 (m, 2H), 3.85 (s, 2H), 4.50 (s, 2H), 6.32 (m, 1H), 6.58 (m, 1H), 6.90 (m, 1H), 7.16 (m, 2H), 7.20 (m, 1H), 7.28-7.36 (m, 5H), 8.44 (d, 1H), LC-MS (m/z): 555.1 (M+H), r.t. 2.95 min. HPLC: 98.9%.

### Example 189

### (E)-1′-(3-(4-chlorophenyl)allyl)-N-((2-chlorothiazol-5-yl)methyl)-5-(trifluoromethoxy)spiro[indoline-3,4′-piperidine]-1-carboxamide

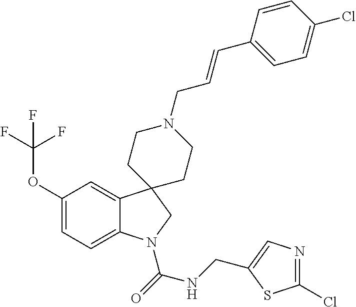 [SMILES: O=C(NCc1cnc(Cl)s1)N1CC2(CCN(C/C=C/c3ccc(Cl)cc3)CC2)c2cc(OC(F)(F)F)ccc21]

Using the same procedures as Step 2 through 6 of Example 56 and replacing (3-Trifluoromethyl-phenyl)-hydrazine with commercially available (4-(trifluoromethoxy)phenyl)hydrazine and (E)-3-(3,4-Dichloro-phenyl)-propenal with commercially available (E)-3-(4-chlorophenyl)acrylaldehyde and (2-chloropyridin-4-yl)methanamine with commercially available (2-chlorothiazol-5-yl)methanamine the title compound is obtained. 1H NMR (400 MHz, DMSO-d6) δ: 1.61 (d, J=12.44 Hz, 2H), 1.82-1.89 (m, 2H), 2.03 (t, J=11.52 Hz, 2H), 2.88 (d, J=11.56 Hz, 2H), 3.13 (d, J=6.32 Hz, 2H), 3.80 (s, 2H), 4.43 (d, J=5.44 Hz, 2H), 6.33-6.39 (m, 1H), 6.55 (d, J=16.04 Hz, 1H), 7.10-7.13 (m, 1H), 7.22 (d, J=1.92 Hz, 1H), 7.37 (d, J=8.52 Hz, 2H), 7.48 (d, J=8.56 Hz, 2H), 7.59 (s, 1H), 7.63 (t, J=5.62 Hz, 1H), 7.91 (d, J=8.80 Hz, 1H). LC-MS (m/z): 597.0 (M+H). HPLC: 99.53%.

### Biological Assays

Spiroindolines have been shown to exert their nematocidal activity through inhibition of the transport activity of vesicular acetylcholine transporters (VAChT) (Sluder et. al., “Spiroindolines identify the vesicular acetylcholine transporter as a novel target for insecticide action”. PLoS One 7(5): e34712, 2012). Biological activity (paralysis and death) in nematodes has been correlated with high affinity binding of spiroindolines to the invertebrate VAChT protein, thus confirming this mechanism of action as an approach to identifying novel parasiticides (Sluder et. al., 2012). However, while attractive as an invertebrate target of action, the VAChT protein is also expressed in mammalian species suggesting the potential for adverse effects in parasite host animals. Indeed, the structurally distinct molecule Vesamicol binds with high affinity to vertebrate VAChT and has been shown to be highly toxic to mammals (Khare et. al., “Multiple protonation states of vesicular acetylcholine transporter detected by binding of [3H]vesamicol”. Biochemistry 48: 8965-8975, 2009; and Brittain et. al., “Observations on the neuromuscular blocking action of 2-(4-phenyl-piperidino)-cyclohexanol (AH 5183)”. Br. J Pharmacol 36:173-174, 1969). Moreover, spiroindolines such as (X1, (E)-N-(2-chloropyridin-4-yl)-5-fluoro-1′-(3-(4-(trifluoromethyl)phenyl)allyl)spiro[indoline-3,4′-piperidine]-1-carboxamide) and and (X2, (E)-1′-(3-(4-chlorophenyl)allyl)-N-(2-chloropyridin-4-yl)-5-fluorospiro[indoline-3,4′-piperidine]-1-carboxamide)

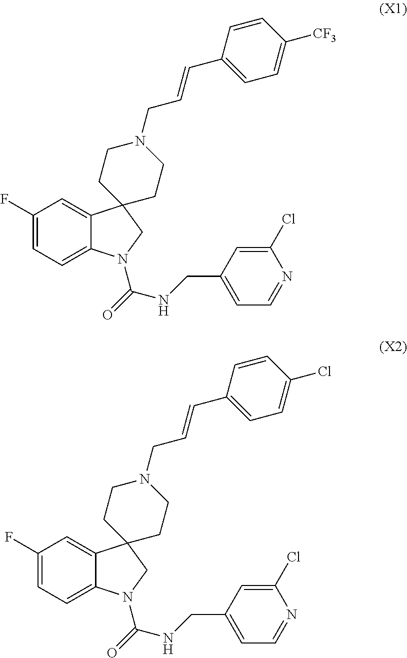 [SMILES: O=C(NCc1ccnc(Cl)c1)N1CC2(CCN(C/C=C/c3ccc(C(F)(F)F)cc3)CC2)c2cc(F)ccc21 ; O=C(NCc1ccnc(Cl)c1)N1CC2(CCN(C/C=C/c3ccc(Cl)cc3)CC2)c2cc(F)ccc21]

that exhibit high affinity (2 nM and 4.2 nM Ki's, respectively) for bovine VAChT have produced acute adverse effects when dosed by subcutaneous injection in rodents (1-3 mg/kg). In vivo, these compounds produced toxicity similar to Vesamicol and therefore, consistent with VAChT inhibition. In addition, the previously published spiroindolines, (X3, (E)-1′-(3-(4-chlorophenyl)allyl)-N-(2,6-dichloropyridin-4-yl)-5-fluorospiro[indoline-3,4′-piperidine]-1-carboxamide); X4, (E)-5-chloro-1′-(3-(4-chlorophenyl)allyl)-N-(2-chloropyridin-4-yl)spiro[indoline-3,4′-piperidine]-1-carboxamide); and X5, (E)-1′-(3-(4-chlorophenyl)allyl)-5-fluoro-N-(pyridin-3-yl)spiro[indoline-3,4′-piperidine]-1-carboxamide), Sluder et. al, example numbers 70, 74, and 76, respectively, have been shown to possess

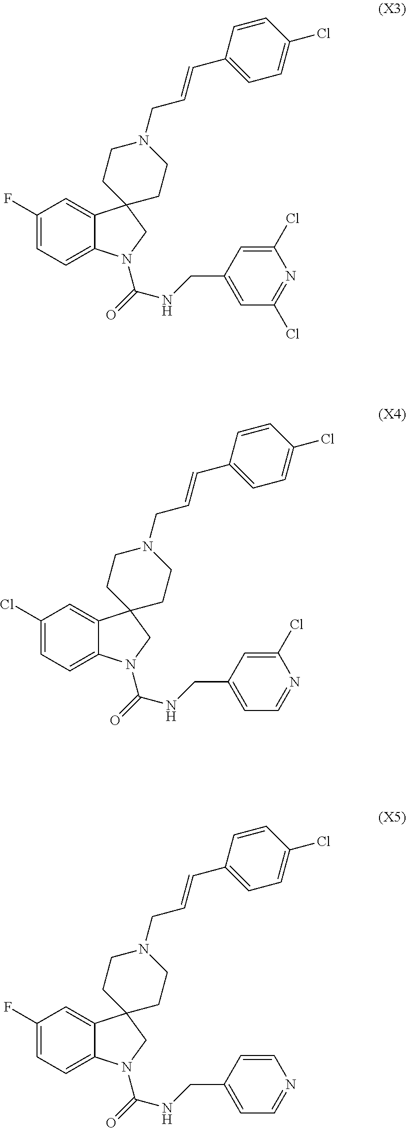 [SMILES: O=C(NCc1cc(Cl)nc(Cl)c1)N1CC2(CCN(C/C=C/c3ccc(Cl)cc3)CC2)c2cc(F)ccc21 ; O=C(NCc1ccnc(Cl)c1)N1CC2(CCN(C/C=C/c3ccc(Cl)cc3)CC2)c2cc(Cl)ccc21 ; O=C(NCc1ccncc1)N1CC2(CCN(C/C=C/c3ccc(Cl)cc3)CC2)c2cc(F)ccc21]

insecticidal activity through the VAChT mechanism of action. When assessed for bovine VAChT activity using methods described in this application, these compounds (X3, X4, and X5) bound to bovine VAChT with comparable or higher affinity (4.6 nM, 1.4 nM and 0.98 nM Ki's, respectively) to the toxic (X1) and (X2) compounds described above. Thus, because of their high affinity for the bovine VAChT protein, it is likely that compounds (X3, X4, and X5) would produce toxicity in mammals similar to (X1) and (X2).

To identify safer parasiticides, novel compounds need to maintain nematocidal activity while reducing the bovine VAChT binding affinity (i.e. higher Ki's). The compounds described herein have demonstrated nematocidal activity against either Haemonchus contortus (L3) and/or Dirofilaria immitis (microfilariae), while exhibiting reduced bovine VAChT binding affinity. These compounds have significantly reduced bovine VAChT binding affinity with Ki's ranging from 4 to 6000-fold higher than the published spiroindolines.

### Methods:

The biological activity Haemonchus contortus (L3), Dirofilaria immitis (Microfilariae) and bovine receptor binding affinities of the compounds of the invention can be measured using the test methods described below.

Haemonchus contortus, Larvae Stage 3 (HcL3) In Vitro Assay

The Haemonchus contortus L3 strain was obtained from the University of Georgia and is a relatively recent multiple-resistant field isolate (International Journal for Parasitology 37 (2007) 795-804). Compounds were dissolved in DMSO to give an initial concentration of 30 mM. The stock concentration was subsequently titrated in basal media to give an eleven point half-log concentration curve for testing. Following the serial dilution, 250 nl of each compound solution was transferred to an assay plate (384-well) whereby 25 μL of exsheathed worms (˜100 larvae/well) were subsequently added. The final compound concentrations in the 384-well plate following worm addition ranged from 0.001-100 μM. Assay plates were incubated at 37° C. and observed at 96 hours for drug effect. Endpoint data (Table 2, HcL3) are recorded as a Minimal Effective Concentration (MEC) whereby worm motility is inhibited by approximately 70%.

Dirofilaria immitis, Microfilariae (DiMF) In Vitro Assay

Compounds were initially dissolved in DMSO. The stock concentration was subsequently diluted in basal media and serially diluted to give a concentration response curve starting at 100 μM (11 total concentrations). Following the serial dilution, compound solution was transferred to an assay plate (384-well) where D.i. microfilariae (˜200/well) that have been purified from microfilaremic canine blood were subsequently added. Assay plates were observed at 72 hours for drug effect. Each compound was evaluated for decrease in microfilariae motility by subjective visual assessment and endpoint data were recorded as minimally effective dose (MEC) in μM following the incubation period.

### Radioligand Binding Assay

Compounds of the invention were measured for their ability to bind to the bovine vesicular acetylcholine transporter (VAChT). To measure binding affinity, CHO-K1 cells recombinantly expressing the bovine VAChT was generated as a stable cell line and subsequently used to produce membrane preparations. Additionally, non-selective ^{[3H]}Compound X6, (E)-N-((2-chloropyridin-4-yl)methyl)-5-fluoro-1′-(3-(4-methoxyphenyl)allyl)spiro[indoline-3,4′-piperidine]-1-carboxamide, was synthesized as a high affinity radioligand for bovine (Kd,

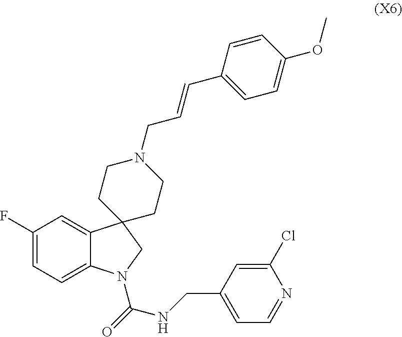 [SMILES: COc1ccc(/C=C/CN2CCC3(CC2)CN(C(=O)NCc2ccnc(Cl)c2)c2ccc(F)cc23)cc1]

0.6 nM) VAChT. Radioligand binding assays were conducted in disposable polypropylene 96-well plates. VAChT competition assays were initiated by the addition of bovine recombinant membrane homogenate (50 μg/well) in assay buffer (50 mM Tris-HCl, 120 mM NaCl, 5 mM KCl, 1 mM MgCl, and 2 mM CaCl, pH 7.4) containing [³H]Compound X6 (0.3 nM) with or without competing ligand. The assay was allowed to equilibrate for 120 minutes at room temperature, before being filtered onto GF/C unifilter plates pre-coated in 0.3% polyethylenimine (Perkin Elmer Life and Analytical Sciences, Boston, Mass.) with a Filtermate 96 harvester (PerkinElmer Life and Analytical Sciences). Filters were washed in ice-cold 50 mM Tris-HCl, pH 7.4 buffer and then counted for radioactivity in a TopCount microplate scintillation counter after addition of 50 μL Microscint 20 (PerkinElmer). Non-specific binding was measured in the presence of 10 μM Compound X6. Apparent dissociation constants (Ki values) from the competition experiments were calculated using an iterative nonlinear regression curve-fitting program (GraphPAD Prism 6.0 Software, San Diego, Calif.) and are reported in Table 2.

TABLE 2
Worm Motility Endpoint Data

| | HcL3 MEC | Di MF | Bovine binding Ki |
|---|---|---|---|
| Example | (μM) | (μM) | (nM) |
| 1 | 1.0 | ND | 70 |
| 2 | 18.2 | 10 | 321.3 |
| 3 | 3.3 | 5.7 | 31.2 |
| 4 | 10 | 10 | 78.7 |
| 5 | 33 | 10 | 70.1 |
| 6 | 3.3 | 10 | 35.4 |
| 7 | 100 | 10 | 515.6 |
| 8 | 100 | 10 | 1520.9 |
| 9 | 33 | 10 | 62.8 |
| 10 | 10 | ND | 425 |
| 11 | 100 | 10 | 178.5 |
| 12 | 2.2 | 10 | 56.3 |
| 13 | 100 | 10 | 378.7 |
| 14 | 100 | 10 | 720.7 |
| 15 | 100 | 4.4 | 610.2 |
| 16 | 100 | 3.3 | 7335.1 |
| 17 | 100 | 10 | 11600 |
| 18 | 100 | 43.5 | 2217.7 |
| 19 | ND | ND | ND |
| 20 | 100 | 13.5 | 2122 |
| 21 | 69.1 | 10 | 104.7 |
| 22 | 100 | 10 | 130.7 |
| 23 | 100.0 | 10.0 | 205.1 |
| 24 | 100.0 | 33 | 1098.3 |
| 25 | 100 | 13.5 | 7589.8 |
| 26 | 10 | 10 | 27.8 |
| 27 | 10 | 10 | 22.6 |
| 28 | 100 | 10 | 511.1 |
| 29 | 100 | 10 | 366.7 |
| 30 | ND | ND | ND |
| 31 | 100 | 3.3 | 846.2 |
| 32 | 100 | 3.3 | 135.7 |
| 33 | 100 | 10 | 1644.2 |
| 34 | 100 | 3.3 | 466.6 |
| 35 | 100 | 3.3 | 731.5 |
| 36 | 100 | 3.3 | 68.2 |
| 37 | 100 | 6.9 | 1213.3 |
| 38 | 10 | 3.3 | 45.6 |
| 39 | 6.9 | 3.3 | 90.0 |
| 40 | 4.8 | 5.7 | 110.0 |
| 41 | >100 | 33 | 428 |
| 42 | >100 | 10.0 | 55.0 |
| 43 | >100 | 10.0 | 264 |
| 44 | >100 | 10.0 | 507 |
| 45 | >100 | 33 | 3216 |
| 46 | >100 | 10 | 883 |
| 47 | 100 | 10 | 919.1 |
| 48 | >100 | 10 | 88.6 |
| 49 | >100 | 33 | 535 |
| 50 | >100 | 10 | 1042 |
| 51 | >100 | 33.3 | 518 |
| 52 | >100 | 10.0 | 778 |
| 53 | >100 | 33 | 1928 |
| 54 | >100 | 33 | 1401 |
| 55 | 3.3 | 5.7 | 876.1 |
| 56 | 4.5 | 9.1 | 432.2 |
| 57 | 70.0 | 4.0 | 953.9 |
| 58 | 100.0 | 10.0 | 1362.5 |
| 59 | 100.0 | 10.0 | 283.8 |
| 60 | 100.0 | 10.0 | 986.3 |
| 61 | 100.0 | 10.0 | 671.0 |
| 62 | 3.3 | 10.0 | 329.1 |
| 63 | 8.2 | 5.7 | 182.3 |
| 64 | 10.0 | 5.7 | 115.9 |
| 65 | 3.3 | 1.8 | 49.4 |
| 66 | 1.8 | 10.0 | 24.1 |
| 67 | 10.0 | 5.7 | 33.5 |
| 68 | 33.0 | 1.3 | 814.6 |
| 69 | 2.5 | 6.5 | 55.2 |
| 70 | 3.3 | 7.6 | 251.8 |
| 71 | 3.3 | 3.3 | 31.4 |
| 72 | 10.0 | 3.3 | 27.0 |
| 73 | 100.0 | 3.3 | 29.5 |
| 74 | 100.0 | 2.4 | 11600.0 |
| 75 | 100.0 | 4.4 | 11600.0 |
| 76 | 100.0 | 1.8 | 1417.6 |
| 77 | 5.7 | 3.3 | 870.4 |
| 78 | 100.0 | 1.0 | 1290.8 |
| 79 | 100.0 | 10.0 | 11250.9 |
| 80 | 100.0 | 10.0 | 190.8 |
| 81 | 100.0 | 7.6 | 307.9 |
| 82 | 7.6 | 4.4 | 571.8 |
| 83 | 100.0 | 6.9 | 11160.0 |
| 84 | 100.0 | 3.3 | 680.0 |
| 85 | 18.2 | 10.0 | 225.6 |
| 86 | 3.3 | 5.7 | 31.7 |
| 87 | 18.2 | 5.7 | 38.1 |
| 88 | 100.0 | 3.3 | 1125.8 |
| 89 | 100.0 | 6.9 | 774.5 |
| 90 | 1.8 | 10.0 | 255.7 |
| 91 | 100.0 | 3.3 | 294.8 |
| 92 | 5.0 | 5.0 | 795.7 |
| 93 | 100.0 | 5.7 | 958.4 |
| 94 | 100.0 | 3.3 | 3932.4 |
| 95 | 100.0 | 3.3 | 1008.3 |
| 96 | 100.0 | 5.7 | 25.4 |
| 97 | 33.0 | 5.7 | 63.7 |
| 98 | 100.0 | 10.0 | 1574.4 |
| 99 | 10.0 | 5.7 | 306.5 |
| 100 | 3.3 | 10.0 | 120.9 |
| 101 | 3.3 | 10.0 | 65.2 |
| 102 | 5.3 | 10.0 | 143.8 |
| 103 | 3.3 | 5.7 | 466.3 |
| 104 | 3.3 | 10.0 | 43.7 |
| 105 | 3.3 | 3.3 | 32.2 |
| 106 | 3.3 | 10.0 | 72.7 |
| 107 | 3.3 | 3.3 | 485.6 |
| 108 | 100.0 | 3.3 | 32553.6 |
| 109 | 3.3 | 1.8 | 50.8 |
| 110 | 69.1 | 3.3 | 7820.3 |
| 111 | 100.0 | 10.0 | 3378.9 |
| 112 | 100.0 | 4.4 | 3341.7 |
| 113 | 1.0 | 2.4 | 66.8 |
| 114 | 5.7 | 10.0 | 241.2 |
| 115 | 100.0 | 7.6 | 3932.0 |
| 116 | 5.0 | 4.0 | 58.5 |
| 117 | 10.0 | 10.0 | 191.1 |
| 118 | 3.3 | 57.4 | 28.4 |
| 119 | 100.0 | 10.0 | 176.7 |
| 120 | 100.0 | 1.2 | 13945.2 |
| 121 | 100.0 | 6.9 | 1263.1 |
| 122 | 0.5 | 10.0 | 32.3 |
| 123 | 1.0 | 10.2 | 20.8 |
| 124 | 1.0 | 10.0 | 106.4 |
| 125 | 1.0 | 10.0 | 20.4 |
| 126 | 1.0 | 10.0 | 33.0 |
| 127 | 1.0 | 10.0 | 78.4 |
| 128 | 1.0 | 3.3 | 44.0 |
| 129 | 1.5 | 10.0 | 32.8 |
| 130 | 1.8 | 18.2 | 192.7 |
| 131 | 3.3 | 10.0 | 184.5 |
| 132 | 3.3 | 3.3 | 49.9 |
| 133 | 3.3 | 7.6 | 546.0 |
| 134 | 2.0 | N.D. | 34.9 |
| 135 | 3.3 | 57.4 | 117.6 |
| 136 | 3.3 | 100.0 | 159.6 |
| 137 | 3.3 | 10.0 | 515.7 |
| 138 | 3.3 | 10.0 | 57.2 |
| 139 | 3.3 | 33.0 | 20.1 |
| 140 | 3.3 | 5.7 | 95.2 |
| 141 | 3.3 | 4.4 | 164.4 |
| 142 | 3.3 | 7.6 | 106.7 |
| 143 | 3.3 | 5.7 | 114.0 |
| 144 | 3.3 | 10.0 | 26.2 |
| 145 | 3.3 | 5.7 | 47.4 |
| 146 | 3.3 | 5.7 | 21.7 |
| 147 | 3.9 | 4.8 | 321.7 |
| 148 | 100.0 | 3.3 | 2482.6 |
| 149 | 5.1 | 8.1 | 395.9 |
| 150 | 5.7 | 18.2 | 297.0 |
| 151 | 5.7 | 4.3 | 518.0 |
| 152 | 5.7 | 4.4 | 1315.7 |
| 153 | 5.7 | 7.6 | 218.5 |
| 154 | 6.6 | 2.8 | 282.9 |
| 155 | 7.3 | 3.3 | 74.8 |
| 156 | 7.8 | 5.7 | 71.5 |
| 157 | 8.1 | 11.0 | 22.7 |
| 158 | 9.9 | 4.3 | 54.8 |
| 159 | 10.0 | 24.0 | 565.1 |
| 160 | 10.0 | 10.0 | 226.0 |
| 161 | 10.0 | 10.0 | 259.1 |
| 162 | 10.0 | 10.0 | 23.4 |
| 163 | 10.1 | 2.5 | 554.3 |
| 164 | 12.4 | 5.3 | 310.5 |
| 165 | 16.7 | 8.3 | 38.4 |
| 166 | 23.5 | 1.5 | 43.5 |
| 167 | 6.9 | 33.0 | 183.8 |
| 168 | 33.0 | 3.3 | 266.1 |
| 169 | 47.8 | 3.3 | 466.7 |
| 170 | 57.4 | 5.7 | 715.7 |
| 171 | 69.1 | 7.6 | 20.9 |
| 172 | 100.0 | 5.7 | 920.7 |
| 173 | 100.0 | 5.7 | 464.7 |
| 174 | 100.0 | 4.4 | 1497.5 |
| 175 | 24.5 | 3.3 | 451.9 |
| 176 | 33.0 | 3.3 | 288.3 |
| 177 | 100.0 | 3.3 | 1665.6 |
| 178 | 100.0 | 3.3 | 759.6 |
| 179 | 7.6 | 3.3 | 371.1 |
| 180 | 10.0 | 10.0 | 437.3 |
| 181 | 100.0 | 10.0 | 816.1 |
| 182 | 10.0 | 10.0 | 1471.4 |
| 183 | 10.0 | 3.3 | 183.6 |
| 184 | 3.3 | 3.3 | 224.1 |
| 185 | 3.3 | 10.0 | 283.2 |
| 186 | 100.0 | 3.3 | 1358.1 |
| 187 | 100.0 | 3.3 | 2466.1 |
| 188 | 2.0 | N.D. | 171.6 |
| 189 | 100 | 14 | 792 |

We claim:

1. A compound of Formula (1A), Formula (1B), and Formula (1C) 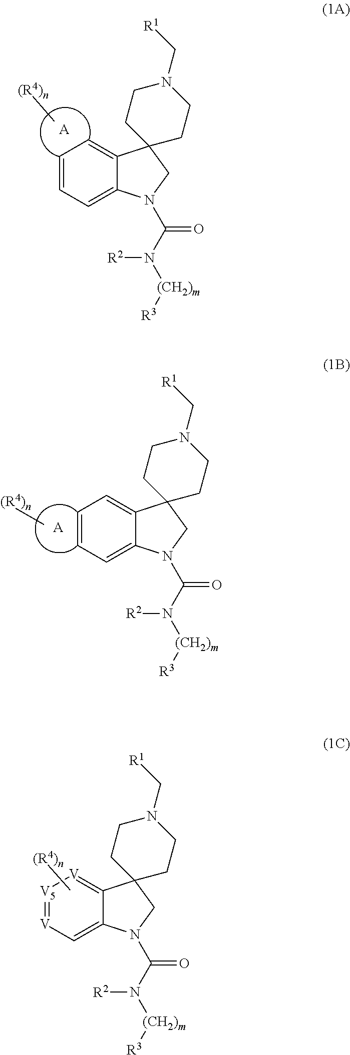
wherein
A is a 5- or 6-membered partially saturated or saturated heterocyclic ring, or a 5- to 6-membered heteroaryl ring, or a 5- to 6-membered partially saturated or saturated carbocyclic ring, wherein the heterocyclic and heteroaryl ring each contain at least 1 to 3 heteroatoms selected from N, O, or S;
v is CH or N, wherein only one of v can be N;
R¹ is selected from the group consisting of C₀-C₃alkylaryl, C₀-C₃alkylheteroaryl, C₀-C₃alkylcycloalkyl, C₀-C₃alkylheterocycle, C₂-C₄alkenylaryl, C₂-C₄alkenylheteroaryl, C₂-C₄alkenylcycloalkyl, and C₂-C₄alkenylheterocycle; wherein each cycloalkyl, aryl, heteroaryl, or heterocycle R¹ moiety is individually and optionally substituted with at least one substituent selected from the group consisting of cyano, halo, C₁-C₆haloalkyl, C₁-C₆alkyl, C₁-C₆alkoxy, and C₁-C₆haloalkoxy;
R² is selected from the group consisting of hydrogen, C₁-C₆alkyl, C₁-C₆haloalkyl, and C₁-C₆haloalkoxy;
R³ is selected from the group consisting of C₁-C₆alkyl, C₃-C₆cycloalkyl, aryl, heteroaryl, and heterocycle, wherein said R³ cycloalkyl, aryl, heteroaryl, and heterocycle moieties are each individually and optionally substituted with at least one substituent selected from the group consisting of halo, hydroxyl, —NR⁵R⁶, nitro, cyano, C₁-C₆haloalkyl, C₁-C₆alkyl, C₁-C₆alkoxy, C₁-C₆haloalkoxy, and isoxazole, wherein the isoxazole can be further substituted with at least one methyl;
R⁴ is halo, C₁-C₆alkyl, C₁-C₆haloalkyl, C₁-C₆alkoxy, C₁-C₆haloalkoxy, cyano, C₃-C₆cycloalkyl, NR⁵R⁶, S(O)₂CF₃, S(O)₂CH₃, SCF₃, SF₅, nitro, phenyl, pyridin-2(1H)-one, heterocycle, and heteroaryl, and wherein the phenyl and heteroaryl moieties can be further optionally substituted with at least one substituent selected from the group consisting of halo, cyano, C₁-C₆haloalkyl, C₁-C₆alkyl, and C₁-C₆alkoxy;
R⁵ and R⁶ are each independently selected from selected from H and C₁-C₆alkyl;
m is the integer 1, 2, 3, or 4;
n is the integer 0, 1, 2, 3, or 4, and when n is 2, 3, or 4, each R⁴ may be identical or different from each other;
stereoisomers thereof, and veterinary acceptable salts thereof, with the proviso that when n is the integer 1, then R⁴ is not fluoro or chloro at ring position 5 of Formula (1C); stereoisomers thereof, and veterinary acceptable salts thereof.

2. The compound of claim 1 wherein ring A is selected from the group consisting of 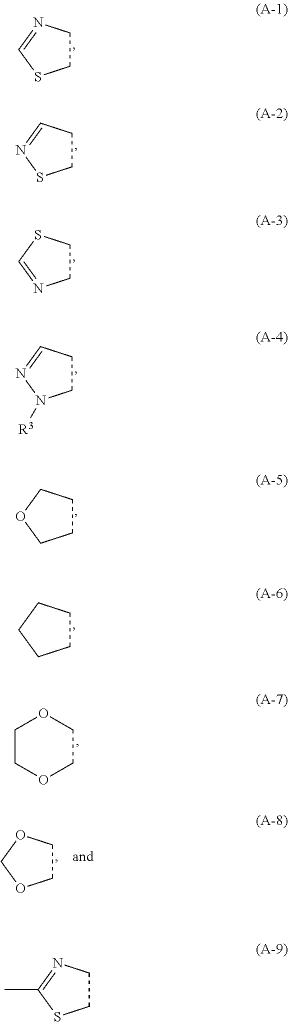
wherein the broken line (----) represents the point of attachment.

3. The compound of claim 2 that is a Formula (1A) compound, wherein
R¹ is C₂alkenylphenyl, C₂alkenylpyridinyl, or quinolinyl, each individually and optionally substituted with at least one substituent selected from the group consisting of cyano, halo, C₁-C₆haloalkyl, C₁-C₆alkyl, C₁-C₆alkoxy, and C₁-C₆haloalkoxy; and
R² is hydrogen; stereoisomers thereof, and veterinary acceptable salt thereof.

4. A compound of claim 3 selected from the group consisting of:
(E)-N-((2-chloropyridin-4-yl)methyl)-1′-(3-(3,4-dichlorophenyl)allyl)-6,8-dihydrospiro[furo[3,4-g]indole-3,4′-piperidine]-1(2H)-carboxamide;
(E)-N-((2-chloropyridin-4-yl)methyl)-1-(3-(3,4-dichlorophenyl)allyl)-2′,3′-dihydrospiro[piperidine-4,9′-[1,4]dioxino[2,3-e]indole]-7′(8′H)-carboxamide;
(E)-1-(3-(3,4-dichlorophenyl)allyl)-N-(pyridin-4-ylmethyl)spiro[piperidine-4,8′-thiazolo[4,5-e]indole]-6′(7′H)-carboxamide;
(E)-1-(3-(3,4-dichlorophenyl)allyl)-N-(pyrimidin-2-ylmethyl)spiro[piperidine-4,8′-thiazolo[4,5-e]indole]-6′(7′H)-carboxamide;
(E)-N-benzyl-1-(3-(3,4-dichlorophenyl)allyl)spiro[piperidine-4,8′-thiazolo[4,5-e]indole]-6′(7′H)-carboxamide;
(E)-1-(3-(3,4-dichlorophenyl)allyl)-N-(4-fluorobenzyl)spiro[piperidine-4,8′-thiazolo[4,5-e]indole]-6′(7′H)-carboxamide;
(E)-1-(3-(3,4-dichlorophenyl)allyl)-N-(4-nitrobenzyl)spiro[piperidine-4,8′-thiazolo[4,5-e]indole]-6′(7′H)-carboxamide;
(E)-1-(3-(3,4-dichlorophenyl)allyl)-N-(4-hydroxybenzyl)spiro[piperidine-4,8′-thiazolo[4,5-e]indole]-6′(7′H)-carboxamide;
(E)-1-(3-(3,4-dichlorophenyl)allyl)-N-(pyrazin-2-ylmethyl)spiro[piperidine-4,8′-thiazolo[4,5-e]indole]-6′(7′H)-carboxamide;
(E)-N-(4-chlorobenzyl)-1-(3-(3,4-dichlorophenyl)allyl)spiro[piperidine-4,8′-thiazolo[4,5-e]indole]-6′(7′H)-carboxamide;
(E)-N-((2-chlorothiazol-5-yl)methyl)-1-(3-(3,4-dichlorophenyl)allyl)spiro[piperidine-4,8′-thiazolo[4,5-e]indole]-6′(7′H)-carboxamide;
(E)-1-(3-(3,4-dichlorophenyl)allyl)-N-(thiazol-5-ylmethyl)spiro[piperidine-4,8′-thiazolo[4,5-e]indole]-6′(7′H)-carboxamide;
(E)-1-(3-(3,4-dichlorophenyl)allyl)-N-(thiazol-2-ylmethyl)spiro[piperidine-4,8′-thiazolo[4,5-e]indole]-6′(7′H)-carboxamide;
(E)-1-(3-(3,4-dichlorophenyl)allyl)-N-(thiazol-4-ylmethyl)spiro[piperidine-4,8′-thiazolo[4,5-e]indole]-6′(7′H)-carboxamide;
(E)-N-(cyclohexylmethyl)-1-(3-(3,4-dichlorophenyl)allyl)spiro[piperidine-4,8′-thiazolo[4,5-e]indole]-6′(7′H)-carboxamide;
(E)-1-(3-(3,4-dichlorophenyl)allyl)-N-((1-methyl-1H-pyrazol-4-yl)methyl)spiro[piperidine-4,8′-thiazolo[4,5-e]indole]-6′(7′H)-carboxamide;
(E)-1-(3-(3,4-dichlorophenyl)allyl)-N-((1-methyl-1H-pyrazol-5-yl)methyl)spiro[piperidine-4,8′-thiazolo[4,5-e]indole]-6′(7′H)-carboxamide;
(E)-1-(3-(3,4-dichlorophenyl)allyl)-N-((1-methyl-1H-pyrazol-3-yl)methyl)spiro[piperidine-4,8′-thiazolo[4,5-e]indole]-6′(7′H)-carboxamide;
(E)-1-(3-(3,4-dichlorophenyl)allyl)-N-(oxazol-4-ylmethyl)spiro[piperidine-4,8′-thiazolo[4,5-e]indole]-6′(7′H)-carboxamide;
(E)-1-(3-(3,4-dichlorophenyl)allyl)-N-(oxazol-5-ylmethyl)spiro[piperidine-4,8′-thiazolo[4,5-e]indole]-6′(7′H)-carboxamide;
(E)-1-(3-(3,4-dichlorophenyl)allyl)-N-isobutylspiro[piperidine-4,8′-thiazolo[4,5-e]indole]-6′(7′H)-carboxamide;
(E)-N-(cyclopropylmethyl)-1-(3-(3,4-dichlorophenyl)allyl)spiro[piperidine-4,8′-thiazolo[4,5-e]indole]-6′(7′H)-carboxamide;
(E)-N-(cyclopentylmethyl)-1-(3-(3,4-dichlorophenyl)allyl)spiro[piperidine-4,8′-thiazolo[4,5-e]indole]-6′(7′H)-carboxamide;
(+/−)(E)-1-(3-(3,4-dichlorophenyl)allyl)-N-((tetrahydrofuran-2-yl)methyl)spiro[piperidine-4,8′-thiazolo[4,5-e]indole]-6′(7′H)-carboxamide;
(+/−)(E)-1-(3-(3,4-dichlorophenyl)allyl)-N-((tetrahydrofuran-3-yl)methyl)spiro[piperidine-4,8′-thiazolo[4,5-e]indole]-6′(7′H)-carboxamide;
(E)-1-(3-(3,4-dichlorophenyl)allyl)-N-(pyridin-3-ylmethyl)spiro[piperidine-4,8′-thiazolo[4,5-e]indole]-6′(7′H)-carboxamide;
(E)-1-(3-(3,4-dichlorophenyl)allyl)-N-(pyridazin-4-ylmethyl)spiro[piperidine-4,8′-thiazolo[4,5-e]indole]-6′(7′H)-carboxamide;
(E)-N-((2-chloropyridin-4-yl)methyl)-1-(3-(4-fluorophenyl)allyl)spiro[piperidine-4,8′-thiazolo[4,5-e]indole]-6′(7′H)-carboxamide;
(E)-1-(3-(4-chlorophenyl)allyl)-N-((2-chloropyridin-4-yl)methyl)spiro[piperidine-4,8′-thiazolo[4,5-e]indole]-6′(7′H)-carboxamide;
(E)-N-((2-chloropyridin-4-yl)methyl)-1-(3-(3,4-dichlorophenyl)allyl)spiro[piperidine-4,8′-thiazolo[4,5-e]indole]-6′(7′H)-carboxamide;
(E)-N-((2-chloropyridin-4-yl)methyl)-1-(3-(5-(trifluoromethyl)pyridin-2-yl)allyl)spiro[piperidine-4,8′-thiazolo[4,5-e]indole]-6′(7′H)-carboxamide;
(E)-N-((2-chloropyridin-4-yl)methyl)-1-(3-(4-methoxyphenyl)allyl)spiro[piperidine-4,8′-thiazolo[4,5-e]indole]-6′(7′H)-carboxamide;
(E)-1-(3-(4-chlorophenyl)allyl)-N-((2-chloropyridin-4-yl)methyl)-2′-methylspiro[piperidine-4,8′-thiazolo[4,5-e]indole]-6′(7′H)-carboxamide;
(E)-N-((2-chloropyridin-4-yl)methyl)-1-(3-(4-fluorophenyl)allyl)-2′-methylspiro[piperidine-4,8′-thiazolo[4,5-e]indole]-6′(7′H)-carboxamide;
(E)-N-((2-chloropyridin-4-yl)methyl)-2′-methyl-1-(3-(5-(trifluoromethyl)pyridin-2-yl)allyl)spiro[piperidine-4,8′-thiazolo[4,5-e]indole]-6′(7′H)-carboxamide;
(E)-N-((2-chloropyridin-4-yl)methyl)-1-(3-(4-methoxyphenyl)allyl)-2′-methylspiro[piperidine-4,8′-thiazolo[4,5-e]indole]-6′(7′H)-carboxamide;
(E)-N-((2-bromopyridin-4-yl)methyl)-1-(3-(3,4-dichlorophenyl)allyl)spiro[piperidine-4,8′-thiazolo[4,5-e]indole]-6′(7′H)-carboxamide;
(E)-N-((2-chloropyridin-4-yl)methyl)-1′-(3-(4-fluorophenyl)allyl)spiro[isothiazolo-[4,5-e]indole-8,4′-piperidine]-6(7H)-carboxamide;
(E)-N-((2-chloropyridin-4-yl)methyl)-1′-(3-(5-(trifluoromethyl)pyridin-2-yl)allyl)spiro[isothiazolo[4,5-e]indole-8,4′-piperidine]-6(7H)-carboxamide;
N-((2-chloropyridin-4-yl)methyl)-1′-((6-fluoroquinolin-2-yl)methyl)spiro[isothiazolo[4,5-e]indole-8,4′-piperidine]-6(7H)-carboxamide;
(E)-1-(3-(4-chlorophenyl)allyl)-N-((2-chloropyridin-4-yl)methyl)-3′-methyl-3′H-spiro[piperidine-4,8′-pyrrolo[3,2-e]indazole]-6′(7′H)-carboxamide;
(E)-N-((2-chloropyridin-4-yl)methyl)-1-(3-(4-fluorophenyl)allyl)-3′-methyl-3′H-spiro[piperidine-4,8′-pyrrolo[3,2-e]indazole]-6′(7′H)-carboxamide;
(E)-N-((2-chloropyridin-4-yl)methyl)-3′-methyl-1-(3-(5-(trifluoromethyl)pyridin-2-yl)allyl)-3′H-spiro[piperidine-4,8′-pyrrolo[3,2-e]indazole]-6′(7′H)-carboxamide; and
(E)-N-((2-chloropyridin-4-yl)methyl)-1-(3-(4-methoxyphenyl)allyl)-3′-methyl-3′H-spiro[piperidine-4,8′-pyrrolo[3,2-e]indazole]-6′(7′H)-carboxamide, stereoisomers thereof, and veterinary acceptable salts thereof.

5. The compound of claim 2 that is a Formula (1B) compound, wherein
R¹ is C₂alkenylphenyl, C₂alkenylpyridinyl, or quinolinyl, each individually and optionally substituted with at least one substituent selected from the group consisting of cyano, halo, C₁-C₆haloalkyl, C₁-C₆alkyl, C₁-C₆alkoxy, and C₁₋C₆haloalkoxy; and
R² is hydrogen; stereoisomers thereof, and veterinary acceptable salt thereof.

6. A compound of claim 5 selected from the group consisting of:
(E)-N-((2-chloropyridin-4-yl)methyl)-1′-(3-(3,4-dichlorophenyl) allyl)-5,7-dihydrospiro[furo[3,4-f]indole-3,4′-piperidine]-1(2H)-carboxamide;
(E)-1′-(3-(4-chlorophenyl)allyl)-N-((2-fluoropyridin-4-yl)methyl)-5,7-dihydrospiro[furo[3,4-f]indole-3,4′-piperidine]-1(2H)-carboxamide;
(E)-1′-(3-(4-chlorophenyl)allyl)-N-((2-chlorothiazol-5-yl)methyl)-5,7-dihydrospiro[furo[3,4-f]indole-3,4′-piperidine]-1(2H)-carboxamide;
(E)-N-((2-chloropyridin-4-yl)methyl)-1′-(3-(4-fluorophenyl)allyl)-5,7-dihydrospiro[furo[3,4-f]indole-3,4′-piperidine]-1(2H)-carboxamide;
(E)-N-((2-chloropyridin-4-yl)methyl)-1′-(3-(4-cyanophenyl)allyl)-5,7-dihydrospiro[furo[3,4-f]indole-3,4′-piperidine]-1(2H)-carboxamide;
(E)-1-(3-(4-chlorophenyl)allyl)-N-((2-chloropyridin-4-yl)methyl)spiro[piperidine-4,7′-[1,3]dioxolo[4,5-f]indole]-5′(6′H)-carboxamide;
(E)-N-((2-chloropyridin-4-yl)methyl)-1-(3-(3,4-dichlorophenyl)allyl)-2′,2′-dimethylspiro[piperidine-4,7′-[1,3]dioxolo[4,5-f]indole]-5′(6′H)-carboxamide;
(E)-1-(3-(4-chlorophenyl)allyl)-N-((2-chloropyridin-4-yl)methyl)-2′,2′-dimethylspiro[piperidine-4,7′-[1,3]dioxolo[4,5-f]indole]-5′(6′H)-carboxamide;
(E)-N-((2-chloropyridin-4-yl)methyl)-1-(3-(3,4-dichlorophenyl)allyl)-2′,3′-dihydrospiro[piperidine-4,8′-[1,4]dioxino[2,3-f]indole]-6′(7′H)-carboxamide; and
(E)-N-((2-chloropyridin-4-yl)methyl)-1′-(3-(3,4-dichloro phenyl)allyl)-2,5,6,7-tetrahydro-1H-spiro[cyclopenta[f]indole-3,4′-piperidine]-1-carboxamide, stereoisomers thereof, and veterinary acceptable salts thereof.

7. The compound of claim 1 that is a Formula (1C) compound, stereoisomers thereof, and veterinary acceptable salts thereof.

8. The compound of Formula (1C) of claim 7 that is a compound of Formula (1C.a), (1C.b), (1C.c) and (1C.d), 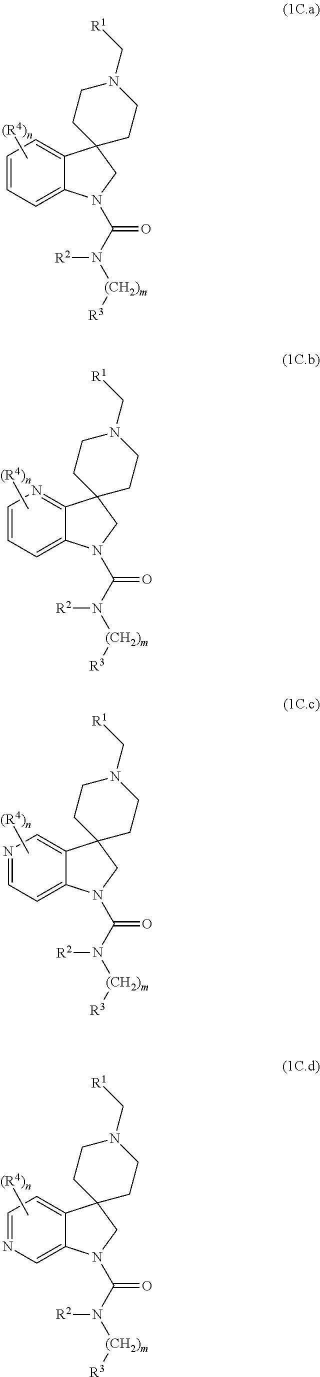
wherein
R¹ is quinolinyl, naphthyl, C₂alkenylphenyl, or C₂alkenylpyridinyl; each individually and optionally substituted with at least one substituent selected from the group consisting of cyano, halo, C₁-C₆haloalkyl, C₁-C₆alkyl, C₁-C₆alkoxy, and C₁-C₆haloalkoxy;
R² is hydrogen or methyl; and
R³ is pyridinyl or thiazolyl, each individually and optionally substituted with at least one substituent selected from the group consisting of halo, hydroxyl, —NR⁵R⁶, nitro, cyano, C₁-C₆haloalkyl, C₁-C₆alkyl, C₁-C₆alkoxy, C₁-C₆haloalkoxy, and isoxazole, wherein the isoxazole can be further substituted with at least one methyl; stereoisomers thereof, and veterinary acceptable salts thereof.

9. A compound of claim 8 that is selected from the group consisting of:
(E)-N-((2-chloropyridin-4-yl)methyl)-1′-(3-(3,4-dichlorophenyl)allyl)-5-(trifluoromethoxy)spiro[indoline-3,4′-piperidine]-1-carboxamide;
(E)-N-((2-chloropyridin-4-yl)methyl)-1′-(3-(3,4-dichlorophenyl)allyl)-4-(trifluoromethyl)spiro[indoline-3,4′-piperidine]-1-carboxamide;
(E)-7-chloro-1′-(3-(4-chlorophenyl)allyl)-N-((2-chlorothiazol-5-yl)methyl)-4-(trifluoromethoxy)spiro[indoline-3,4′-piperidine]-1-carboxamide;
(E)-7-chloro-N-((2-chloropyridin-4-yl)methyl)-1′-(3-(4-fluorophenyl)allyl)-4-(trifluoromethoxy)spiro[indoline-3,4′-piperidine]-1-carboxamide;
(E)-4,6-dichloro-1′-(3-(4-chlorophenyl)allyl)-N-((2-chloropyridin-4-yl)methyl)spiro[indoline-3,4′-piperidine]-1-carboxamide;
(E)-4,6-dichloro-1′-(3-(4-chlorophenyl)allyl)-N-((2-fluoropyridin-4-yl)methyl)spiro[indoline-3,4′-piperidine]-1-carboxamide;
(E)-4,6-dichloro-1′-(3-(4-chlorophenyl)allyl)-5-fluoro-N-((2-fluoropyridin-4-yl)methyl)spiro[indoline-3,4′-piperidine]-1-carboxamide;
(E)-4,5-dichloro-1′-(3-(4-chlorophenyl)allyl)-N-((2-fluoropyridin-4-yl)methyl)spiro[indoline-3,4′-piperidine]-1-carboxamide;
(E)-4-chloro-1′-(3-(4-chlorophenyl)allyl)-5-fluoro-N-((2-fluoropyridin-4-yl)methyl)spiro[indoline-3,4′-piperidine]-1-carboxamide;
(E)-4,5-dichloro-1′-(3-(4-chlorophenyl)allyl)-N-((2-chlorothiazol-5-yl)methyl)spiro[indoline-3,4′-piperidine]-1-carboxamide;
(E)-N-((2-chloropyridin-4-yl)methyl)-1-(3-(3,4-dichlorophenyl)allyl)-6′-methoxyspiro[piperidine-4,3′-pyrrolo[3,2-c]pyridine]-1′(2′H)-carboxamide;
(E)-N-((2-chloropyridin-4-yl)methyl)-5′-cyano-1-(3-(3,4-dichlorophenyl)allyl)-spiro[piperidine-4,3′-pyrrolo[2,3-c]pyridine]-1′(2′H)-carboxamide;
(E)-N-((2-chloropyridin-4-yl)methyl)-5′-cyano-1-(3-(3,4-dichlorophenyl)allyl)spiro[piperidine-4,3′-pyrrolo[3,2-b]pyridine]-1′(2′H)-carboxamide;
(E)-N-((2-chloropyridin-4-yl)methyl)-1-(3-(3,4-dichlorophenyl)allyl)-4′-methoxyspiro[piperidine-4,3′-pyrrolo[3,2-c]pyridine]-1′(2′H)-carboxamide;
(E)-6-chloro-1′-(3-(4-chlorophenyl)allyl)-5-fluoro-N-((2-fluoropyridin-4-yl)methyl)spiro[indoline-3,4′-piperidine]-1-carboxamide;
(E)-5,6-dichloro-1′-(3-(4-chlorophenyl)allyl)-N-((2-fluoropyridin-4-yl)methyl)spiro[indoline-3,4′-piperidine]-1-carboxamide;
(E)-5-chloro-1′-(3-(4-chlorophenyl)allyl)-6-fluoro-N-((2-fluoropyridin-4-yl)methyl)spiro[indoline-3,4′-piperidine]-1-carboxamide;
(E)-5-chloro-N-((2-chloropyridin-4-yl)methyl)-1′-(3-(4-cyanophenyl)allyl)-6-(trifluoromethyl)spiro[indoline-3,4′-piperidine]-1-carboxamide;
(E)-1′-(3-(4-chlorophenyl)allyl)-N-((2-fluoropyridin-4-yl)methyl)-6-(2-(trifluoromethoxy)phenyl)spiro[indoline-3,4′-piperidine]-1-carboxamide;
(E)-1′-(3-(4-chlorophenyl)allyl)-N-((2-chloropyridin-4-yl)methyl)-5-(2-oxopyridin-1(2H)-yl)spiro[indoline-3,4′-piperidine]-1-carboxamide;
(E)-1′-(3-(4-chlorophenyl)allyl)-N-((2-fluoropyridin-4-yl)methyl)-5-(2-oxopyridin-1(2H)-yl)spiro[indoline-3,4′-piperidine]-1-carboxamide;
(E)-1′-(3-(4-chlorophenyl)allyl)-N-((2-chloropyridin-4-yl)methyl)-5-(pyrrolidin-1-yl)spiro[indoline-3,4′-piperidine]-1-carboxamide;
(E)-1′-(3-(4-chlorophenyl)allyl)-N-((2-fluoropyridin-4-yl)methyl)-5-(trifluoromethoxy)spiro[indoline-3,4′-piperidine]-1-carboxamide;
(E)-1′-(3-(4-chlorophenyl)allyl)-N-((2-chloropyridin-4-yl)methyl)-5-(trifluoromethylsulfonyl)spiro[indoline-3,4′-piperidine]-1-carboxamide;
(E)-1′-(3-(4-chlorophenyl)allyl)-N-((2-chloropyridin-4-yl)methyl)-5-(methylsulfonyl)spiro[indoline-3,4′-piperidine]-1-carboxamide;
(E)-1′-(3-(4-chlorophenyl)allyl)-N-((2-chloropyridin-4-yl)methyl)-5-(trifluoromethylthio)spiro[indoline-3,4′-piperidine]-1-carboxamide;
(E)-1′-(3-(4-chlorophenyl)allyl)-N-((2-chloropyridin-4-yl)methyl)-5-(thiazol-2-yl)spiro[indoline-3,4′-piperidine]-1-carboxamide;
(E)-1′-(3-(4-chlorophenyl)allyl)-N-((2-fluoropyridin-4-yl)methyl)-5-(trifluoromethyl)spiro[indoline-3,4′-piperidine]-1-carboxamide;
(E)-1′-(3-(4-chlorophenyl)allyl)-N-((2-fluoropyridin-4-yl)methyl)-5-(2-oxo-1,2-dihydropyridin-4-yl)spiro[indoline-3,4′-piperidine]-1-carboxamide;
(E)-1′-(3-(4-chlorophenyl)allyl)-5-cyclopropyl-N-((2-fluoropyridin-4-yl)methyl)spiro[indoline-3,4′-piperidine]-1-carboxamide;
(E)-1′-(3-(4-chlorophenyl)allyl)-N-((2-chlorothiazol-5-yl)methyl)-5-(trifluoromethyl)spiro[indoline-3,4′-piperidine]-1-carboxamide;
(E)-1-(3-(4-chlorophenyl)allyl)-N-((2-chloropyridin-4-yl)methyl)-5′-cyanospiro[piperidine-4,3′-pyrrolo[2,3-c]pyridine]-1′(2′H)-carboxamide;
(E)-1-(3-(4-chlorophenyl)allyl)-N-((2-chloropyridin-4-yl)methyl)-5′-cyanospiro[piperidine-4,3′-pyrrolo[3,2-b]pyridine]-1′(2′H)-carboxamide;
(E)-1′-(3-(4-chlorophenyl)allyl)-N-((2-chloropyridin-4-yl)methyl)-4-(trifluoromethoxy)spiro[indoline-3,4′-piperidine]-1-carboxamide;
(E)-1′-(3-(4-chlorophenyl)allyl)-N-((2-chloropyridin-4-yl)methyl)-4-(trifluoromethylthio)spiro[indoline-3,4′-piperidine]-1-carboxamide;
(E)-1′-(3-(4-chlorophenyl)allyl)-4-fluoro-N-((2-fluoropyridin-4-yl)methyl)-6-(trifluoromethyl)spiro[indoline-3,4′-piperidine]-1-carboxamide;
(E)-1′-(3-(4-chlorophenyl)allyl)-N-((2-fluoropyridin-4-yl)methyl)-4-phenylspiro[indoline-3,4′-piperidine]-1-carboxamide;
(E)-1′-(3-(4-chlorophenyl)allyl)-N-((2-fluoropyridin-4-yl)methyl)-4-(trifluoromethyl)spiro[indoline-3,4′-piperidine]-1-carboxamide;
(E)-1′-(3-(4-chlorophenyl)allyl)-4-cyclopropyl-N-((2-fluoropyridin-4-yl)methyl)spiro[indoline-3,4′-piperidine]-1-carboxamide;
(E)-1′-(3-(4-chlorophenyl)allyl)-N-((2-fluoropyridin-4-yl)methyl)-4-(trifluoromethoxy)spiro[indoline-3,4′-piperidine]-1-carboxamide;
(E)-1′-(3-(4-chlorophenyl)allyl)-N-((2-chlorothiazol-5-yl)methyl)-4-(trifluoromethoxy)spiro[indoline-3,4′-piperidine]-1-carboxamide;
(E)-N-((2-chloropyridin-4-yl)methyl)-1′-(3-(4-cyanophenyl)allyl)-6-(2-(trifluoromethyl)phenyl)spiro[indoline-3,4′-piperidine]-1-carboxamide;
(E)-N-((2-chloropyridin-4-yl)methyl)-1′-(3-(4-cyanophenyl)allyl)-5-nitrospiro[indoline-3,4′-piperidine]-1-carboxamide;
(E)-N-((2-chloropyridin-4-yl)methyl)-1′-(3-(4-cyanophenyl)allyl)-7-fluoro-4-(trifluoromethyl)spiro[indoline-3,4′-piperidine]-1-carboxamide;
(E)-N-((2-chloropyridin-4-yl)methyl)-1′-(3-(4-cyanophenyl)allyl)-4-(trifluoromethyl)spiro-[indoline-3,4′-piperidine]-1-carboxamide;
(E)-1′-(3-(4-fluorophenyl)allyl)-N-((2-fluoropyridin-4-yl)methyl)-6-(trifluoromethoxy)-spiro[indoline-3,4′-piperidine]-1-carboxamide;
(E)-1′-(3-(4-fluorophenyl)allyl)-N-((2-fluoropyridin-4-yl)methyl)-6-(trifluoromethyl)spiro-[indoline-3,4′-piperidine]-1-carboxamide;
(E)-4-bromo-N-((2-chloropyridin-4-yl)methyl)-1′-(3-(3,4-dichlorophenyl)allyl)spiro-[indoline-3,4′-piperidine]-1-carboxamide;
4-bromo-1′-[(E)-3-(3,4-dichlorophenyl)allyl]-N-[(2-fluoro-4-pyridyl)methyl]spiro[indoline-3,4′-piperidine]-1-carboxamide;
5-bromo-1′-[(E)-3-(3,4-dichlorophenyl)allyl]-N-[(2-fluoro-4-pyridyl)methyl]spiro[indoline-3,4′-piperidine]-1-carboxamide;
5-bromo-1′-[(E)-3-(4-chlorophenyl)allyl]-N-[(2-fluoro-4-pyridyl)methyl]spiro[indoline-3,4′-piperidine]-1-carboxamide;
5-bromo-1′-[(E)-3-(4-fluorophenyl)allyl]-N-[(2-fluoro-4-pyridyl)methyl]spiro[indoline-3,4′-piperidine]-1-carboxamide;
5-bromo-N-[(2-fluoro-4-pyridyl)methyl]-1′-[(E)-3-[4-(trifluoromethyl)phenyl]allyl]-spiro[indoline-3,4′-piperidine]-1-carboxamide;
5-(3-cyanophenyl)-1′-[(E)-3-(3,4-dichlorophenyl)allyl]-N-[(2-fluoro-4-pyridyl)methyl]spiro-[indoline-3,4′-piperidine]-1-carboxamide
1′-[(E)-3-(4-chlorophenyl)allyl]-5-(6-cyano-3-pyridyl)-N-[(2-fluoro-4-pyridyl)methyl]spiro-[indoline-3,4′-piperidine]-1-carboxamide;
1′-[(E)-3-(3,4-dichlorophenyl)allyl]-N-[(2-fluoro-4-pyridyl)methyl]-5-(3-pyridyl)spiro-[indoline-3,4′-piperidine]-1-carboxamide;
1′-[(E)-3-(3,4-dichlorophenyl)allyl]-N-[(2-fluoro-4-pyridyl)methyl]-5-(1H-pyrazol-4-yl)spiro[indoline-3,4′-piperidine]-1-carboxamide;
1′-[(E)-3-(3,4-dichlorophenyl)allyl]-N-[(2-fluoro-4-pyridyl)methyl]-5-pyrimidin-5-yl-spiro[indoline-3,4′-piperidine]-1-carboxamide;
5-cyano-1′-[(E)-3-(3, 4-dichlorophenyl)allyl]-N-[(2-fluoro-4-pyridyl)methyl]spiro[indoline-3,4′-piperidine]-1-carboxamide;
1′-[(E)-3-(3, 4-dichlorophenyl)allyl]-N-[(2-fluoro-4-pyridyl)methyl]-4-methyl-spiro[indoline-3,4′-piperidine]-1-carboxamide;
1′-[(E)-3-(4-chlorophenyl)allyl]-5-(5-cyano-3-pyridyl)-N-[(2-fluoro-4-pyridyl)methyl]spiro[indoline-3,4′-piperidine]-1-carboxamide;
5-(6-cyano-3-pyridyl)-1′-[(E)-3-(3,4-dichlorophenyl)allyl]-N-[(2-fluoro-4-pyridyl)methyl]spiro[indoline-3,4′-piperidine]-1-carboxamide;
5-cyano-N-[(2-fluoro-4-pyridyl)methyl]-1′-[(2-methoxy-8-methyl-7-quinolyl)methyl]spiro[indoline-3,4′-piperidine]-1-carboxamide;
N-[(2-chloro-4-pyridyl)methyl]-5-cyano-1′-[(2-methoxy-8-methyl-7-quinolyl)methyl]spiro[indoline-3,4′-piperidine]-1-carboxamide;
1′-[(E)-3-(4-chlorophenyl)allyl]-N-[(2-chloro-4-pyridyl)methyl]-5-(6-methoxy-3-pyridyl)spiro[indoline-3,4′-piperidine]-1-carboxamide;
N-[(2-chloro-4-pyridyl)methyl]-5-(6-cyano-3-pyridyl)-1′-[(E)-3-[4-(trifluoromethyl)phenyl]allyl]spiro[indoline-3,4′-piperidine]-1-carboxamide;
(E)-7-chloro-1′-(3-(4-chlorophenyl)allyl)-N-((2-chloropyridin-4-yl)methyl)-4-(trifluoromethoxy)spiro[indoline-3,4′-piperidine]-1-carboxamide;
(E)-1′-(3-(4-chlorophenyl)allyl)-N-((2-chloropyridin-4-yl)methyl)-4-fluoro-6-(trifluoromethyl)spiro[indoline-3,4′-piperidine]-1-carboxamide;
(E)-N-((2-chloropyridin-4-yl)methyl)-1′-(3-(3,4-dichlorophenyl)allyl)-5-methylspiro[indoline-3,4′-piperidine]-1-carboxamide;
(E)-N-((2-chloropyridin-4-yl)methyl)-6-(trifluoromethyl)-1′-(3-(5-(trifluoromethyl)pyridin-2-yl)allyl)spiro[indoline-3,4′-piperidine]-1-carboxamide;
(E)-N-((2-chloropyridin-4-yl)methyl)-1′-(3-(4-methoxyphenyl)allyl)-6-(trifluoromethyl)spiro[indoline-3,4′-piperidine]-1-carboxamide;
(E)-1′-(3-(4-chlorophenyl)allyl)-N-((2-chloropyridin-4-yl)methyl)-5-fluoro-6-(trifluoromethyl)spiro[indoline-3,4′-piperidine]-1-carboxamide;
(E)-1′-(3-(4-chlorophenyl)allyl)-N-((2-fluoropyridin-4-yl)methyl)-6-(trifluoromethoxy)spiro[indoline-3,4′-pipendine]-1-carboxamide;
(E)-5-chloro-N-((2-chloropyridin-4-yl)methyl)-6-fluoro-1′-(3-(4-(trifluoromethyl)phenyl)allyl)spiro[indoline-3,4′-pipendine]-1-carboxamide;
(E)-N-((2-chloropyridin-4-yl)methyl)-1′-(3-(3,4-dichlorophenyl)allyl)-5-fluoro-6-(trifluoromethyl)spiro[indoline-3,4′-pipendine]-1-carboxamide;
(E)-N-((2-chloropyridin-4-yl)methyl)-1′-(3-(4-(trifluoromethoxy)phenyl)allyl)-6-(trifluoromethyl)spiro[indoline-3,4′-pipendine]-1-carboxamide;
(E)-4-chloro-N-((2-chloropyridin-4-yl)methyl)-1′-(3-(3,4-dichlorophenyl)allyl) spiro[indoline-3,4′-piperidine]-1-carboxamide;
(E)-N-((2-chloropyridin-4-yl)methyl)-5-cyano-1′-(3-(3,4-dichlorophenyl)allyl)-6-(trifluoromethyl)spiro[indoline-3,4′-pipendine]-1-carboxamide;
(E)-N-((2-chloropyridin-4-yl)methyl)-1′-(3-(3,4-dichlorophenyl)allyl)-5-(trifluoromethyl)spiro[indoline-3,4′-pipendine]-1-carboxamide;
(E)-5-chloro-N-((2-chloropyridin-4-yl)methyl)-1′-(3-(3,4-dichlorophenyl)allyl)-6-(trifluoromethyl)spiro[indoline-3,4′-pipendine]-1-carboxamide;
(E)-4,5-dichloro-N-((2-chloropyridin-4-yl)methyl)-1′-(3-(3,4-dichlorophenyl)allyl)spiro[indoline-3,4′-pipendine]-1-carboxamide;
(E)-N-((2-chloropyridin-4-yl)methyl)-1′-(3-(3,4-dichlorophenyl)allyl)-5-(dimethylamino)spiro[indoline-3,4′-piperidine]-1-carboxamide;
(E)-N-((2-chloropyridin-4-yl)methyl)-6-(trifluoromethyl)-1′-(3-(4-(trifluoromethyl)phenyl)allyl)spiro[indoline-3,4′-pipendine]-1-carboxamide;
(E)-4-chloro-1′-(3-(4-chlorophenyl)allyl)-N-((2-chloropyridin-4-yl)methyl)-5-fluorospiro[indoline-3,4′-pipendine]-1-carboxamide;
(E)-5,6-dichloro-N-((2-chloropyridin-4-yl)methyl)-1′-(3-(4-fluorophenyl)allyl)spiro[indoline-3,4′-pipendine]-1-carboxamide;
(E)-5,6-dichloro-1′-(3-(4-chlorophenyl)allyl)-N-((2-chloropyridin-4-yl)methyl)spiro[indoline-3,4′-pipendine]-1-carboxamide;
(E)-4,5-dichloro-N-((2-chloropyridin-4-yl)methyl)-1′-(3-(4-fluorophenyl)allyl)spiro[indoline-3,4′-pipendine]-1-carboxamide;
(E)-4,5-dichloro-1′-(3-(4-chlorophenyl)allyl)-N-((2-chloropyridin-4-yl)methyl)spiro[indoline-3,4′-pipendine]-1-carboxamide;
(E)-6-chloro-N-((2-chloropyridin-4-yl)methyl)-5-fluoro-1′-(3-(4-(trifluoromethyl)phenyl)allyl)spiro[indoline-3,4′-pipendine]-1-carboxamide;
(E)-4-chloro-N-((2-chloropyridin-4-yl)methyl)-1′-(3-(3,4-dichlorophenyl)allyl)-5-fluorospiro[indoline-3,4′-piperidine]-1-carboxamide;
(E)-1′-(3-(4-chlorophenyl)allyl)-N-((2-chloropyridin-4-yl)methyl)-5-(trifluoromethoxy)spiro[indoline-3,4′-piperidine]-1-carboxamide;
(E)-7-chloro-1′-(3-(4-chlorophenyl)allyl)-N-((2-fluoropyridin-4-yl)methyl)-4-(trifluoromethoxy)spiro[indoline-3,4′-piperidine]-1-carboxamide;
(E)-1′-(3-(4-chlorophenyl)allyl)-N-((2-chloropyridin-4-yl)methyl)-4-(trifluoromethyl)spiro[indoline-3,4′-piperidine]-1-carboxamide;
(E)-6-chloro-1′-(3-(4-chlorophenyl)allyl)-N-((2-chloropyridin-4-yl)methyl)-5-(trifluoromethoxy)spiro[indoline-3,4′-piperidine]-1-carboxamide;
(E)-N-((2-chloropyridin-4-yl)methyl)-5-(trifluoromethyl)-1′-(3-(5-(trifluoromethyl)pyridin-2-yl)allyl)spiro[indoline-3,4′-piperidine]-1-carboxamide;
(E)-N-((2-chloropyridin-4-yl)methyl)-4-(trifluoromethyl)-1′-(3-(4-(trifluoromethyl)phenyl)allyl)spiro[indoline-3,4′-piperidine]-1-carboxamide;
(E)-N-((2-chloropyridin-4-yl)methyl)-1′-(3-(4-fluorophenyl)allyl)-4-(trifluoromethyl)spiro[indoline-3,4′-piperidine]-1-carboxamide;
(E)-N-((2-chloropyridin-4-yl)methyl)-1′-(3-(4-fluorophenyl)allyl)-5-(trifluoromethyl)spiro[indoline-3,4′-piperidine]-1-carboxamide;
(E)-1′-(3-(4-chlorophenyl)allyl)-N-((2-chloropyridin-4-yl)methyl)-5-fluoro-4-methylspiro[indoline-3,4′-piperidine]-1-carboxamide;
(E)-4-bromo-1′-(3-(4-chlorophenyl)allyl)-N-((2-chloropyridin-4-yl)methyl)spiro[indoline-3,4′-piperidine]-1-carboxamide;
(E)-4-chloro-N-((2-chloropyridin-4-yl)methyl)-1′-(3-(4-cyanophenyl)allyl)-5-fluorospiro[indoline-3,4′-piperidine]-1-carboxamide;
(E)-4-chloro-N-((2-chloropyridin-4-yl)methyl)-5-fluoro-1′-(3-(4-fluorophenyl)allyl)spiro[indoline-3,4′-piperidine]-1-carboxamide;
(E)-4-chloro-1′-(3-(4-chlorophenyl)allyl)-N-((2-chloropyridin-4-yl)methyl)-5-(trifluoromethoxy)spiro[indoline-3,4′-piperidine]-1-carboxamide;
(E)-1′-(3-(4-chlorophenyl)allyl)-N-((2-chloropyridin-4-yl)methyl)-5-(trifluoromethyl)spiro[indoline-3,4′-piperidine]-1-carboxamide;
(E)-4,6-dichloro-N-((2-chloropyridin-4-yl)methyl)-1′-(3-(4-fluorophenyl)allyl)spiro[indoline-3,4′-piperidine]-1-carboxamide;
(E)-1′-(3-(4-chlorophenyl)allyl)-N-((2-chloropyridin-4-yl)methyl)-6-(methylsulfonyl)spiro[indoline-3,4′-piperidine]-1-carboxamide;
(E)-N-((2-chloropyridin-4-yl)methyl)-1′-(3-(4-fluorophenyl)allyl)-5-(trifluoromethoxy)spiro[indoline-3,4′-piperidine]-1-carboxamide;
(E)-4-chloro-N-((2-chloropyridin-4-yl)methyl)-5-fluoro-1′-(3-(4-(trifluoromethyl)phenyl)allyl)spiro[indoline-3,4′-piperidine]-1-carboxamide;
(E)-1′-(3-(4-chlorophenyl)allyl)-N-((2-chloropyridin-4-yl)methyl)-4-cyanospiro[indoline-3,4′-piperidine]-1-carboxamide;
(E)-1′-(3-(4-chlorophenyl)allyl)-N-((2-chloropyridin-4-yl)methyl)-4-cyano-5-fluorospiro[indoline-3,4′-piperidine]-1-carboxamide;
(E)-N-((2-chloropyridin-4-yl)methyl)-1′-(3-(3,4-dichlorophenyl)allyl)-5-methoxyspiro[indoline-3,4′-piperidine]-1-carboxamide;
(E)-N-((2-chloropyridin-4-yl)methyl)-1′-(3-(4-methoxyphenyl)allyl)-5-(trifluoromethyl)spiro[indoline-3,4′-piperidine]-1-carboxamide;
(E)-1′-(3-(4-chlorophenyl)allyl)-N-((2-chloropyridin-4-yl)methyl)-5-(pentafluorosulfide)spiro[indoline-3,4′-piperidine]-1-carboxamide;
(E)-N-((2-chloropyridin-4-yl)methyl)-5-(trifluoromethyl)-1′-(3-(4-(trifluoromethyl)phenyl)allyl)spiro[indoline-3,4′-piperidine]-1-carboxamide;
(E)-5,7-dichloro-N-((2-chloropyridin-4-yl)methyl)-1′-(3-(3,4-dichlorophenyl)allyl)spiro[indoline-3,4′-piperidine]-1-carboxamide;
(E)-N-((2-chloropyridin-4-yl)methyl)-1′-(3-(4-(trifluoromethoxy)phenyl)allyl)-5-(trifluoromethyl)spiro[indoline-3,4′-piperidine]-1-carboxamide;
(E)-1′-(3-(4-chlorophenyl)allyl)-N-((2-chloropyridin-4-yl)methyl)-5-fluoro-4-(trifluoromethyl)spiro[indoline-3,4′-piperidine]-1-carboxamide;
(E)-N-((2-chloropyridin-4-yl)methyl)-1′-(3-(4-fluorophenyl)allyl)-4-(trifluoromethoxy)spiro[indoline-3,4′-piperidine]-1-carboxamide;
(E)-1′-(3-(4-fluorophenyl)allyl)-5-(trifluoromethoxy)-N-((2-(trifluoromethyl)pyridin-4-yl)methyl)spiro[indoline-3,4′-piperidine]-1-carboxamide;
(E)-1′-(3-(4-cyanophenyl)allyl)-5-(trifluoromethoxy)-N-((2-(trifluoromethyl)pyridin-4-yl)methyl)spiro[indoline-3,4′-piperidine]-1-carboxamide;
(E)-5-(trifluoromethoxy)-1′-(3-(4-(trifluoromethyl)phenyl)allyl)-N-((2-(trifluoromethyl)pyridin-4-yl)methyl)spiro[indoline-3,4′-piperidine]-1-carboxamide;
(E)-5-(trifluoromethoxy)-1′-(3-(4-(trifluoromethoxy)phenyl)allyl)-N-((2-(trifluoromethyl)pyridin-4-yl)methyl)spiro[indoline-3,4′-piperidine]-1-carboxamide;
(E)-1′-(3-(4-chlorophenyl)allyl)-5-(trifluoromethoxy)-N-((2-(trifluoromethyl)pyridin-4-yl)methyl)spiro[indoline-3,4′-piperidine]-1-carboxamide;
(E)-N-((2-chloropyridin-4-yl)methyl)-5-(trifluoromethoxy)-1′-(3-(4-(trifluoromethoxy)phenyl)allyl)spiro[indoline-3,4′-piperidine]-1-carboxamide;
(E)-N-((2-chloropyridin-4-yl)methyl)-5-(trifluoromethoxy)-1′-(3-(4-(trifluoromethyl)phenyl)allyl)spiro[indoline-3,4′-piperidine]-1-carboxamide;
(E)-N-((2-chloropyridin-4-yl)methyl)-5-(trifluoromethoxy)-1′-(3-(5-(trifluoromethyl)pyridin-2-yl)allyl)spiro[indoline-3,4′-piperidine]-1-carboxamide;
(E)-1′-(3-(4-chlorophenyl)allyl)-N-((2-chloropyridin-4-yl)methyl)-5-(trifluoromethoxy)spiro[indoline-3,4′-piperidine]-1-carboxamide;
(E)-1′-(3-(4-chlorophenyl)allyl)-5-(3,5-dimethylisoxazol-4-yl)-N-((2-(3,5-dimethylisoxazol-4-yl)pyridin-4-yl)methyl)spiro[indoline-3,4′-piperidine]-1-carboxamide;
(E)-1′-(3-(4-chlorophenyl)allyl)-N-((2-chloropyridin-4-yl)methyl)-5-(3,5-dimethylisoxazol-4-yl)spiro[indoline-3,4′-piperidine]-1-carboxamide; and
(E)-1′-(3-(4-chlorophenyl)allyl)-N-((2-chlorothiazol-5-yl)methyl)-5-(trifluoromethoxy)spiro[indoline-3,4′-piperidine]-1-carboxamide, stereoisomers thereof, and veterinary acceptable salts thereof.

10. A composition comprising a compound of Formula (1A), Formula (1B) or Formula (1C) 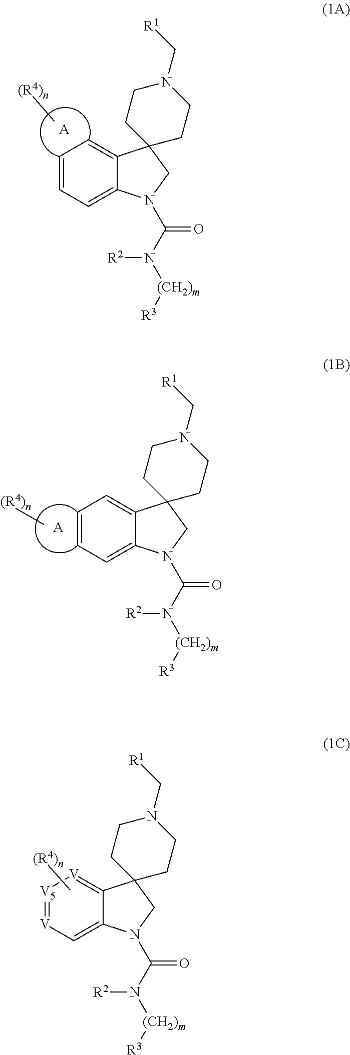
wherein
A is a 5- or 6-membered partially saturated or saturated heterocyclic ring, or a 5- to 6-membered heteroaryl ring, or a 5- to 6-membered partially saturated or saturated carbocyclic ring, wherein the heterocyclic and heteroaryl ring each contain at least 1 to 3 heteroatoms selected from N, O, or S;
v is CH or N, wherein only one of v can be N;
R¹ is selected from the group consisting of C₀-C₃alkylaryl, C₀-C₃alkylheteroaryl, C₀-C₃alkylcycloalkyl, C₀-C₃alkylheterocycle, C₂-C₄alkenylaryl, C₂-C₄alkenylheteroaryl, C₂-C₄alkenylcycloalkyl, and C₂-C₄alkenylheterocycle; wherein each cycloalkyl, aryl, heteroaryl, or heterocycle R¹ moiety is individually and optionally substituted with at least one substituent selected from the group consisting of cyano, halo, C₁-C₆haloalkyl, C₁-C₆alkyl, C₁-C₆alkoxy, and C₁-C₆haloalkoxy;
R² is selected from the group consisting of hydrogen, C₁-C₆alkyl, C₁-C₆haloalkyl, and C₁-C₆haloalkoxy;
R³ is selected from the group consisting of C₁-C₆alkyl, C₃-C₆cycloalkyl, aryl, heteroaryl, and heterocycle, wherein said R³ cycloalkyl, aryl, heteroaryl, and heterocycle moieties are each individually and optionally substituted with at least one substituent selected from the group consisting of halo, hydroxyl, —NR⁵R⁶, nitro, cyano, C₁-C₆haloalkyl, C₁-C₆alkyl, C₁-C₆alkoxy, C₁-C₆haloalkoxy, and isoxazole, wherein the isoxazole can be further substituted with at least one methyl;
R⁴ is halo, C₁-C₆alkyl, C₁-C₆haloalkyl, C₁-C₆alkoxy, C₁-C₆haloalkoxy, cyano, C₃-C₆cycloalkyl, NR⁵R⁶, S(O)₂CF₃, S(O)₂CH₃, SCF₃, SF₅, nitro, phenyl, pyridin-2(1H)-one, heterocycle, and heteroaryl, and wherein the phenyl and heteroaryl moieties can be further optionally substituted with at least one substituent selected from the group consisting of halo, cyano, C₁-C₆haloalkyl, C₁-C₆alkyl, and C₁-C₆alkoxy;
R⁵ and R⁶ are each independently selected from selected from H and C₁-C₆alkyl;
m is the integer 1, 2, 3, or 4;
n is the integer 0, 1, 2, 3, or 4 and when n is 2, 3, or 4, each R⁴ may be identical or different from each other;
stereoisomers thereof, and veterinary acceptable salts thereof, with the proviso that when n is the integer 1, then R⁴ is not fluoro or chloro at ring position 5 of Formula (1C); stereoisomers thereof, and veterinary acceptable salts thereof.

11. The composition of claim 10 further comprising at least one veterinary acceptable excipient.

12. The composition of claim 11 further comprising at least one additional veterinary agent.

13. The composition of claim 12 wherein said additional veterinary agent is selected from the group consisting of abamectin, ivermectin, avermectin, moxidectin, emamectin, eprinomectin, selamectin, doramectin, nemadectin, albendazole, cambendazole, fenbendazole, flubendazole, levamisole, mebendazole, oxfenbendazole, oxibendazole, parbendazole, tetramisole, levamisole, pyrantel, oxantel, morantel, indoxacarb, novaluron, closantel, triclabendazole, clorsulon, refoxanide, niclosamide, praziquantel, epsiprantel, 2-desoxoparaherquamide, pyripole, pyrafluprole, lufenuron, spiromesifen, tebufenozide, spinosad, spinetoram, imidacloprid, dinotefuran, metaflumizone, thibendiamide, chlorantraniliprole, indoxacarb, pyridalyl, pyrimidifen, pyrifluquinazon, milbemycin oxime, milbemycin, sarolaner, afoxolaner, fluralaner, lotilaner, demiditraz, amitraz, fipronil, methoprene, hydroprene, kinoprene, permethrin, and pyrethrin, or mixtures thereof.

14. A method of treating an animal with a parasitic infection by administering a composition comprising a compound of Formula (1A), Formula (1B), or Formula (1C) 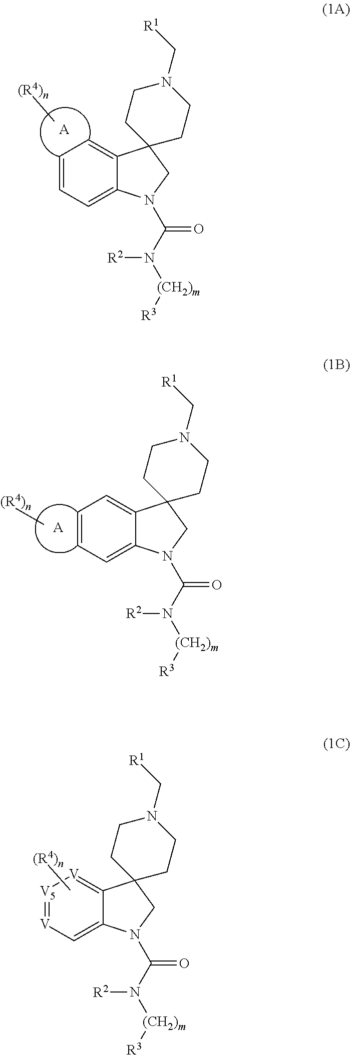
wherein
A is a 5- or 6-membered partially saturated or saturated heterocyclic ring, or a 5- to 6-membered heteroaryl ring, or a 5- to 6-membered partially saturated or saturated carbocyclic ring, wherein the heterocyclic and heteroaryl ring each contain at least 1 to 3 heteroatoms selected from N, O, or S;
v is CH or N, wherein only one of v can be N;
R¹ is selected from the group consisting of C₀-C₃alkylaryl, C₀-C₃alkylheteroaryl, C₀-C₃alkylcycloalkyl, C₀-C₃alkylheterocycle, C₂-C₄alkenylaryl, C₂-C₄alkenylheteroaryl, C₂-C₄alkenylcycloalkyl, and C₂-C₄alkenylheterocycle; wherein each cycloalkyl, aryl, heteroaryl, or heterocycle R¹ moiety is individually and optionally substituted with at least one substituent selected from the group consisting of cyano, halo, C₁-C₆haloalkyl, C₁-C₆alkyl, C₁-C₀alkoxy, and C₁-C₆haloalkoxy;
R² is selected from the group consisting of hydrogen, C₁-C₆alkyl, C₁-C₆haloalkyl, and C₁-C₆haloalkoxy;
R³ is selected from the group consisting of C₁-C₆alkyl, C₃-C₆cycloalkyl, aryl, heteroaryl, and heterocycle, wherein said R³ cycloalkyl, aryl, heteroaryl, and heterocycle moieties are each individually and optionally substituted with at least one substituent selected from the group consisting of halo, hydroxyl, —NR⁵R⁶, nitro, cyano, C₁-C₆haloalkyl, C₁-C₆alkyl, C₁-C₆alkoxy, C₁-C₆haloalkoxy, and isoxazole, wherein the isoxazole can be further substituted with at least one methyl;
R⁴ is halo, C₁-C₆alkyl, C₁-C₆haloalkyl, C₁-C₆alkoxy, C₁-C₆haloalkoxy, cyano, C₃-C₆cycloalkyl, NR⁵R⁶, S(O)₂CF₃, S(O)₂CH₃, SCF₃, SF₅, nitro, phenyl, pyridin-2(1H)-one, heterocycle, and heteroaryl, and wherein the phenyl and heteroaryl moieties can be further optionally substituted with at least one substituent selected from the group consisting of halo, cyano, C₁-C₆haloalkyl, C₁-C₆alkyl, and C₁-C₆alkoxy;
R⁵ and R⁶ are each independently selected from selected from H and C₁-C₆alkyl;
m is the integer 1, 2, 3, or 4;
n is the integer 0, 1, 2, 3, or 4 and when n is 2, 3, or 4, each R⁴ may be identical or different from each other;
stereoisomers thereof, and veterinary acceptable salts thereof, with the proviso that when n is the integer 1, then R⁴ is not fluoro or chloro at ring position 5 of Formula (1C); stereoisomers thereof, and veterinary acceptable salts thereof.

15. The method of claim 14 wherein the composition is administered to the animal orally, topically, or by injection and the animal is a companion animal or livestock.
